(19) United States
(12) Patent Application Publication
(10) Pub. No.: US 2007/0197577 A1
(43) Pub. Date: Aug. 23, 2007

(54) Inhibitors of anthrax lethal factor

(75) Inventors: Darryl Rideout, San Diego, CA (US); Tseitin Vladimir, San Diego, CA (US); Mark Shenderovich, Salt Lake City, UT (US); Edward Semple, San Diego, CA (US); Ruth F. Nutt, Santa Fe, NM (US); Yalamoori Venkatachalapathl, San Diego, CA (US); Tsai Chung-Ying, San Diego, CA (US); Michelle G. De Luna, San Diego, CA (US); Thomas P. Brady, San Diego, CA (US); Feiyue Wu, Poway, CA (US)

Correspondence Address: BUCHANAN, INGERSOLL & ROONEY LLP, P.O. BOX 1404, ALEXANDRIA, VA, 22313-1404, US

(73) Assignee: Cengent Therapeutics, San Diego, CA (US)

(21) Appl. No.: 10/550,372

(22) PCT Filed: Mar. 17, 2004
(86) PCT No.: PCT/US04/08293

§ 371 Date: Sep. 18, 2006

Related U.S. Application Data
(60) Provisional application No. 60/456,000, filed on Mar. 17, 2003.

(51) Int. Cl. A61K 31/4745; A61K 31/4436; A61K 31/403; A61K 31/405; A61K 31/40; A61K 31/445; A61K 31/19; A61K 31/277

(52) U.S. Cl. 514/291; 514/317; 514/362; 514/342; 514/419; 514/443; 514/408; 514/575; 514/411; 514/522

(57) ABSTRACT

Methods, compounds and compositions for preventing and treating anthrax infections by inhibiting Anthrax Lethal Factor (LF) activity.

### FIELD OF THE INVENTION

The present invention relates to the prophylaxis and treatment of anthrax infections and, more particularly, to compounds that act as specific inhibitors of Anthrax Lethal Factor (LF) activity, methods and means for making such inhibitors and their use as pharmaceuticals.

### BACKGROUND OF THE INVENTION

Anthrax is a zoonotic illness recognized since antiquity. In the 1870s, Robert Koch demonstrated for the first time the bacterial origin of a specific disease, with his studies on experimental anthrax, and also discovered the spore stage that allows persistence of the organism in the environment. Shortly afterward, Bacillus anthracis was recognized as the cause of woolsorter disease (inhalational anthrax). William Greenfield's successful immunization of livestock against anthrax soon followed in 1880, although Louis Pasteur's 1881 trial of a heat-cured anthrax vaccine in sheep is usually remembered as the initial use of a live vaccine.

Human cases of anthrax are invariably zoonotic in origin, with no convincing data to suggest that human-to-human transmission has ever taken place. Primary disease takes one of three forms: (1) Cutaneous, the most common, results from contact with an infected animal or animal products; (2) Inhalational is much less common and a result of spore deposition in the lungs, while (3) Gastrointestinal is due to ingestion of infected meat. Most literature cites cutaneous disease as constituting the large majority (up to 95%) of cases.

Bacillus anthracis is a large, gram-positive, sporulating rod, with square or concave ends. Growing readily on sheep blood agar, B. anthracis forms rough, gray-white colonies of four to five mm, with characteristic comma-shaped or “comet-tail” protrusions. Several tests are helpful in differentiating B. anthracis from other Bacillus species. Bacillus anthracis is characterized by an absence of the following: Hemolysis, motility, growth on phenylethyl alcohol blood agar, gelatin hydrolysis, and salicin fermentation. Bacillus anthracis may also be identified by the API-20E and API-50CHB systems used in conjunction with the previously mentioned biochemical tests. Definitive identification is based on immunological demonstration of the production of protein toxin components and the poly-D-glutamic acid capsule, susceptibility to a specific bacteriophage, and virulence for mice and guinea pigs.

The virulence of B anthracis is dependent on two toxins, lethal toxin and edema toxin, as well as on the bacterial capsule. The importance of a toxin in pathogenesis was demonstrated in the early 1950s, when sterile plasma from anthrax-infected guinea pigs caused disease when injected into other animals (Smith, H. and J. Keppie, Nature 173:869-870 (1954)). It has since been shown that the anthrax toxins are composed of three entities, which in concert lead to some of the clinical effects of anthrax (Stanley, J. L. and H. Smith, J. Gen Microbiol 26:49-66 (1961); Beall, F. A. et al., J. Bacteriol 83:1274-1280 (1962)). The first of these, protective antigen, is an 83 kd protein so named because it is the main protective constituent of anthrax vaccines. The protective antigen binds to target cell receptors and is then proteolytically cleaved of a 20 kd fragment. A second binding domain is then exposed on the 63 kd remnant, which combines with either edema factor, an 89 kd protein, to form edema toxin, or lethal factor, a 90 kd protein, to form lethal toxin (Leppla, S. H. et al., Salisbury Med Bull Suppl., 68:41-43 (1990)). The respective toxins are then transported across the cell membrane, and the factors are released into the cytosol where they exert their effects. Edema factor, a calmodulin-dependent adenylate cyclase, acts by converting adenosine triphosphate to cyclic adenosine monophosphate. Intracellular cyclic adenosine monophosphate levels are thereby increased, leading to the edema characteristic of the disease (Leppla, S. H., Proc Natl Acad Sci USA 79:3162-3166 (1982)). The action of lethal factor, believed to be a metalloprotease, is less well understood. Lethal toxin has been demonstrated to lyse macrophages at high concentration, while inducing the release of tumor necrosis factor and interleukin 1 at lower concentrations (Hanna, P. C. et al., Proc Natl Acad Sci USA 90:10198-10201 (1993); Freidlander, A. M., J. Biol Chem. 261:7123-7126 (1986)).

It has been shown that a combination of antibodies to interleukin 1 and tumor necrosis factor was protective against a lethal challenge of anthrax toxin in mice, as was the human interleukin 1 receptor antagonist (Hanna, P. C. et al., Proc Natl Acad Sci USA 90:10198-10201 (1993)). Macrophage-depleted mice were shown to resist lethal toxin challenge, but to succumb when macrophages were reconstituted. The genes for both the toxin and the capsule are carried by plasmids, designated pXO1 [33] and pX02, respectively (Green, B. D. et al., Bacillus anthracis Infect Immunol 49:291-297 (1985); Uchida, I. et al., J Gen Microbiol. 131:363-367 (1985)).

Disease occurs when spores enter the body, germinate to the bacillary form, and multiply. In cutaneous disease, spores gain entry through cuts, abrasions, or in some cases through certain species of biting flies. Germination is thought to take place in macrophages, and toxin release results in edema and tissue necrosis but little or no purulence, probably because of inhibitory effects of the toxins on leukocytes. Generally, cutaneous disease remains localized, although if untreated it may become systemic in up to 20% of cases, with dissemination via the lymphatic system. In the gastrointestinal form, B. anthracis is ingested in spore-contaminated meat, and may invade anywhere in the gastrointestinal tract. Transport to mesenteric or other regional lymph nodes and replication occur, resulting in dissemination, bacteremia, and a high mortality rate. As in other forms of anthrax, involved nodes show an impressive degree of hemorrhage and necrosis.

The pathogenesis of inhalational anthrax is more fully studied and understood. Inhaled spores are ingested by pulmonary macrophages and carried to hilar and mediastinal lymph nodes, where they germinate and multiply, elaborating toxins and overwhelming the clearance ability of the regional nodes. Bacteremia occurs, and death soon follows.

Penicillin remains the drug of choice for treatment of susceptible strains of anthrax, with ciprofloxacin and doxycycline employed as suitable alternatives. Some data in experimental models of infection suggest that the addition of streptomycin to penicillin may also be helpful. Penicillin resistance remains extremely rare in naturally occurring strains; however, the possibility of resistance should be suspected in a biological warfare attack. Cutaneous anthrax may be treated orally, while gastrointestinal or inhalational disease ordinarily should receive high doses of intravenous antibiotics (penicillin G, 4 million units every 4 hours; ciprofloxacin, 400 mg every 12 hours; or doxycycline hyclate, 100 mg every 12 hours). The more severe forms require intensive supportive care and have a high-mortality rate despite optimal therapy. The use of anti-anthrax serum, while no longer available for human use except in the former Soviet Union, was thought to be of some use in the pre-antibiotic era, although no controlled studies were performed.

Although anthrax vaccination dates to the early studies of Greenfield and Pasteur, the “modern” era of anthrax vaccine development began with a toxin-producing, unencapsulated (attenuated) strain in the 1930s. Administered to livestock as a single dose with a yearly booster, the vaccine was highly immunogenic and well tolerated in most species, although somewhat virulent in goats and llamas. This preparation is essentially the same as that administered to livestock around the world today. The first human vaccine was developed in the 1940s from nonencapsulated strains. This live spore vaccine, similar to Steme's product, is administered by scarification with a yearly booster. Studies show a reduced risk of 5-to-15-fold in occupationally exposed workers (Shlyakhov, E. N and E. Rubenstein, Vaccine 12:727-730 (1994)).

British and U.S. vaccines were developed in the 1950s and early 1960s, with the U.S. product an aluminum hydroxide-adsorbed, cell-free culture filtrate of an unencapsulated strain (V770-NP1-R), and the British vaccine an alum-precipitated, cell-free filtrate of a Sterne strain culture. The U.S. vaccine has been shown to induce high levels of antibody only to protective antigen, while the British vaccine induces lower levels of antibody to protective antigen but measurable antibodies against lethal factor and edema factor (Turnbull, P. C. B. et al., Infect Immunol 52:356-363 (1986); Turnbull, P. C. B. et al., Med Microbiol Immunol. 177:293-303 (1988)). Neither vaccine has been examined in a human clinical efficacy trial. A high number of the recipients of the vaccine have reported some type of reaction to vaccination. The preponderance of these events was minor. Manufacturer labeling for the current Michigan Department of Public Health anthrax vaccine adsorbed (AVA) product cites a 30% rate of mild local reactions and a 4% rate of moderate local reactions with a second dose. The current complex dosing schedule for the AVA vaccine consists of 0.5 mL administered subcutaneously at 0, 2, and 4 weeks, and 6, 12, and 18 months, followed by yearly boosters.

Animal studies examining the efficacy of available anthrax vaccines against aerosolized exposure have been performed. While some guinea pig studies question vaccine efficacy, primate studies have support its role. In recent work, rhesus monkeys immunized with 2 doses of the AVA vaccine were challenged with lethal doses of aerosolized B anthracis spores. All monkeys in the control group died 3 to 5 days after exposure, while the vaccinated monkeys were protected up to 2 years after immunization (Ivins, B. E. et al., Salisbury Med Bull Suppl. 87:125-126(1996)). Another trial used the AVA vaccine in a 2-dose series with a slightly different dosing interval, and again found it to be protective in all rhesus monkeys exposed to lethal aerosol challenge (Pitt, M. L. M. et al., Salisbury Med Bull Suppl. 87:130 (1996)). Thus, available evidence suggests that two doses of the current AVA vaccine should be efficacious against an aerosol exposure to anthrax spores. In addition, a highly purified, minimally reactogenic, recombinant protective antigen vaccine has been investigated, using aluminum as well as other adjuvants. Other approaches include cloning the protective antigen gene into a variety of bacteria and viruses, and the development of mutant, avirulent strains of B anthracis. One significant limitation on the use of vaccines is that existing vaccines provide no protection against a number of strains of B. anthracis.

Recent incidents, such as the suspected use of biological and chemical weapons during the Persian Gulf War, underscore the threat of biological warfare either on the battlefield or by terrorists. Anthrax has been the focus of much attention as a potential biological warfare agent for at least six decades, and modeling studies have shown the potential for use in an offensive capacity. Dispersal experiments with the simulant Bacillus globigii in the New York subway system in the 1960s suggested that release of a similar amount of B. anthracis during rush hour would result in 10,000 deaths. On a larger scale, the World Health Organization estimated that 50 kg of B anthracis released upwind of a population center of 500,000 would result in up to 95,000 fatalities, with an additional 125,000 persons incapacitated (Huxsoll, D. L. et al., JAMA 262:677-679 (1989)). Both on the battlefield and in a terrorist strike, B. anthracis has the attribute of being potentially undetectable until large numbers of seriously ill individuals present with characteristic signs and symptoms of inhalational anthrax.

Given these findings, efforts to prevent the disease or to ameliorate or treat its effects are of obvious importance. The U.S. military's current M17 and M40 gas masks provide excellent protection against the 1 to 5 μm particulates needed for a successful aerosol attack. Assuming a correct fit, these masks would be highly effective if in use at the time of exposure. Some protection might also be afforded by various forms of shelter. The pre-exposure use of the current AVA anthrax vaccine, which is approved by the U.S. Food and Drug Administration, appears to be an important adjunct. Results of primate studies also support the concept of post-exposure antibiotic prophylaxis. One study showed that 7 of 10 monkeys given penicillin, 8 of 9 given ciprofloxacin, 9 of 10 treated with doxycycline, and all 9 receiving doxycycline plus post-exposure vaccination survived a lethal aerosol challenge, with all animals receiving antibiotics for 30 days following exposure (Friedlander, A. M. et al., J. Infect Dis. 167:1239-1242 (1993). Earlier research suggested that short courses of prophylacetic antibiotics delayed but did not prevent clinical disease (Henderson, D. W. et al., J Hyg. 54:28-36 (1956). Accordingly, in the event of documented exposure, prolonged prophylacetic antibiotic use, as well as vaccination, would be mandatory. In the biological warfare setting, the differential diagnosis of inhalational anthrax would include plague and tularemia. Fluoroquinolones also have activity against these diseases, supporting the use of ciprofloxacin and perhaps other drugs of this class as either a pre-exposure or post-exposure measure.

It is therefore apparent that while certain prophylacetic and treatment schemes may prove useful in preventing or ameliorating anthrax infections, there remains a compelling need to improve the arsenal of techniques and agents available for this purpose.

### DISCLOSURE OF THE INVENTION

The present invention provides methods, compounds and compositions for inhibiting Anthrax Lethal Factor activity, and for preventing and/or treating anthrax infections. In one aspect, the invention provides a compound in accordance with the formula: 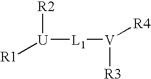
Wherein U and V are, independently, C, N, or C(CH₃), L1 is a linker and R1, R2, R3 and R4 are each independently selected substituent groups as hereinafter more fully defined.

Other aspects of the present invention provide pharmaceutical compositions comprising such compounds, and methods of synthesizing and using such compounds and compositions in prophylacetic and treatment schemes useful in preventing or ameliorating anthrax infections.

### BRIEF DESCRIPTION OF THE DRAWING

FIG. 1 is a graphic depiction of selected compounds of the present invention, together with comparative activities in inhibiting LF and MMP1.

### DETAILED DESCRIPTION OF THE INVENTION

The present invention provides methods, compounds and compositions for treating anthrax infections by inhibiting Anthrax Lethal Factor (LF) activity. The novel compositions for use herein are LF inhibitors. These substances function by binding to the LF cleavage site, and preventing the LF from catalyzing its physiological substrate. LF inhibitors are useful, either alone or together with other therapeutic compositions, in the prevention and treatment of anthrax infections. Although the term “infection” is ordinarily used in its epidemiological sense, it will readily be recognized that “infections” by Bacillus anthracis spp., or invasions by LF, can occur naturally or be purposefully induced.

Anthrax toxin, produced by Bacillus anthracis, is composed of three proteins: Protective antigen (PA), edema factor (EF) and LF. Protective antigen is an 83 kd protein that binds to specific cell surface receptors and is then proteolytically activated to a 63 kd fragment (PA63), which forms a membrane channel that mediates entry of EF or LF into the cell. PA combines with either EF, an 89 kd protein, to form edema toxin, or LF, a 90 kd protein, to form lethal toxin (Leppla, S. H. et al., Salisbury Med Bull Suppl., 68:41-43 (1990)). The respective toxins are then transported across the cell membrane, and the factors are released into the cytosol where they exert their effects. EF, a calmodulin-dependent adenylate cyclase, acts by converting adenosine triphosphate to cyclic adenosine monophosphate. Intracellular cyclic adenosine monophosphate levels are thereby increased, leading to the edema characteristic of the disease (Leppla, S. H., Proc Natl Acad Sci USA 79:3162-3166 (1982)).

The action of LF, the dominant virulence factor produced by Bacillus anthracis, and believed to be a metalloprotease, is less well understood. Lethal toxin has been demonstrated at high concentration to lyse macrophages, while inducing the release of tumor necrosis factor and interleukin 1 at lower concentrations manna, P. C. et al., Proc Natl Acad Sci USA 90:10198-10201 (1993); Freidlander, A. M., J Biol. Chem. 261:7123-7126 (1986)). LF is a 776 amino acid protein that contains a putative zinc-binding site (HEFGF) at residues 686-690, a characteristic of metalloproteases. Mutation of the H or E residues is reported to inactivate LF, and reduces its zinc-binding activity.

One useful approach to, providing agents, which will serve as inhibitors of LF activity, is to model the protein surface structure of MAP kinase kinase 1 (MAPKK1), a physiological substrate cleaved by LF. In conjunction, the consensus structural features of MAPKK1 and MAPKK2 that contain the LF cleavage site will provide a basis for designing non-peptide inhibitors of LF activity.

Thus, in one aspect, the invention provides a compound in accordance with the formula: 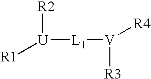
Wherein U and V are, independently, C, N, or C(CH₃), L1 is a linker and R1, R2, R3 and R4 are each independently selected substituent groups as hereinafter more fully defined:

R1 is Z(CBR5)ₙY where n 0 to 4, Z is a bond, SI CO, O, SO, SO₂, NH, NR11, SO₂NR11, NR11SO₂, 1,2-phenylene, 1,3-phenylene, 1,4-phenylene, 1,2-cyclohexylidene; Y is a group known to bind to zinc, including CONR11OH, COOH, SH, ArSH, NHCOCH₂SH, 2-hydroxybenzoate (linked at the 3,4,5, or 6-position), 2-hydroxypyridinecarboxylate (linked at the 3,4,5, or 6-position, with the ring nitrogen at any unsubstituted position), CF₂P═O(OH)₂, C(CH₃)—NOCH₂COOH, C(CH₂OH)═NOCH₂COOH, NHCO(CHR11)ₘSH (where m=1 to 4), PO(OH)₂, PO(R11)OH, SO₂NR11OH, or NH(OH)COR11.

Additional structures for Y are shown in Figure A. Y can optionally be derivatized to form a prodrug that is capable of undergoing conversion to a zinc-binding moiety after administration of the agent to a mammal. For example, SCOR11 (as a prodrug for SH), COOR11 (as a prodrug for COOH), C═OOCH₂OC═OR11 (as a prodrug for COOH), C═ONR11OC═OR11 (as a prodrug for C═ONR11OH).

R5 and R11 are, independently, H, CH₃, amino, hydroxy, alkoxy, alkylthio, alkyl (C2-C10), branched alkyl (C3-C10), alkylthio (C1-C7), alkylthioalkyl (C2-C8), arylthio, alkylamino(C1-C7), amino, arylamino, aryl, heteroaryl, arylalkyl, heterarylalkyl, arylalkenyl, heterarylalkenyl, arylalkynyl, or heterarylalkynyl.

R1 is optionally further substituted with one or more of the following: NH₂, OH, halogen, alkyl, CONH₂, CONHOH, C(NH)NH₂, C(NH)NHOH, NHC(NH₂, CN, NO₂, NR6R7 where R6 and R7 are H or alkyl and optionally form a ring. R5 can optionally form a ring with R2 or with R11.

[image]

R2 is H, isobutyl, n-butyl, pentyl, methyl, alkyl (C1-C10), branched alkyl (C3-C10), cycloalkyl, cycloalkylmethyl (C3-C9 cycle), Ar(CH₂)ₙ (where n=0 to 4, Ar is phenyl, aryl, heteroaryl), phenethyl, arylalkenyl, heterarylalkenyl, arylalkynyl, heterarylalkynyl, alkenyl (C2-C8), alkynyl (C2-C8), pentafluorophenoxyethyl, pentafluorophenylmethyl, cycloalkenyl (C4-C10), alkylthio, arylthio, alkylamino, arylamino, aryl, dichlorophenyl. R2 can optionally form a ring with R5, R11, L1, or R3. R2, R5 and R11 are optionally substituted with one or more of the following: NH₂, OH, halogen, alkyl, CF₃, CF₃O, CF₃S, alkoxy, alkylthio, SO₂alkyl (C1-C4), CONH₂, CONHOH, C(NH)NH₂, CN, NO₂, C(NH)NHOH, NHC(NH)NH₂, or NR6R7 where R6 and R7 are H or alkyl and optionally form a ring.

R1, R2 and U can optionally form a ring, including but not limited to the structures depicted in Figure B.

R11 in Figures B, C and D can be H, ethyl, methyl, isobutyl, sec-butyl, phenyl, phenethyl, benzyl, phenethyl, indolylmethyl, benzoethiophenylmethyl, hydroxyalkyl, alkyl (C1-C10), branched alkyl (C3-C10), cycloalkyl (C3-C10), aryl, 1-arylethenyl, 2-arylethenyl, heteroaryl, arylalkyl, heteroarylalkyl.

[image]

R3 is H, phenethyl, alkyl (C1-C10), branched alkyl (C1-C10), aryl, phenyl substituted with aryl or heteroaryl at the 2-, 3-, or 4-positions, benzyloxy, pyrrolyl substituted with 1-2 aryl groups, 2-aryl-1,3,4 thiadiazolyl, heteroaryl (including thiophenyl), -L2Ar where Ar includes 1-naphthyl, 2-naphthyl, 4-phenylphenyl, 5-(2-thienyl)-2-thienyl, 4-(3′-methoxyphenyl)phenyl, 4-(4′-methoxyphenyl)phenyl, 3-indolyl, phenyl, t-butyl, indolyl 3-phenylphenyl, indolyl, 2,3-dimethyl-5-indolyl, benzothiophenyl, 4-(1,2,3-thiadiazol-4-yl)phenyl, 4-(2-thienyl)phenyl, 5-(2-pyridyl)-2-thienyl, 1-(2-napthyl)vinylaminoalkyl, N-hydroxybenzamidin-4-yl, 2-methylcarbazol-3-yl, 2-ethylcarbazol-3-yl, aryl or heteroaryl and L2 is a linker chosen from the following, in both orientations: bond, CH₂, (CH₂)₂, CH₂NHCH₂, CH₂CH₂CONHCH₂, CH₂CH₂CONHCH₂CH₂, 1,1 vinylidene, 1,2-vinylidene, CO, CH₂CH₂NHCH₂, CH₂CH₂CH₂NHCH₂, CH₂NHCH₂CH₂, (CH₂)_{q} where q=3 to 7, (CHR9)ᵣ where r=1 to 7 and R9 is independently H, alkyl (C1-C10), branched alkyl (C3-C10), cycloalkyl (C3-C10), cycloalkylalkyl (C4-C14), alkyl thio, amino, alkyl amino, dialkylamino, (CHR9)sX(CHR9)ₜ where s+t=0 to 8, X is O S, CO, SO, SO₂, NH, CONH, NHCO, SO₂NH, NHSO₂ or NR9 and R9 is independently H, alkyl (C1-C10), branched alkyl (C3-C10), cycloalkyl (C3-C10), cycloalkylalkyl (C4-C14), acyl, alkyl thio, amino, alkyl amino, or dialkylamino. R9 also includes N-linked heterocycles such as piperidine, pyrroline, (1,2,3,4-)tetahydrobetacarbolin-2-yl, R15 is H, alkyl (C1-C4), branched alkyl (C3-C5), or cycloalkyl(C3-C5). Carbon-carbon single bonds in R8 can optionally be substituted with double or triple bonds. R3 can optionally form a ring with R2, L1, or R4. Such rings include, without limitation, those depicted in Figure C. R3, R9 and R15 are optionally further substituted with one or more of the following NH₂, OH, halogen, N(CH₃)₂, alkyl, CF₃, CF₃O, CF₃S, alkoxy, alkylthio, CONH₂, CONHOH, C(NH)NH₂, CN, NO₂, C(NH)NHOH, NHC(NH)NH₂, aryloxy, trifluoromethylphenyloxy, carboxyalkyl (C2-C8), (Carboxyphenyl)methylthio, carboxyalkylthio (C2-C8), carboxyphenyl, NR6R7 where R6 and R7 are H or alkyl and optionally form a ring.

[image]

R4 is H, alkyl (C1-C10), branched alkyl (C1-C10), arylalkyl, heteroarylalkyl, CONR10R16 where R10 is H, methyl, alkyl (C2-C10), branched alkyl (C3-C10), benzyl, phenethyl, arylalkyl, heteroarylalkyl, alkanoyl (C2-C8), branched alkanoyl, aroyl (C6-C12), heteroaroyl (C2-C10), isopropyl, CONR16R12; and where R12 and R16 are, independently, H, methyl, alkyl, benzyl, 2-phenylethyl, 2-indanyl, 2-morpholinylethyl, (2,6)-dimethoxylbenzyl, dimethylaminoethyl, (2-pyridyl)methyl, 2-(2-pyridyl)ethyl, 4-carboxybenzyl, 1-phenylethyl, CH(CONH₂)CH₂C6H₅, CH(CONH₂)CH₂CH(CH₃)₂, CH(CONH₂)CH(CH₃)CH₂CH₃, CH(CONH₂)CHCH₃ CH(CH₂OCH₃)CH₂C6H₅, CH(CONHCH₂CH₂OCH₃)CH₂cyclohexyl, aryl, arylalkyl, heteroaryl, heteroarylalkyl, aminoalkyl, hydroxyalkyl, (trifluoromethylphenoxy)phenyl. NR16R12 can optionally form an N-linked monocyclic or bicyclic heterocyclic ring, including but not limited to 1,2-dihydroisoindole, octahydroisoindole, morpholine, piperidine, piperazine, N-alkyl piperazine (C1-C4), homopiperazine, 3-pyrroline, pyrrolidine, tetrahydroisoquinoline, octahydropyrrolo[3,4-C]pyrrole, L-proline, L-proline dimethylamide, D-proline, D-proline dimethylamide, and thiazolidine.

R4 can optionally form a ring with L1 or R3. R4, R6, R7, R10, R11, R12 and R16 are optionally further substituted, independently, with 1 to 3 of the following substitutents: NH₂, OH, F, Cl, Br, methyl, alkyl, aryl, cycloalkyl (C3-C6), heterocycloalkyl, heteroaryl, CF₃, CF₃O, CF₃S, CF₃, aryloxy, trifluoromethylphenoxy, alkoxy, alkylthio, CONH₂, CN, NO₂, CONHOH, C(NH₂, C(NH)NHOH, NHC(NH)NH₂, NR6R7 where R6 and R7 are H or alkyl and optionally form a ring.

R3 and R4 can optionally form a ring, including but not limited to those depicted in Figure D.

[image]

L1 is a linker including the following, in either orientation: single bond, double bond, CONH, NHCO, N(CH₃)CO, CON(CH₃), CH₂NH, NHCH₂, CH═CH, C(NH)═N, N═C(NH₂), arylene (linked 1,2-; 1,3-; or 1,4), heteroarylene (linked 1,2-; 1,3-; or 1,4), ethynyl, CH═CF, CF═CH, CF═CF, CH₂CH₂, C(CH₃)═CH, CH═C(CH₃), SO₂NH, SO₂, COCH₂, CH₂CO, CNOHCH₂, CH₂CNOH, C(CF₃)═CH, CH═C(CF₃), SO₂CH₂, CH₂SO₂, SOCH₂, CH₂SO, CH₂CHOH, CHOHCH₂, lower cycloalkyl (C3-C6), or CHOHCHOH. L1 is optionally substituted with one or more of the following: NH₂, OH, halogen, alkyl, CF₃, CF₃O, CF₃S, alkoxy, alkylthio, CONH₂, CONHOH, C(NH)NH₂, C(NH)NHOH, NHC(NH)NH₂, NR6R7 where R6 and R7 are H or alkyl and optionally form a ring. L1, U and V can optionally form a cycloaliphatic (C3-C6) or heterocyclic (4 to 6 atom) ring, optionally substituted with F, OCH₃, OH, or NH₂.

For all chiral centers on the scaffold, in the linker L1, and in substituents R1 through R4, both R and S stereochemistry are contemplated. For all double bonds in the linker L1, and in substituents R1 through R4, both E and Z stereochemistry are contemplated.

The symbol “Ar” represents any aryl group. “Aryl” includes phenyl, naphthyl, phenanthrenyl, anthracenyl, biphenyl, terphenyl, phenylnaphthyl and azulenyl linked from any position. “Heteroaryl” is any monocyclic, fused bicyclic or fused tricyclic aromatic system for which at least one ring atom is O, N, or S, including thiophene, pyrrole, noxazole, furan, thiazole, imidazole, pyrazole, isoxazole, isothiazole, oxadiazole, triazole, tetrazole, thidiazole, pyridazine, pyrimidine, pyrazine, thiadiazole, triazine, indolizine, indole, benzofuran, benzothiophene, benzimidazole, benzthiazole, purine, quinoline, isoquinoline, cinnoline, phtalazine, quinazoline, naphthyridine, pteridine, carbazole, acridine, phenazine, dibenzofuran, dibenzothiophene, isomers of these, and fused aromatic ring systems (up to 3 rings) containing these, heteroaryl-aryls (up to 4 rings), aryl-heteroaryls (up to 4 rings) and heteroaryl-heteroaryls (up to 4 rings) attached from any position. Examples of heteroaryl-aryls: thienylphenyl, pyridylnaphthyl. Examples of aryl heteroaryls: biphenylthiazolyl, napthyl pyrmidinyl.

All aromatic and heteroaromatic rings can be optionally and independently further substituted with one to four of the following groups: R13, R130, R13S, R13CO, R13O —CO, R13SO, R13SO₂, R13SO₂NH, R13NHSO₂ in which R13 is H, aryl, heteroaryl, NH₂, OH, halogen, alkyl (C1-C10), methyl, fluoro, chloro, bromo, iodo, heterocycloalkyl, heterocycloalkenyl, branched alkyl (C3-C8), cycloalkyl (C3-C8), bicycloalkyl (C4-C12), cycloalkenyl (C4-C9), bicycloalkenyl (C6-C12), arylalkyl, arylalkenyl, arylalkynyl, heteroarylalkyl, heteroarylalkenyl, heteroarylalkynyl, alkenyl, alkynyl, CONH₂, CONHOH, C(H)NH₂, C(NH)NHOH, NHC(NH)NH₂, CN, NO₂, CF₃, OCF₃, SCF₃, CH₂CF₃, CH₃, perfluorinated alkyl (C1-C5), perfluorinated branched alkyl (C3-C5), perfluorinated cyclic alkyl (C3-C5), alkyl (C1-C10), alkoxy (C1-C10), alkylthio (C1-C9), arylthio, heteroarylthio, arylalkylthio, 2′-hydroxyethoxy, alkoxycarbonylmethoxy (C1-C4), dialkylamino (C1-C4 where the 2 alkyls optionally form a heteroalicyclic ring), difluoromethoxy, guanine, guanidinoalkyl (C1-C5), H₂N(NH)C(CH₂)ₕ where h=0 to 6, H₂N(NB)CNHO(CH₂)ⱼ where j=0 to 6, (2-pyridyl)amino, (2-pyridyl)aminoalkyl (C1-C6), perfluoroalkyl (C1-C4), perfluoroalkylthio (C1-C4), perfluoroalkoxy (C1-C4), 2-carboxyvinyl, alkanoyl (C1-C5), alkoxycarbonyl (C1-C4), or alkanoylamino (C1-C8). R13 may also be CONR7R7 or NR6R7 or SO₂NR6R7 or NR6COR7 or NR6SO₂R7 where R6 and R7 are, independently, H, alkyl (C1-C10), branched alkyl (C3-C8), cycloalkyl (C3-C8), aryl, arylalkyl, arylalkenyl, arylalkynyl, alkenyl, alkynyl, heteroarylalkyl, heteroarylalkenyl, heteroarylalkynyl, and where R6 and R7 optionally form a ring.

In the present disclosure, whenever a structure or substructure is depicted with 2 two or more nominally identical groups Rn or Ar {e.g. R5 in the substructure Z(CHR5)ₙY where n>1}, each Rn or Ar represents, independently, the entire range of substitutents provided for Rn or Ar unless otherwise indicated.

### Certain Preferred Embodiments

Among the numerous compounds described above as useful for inhibiting LF activity, certain compounds are considered to be preferred due to one of more beneficial properties such as increased inhibitory activity, increases solubility or bioavailability, persistence in vivo, ease of synthesis, and the like. Certain of such preferred compounds, and the compositions containing such compounds, include the following:

Compounds in the presently preferred embodiments will contain at least two ring moieties, either aromatic rings, heteroaromatic rings, or both aromatic and heteroaromatic rings. In the present embodiments U and V are, independently, C, N, or C(CH₃).

R1 is Z(CHR5)ₙY where n is 0 to 4, Z=is a bond, S, CO, O, 1,2-phenylene, 1,3-phenylene, 1,4-phenylene; Y is a group known to bind to zinc, including CONR11OH, COOH, 2-hydroxybenzoate (linked at the 3,4,5, or 6-position), 2-hydroxypyridinecarboxylate (linked at the 3,4,5, or 6-position, with the ring nitrogen at any unsubstituted position), C(CH₃)═NOCH₂COOH, or C(CH₂OH)═NOCH₂COOH. Y can optionally be derivatized to form a prodrug which is capable of undergoing conversion to a zinc-binding moiety after administration of the agent to a mammal. For example, COOR11 (as a prodrug for COOH), C═OOCH₂OC═OR11 (as a prodrug for COOH), C═ONR11OC═OR11 (as a prodrug for C═ONR110H). R5 and R11 are, independently, H, CH₃, amino, hydroxy, alkoxy, alkylthio, alkyl (C2-C10), butyl, isobutyl, methyl, branched alkyl (C3-C10), alkylthio (C1-C7), alkylthioalkyl (C2-C8), alkylamino(C1-C7), amino.

R1 is optionally further substituted with one or more of the following: NH₂, OH, halogen, alkyl, CON, CONHOH, —C(NH)NH₂, NR6R7 where R6 and R7 are H or alkyl and optionally form a ring. R5 can optionally form a ring with R2 or with R11.

R2 is H, isobutyl, n-butyl, pentyl, methyl, alkyl (C1-C10), branched alkyl (C3-C10), cycloalkyl, cycloalkylmethyl (C3-C9 cycle), Ar(CH₂)ₙ (where n is 0 to 4, Ar-phenyl, aryl, heteroaryl), phenethyl, arylalkenyl, heterarylalkenyl, arylalkynyl, heterarylalkynyl, alkenyl (C2-C8), alkynyl (C2-C8), pentafluorophenoxyethyl, pentafluorophenylmethyl, cycloalkenyl (C4-C10), alkylthio, arylthio, alkylamino, arylamino, aryl, dichlorophenyl. R2 can optionally form a ring with R5, R11, L1, or R3. R2, R5 and R11 are optionally substituted with one or more of the following: NH₂, OH, halogen, alkyl, CF₃, CF₃O, CF₃S, alkoxy, alkylthio, SO₂alkyl (C1-C4), CONH₂, CONHOH, C(NH)NH₂, CN, NO₂, C(NH)NHOH, NHC(NH)NH₂, or NR6R7 where R6 and R7 are H or alkyl and optionally form a ring.

R1, R2 and U can optionally form a ring, including the thiadiazole-containing structures in Figure B or a cycloaliphatic or heterocycloaliphatic ring. R11 in Figure B is H, ethyl, methyl, isobutyl, phenethyl, benzyl, phenethyl, hydroxyalkyl, alkyl (C1-C10), branched alkyl (C3-C10), cycloalkyl (C3-C10), aryl, 1-arylethenyl, 2-arylethenyl, heteroaryl, arylalkyl, heteroarylalkyl.

R3 is H, phenethyl, alkyl (C1-C10), branched alkyl (C1-C10), aryl, phenyl substituted with aryl or heteroaryl at the 2-, 3-, or 4-positions, benzyloxy, pyrrolyl substituted with 1-2 aryl groups, 2-aryl-1,3,4 thiadiazolyl, heteroaryl (including thiophenyl), -L2Ar where Ar includes 1-naphthyl, 2-naphthyl, 4-phenylphenyl, 5-(2-thienyl)-2-thienyl, 4-(3′-methoxyphenyl)phenyl, 4-(4′-methoxyphenyl)phenyl, 3-indolyl, phenyl, t-butyl, indolyl 3-phenylphenyl, indolyl, 2,3-dimethyl-5-indolyl, benzothiophenyl, 4-(1,2,3-thiadiazol-4-yl)phenyl, 4-(2-thienyl)phenyl, 5-(2-pyridyl)-2-thienyl, 1-(2-napthyl)vinylaminoalkyl, N-hydroxybenzamidin-4-yl, 2-methylcarbazol-3-yl, 2-ethylcarbazol-3-yl, aryl or heteroaryl and L2 is a linker chosen from the following, in both orientations: bond, CH₂, (CH₂)₂, CH₂NHCH₂, CH₂CH₂CONHCH₂, CH₂CH₂CONHCH₂CH₂, 1,1 vinylidene, 1,2-vinylidene, CO, (CHR9)ᵣ where r is 1 to 3 and R9 is independently H, alkyl (C1-C10), branched alkyl (C3-C10), cycloalkyl (C3-C10), cycloalkylalkyl (C4-C14), alkyl thio, amino, alkyl amino, dialkylamino, (CHR9)sX(CHR9)t where s+t is 0 to 8, X is O, S, CO, NH, CONH, NHCO, SO₂NH, NHSO₂ or NR9 and R9 is independently H, alkyl (C1-C10), branched alkyl (C3-C10), cycloalkyl (C3-C10), cycloalkylalkyl (C4-C14), acyl, alkyl thio, amino, alkyl amino, or dialkylamino. R9 also includes N-linked heterocycles such as piperidine, pyrroline, (1,2,3,4-)tetahydrobetacarbolin-2-yl, R15 is H, alkyl (C1-C4), branched alkyl (C3-C5), or cycloalkyl(C3-C5). Carbon-carbon single bonds in R8 can optionally be substituted with double or triple bonds. R3 can optionally form a ring with R2, L1, or R4. Such rings include, but are not limited to, those depicted in Figure C. R3, R9 and R15 are optionally further substituted with one or more of the following NH₂, OH, halogen, N(CH₃)₂, alkyl, CF₃, CF₃O, CF₃S, alkoxy, alkylthio, CONH₂, CONHOH, C(NH)NH₂, CN, NO₂, C(NH)NHOH, NHC(NH)NH₂, aryloxy, trifluoromethylphenyloxy, carboxyalkyl (C2-C8), (Carboxyphenyl)methylthio, carboxyalkylthio (C2-C8), carboxyphenyl, NR6R7 where R6 and R7 are H or alkyl and optionally form a ring.

R4 is H, alkyl (C1-C10), branched alkyl (C1-C10), arylalkyl, heteroarylalkyl, CONR10R16 where R10 is H, methyl, alkyl (C2-C10), branched alkyl (C3-C10), benzyl, phenethyl, arylalkyl, heteroarylalkyl, alkanoyl (C2-C8), branched alkanoyl, aroyl (C6-C12), heteroaroyl (C2-C10), isopropyl, CONR16R12; and where R12 and R16 are, independently, H, methyl, alkyl, benzyl, 2-phenylethyl, 2-indanyl, 2-morpholinylethyl, (2,6)-dimethoxylbenzyl, dimethylaminoethyl, (2-pyridyl)methyl, 2-(2-pyridyl)ethyl, 4-carboxybenzyl, 1-phenylethyl, CH(CONH₂)CH₂C₆H₅, CH(CONH₂)CH₂CH(CH₃)₂, CH(CONH₂)CH(CH₃)CH₂CH₃, CH(CONH₂)CHCH₃ CH(CH₂OCH₃)CH₂C6H₅, CH(CONHCH₂CH₂OCH₃)CH₂cyclohexyl, aryl, arylalkyl, heteroaryl, heteroarylalkyl, aminoalkyl, hydroxyalkyl, (trifluoromethylphenoxy)phenyl. NR16R12 can optionally form an N-linked monocyclic or bicyclic heterocyclic ring, including but not limited to 1,2-dihydroisoindole, morpholine, piperidine, piperazine, N-alkyl piperazine (C1-C4), homopiperazine, 3-pyrroline, pyrrolidine, tetrahydroisoquinoline, L-proline dimethylamide, and D-proline dimethylamide. R4 can optionally form a ring with L1 or R3. R4, R6, R7, R10, R10, R12 and R16 are optionally further substituted, independently, with 1 to 3 of the following substitutents: NH₂, OH, F, Cl, Br, methyl, alkyl, aryl, cycloalkyl (C3-C6), heterocycloalkyl, CF₃, CF₃O, CF₃S, CF₃, aryloxy, trifluoromethylphenoxy, alkoxy, alkylthio, CONH₂, CN, NO₂, CONHOH, C(NH)NH₂, C(NH)NHOH, NHC(NH)NH₂, NR6R7 where R6 and R7 are H or alkyl and optionally form a ring.

R3 and R4 can optionally form a ring, including but not limited to those depicted in Figure D.

L1 is a linker including the following, in either orientation: single bond, double bond, CONH, NHCO, N(CH₃)CO, CON(CH₃), CH₂NH, NHCH₂, CH═CH, arylene (linked 1,2-; 1,3-; or 1,4), heteroarylene (linked 1,2-; 1,3-; or 1,4), SO₂NH, SO₂, COCH₂, CH₂CO, CNOHCH₂, CH₂CNOH, SO₂CH₂, CHOHCHOH. L1, U and V can optionally form a cycloaliphatic (C3-C6) or heterocyclic (4 to 6 atom) ring, optionally substituted with F, OCH₃, OH, or NH₂.

For compounds in the most preferred embodiments, U and V are independently CH or CCH₃, L1 is CONH or CONCH₃ in either orientation, R1 is CH₂CONHOH, CH(CH₃)CONHOH, CH₂N(CHO)OH, CH(CH₃)N(CHO)OH. R2 is methyl, isobutyl, ethyl, n-propyl, n-butyl, cyclobutylmethyl, cyclopropylmethyl, 3-propenyl, 2-methyl-3-propenyl, 2-buten-1-yl, 2-butyn-1-yl, 3-propynyl, or cyclobutylmethyl substituted on the 3-position with methyl, ethyl, n-propyl, methoxy, hydroxymethyl, or aminomethyl. R3 is L2Ar where L2 is bond, or CH₂, (CH₂)₂, CO, or 1,1-vinylidene, and Ar is a group containing 2-3 aromatic/heteroaromatic rings (fused or directly linked). Ar can be naphthyl, benzothiophenyl, indolyl, quinolinyl, isoquinolinyl or carbazolyl linked from any free position, or a biaryl consisting of phenyl, thienyl, or pyridyl linked from any position and substituted on the 3 or 4 position with phenyl, pyridyl, thienyl, 3-substituted phenyl. The aromatic rings can be optionally further substituted with methoxy, methyl, fluoro, ethyl, hydroxyl, hydroxymethyl, aminomethyl, 2-aminoethyl, 3-aminopropyl, 2-dimethylaminoethyl, or 3-dimethylaminopropyl. R4 is CONR16R12 or CH₂CONR16R12, with R12 is independently, H, benzyl, 2-phenylethyl, 2-indanyl, 2-morpholinylethyl, (2-pyridyl)methyl, 2-(2-pyridyl)ethyl, 1-phenylethyl, CH(CONH₂)CH₂C₆H₅, CH(CONHOCH₂CH(CH₃)₂, CH(CONH₂)CH(CH₃)CH₂CH₃, and R16 is H, methyl, ethyl, or 2-aminoethyl. NR16R12 can optionally form an N-linked monocyclic or bicyclic heterocyclic ring, including but not limited to 1,2-dihydroisoindole, morpholine, piperidine, piperazine, N-alkyl piperazine (C1-C4), 3-pyrroline, pyrrolidine, or tetrahydroisoquinoline. R4, R12 and R16 are optionally further substituted, independently, with 1 to 3 of the following substitutents: OH, F, Cl, Br, methyl, CF₃, CF₃₀, methoxy, alkylthio, CONH₂, C(NH)NH₂, NHC(NH)NH₂, NR6R7 where R6 and R7 are H or alkyl and optionally form a ring. Unsubstituted carbons in aromatic rings are optionally substituted with N.

Molecular Design Considerations

The docking models of the MAPKK1 fragment and the In-2-LF inhibitor are used for the improvement of existing small molecule inhibitors and the de-novo design of new inhibitors. The resulting MD trajectory of the LF-MAPKK1 fragment complex is currently being used to provide the basis for the design of an improved DynaPharm® pharmacophore template, which is a central part of a virtual library screening strategy for discovery and optimization of more potent inhibitors.

First, flexible and rigid regions on the surfaces of LF and MAPKK1 in the cleavage region of the complex model are being determined from the MD trajectory. Detailed analyses are being carried out at the surface of the N-terminal portion of MAPPK1 residing in the LF active site in order to extract characteristics of the interacting residues over the trajectory. Residues at the interface are identified and grouped using the following criteria: 1) contribution to the energetics of its binding to LF and 2) analysis of hydrophobicity. After grouping residues, the distances and angles between each residue in the group are measured and tabulated. Whenever aromatic or non-aromatic rings are involved, the centers of the rings are used for distance evaluation. For side chains longer than Alanine, the center of mass of the residue are used as the reference point for measuring the distances and angles. This will yield the desired virtual constructs of the residues (including dynamic motion) for constructing a DynaPharm® template and for more refined docking-based approaches.

The new docking models have been applied to a 1-hydroxyhydropyrazin-2-one scaffold identified previously. Computational docking studies on hydroxypyrazinones using the LF structure suggested that the ring hydroxamic acid group would be prevented from chelating zinc because of unfavorable steric interactions between ligand and protein.

However, these studies also suggested that derivatives of these structures tethered to other zinc-binding groups (such as carboxylic acid or thiol) could show activity. Figure C-3 shows a few examples of hydroxypyrazinones exhibiting activity in the Western Blot assay. Only structure SBI-031592, which contains an additional carboxylic acid moiety, showed activity in the FRET assay. The methyl ester of SBI-031592 was inactive in this assay, suggesting that the carboxylic acid moiety in itself is important for activity, while the hydroxamic acid groups in the pyrazinone ring are insignificant. Analogs of hydroxypyrazinones without hydroxyl groups (pyrazinones and alkoxypyrazinones) did not differ significantly from hydroxypyrazinones in the Western Blot assay, a result that is also inconsistent with a model involving zinc binding to the ring hydroxamic acid group in these compounds.

In light of these SAR results for hydroxypyrazinones and the computational predictions, attention was given to other scaffolds, including hydroxamic acids, carboxylic acids, thiols and barbituric acids. Computational docking studies and similarity searching helped to identify scaffolds related to SBI-031592, containing nitrogen heterocycles linked to 2 or 3 phenyl rings, and exemplified by scaffolds B, E, F, G, H, J and K in Figure C-4. Computational studies based on interactions of the MAPKK1 peptide with LF and further similarity searching helped to identify scaffolds A, C, and D. Based on both computational and assay data, scaffolds C and J in Figure C-4 were identified as of particular interest. Preliminary results suggest that some inhibitors with scaffold J exhibit selectivity against LF versus MMP-1 (IC₅₀ (MB-1)/IC₅₀ (LF)>6). Scaffold C is particularly interesting because of its relative potency and drug-like nature. The drug-like nature of scaffold C derives from the fact that one molecule in this class has been tested in mice as a candidate inhibitor of another target (TNF sheddase) and successfully prevented the lethal effects of lipopolysaccharide+galactosamine by blocking TNF synthesis (Mohler, K. M. et al., Nature. 370:218 (1994). The compound thus appears to be non-toxic in mice and sufficiently bioavailable and stable to reach the target enzyme. Derivatives of scaffold C will be examined in an effort to improve its potency.

Structures and measured IC₅₀ values FRET assay) for some of the hydroxamic acid derivatives are presented in Figure C-5. Note that three of the derivatives have single digit micromolar IC₅₀ values and one has an IC₅₀ value of 1.6 μM. While more analog compounds are still being designed, synthesized and tested to gain a better understanding of structure/activity relationships, some preliminary conclusions can nevertheless be made. Scaffold B is an inhibitor, while its enantiomer is inactive, consistent with a specific interaction with LF near the binding site as opposed to nonspecific protein binding. For scaffold C, the most promising to date, the 2 fused aromatic rings (naphthyl and indole) and the alkyl group on the succinic diamide appear to contribute significantly to binding, while R3 is less critical. Intermediates for the synthesis of more than 40 other members of the C scaffold family have recently been prepared, using the docking model depicted in Figure C-6 as a guide.

Structures and measured IC₅₀ values for some of the carboxylic acid derivatives examined to date are presented in Figure C-7. Three of these derivatives are active in the single digit μM range. For the carboxylic acid scaffold series G, although there is little dependence on chain length, n=5 appears to be close to optimal. Competitive inhibition has been observed for individual compounds in the G and J scaffolds, consistent with binding near the active site. A docking model of a member of the J series bound to the LF active site is depicted in Figure C-6. Note that direct binding to zinc occurs for the hydroxamic acid moiety or the carboxylate moiety in both docking models shown in Figure C-6.

The X-ray crystal structure of LF has been computationally refined and combined with the AHM model of MAPKK1 to derive a full solvated MD trajectory of a bound LF-MAPKK2 fragment complex, and a bound complex of LF and the inhibitor In-2-LF. The bound complex, models have been used to design and screen new scaffolds and derivatives of candidate LF inhibitors. Compounds with IC₅₀ activities in the single digit micromolar range have been discovered for 3 novel scaffold families, with the most active to date being 1.6 μM.

At that point where 100 nM range compounds are identified, the co-crystallization task will be initiated for the most promising compounds, which will provide even more accurate structural information with which to further optimize the LF inhibitor candidates toward the 1-10 nM activity goal.

Strategies for improving stability to enzymatic degradation will include amide replacement by C—C bond containing moieties or heterocycles, replacements of readily oxidized sites (e.g. replacement of phenyl by 4-fluorophenyl, 1-butyl by 2-fluoro-1-butyl). Strategies to minimize toxicity will include replacement of potentially toxophoric groups by less toxic bioisosteres (e.g. replace 3-nitrophenyl with 3-aminosulfonylphenyl or 3-acetylphenyl) and changes that improve selectivity for LF versus other metalloenzymes. Strategies to maximize selectivity against LF compared to other enzymes will include computationally guided alterations in the size of appropriate moieties. Using this same approach, extension of methyl groups to heptyl or benzhydryl has been used to increase the selectivity of certain hydroxamic acids between matrix metalloprotease subtypes by >500 fold (Whittaker et al., Chem. Rev. V. 99, 2735-2776 (1999); Miller et al., Bioorg. Med. Chem. Lett 7:193 (1997)). Strategies for improving bioavailability will include enhancing solubility by decreasing symmetry, introducing branching, reducing molecular weight, and substituting hydrophobic groups with polar groups such as alkoxy and aliphatic amines. Both solubility and membrane permeability are enhanced as needed by making substitutions that optimize log P and log D values, such as replacing arginine side chains with less polar 2-aminopyridines, replace amide CONH with COCH₂, thiazole, oxadiazole, oxazole, alkene, etc. Figure D-4 shows examples of how some of these strategies are applied, using the LF inhibitor scaffolds C and J as starting points. The structures of those more promising inhibitors are treated similarly as for scaffolds C and J in Figure D4 using similar types of structural alterations and bioisosteric replacements.

Prodrug strategies are applied to agents that are predicted to show poor oral bioavailability, but are otherwise promising in terms of ADMET properties and potency when adminstered subcutaneously (s.c.) to mice. These will include strategies that have proven useful for other metalloproteinase inhibitors, e.g. ethyl esters as prodrugs of carboxylic acids and thioethers as prodrugs of thiols (Alton et al., J. Chromatogr 579:307-317 (1992); Noble et al., J Pharmacol Exp Ther 261:181-90 (1992); Skiles et al., Current Medicinal Chemistry 8:425-474 (2001)).

The basis for the substitutions proposed in analogs depicted in Figure D-4 is as follows (small letter designations in the list correspond to the letters in Figure D-4):
a) CH>>CF to improve metabolic stability.
b) Increased steric bulk within cavity to improve selectivity against LF versus other metalloenzymes.
c) CH>>N to optimize log D for bioavailability.
d) Decrease rotatable bonds through structural constraints for improved bioavailability.
e) α-alkylation enhances hydroxamic acid metabolic stability.
f) H>>CF₃ to adjust log D for bioavailability, and to improve metabolic stability.
g) Replace amide with heterocycle for improved metabolic stability, optimization of log D for bioavailability, decrease in NH bond count (Lipinski et al., Adv. Drug Del. Rev. 23:3-25 (1997)).
h) Replace amide with C—C for improved metabolic stability, optimize log D for bioavailability, decrease in NH bond count (Lipinski's rules). One example of significant improvement in oral bioavailability of a metalloprotease inhibitor through replacement of NH with CH₂ has been described by Chapman et al. Bioorg. Med. Chem. Lett. 6:803 (1996) (Lipinski et al., 1997).
j) Replace with alternate heterocycle for improved solubility and drug-lice character.
k) CF₃>>OCH₃ for improved solubility, optimization of log D for bioavailability.
m) Eliminate phenyl group for improved solubility, optimization of log D for bioavailability, and lower molecular weight.
n) CF₃>>F for lower molecular weight.
p) Replace hydroxamic acid moiety with thiol for decreased mutagenicity; thio ester pro drug for increased bioavailability.
q) Create acetoxymethyl ester of carboxylic acid as prodrug to improve oral bioavailability.
r) Append tertiary amine for improved solubility.
   Inhibitor Compounds of the Invention

According to the design considerations and strategies described above, the compounds according to the following structural formula will find use as inhibitor compounds useful for treating anthrax infections by inhibiting Anthrax Lethal Factor (LF) activity. LF inhibitors are useful, either alone or together with other therapeutic compositions, in the prevention and treatment of anthrax infections, whether resulting from infection by Bacillus anthracis spp., or purposefully induced invasions by LF.

Synthesis of Inhibitor Compounds of the Invention

In general, the compounds of the present invention can be prepared in accordance with chemical synthetic protocols well known to those of skill in this art. One desirable category of such techniques is known by the generic term “combinatorial chemistry.” Such techniques are well know in the art, and can be generally summarized as follows: For example, preparation of libraries can be by the “split synthesis” method, as described in Gallop et al., J. Med. Chem., 37:1233-1251 (1994). The split synthesis' procedure involves dividing a resin support into n equal fractions, in a separate reaction carry out a single reaction to each aliquot, and then thoroughly mixing all the resin particles together. Repeating the protocol for a total of x cycles can produce a stochastic collection of up to n^{x} different compounds. An alternative format is by preparing sub-libraries in the O₃O₂X₁ format, wherein two positions on the compounds, O₃ and O₂ are explicitly defined and a third position, X₁, varies. Such sub-libraries can be conveniently prepared by the tea-bag technique, as is known in the art, and described, for example in U.S. Pat. No. 4,631,211 and Houghten et al., Proc. Natl. Acad. Sci., 82:5131-5135 (1985).

Alternatively, or in addition thereto, the iterative selection and enhancement process of screening and sub-library resynthesis can be employed. For example, a sub-library of various R1 substituents can be screened to select the most active R1 substituent. The compound having the most active R1 is then resynthesized and with the R1 position being defined, a new R2 position mixture library is prepared, screened, and the most active R2 selected. The above process can then be repeated to identify the most active R substituents on the backbone structure.

In yet another approach, the positional scanning technique, only a single position is defined in a given sub-library and the most preferred substituent at each position of the compound is identified.

The advantage of synthetic combinatorial libraries (SCLs) made up of mixtures of tens of millions of different compounds is that they can be used to rapidly identify individual, active compounds without the need to individually synthesize, purify, and test every single compound. Since the libraries are in solution (i.e., not attached to a bead, pin, phage, glass, etc.) they can be screened in virtually any assay system.

Solution phase combinatorial chemistry methods can be used when the product can be separated from side products and starting materials through rapid techniques. Examples of these are: (1) selective precipitation of product and removal of byproducts and precursors by washing, (2) selective removal of side products and starting materials using chemically reactive polymers and/or ion exchange polymers (“scavenge”), (3) selective binding of product to a chemically reactive polymer, followed by removal of the product through a second chemical reaction (“capture”) (4) selective binding of product to an ion exchange polymer, followed by removal with acid, base, or high salt buffer (“capture”), and (5) selective solubilization of product. Solution phase combinatorial chemistry approaches are covered in a recent set of reviews (Tetrahedron, 54:3955-4150 (1998)).

The synthetic approaches can be optimally carried out using solution phase combinatorial chemistry. Several reactions are carried out simultaneously using a multiple reaction vessel block such as, but not limited to, the Charybdis Calypso™ temperature controlled blocks, with gas manifolds to maintain an argon or nitrogen atmosphere. Alternately, the reactions can be carried out simultaneously in multiple vials filled with argon or nitrogen and fitted with magnetic stirbars and polytetrafluoroethylene-lined, sealed caps, by heating and stirring them simultaneously in a magnetic stirrer/heater such as, but not limited to, the Pierce ReactTherm™ III Heating/Stirring Module. The products are isolated by addition of water and filtration using a system such as, but not limited to, the Charybdis Calypso™ filtration block or polypropylene syringes fitted with filter disks made from polyethylene, polytetrafluoroethylene, or glass and attached to a vacuum manifold.

Representative synthetic schemes for some of the structures proposed in Figure D-4 are depicted in Figure D-5. Representative combinatorial libraries and their synthetic schemes are shown in Figure D-6. Individual compounds are synthesized using high-throughput methods and screened to determine synthetic feasibility and the activity of a representative structure. (High throughput procedures will include solid phase chemistry and solution phase chemistry with solid-phase reagents and scavengers. Where appropriate, microwave chemistry using Personal Chemistry Synthesizer instrumentation is carried out to increase the efficiency of library synthesis.) If the synthetic accessibility and potency are adequate, a virtual library (100-1000 structures) are constructed and added to the set of libraries to be used for the second round of in silico ADMET screening. After in silico screening has been used to remove structures that are unlikely to exhibit favorable ADMET profiles, the structures remaining in the virtual library are synthesized using high-throughput procedures.

For certain compounds of the present invention, synthesis can be readily accomplished by resort to the following general protocols:

The procedures in Schemes 1 through 4 can be used to make the subset of claimed structures in which:

U is CH or C(CH₃),

R1 is R16Y where

R16 is Z(CHR5)ₙ, where n is 0 to 5, Z is a bond, Y is CONR11OH, L1 is CONH or CON(R14) where R14 is H or alkyl

where R15 is H or alkyl, and R2, R3, R4, R5, R11 and V are as described previously in the more general embodiments.

The protocols also can be used for those structures in which U, R5, Y and R11 form a ring, as described in the text and depicted in Figure B.

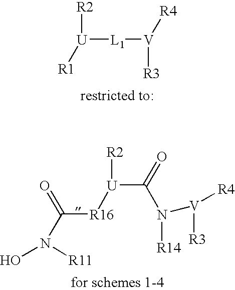

In the synthetic schemes shown here, if any of the Rn groups contain functionality that may interfere with or become chemically changed by the synthetic procedures shown, then these will be derivatized with appropriate, standard protecting groups that are not cleaved during the synthetic procedures, and which can be removed when needed without affecting other functionality.

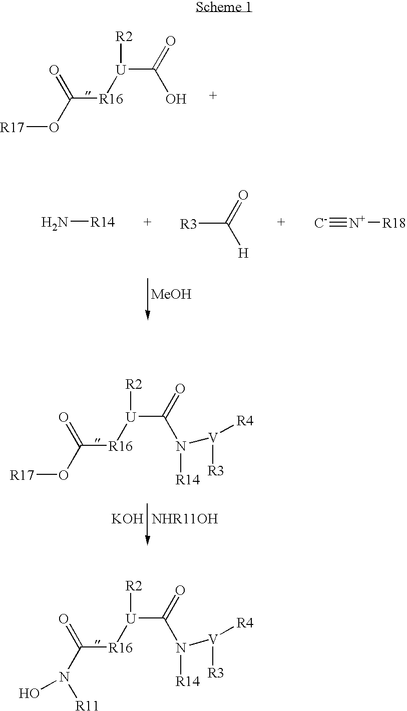

In Scheme 1, the structure is further restricted such that R17 is alkyl or benzyl, R14 is H or primary alkyl, R3 is a substituent linked through carbon (including Aryl, arylaryl, alkyl, arylalkyl, R18 is alkyl, aryl, arylalkyl, V is CH, and R4 is CONHR18.

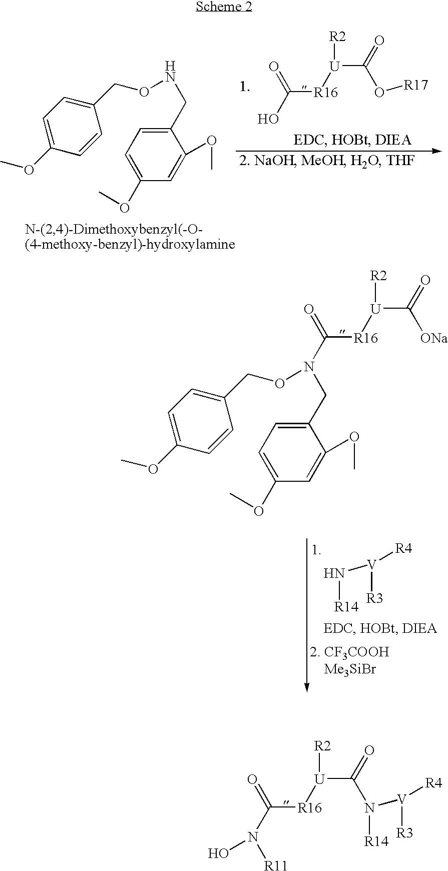

In Scheme 2, the structure is further restricted such that R11 is H.

Synthesis of N-(2,4)-Dimethoxybenzyl)-O-(4-methoxy-benzyl)-hydroxylamine is detailed in Examples 10 and 11.

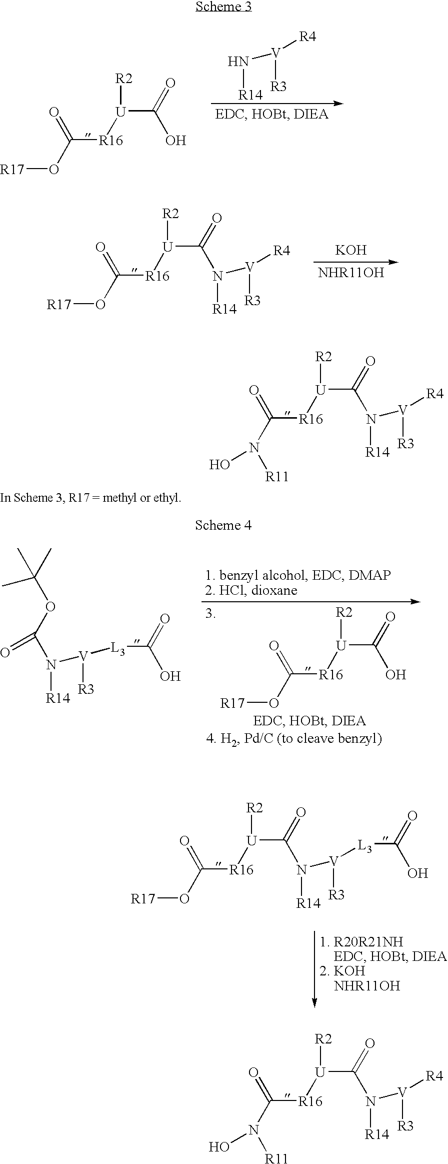

In scheme 4, the structures are further restricted such that R11 is H, R17 is methyl or ethyl, R4 is L3CONR2OR21 in which L3 is a bond, CH₂, CH₂CH₂, CH═CH, or cycloalkylidene (C3-C6), optionally further substituted with 1 or more alkyl, aryl, heteroaryl, heterocycloalkyl, OH, amine, or fluorine substituents, R20 and R21 are, independently, methyl, alkyl, benzyl, indanyl, arylalkyl, heteroarylalkyl, heterocycloalkyl, optionally further substituted with 1 or more alkyl, aryl, heteroaryl, alkoxy, carboxyl, heterocycloalkyl, OH, amine, or fluorine substituents. The procedures in Scheme 5 can be used to make the subset of claimed structures in which U is CH, R1 is R16Y where R16 is Z(CHR5)ₙ, where Z is a bond, Y is CONR11OH, L1 is CH═N, CH═NO, CH₂NH or CH═CH, R17 is alkyl or benzyl, and R2, R3, R4, R5, R11 and V are as described previously in the more general embodiments.

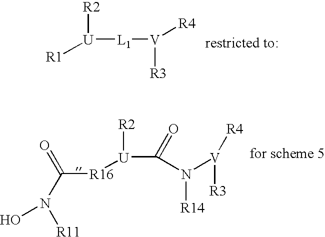

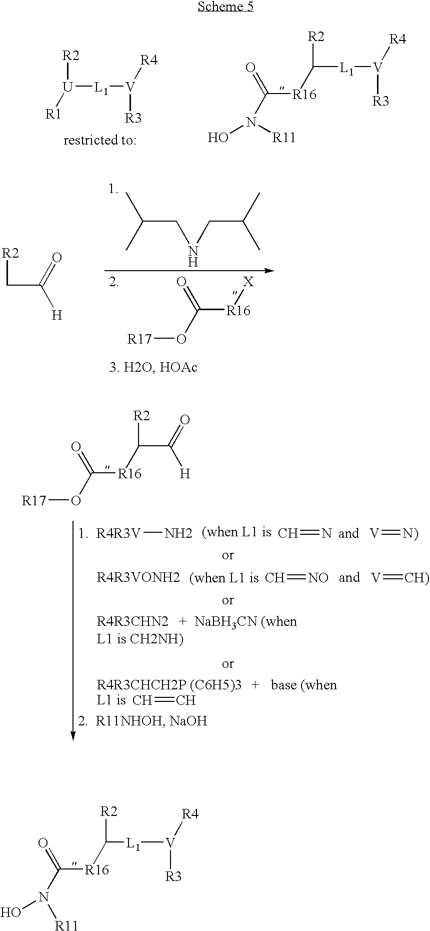

In scheme 6, R16 is Z(CHR5)ₙ where n is 0 to 4, Z is a bond, aromatic ring (1,2 or 1, 3 or 1,4-linking), heteroaromatic ring 1, 2 or 1, 3 or 1,4-linking), R2=R11 is primary alkyl, arylalkyl, heteroarylalkyl, cycloalkylalkyl; U is N, L1 is a single bond, V is C, where V, R3 and R4 form a 1,3,4-thiadiazole ring as shown above, R5 and Y substituents are as described in the description of the more general embodiments, and R12 is aryl, arylaryl, heteroarylaryl, aryloxyaryl, arylthioaryl, arylketoaryl, and heteroaryl analogs of these. Z, R5, R2, and R12 are optionally further substituted with one or more trifluoromethyl, alkyl, alkoxy, hydroxy, carboxyl, amine, aminoalkyl, cycloalkyl, heteroaryl, or aryl groups. Pro is protecting group, e.g. ethyl (in ester) for carboxylic acid.

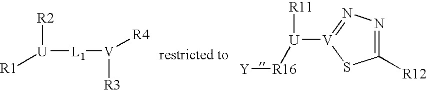

In scheme 7, R16 is Z(CHR5)ₙ where n is 0 to 4, Z is a bond, aromatic ring (1, 2 or 1,3 or 1,4-linking), heteroaromatic ring 1, 2 or 1, 3 or 1,4-linking), R2 is no substituent, U is N, L1 is a double bond, V is C, where U, V, R3 and R4 form an amino-thiadiazoline ring as shown above, R5 and Y substituents are as described in the description of the more general embodiments, and R12 is aryl, arylaryl, heteroarylaryl, aryloxyaryl, arylthioaryl, arylketoaryl, and heteroaryl analogs of these. Z, R5, R2, and R12 are optionally further substituted with one or more trifluoromethyl, alkyl, alkoxy, hydroxy, carboxyl, amine, aminoalkyl, cycloalkyl, heteroaryl, or aryl groups. Pro is a protecting group, e.g. ethyl (in ester) for carboxylic acid.

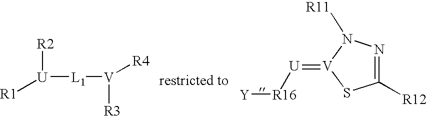

The products of schemes 6 and 7 are isomers made through identical routes, with separation of isomers before the final deprotection.

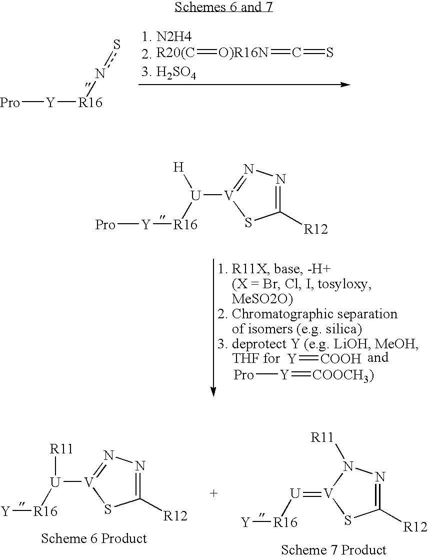

In scheme 8, R16 is Z(CHR5)ₙ where n is 0 to 4, Z is a bond, aromatic ring (1,2 or 1, 3 or 1,4-linking), heteroaromatic ring 1, 2 or 1, 3 or 1,4-linking), R2=R11 is primary ally, arylalkyl, heteroarylalkyl, cycloalkylalkyl; U is N, L1 is a single bond, V is C, where V, R3 and R4 form a 1,3,4-thiadiazole ring as shown above, Y is C(R20)=NO(CH₂)ₘCOOH where R20 is methyl, hydroxymethyl, alkyl, or cycloalkyl; m is 1 to 5; R5 is as described in the description of the more general embodiments, and R12 is aryl, arylaryl, heteroarylaryl, aryloxyaryl, arylthioaryl, arylketoaryl, and heteroaryl analogs of these. Z, R5, R2, R20 and R12 are optionally further substituted with one or, more trifluoromethyl, alkyl, alkoxy, hydroxy, carboxyl, amine, aminoalkyl, cycloalkyl, heteroaryl, or aryl groups. Pro is a protecting group, e.g. ethyl (in ester) for carboxylic acid. R17 is alkyl or benzyl.

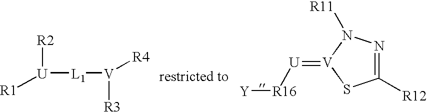

In scheme 9, R16 is Z(CHR5)ₙ where n is 0 to 4, Z is a bond, aromatic ring (1, 2 or 1,3 or 1,4-linking), heteroaromatic ring 1, 2 or 1, 3 or 1,4-linking), R2 is no substituent, U is N, L1 is a double bond, V is C, where U, V, R3 and R4 form an imino-thiadiazoline ring as shown above, Y is C(R20)=NO(CH₂)ₘCOOH where R20 is methyl, hydroxymethyl, alkyl, or cycloalkyl; m is 1 to 5; R5 is as described in the more general embodiments, and R12 is aryl, arylaryl, heteroarylaryl, aryloxyaryl, arylthioaryl, arylketoaryl, and heteroaryl analogs of these. Z, R5, R2, and R12 are optionally further substituted with one or more trifluoromethyl, alkyl, alkoxy, hydroxy, carboxyl, amine, aminoalkyl, cycloalkyl, heteroaryl, or aryl groups. Pro is a protecting group, e.g. ethyl (in ester) for carboxylic acid. R17 is alkyl or benzyl.

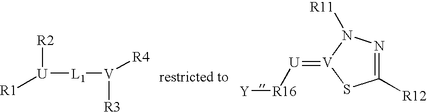

The products of schemes 8 and 9 are isomers made through identical routes, with separation of isomers before the final deprotection. 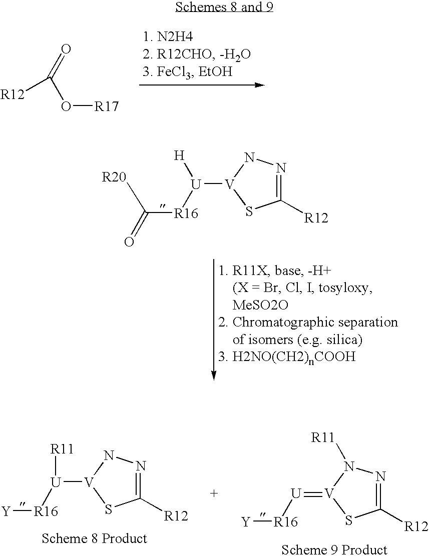
Other In Vitro Testing:

Other in vitro studies will be desirable for full assessment of candidate LF inhibitors. These studies, listed here for the sake of completeness, are: (1) LF inhibitory activity in vitro; (2) Efficacy against LT cytotoxicity in macrophages (external contract); (3) Ames test for mutagenicity (external contract—if the Ames test proves positive for compounds with otherwise favorable ADMET and activity profiles, more extensive genotoxicity and carcinogenicity studies in rats are carried out as needed); (4) set of PanLabs screens (external contract); and (5) cytotoxicity tests in human and rodent monocytic and hepatocyte cell lines (6) stability studies and (7) formulation.

Anthrax Lethal Factor FRET-Based, Enzymatic Assay

Materials:

Peptide Substrate [(Cou)Consensus(K(QSY-35)GG)-NH₂] (MW=2533): 1 mg/mL in Hepes buffer. (395M).
Lethal Factor Protease (MW=90,000): 1 mg/mL (10 μL/tube; 11 μM).
Assay Buffer: 20 mM Hepes, pH7.0, 1 mM CaCl2, 0.1 mg/mL BSA, 0.01% Tween-20.
Stop Solution: 4 mM 1,10-phenanthroline/40 mM EDTA.
   Enzymatic Assay Protocol for Compound Screen:

To each well of a 96-well flat-bottomed black plate, add the following:

25 μL of 12 μM peptide substrate in assay buffer (final conc. 4 μM),

20 μL: 1.5 μL compounds in DMSO mixed with 18.5 μL assay buffer,

30 μL of 20 nM of LF in assay buffer (final conc. 8 nM)
Mixed well, and incubate at 25° C. for 15 minutes

25 μL of Stop Solution added to terminate the reaction.

The fluorescence was read on a Victor 1420 plate reader with the umbelliferone protocol (excitation 355 nm/emission 460 nm).

Background wells use reactions without enzyme.

Reference: Cummings et al., Proc. Natl. Acad. Sci., 99(10):6603-6 (2002)

Assay for Matrix Metalloproteinase-1 (MMP1)

TABLE 1

| | | |
|---|---|---|
| 1) Prepare the | 50 mM HEPES pH 7.5 | 2.5 mL 0.5M HEPES pH 7.5 |
| reaction buffer: | 10 mM CaCl₂ | 0.25 mL 1.0M CaCl₂ |
| | 0.01% Tween20 | 50 μL 5% Tween |
| | ±0.01% BSA | 33 μL 7.5% BSA |
| | | Add H₂O to 25 mL |

2) Dilute the enzyme in reaction buffer to 15 units/μL (10 μL of MMP1
stock to 1 ml of reaction buffer) and distribute to 96-well plate 80 μL/well
3) Dilute Substrate stock 20 x in the reaction buffer→ Add 20 μL of
substrate to Enzyme solution→ mix→ Incubate the plate @30° C. for
time course→ Read Umbiliferone protocol 360/460→ and process the
data.

| | |
|---|---|
| Enzyme- | Matrix Metalloproteinase-1 (MMP1, intestinal collagenase |
| | human, recombinant with C-terminal purification tag, E. coli |
| | expressed) catalytic domain 81-249aa, MW = 19.9 kDa. |
| | Biomol #SE-180 11,547 u/μg, total of 10 μg in 19 μL. |
| Substrate- | Fluorescent MMP Substrate, [DNP-PChaGCHAK(Nma)], |
| | MW = 1077.2, Biomol CATALOG NO: P-128, Km = 10 μM |
| | for MMP1, 1 mg of net peptide/vital diluted in 1 mL of |
| | DMSO as 1 mM, store @ −20° C. Protocol 360/460 nm |
| Plates- | Corning#3656 96well Non-binding surface black plates |

Note:
Preparing Enzyme Stock (150units/μL): original MMP1 stock (0.53 μg/μL,
or 6120units/μL) dilute 1 μL into 41 μL of Enzyme buffer→ aliquot
10 μL/tube (150units/μL, 76 tubes total)→ Store the tubes @ −70° C.

| | | |
|---|---|---|
| MMP1 Enzyme | 50 mM Tris-HCL pH 7.5 | 0.5 mL 1 M stock |
| Buffer- | 5 mM CaCl₂ | 0.05 mL 1 M CaCl₂ |
| | 300 mM NaCl | 0.6 mL 5 M NaCl |
| | 20 μM ZnCl₂ | 0.02 mL 10 mM ZnCl₂ |
| | 0.5% Brij35 | 0.5 mL 10% Brij35 |
| | 30% Glycerol | 3 mL 100% Glycerol |
| | | Add H₂O to 10 mL |

Preparing of Substrate Stock (20 mM in DMSO)- 1 mg solid dissolved in
50 μL of DMSO→ Store @ −20° C.

Preclinical Toxicity and Efficacy Studies in Mice Using LT Challenge

In this task, approximately 20 compounds are chosen, on the basis of the best overall performance in the set of in vitro ADMET screens in Task 2, for further evaluation in live animals using Lethal Toxin (LT) challenge (LT is the toxic combination of LF and the permeabilizing factor, PA). The goal is for this set of compounds to consist of at least 2 representatives of each structural subclass.

Mouse studies described in this section (acute toxicity studies and efficacy studies involving LT-injected mice) are carried out in female mice, A/J strain, 6 weeks of age, weighing approximately 20 g each. The strain, gender and age were chosen based on a mean lifespan when exposed to 4×LD50 of anthrax LT that is sufficiently long (mean 3.7 days) to allow the possibility of post-toxin treatment as well as prophylaxis (Welkos et al., Infection and Immunity 51:795-800 (1986)). This 3,7-day lifespan is also similar to the mean lifespan (3 days) of mice infected with 5000 cfu of bacillus anthracis spores. The planned trials, and associated schedules and protocols are presented in the following sub-sections.

Acute Toxicity in Mice

Single doses of drug candidates are injected s.c. into sets of 5 mice per dose level using 0.1, 0.3, 1, 3, and 10 mg/kg. Animals are observed for 14 days to estimate the MTD or to determine the lower limit of the MTD (the highest dose at which no more than 10% of the mice show clear signs of toxicity). Mice are weighed daily and their food consumption measured. For this preliminary study, the signs of toxicity are limited to nausea, lethargy, anorexia, weight loss, abnormal fur texture, diarrhea or mortality within the 14-day observation period. Mice showing signs of pain due to toxic effects are euthanized immediately. Combination toxicity studies of each candidate at its MTD with ciprofloxacin will also be carried out, because compounds that exhibit significant adverse interactions with ciprofloxacin are not worthy of further consideration. Compounds must have maximum tolerated doses above 1 mg s.c. for further consideration (the dose used for inhibition of the TACE metalloprotease by the compound). For mice with an MTD>1 mg/kg, postmortem gross necropsy is carried out on 5 mice from the group with the highest tolerated concentration on day 14. If no toxicity is observed at 10 mg/kg, the dose is increased until the maximum tolerated dose is determined (˜10% incidence of clear toxicity).

Prophylacetic Efficacy Against LT in Mice (Mortality Endpoint)

Initially, efficacy studies involving single injections of LT and single s.c. doses of drug candidate are carried out to eliminate molecules that have insufficient efficacy for further study. For each experiment 10 mice (control group) are injected s.c. with 0.3 mL saline, and 10 (treated group) s.c. with drug candidate in 0.3 mL saline, to be administered 5 minutes prior to the LT injection. All 20 animals will then be injected with 50 μg of PA combined with long of LF (4×LD50). Surviving mice are observed for 14 days for signs of LT-induced nonlethal toxicity.

Prophylacetic Efficacy Against LT in Mice (MEK-1 Cleavage Endpoint)

Related experiments will use the ratio of LF-cleaved to uncleaved MEK-1 in macrophages as an endpoint. Because it is difficult to isolate sufficient numbers of monocytes from peripheral blood in mice, uninduced peritoneal macrophages are used. For each experiment 10 mice (control group) are injected s.c. with 0.3 mL saline, and 10 (treated group) s.c. with drug candidate in 0.3 mL saline, to be administered 5 minutes prior to an i.p. LT injection. At t=2 hours after injection, mice are sacrificed and peritoneal macrophage isolated by flushing the peritoneal cavity with 4 mL of 0.34 M sterile-filtered sucrose. Each mouse is expected to yield roughly 3×10⁶ macrophages (Lefkovits and Benvenuto, Immunological Methods, Vol II, Academic Press, New York, p. 291 (1981). The suspension is immediately combined with an equal volume of 2% sodium octadecylsulfate containing 2 mM EDTA and 2 mM phenantbroline in order to lyse the macrophages and stop LF activity. The ratio of cleaved to uncleaved MEK-1 is determined using Western Blot analysis with a specific anti-MEK-1 monoclonal antibody. Based on the kinetics of NMK cleavage in macrophages and the rapid activity of lethal toxin rodents, 2 hours of exposure to LT should be sufficient time for measurable MEK cleavage to occur in macrophages (Tonello et al., Nature 418:386 (2002), Fish et al., J. Infect. Dis. 118:114-124 (1968); Welkos et al., (1986)). Western Blot analysis should be sufficient for determining the cleaved to uncleaved MEK-1 ratio because it has been used to determine inhibition of LF activity inside live culture macrophage cell lines (Tonello et al., 2002), and 20 ng of MEK-1 (cleaved+uncleaved) has been found more than sufficient for quantitation using this technique.

Usually, drug candidates that cause >4-fold increase in lifespan relative to controls and a statistically significant (P>0.95) decrease in the cleaved/uncleaved MEK-1 ratio in the prophylacetic studies are carried forward. Approximately 12 such molecules are chosen for further studies.

Efficacy Against LT in Mice at t=1 Hour Post Injection:

The 12 candidates that meet criteria outlined in the prophylaxis study with MEK-1 cleavage endpoint will undergo a study in mice in which the candidates are administered at t=1 hour, 2 hours, and 3 hours after injection using 10 control mice and 20 treated mice. (Sets of 20 treated mice are used for each candidate and dose schedule to allow statistical significance even if 50% of the treated mice die before the t=3 hour injection time). Surviving mice from treated groups are observed for 14 days after the first injection of LF+PA.

Only drug candidates that increase lifespan at least 2-fold relative to controls (with statistical significance, P>0.95) are considered for the oral prophylaxis studies. However, compounds with very strong prophylacetic activity will also be included for further study in the live challenge experiments, even if they are not very active in the therapeutic efficacy study, because the LT concentrations are well below 4×LD₅₀ until a very late stage of the infection. Approximately 8 compounds are chosen for the next step.

Oral Prophylaxis in LT-Treated Mice:

Compounds active in the mortality endpoint prophylaxis study with adequate PK are tested for oral prophylaxis in mice. “Adequate oral PK” is defined as >40% oral bioavailability and a serum half-life >2:5 hours. Both oral and s.c. PK are determined for the compounds on a contract basis by Cerep, Inc., enabling calculation of the serum half-life and % oral bioavailability.

Approximately 6 compounds are chosen for oral activity studies in mice, based on their optimal performance in the oral PK studies. The procedure for these studies is very similar to the s.c. studies in the prophylaxis study with a mortality endpoint: for each experiment 10 mice (control group) are treated p.o. with 0.1 mL vehicle, and 10 mice (treated group) s.c. with a selected drug candidate in 0.1 mL vehicle, to be administered t minutes prior to the LT injection. The value of t will be such that the time between agent administration (oral gavage) and LT injection will be longer so that the mean peak concentration of agent in plasma corresponds with the time of LT injection (based on the oral PK data). All 20 animals will then be injected with 50 μg of PA combined with long of LF (4×LD₅₀). Surviving mice are observed for 14 days for signs of LT-induced nonlethal toxicity.

Long-Term Non-GLP Toxicity Studies:

Six of the most promising candidates, chosen based on the mouse and rat efficacy, acute toxicology and PK studies, will undergo extensive, long-term, non-GLP toxicity studies in mice, with complete blood workup and postmortem organ histopathology based on a multiple s.c. injection schedule (2× daily for 5 days) suitable for live bacillus anthracis experiments. To the extent possible, the candidate compounds are chosen to represent a variety of structural subclasses. The maximum tolerated dose at this schedule will be determined in this way.

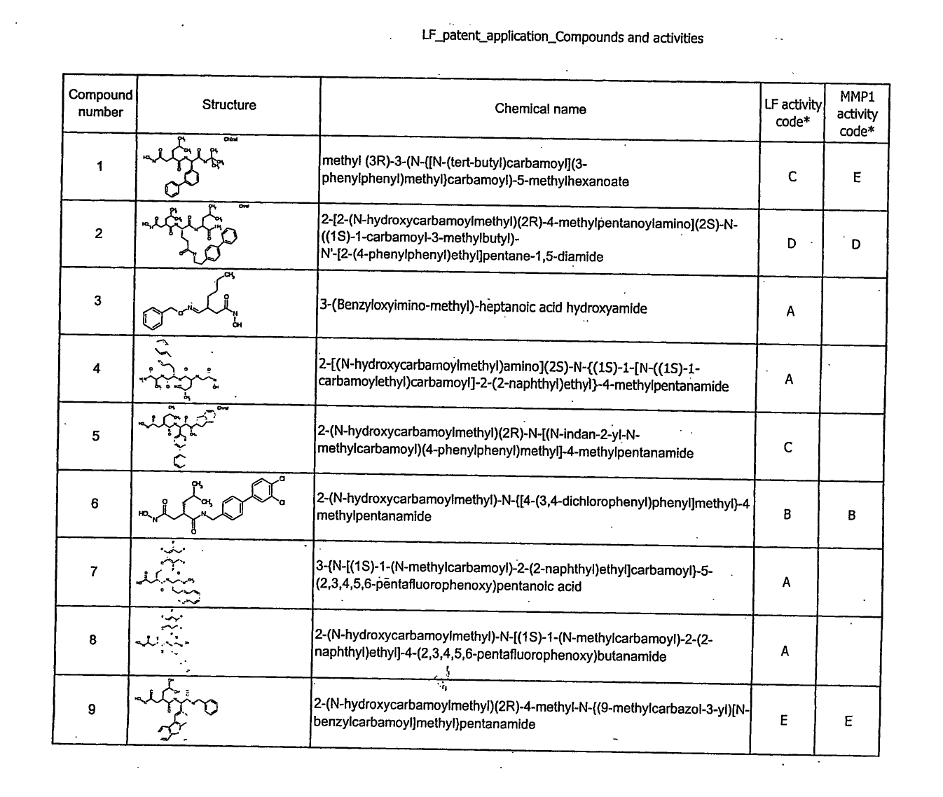

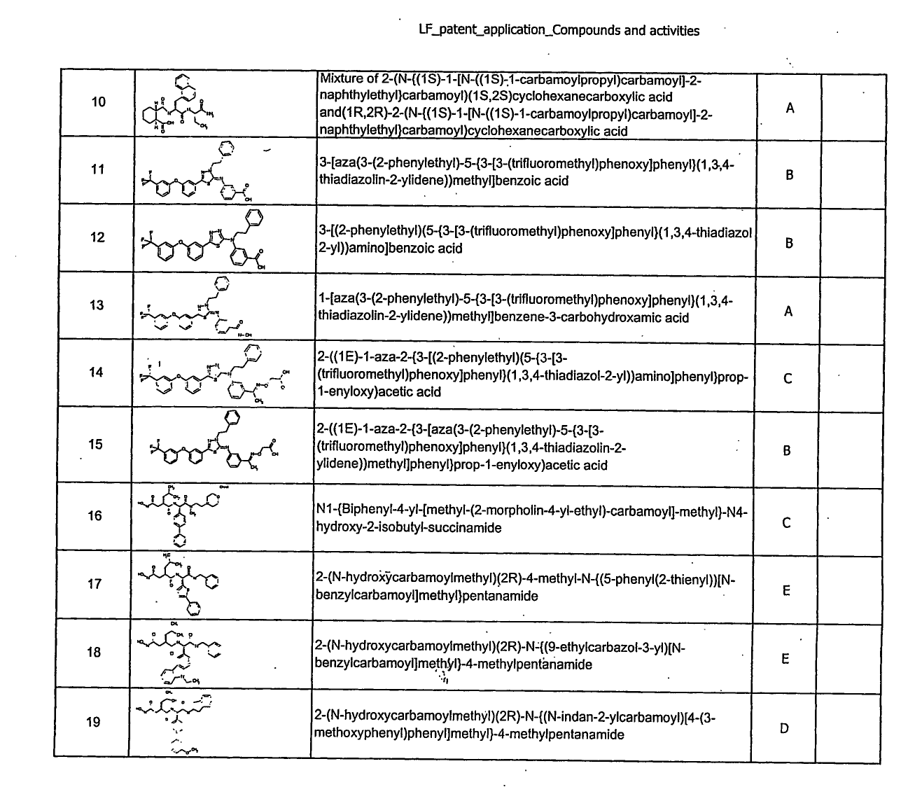

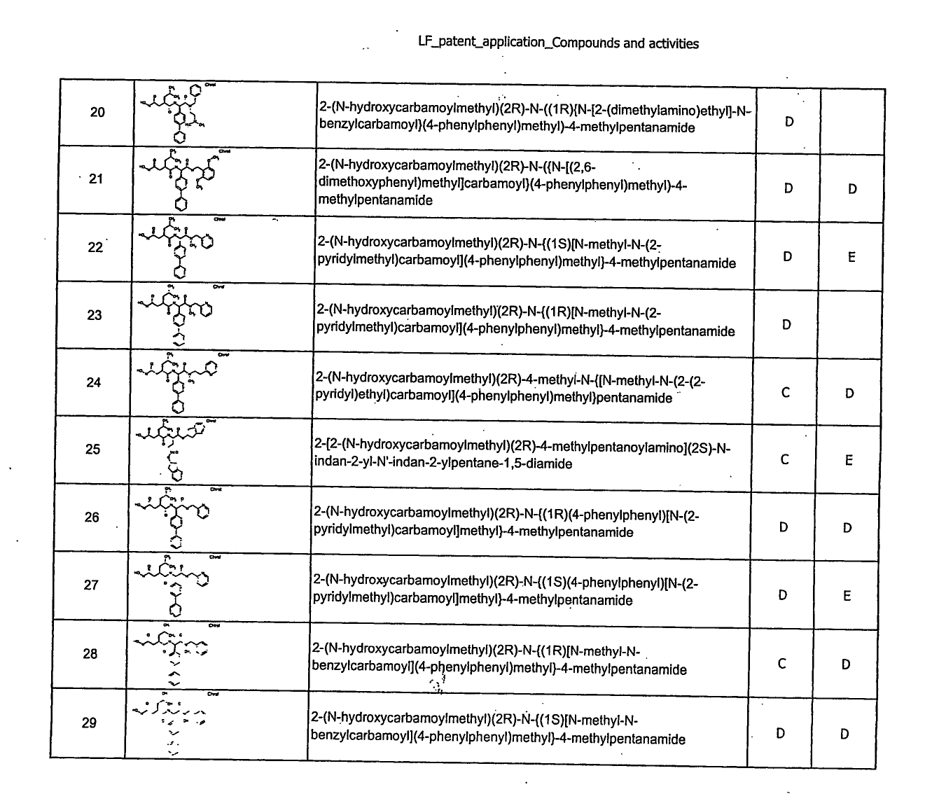

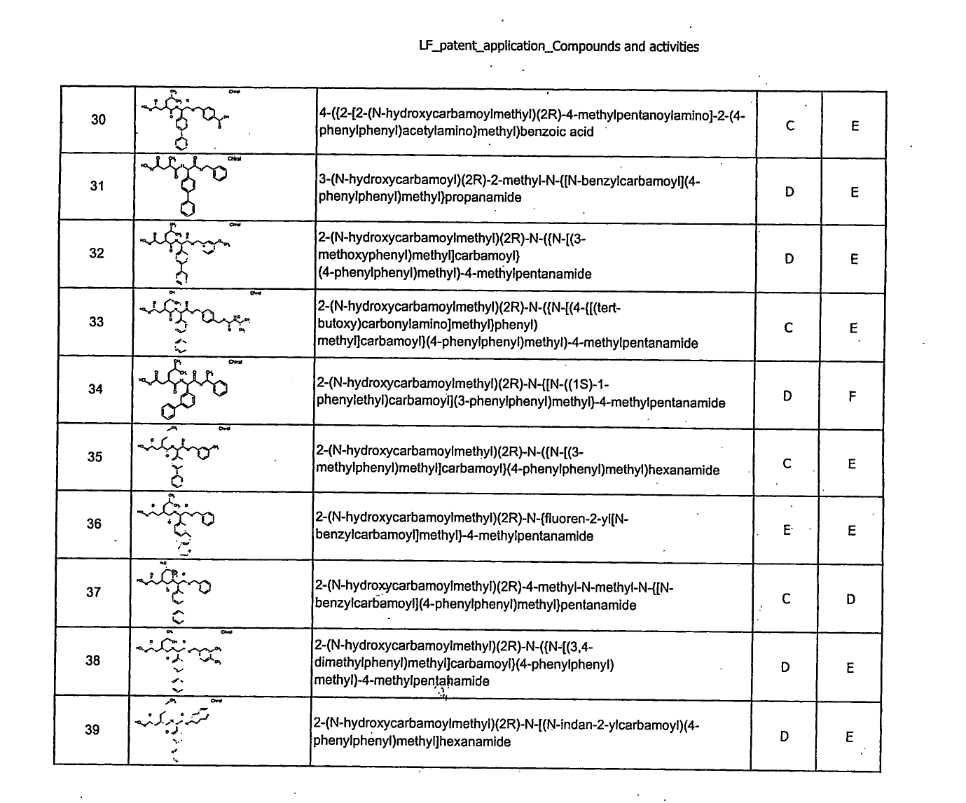

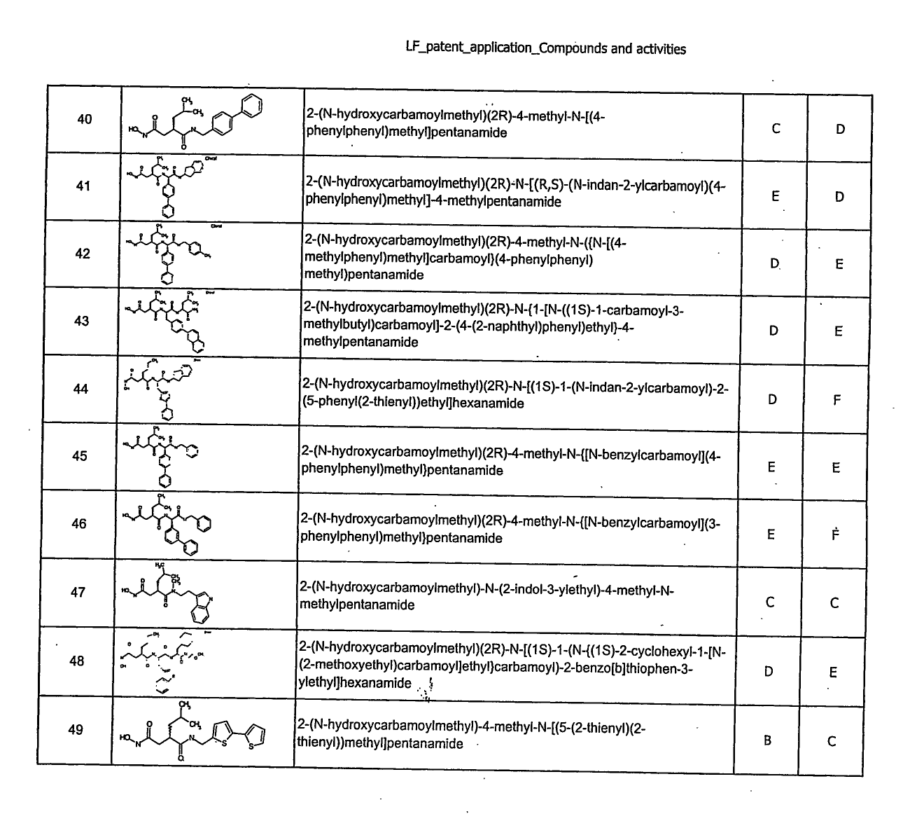

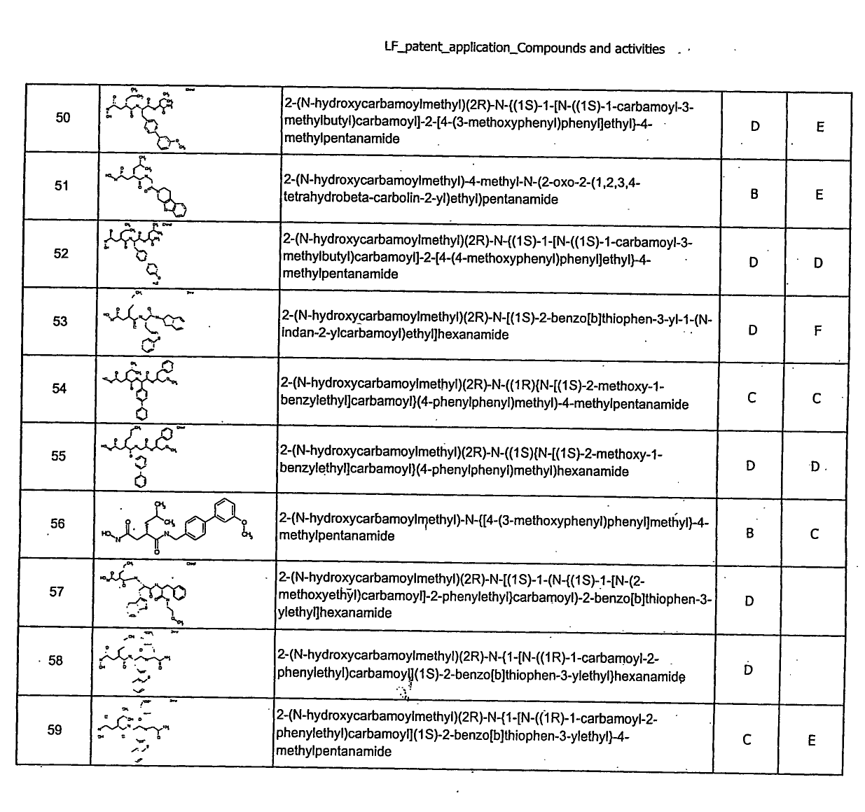

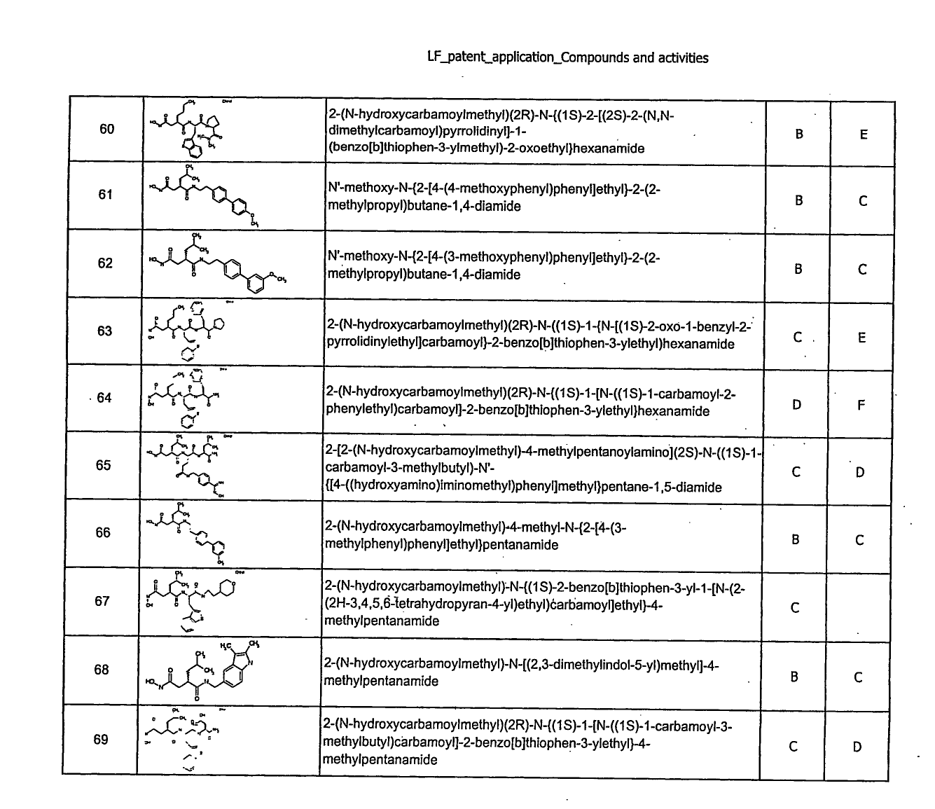

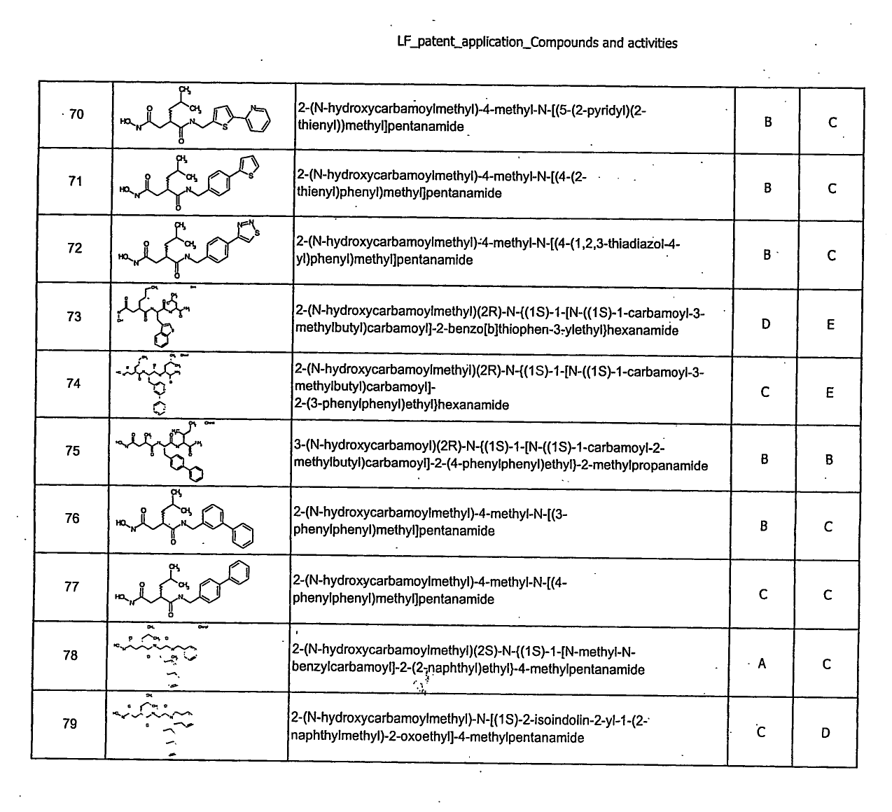

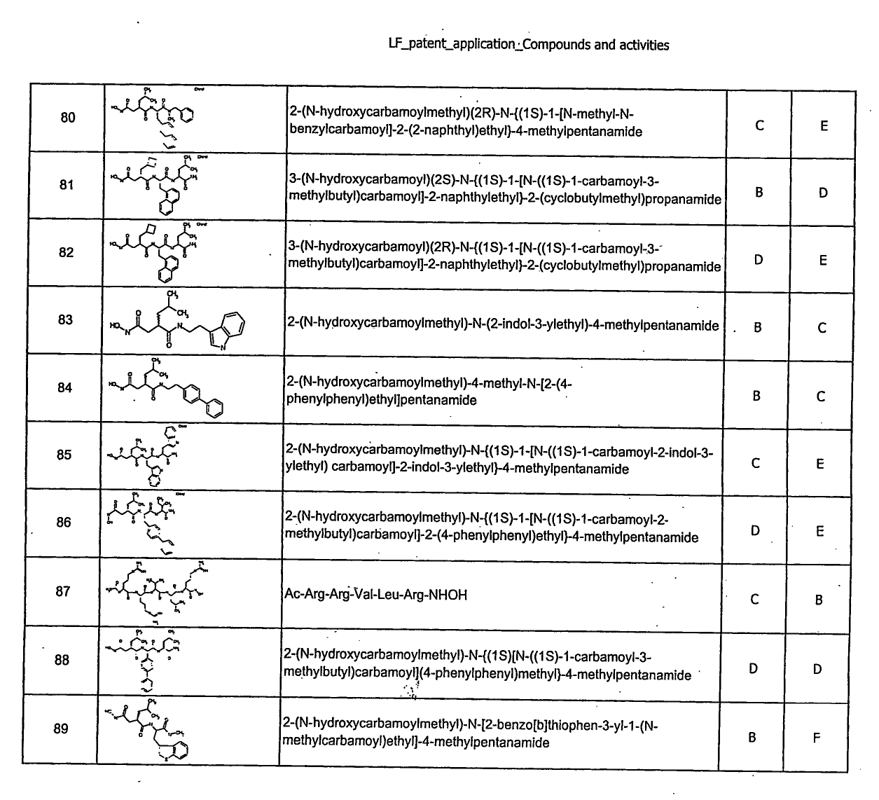

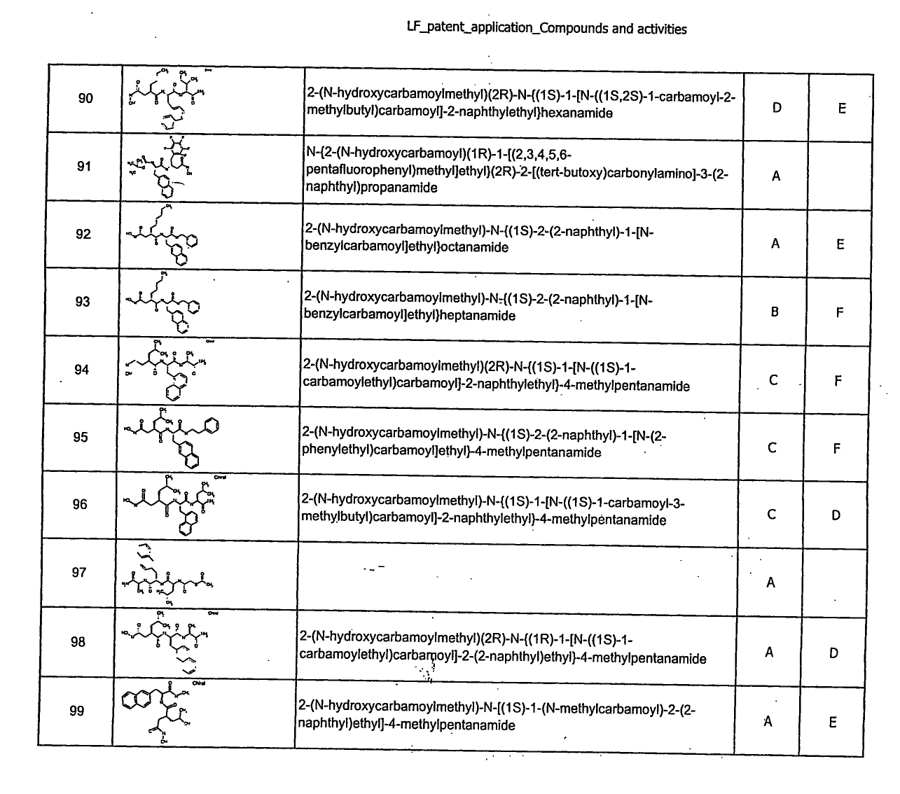

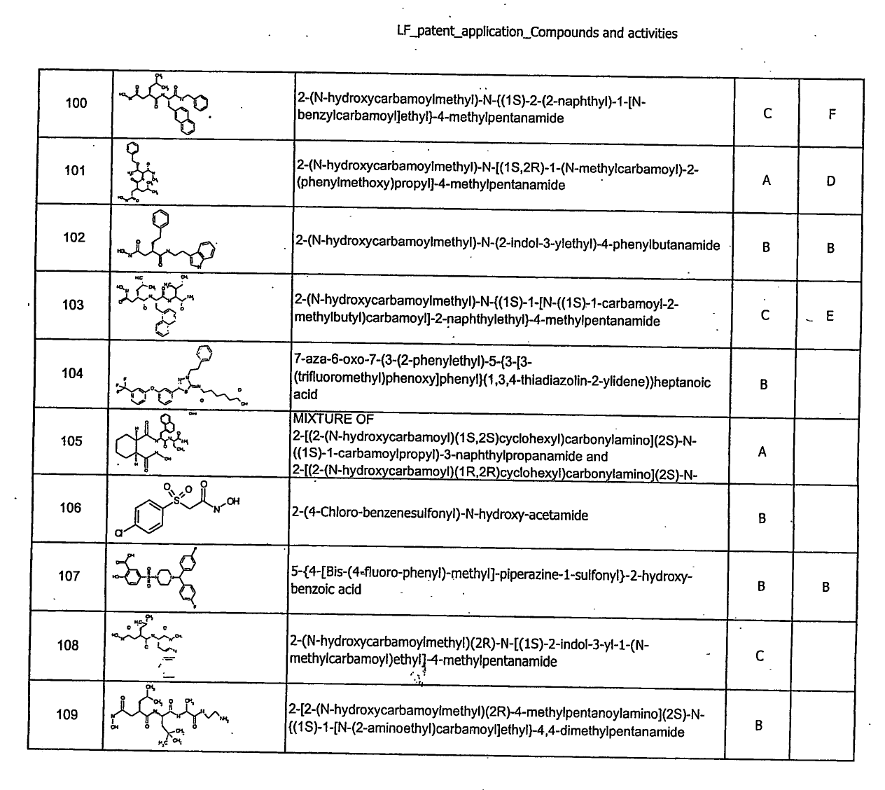

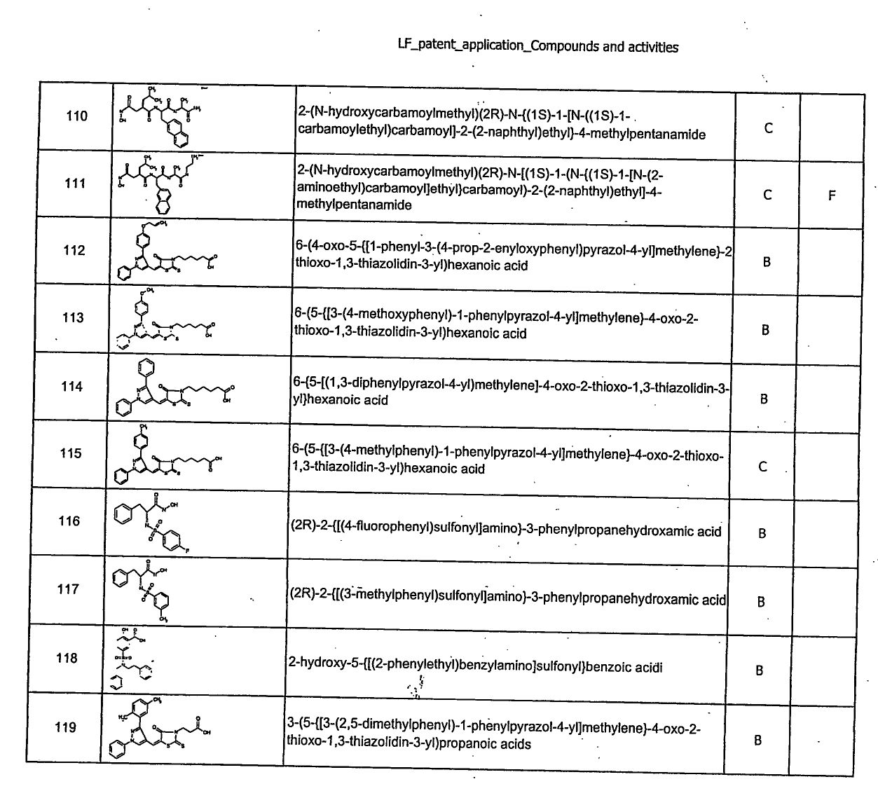

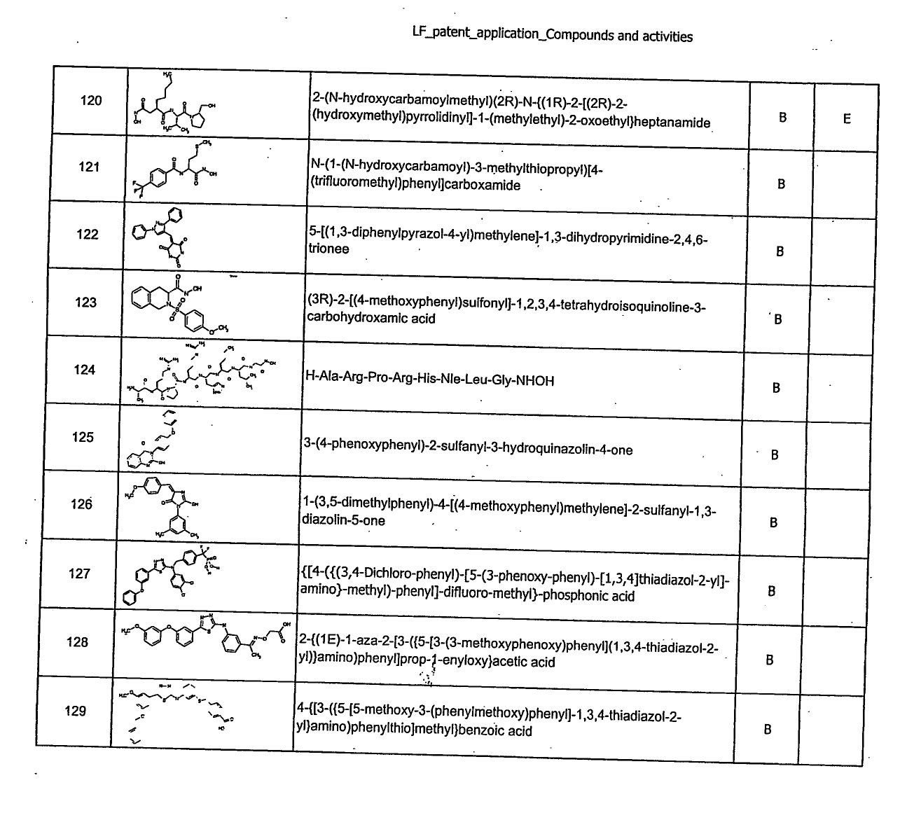

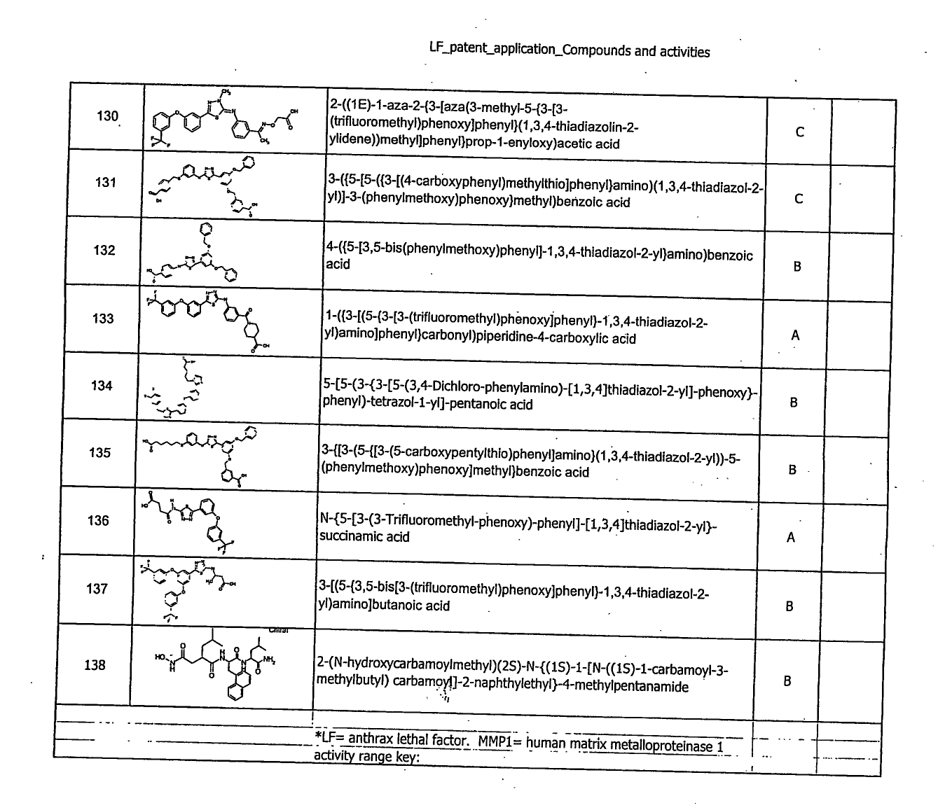

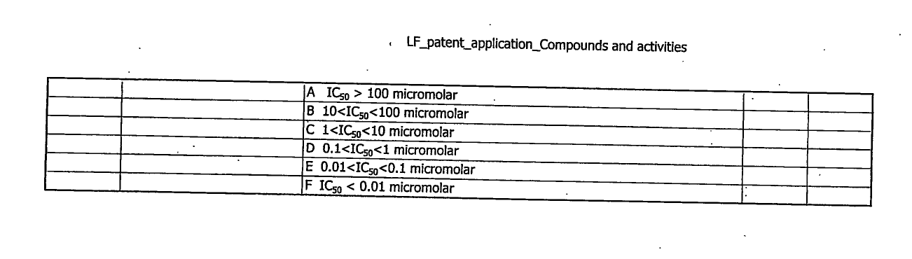

The following Examples serve to illustrate certain preferred embodiments and aspects of the present invention and are not to be construed as limiting the scope thereof. The structures of various of the disclosed compounds will be found depicted in FIG. 1.

### EXPERIMENTAL

In the experimental disclosure which follows, all weights are given in grams (g), milligrams (mg), micrograms (fig), nanograms (ng), or picograms (pg), all amounts are given in moles, millimoles (mmol), micromoles (μmol), nanomoles (nmol), picomoles (pmol), or femtomoles (fmol), all concentrations are given as percent by volume (%), proportion by volume (v:v), molar (M), millimolar (mM), micromolar (μM), nanomolar (nM), picomolar (pM), femtomolar (fM), or normal (N), all volumes are given in liters (L), milliliters (mL), or microliters (μL), and linear measurements are given in millimeters (mm), or nanometers (nm) unless otherwise indicated.

The following Examples demonstrate the practice of the present invention in synthesizing compounds according to the invention, generally as depicted in FIG. 1, and in methods by which drugs having the formulas shown can be readily identified by routine assay procedures to demonstrate that they possess the desired activity.

### Example 1

### Methyl (3R)-3-(N-{[N-(tert-butyl)carbamoyl](3-phenylphenyl)methyl}carbamoyl)-5-methylhexanoate

In an ice bath under dry conditions, added 2M NH₃/CH₃OH (1 mL). A solution of 3-phenylbenzaldehyde-(0.14 g, 0.77 mmol) in 2 mL MeOH was added to the mixture and stirred for 5 min in cold, and 10 min at room temperature. Added 2-aza-3,3-dimethylbut-1-ene (0.064 g, 0.77 mmol) and (2R)-2-[(methoxycarbonyl)methyl]-4-methylpentanoic acid (0.145 g, 0.77 mmol) in 3 mL MeOH to the mixture and refluxed (80-85° C.) for overnight. The title product precipitated out and was collected by filtration, washing with MeOH and hexanes and drying in vacuo to yield 0.068 g of the desired compound (20% yield). MS (M+H)⁺:453.

### Example 2

### Compound 1: Methyl (3R)-3-(N-{[N-(tert-butyl)carbamoyl](3-phenylphenyl)methyl}carbamoyl)-5-methylhexanoate

KOH (1.60 g, 28.5 mmol) was dissolved in dry MeOH(8 mL). NH₂OH—HCl (1.251 g, mmol) was dissolved in dry MeOH (12 mL) and cooled to 0° C. The KOH solution was poured into the NH₂OH—HCl solution and stirred for 1 hour. The product from Example 1 (67 mg, 0.148 mmol) was dissolved in dry MeOH (0.3 mL). The KOH/NH₂OH—HCl solution (1.19 mL) was filtered into this solution and stirred for 1-2 hours at room temperature. Reaction completion was detected by LC/MS. The reaction mixture was concentrated in the removal of MeOH. The residue was dissolved in H₂O (3 mL) and acidified to pH=6 with 6N HCl, and neutralized with saturated NaHCO₃ (pH=9). The product precipitated and was collected by filtration. Purification of the product was either done by recrystallization or C18 silica gel reverse phase chromatography (filter funnel) with water/methanol mixtures, yielding the title product (0.051 g) in 76% yield (diastereoisomeric mixture of 23/77 ratio), R_{f}=0.72 (ethyl acetate/methanol, 9:1). MS (M−H)-452.

### Example 3

### (2S)—N-((1S)-1-carbamoyl-2-methylpropyl)-2-[(tert-butoxy)carbonylamino]-3-naphthylpropanamide

A solution of (S)—N-Boc-1-Naphthylalanine (630 mg, 2 mmol), L-valineamide hydrochloride (306 mg, 2 mmol), 1-hydroxybenzotriazole (306 mg, 2 mmol) in dichloromethane was treated with EDC HCl (768 mg, 4 mmol) and diisopropylethylamine (1.216 mL, 7 mmol) and the mixture was stirred overnight. The dichloromethane was rotovaped, the residue was taken in ethylacetate. The ethylacetate solution washed with 1N HCl (2×10 mL), saturated sodiumbicarbonate solution (2×10 mL) and finally with brine (2×10 mL). The ethylacetate solution was dried over anhydrous sodium sulphate and rotovaped. The residue on trituration with hexanes gave a whit solid. Yield: 625 mg. (75%). MS (M+Na)⁺:436

### Example 4

### Synthesis of (2S)—N-((1S)-1-carbamoyl-2-methylpropyl)-2-amino-3-Naphthylpropanamide, chloride

(2S)—N-((1S)-1-carbamoyl-2-methylpropyl)-2-amino-3-Naphthylpropanamide, chloride was prepared by treating product from Example 3 (0.62 g, 1.5 mmol) with 4N HCl/Dioxane for 30 minutes. The dioxane was rotovaped and the residue was triturated with ether and dried under vacuum. Yield: 0.48 g. (93%). This was used without further purification.

### Example 5

### phenylmethyl (4S)-4-[N-((1S)-1-carbamoyl-3-methylbutyl)carbamoyl]-4-[(tert-butoxy)carbonyl amino]butanoate

Phenylmethyl (4S)-4-[N-((1S)-4-carbamoyl-3-methylbutyl)carbamoyl]-4-[(tert-butoxy)carbonyl amino]butanoate was prepared using the procedure in Example 3 from Boc-L-Glu(Obzl)-OH (3.37 g 10 mmol), L-leucinamide (1.43 g, 11 mmol), EDC HCl (3.84 g, 20 mmol), anhydrous hydroxy benzotriazole (1.35 g, 10 mmol) and diisopropylethylamine (3.48 mL, 20 mmol). Yield: 3.8 g (88%).

MS (M+H⁺-Boc Group) 350.

### Example 6

### (4S)-4-[N-((1S)-1-carbamoyl-3-methylbutyl)carbamoyl]-4-[(tert-butoxy)carbonylamino]butanoic acid

(4S)-4-[N-((1S)-1-carbamoyl-3-methylbutyl)carbamoyl]-4-[(tert-butoxy-3) carbonylamino]butanoic acid was prepared by dissolving the product from Example 5 (3.6 g, 8 mmol) in a mixture of MeOH (20 mL), THF (5 mL) and 1N sodiumhydroxide (20 mL). The mixture was stirred until the TLC shows the absence of starting material. Methanol and THF were rotovaped, the residue was diluted with water and washed with ethylacetate (2×20 mL). The aqueous layer was cooled in ice bath and acidified with 1N hydrochloric acid to pH of 3. Now the compound was extracted with ethylacetate (3×500 mL). The combined ethylacetate extracts were washed with brine (2×10 mL) and dried over anhydrous sodium sulphate and ethylacetate was rotovaped. The residue on trituration gave a white solid. Yield 2.7 g (96%):

¹H NMR: (300 MHz, CDCl₃): 12.09 δ (1H bs); 7.71 δ (1H d); 7.30 δ (1H d); 7.10 δ (2H dm); 4.24 δ (1H m) 3.88 δ (1H m); 1.84 δ (6H m); 1.43 δ (10H m); 0.94 δ (6H m).

### Example 7

### (2S)—N-((1S)-1-carbamoyl-3-methylbutyl)-2-[(tert-butoxy)carbonylamino]-N′-[2-(4-phenylphenyl)ethyl]pentane-1,5-diamide

A solution of (4S)-4-[N-((1S)-1-carbamoyl-3-methylbutyl)carbamoyl]-4-[(tert-butoxy)carbonylamino]butanoic acid (1.08 g, 3 mmol) in DMF (20 mL) was treated with 2-(4-phenyl phenyl)ethylamine (0.985 g, 5 mmol) anhydrous hydroxylbenzotriazole (0.405 g, 3 mmol), 1-[3-Dimethyl amino)propyl]-3-ethylcarbodiimide hydrochloride (1.152 g, 6 mmol) and diisopropyl-ethylamine (1.04 mL, 6 mmol). The mixture was stirred for overnight at room temperature. Next day, DMF was rotovaped under reduced pressure and the residue was taken in ethylacetate. The ethylacetate washed with 1N HCl (2×15 mL), Saturated sodium carbonate (2×15 mL) and brine (2×15 mL), and ethylacetate layer was dried over anhydrous sodium sulphate and rotovaped. The residue on triturating with hexane gave a solid. Yield: 1.2 g (74%).

¹H NMR: (300 MHz, DMSO-d6): 7.91 δ (1H t); 7.73 δ (1H d); 7.65 δ (5H m); 7.46 δ (2H m); 7.30 δ (4H m) 7.01 δ (1H d); 4.26 δ (1H m); 3.86 δ (1H m); 3.87 δ (2H m); 2.75 δ (2H t); 2.50 δ (2H m); 1.50 δ (5H m); 1.38 δ (9H s); 0.84 δ (6H m).

### Example 8

### (2S)—N-((1S)-1-carbamoyl-3-methylbutyl)-2-amino-N′-[2-(4-phenylphenyl)ethyl]pentane-1,5-diamide, chloride

(2S)—N-((1S)-1-carbamoyl-3-methylbutyl)-2-amino-N′-[2-(4-phenylphenyl)ethyl]pentane-1,5-diamide, chloride was prepared by treating (2S)—N-((1S)-1-carbamoyl-3-methylbutyl)-2-[(tert-butoxy)carbonylamino]-N′-[2-(4-phenylphenyl)ethyl]pentane-1,5-diamide (1.08 g, 2 mmol) with 4N HCl in dry dioxane (10 mL). The mixture was stirred for 30 minutes, the dioxane was rotovaped, and the residue was triturated with ether and dried under vacuum. Yield: 0.87 g (91%). This material used without further purification.

### Example 9

### 2,4-Dimethoxy benzaldehyde oxime

2,4-Dimethoxy Benzaldehyde (8.3 g, 50 mmol) was dissolved in 150 mL of Hydroxylamine hydrochloride (4.1 g, 60 mmol) and 15 mL of pyridine were added and the mixture was stirred at ambient temperature for one hour. The solution was diluted with 250 mL of water and extracted with ethyl acetate (150 mL, 3 times). The organic extracts were dried over magnesium sulfate, concentrated on the rotary evaporator and excess pyridine was removed on the high vacuum pump to afford 9 g (˜99%) of the title compound as a white solid.

¹HNMR (CDCl₃, 300 MHz) δ 10.9 (s, 1H), 8.15 (s, 1H), 7.53 (d, J=8.4 Hz, 2H), 6.53 (m, 2H), 3.77 (s, 3H), 3.75 (s, 3H). LS/MS C9H11NO3 calculated for: (M+H⁺) 182, found: 182.

### Example 10

### 1-{(1E)-2-aza-2-[(4-methoxyphenyl)methoxy]vinyl}-2,4-dimethoxybenzene

Method 10A: 2,4-Dimethoxy benzaldehyde oxime (3.6 g, 20 mmol, see Example 9), 4-methoxy Benzyl chloride (3.4 g, 22 mmol), and tetra-butyl ammonium iodide (1.1 g, 3 mmol) were dissolved in 200 mL of TMF and cooled to 0° C. by an ice bath. Sodium Hydride (1.1 g, 26 mmol, 60% dispersion) was added in 4 portions to the stirring mixture. The ice bath was removed and the reaction was stirred for 2 hours and when the starting oxime had been completely consumed (as judged by tlc analysis) the reaction was quenched by the addition of 200 mL of saturated ammonium chloride. The aqueous phase was extracted three times with 15 mL of ethyl acetate. The combined organics were dried over magnesium sulfate, concentrated on the rotary evaporator and subjected to silica gel chromatography (hexanes:ethyl acetate; 6/4). The title compound, 5.4 g, was isolated in 90% yield.

¹HNMR (CDCl₃, 300 MHz) δ 8.22 (s, 1H), 7.53 (d, J=8.1 Hz, 1H), 7.27 (d, J=8.7 Hz, 2H), 6.87 (d, J=8.7 Hz, 2H), 6.54 (m, 2H), 4.99 (s, 2H), 3.77 (s, 3H), 3.75 (s, 3H), 3.70 (s, 3H). LS/MS C17H19NO4 calculated for: (M+H⁺)302, found: 302.

Method 10B: Under dry conditions, 2,4-dimethoxybenzaldehyde (4.3 g, 26 mmol) and [(4-methoxyphenyl)methyl]oxyamine hydrochloride (5 g, 26 mmol) were stirred in dichloroethane (90 mL) for 10 min. Added sodium triacetoxyborohydride (8.3 g, 39 mmol) and stirred for overnight. Quenched with sodium hydrogen carbonate (saturated) until pH=8. Extracted with ethyl acetate. Ethyl acetate was dried over sodium sulfate, filtered, and concentrated give the title compound in 69% yield. MS (M+H)⁺:302.

### Example 11

### N-(2,4-Dimethoxy-benzyl)-O-(4-methoxy-benzyl)-hydroxylamine

2,4-Dimethoxy-benzaldehyde O-(4-methoxy-benzyl)-oxime (5.4 g, 18 mmol, see Example 10) was dissolve in methanol and sodium cyanoborohydride (60 mmol) was added. The reaction mixture was stirred and concentrated HCl was added dropwise until the pH was maintained 3. The reaction was stirred for 2 hours maintaining the pH below 3 by adding additional HCl as necessary. The solution was carefully neutralized with saturated sodium hydrogen carbonate and the amine was extracted with ethyl acetate (150 mL, three times). The combined organics were dried over sodium sulfate and concentrated on the rotary evaporator. The crude amine was purified on silica gel (hexanes:ethyl acetate; 2/8) to provide 4.9 g (90%) of the title compound as an amorphous white solid, Mp 52-55° C. (ethyl acetate/hexanes).

¹HNMR (DMSO, 300 MHz) δ 7.28 (d, J=9.0 Hz, 2H), 7.13 (d, J=9.0 Hz, 1H), 6.87 (d, J=9.0 Hz, 2H), 6.43 (m, 2H), 4.64 (s, 2H), 3.99 (s, 2H), 3.80 (s, 3H), 3.79 (s, 3H), 3.78 (s, 3H). LS/MS C17H21NO4 calculated for: (M+H⁺) 304, found: 304.

### Example 12

### Methyl (2R)-2-({N-[(2,4-dimethoxyphenyl)methyl]-N-[(4-methoxyphenyl)methoxy]carbamoyl}methyl)-4-methylpentanoate

Methyl (2R)-2-({N-[(2,4-dimethoxyphenyl)methyl]-N-[(4-methoxyphenyl)methoxy]carbamoyl}methyl)-4-methylpentanoate was prepared using the procedure in Example 7 from (R)-2-Isobutyl succinic acid 1-methyl ester (11.0 g, 5.3 mmol), [(2,4-dimethoxy phenyl)methyl][(4-methoxyphenyl)methoxy]amine (1.82 g, 6 mmol), EDC HCl (2.03 g 10.6 mmol), anhydrous hydroxylbenzotriazole (0.735 g, 5.3 mmol), Diisopropylethylamine (1.84 mL, 10.6 mmol) and methylenechloride (30 mL). Yield: 1.8 g (76%). MS (M+H⁺) 474.

### Example 13

### (2R)-2-({N-[(2,4-dimethoxyphenyl)methyl]-N-[(4-methoxyphenyl)methoxy]carbamoyl}methyl)-4-methylpentanoic acid, sodium salt

(2R)-2-({N-[(2,4-dimethoxyphenyl)methyl]-N-[(4-methoxyphenyl)methoxy]carbamoyl}methyl)-4-methylpentanoic acid, sodium salt was prepared from Methyl (2R)-2-({N-[(2,4-dimethoxyphenyl)methyl]-N-[(4-methoxyphenyl)methoxy]carbamoyl}methyl)-4-methylpentanoate (1.51 g, 3.2 mmol) using the procedure similar to Example 6. After completion of the reaction, the reaction mixture was cooled in ice bath and the neutralized with 1N HCl to pH=7 and then basified with sodium bicarbonate solution. The mixture was purified using a short column of RP-C-18 silica gel, and eluted with 30% Methanol in water. Yield: 0.95 g (57%). MS (M−H⁺)=458.

### Example 14

### 2-[(2R)-2-({N-[(2,4-dimethoxyphenyl)methyl]-N-[(4-methoxyphenyl)methoxy]carbamoyl}methyl)-4-methylpentanoylamino](2S)—N-((1S)-1-carbamoyl-3-methylbutyl)-N′-[2-(4-phenylphenyl)ethyl]pentane-1,5-diamide

2-[(2R)-2-({N-[(2,4-dimethoxyphenyl)methyl]-N-[(4-methoxyphenyl)methoxy]carbamoyl}methyl)-4-methylpentanoylamino](2S)—N-((1S)-1-carbamoyl-3-methylbutyl)-N-[2-(4-phenyl phenyl)ethyl]pentane-1,5-diamide was prepared from (2S)—N-((1S)-1-carbamoyl-3-methylbutyl)-2-amino-N′-[2-(4-phenylphenyl)ethyl]pentane-1,5-diamide (285 mg, 0.6 mmol), Methyl (2R)-2-({N-[(2,4-dimethoxyphenyl)methyl]-N-[(4-methoxyphenyl)methoxy]carbamoyl}methyl)-4-methylpentanoate (240 mg, 0.5 mmol), EDC HCl (192 mg, 1 mmol), anhydrous hydroxybenzotriazole (68 mg, 0.5 mmol), diisopropylethylamine (191 μL, 1.1 mmol), and DMF (4 mL) using the procedure as in Example 7. Yield: 395 mg (88%).

¹H NMR: (300 MHz, CDCl₃): 8.42 δ (1H bs); 7.78 δ (1H d); 7.40 δ (12H m); 6.86 δ (2H d); 6.42 δ (2H m) 6.08 δ (1H b); 5.26 δ (1H m); 4.73 δ (4H m); 4.20 δ (1H m) 3.76-6 (9H m); 3.67 δ (2H b); 2.80 δ (4H m); 2.62 δ (1H dm); 2.37 δ (2H m) 3.17 δ (1H m); 1.68 δ (6H m); 1.38 δ (2H m); 0.94 δ (6H m).

### Example 15

### Synthesis of Compound 2: {2-[2-N-hydroxycarbamoylmethyl)(2R)-4-methylpentanoylamino](2S)—N-((1S)-1-carbamoyl-3-methylbutyl)-N′-[2-(4-phenylphenyl)ethyl]pentane-1,5-diamide}

The product from Example 14 (0.3 g, 0.34 mmoles) was treated with a 4/1 (v/v) mixture of trifluoroacetic acid and trimethylsilyl bromide under drying tube and the mixture was stirred for two hours. The solvent was rotovaped, the residue was triturated with ethylacetate and the residue was put on a short column of RP C-18 silica gel and eluted with a mixture of water and methanol, increasing the methanol concentration from zero to 80%. The compound was eluted in 80%. Yield 25 mg (12.5%).

¹H NMR: (300 MHz, DMSO-d6): 10.46 δ (1H s); 8.80 δ (1H s); 8.16 δ (1H d); 7.92 δ (1H t); 7.63 δ (5H m) 7.48 δ (2H t); 7.34 δ (4H m); 7.00 δ (1H s); 4.20 δ (2H m); 3.30 δ (2H t); 2.74 δ (3H t); 2.16 δ (6H m); 1.56 δ (5H s); 0.90 δ (12H m).

### Example 16

### 3-Formyl-heptanoic acid ethyl ester

Hexanal (5 g, 50 mmol) and diisobutyl amine (6.5 g, 50 mmol) were dissolved in 200 mL of benzene and refluxed for 8 hours under a Dean Stark apparatus. The solution was cooled to room temperature and bromo ethyl acetate (12.5 g, 75 mmol) was added and the reaction was refluxed for 20 hours. The reaction was cooled to room temperature and 20 mL of a 3/1 (water/acetic acid) solution was added and the mixture was heating under reflux for two hours. The mixture was cooled diluted with saturated sodium carbonate and the organic layer was collected. The aqueous layer was extracted two times with ether. The combined organics were dried over sodium sulfate, filtered and concentrated on the rotary evaporator. The crude residue was purified on silica gel (Ethyl acetate/hexane: 5/95) to afford 7 g of the title compound (75%).

¹HNMR (CDCl₃, 300 MHz) δ 9.75 (s, 1H), 4.17 (q, J=6.9 Hz, 2H), 2.86-2.70 (m, 2H), 2.43 (dd, J=5.1, 5.1 Hz, 1H), 1.78-1.27 (m, 9H), 0.94 (t, J=6.3 Hz, 3H).

### Example 17

### Compound 3: (3-(Benzyloximino-methyl)-heptanoic acid hydroxyamide)

3-Formyl-heptanoic acid ethyl ester (300 mg, 1.7 mmol) and O-Benzyl hydroxylamine hydrochloride (270 mg, 1.7 mmol) were dissolved in THF. Pyridine (268 mg, 3.4 mmol) was added and the mixture was stirred for 30 minutes. The reaction was diluted with saturated ammonium chloride and extracted three times with 50 mL of ethyl acetate. The combined organics were dried over sodium sulfate, filtered and concentrated on the rotary evaporator. The residue was purified on silica gel (ethyl acetate/hexanes; 317) to give 150 mg of 3-(Benzyloxyimino-methyl)-heptanoic acid ethyl ester (30%). 3-(Benzyloxyimino-methyl)-heptanoic acid ethyl ester (150 mg, 0.49 mmol) was converted to the title compound using the procedure in Example 2 (hydroxylamine hydrochloride/KOH in dry methanol) to yield 72 mg (53%) of Compound 3. LC/MS C15H₂₂N₂O₃, calculated for M−H⁻): 277, found: 277.

### Example 18

### Compound 4: {2-(Hydroxycarbamoylmethyl-amino)-4-methyl-pentanoic acid [1-(1-carbamoyl-ethylcarbamoyl)-2-naphthalen-2-yl-ethyl]-amide}

2-Amino-propionamide (L isomer) (1.2 g, 9.5 mmol) and 2-tert-butoxycarbony-(L)-amino-3-naphthalen-2-yl-propionic acid (3 g, 9.5 mmol were coupled using the procedure in Example 3. The compound was isolated, after aqueous wash, by crystallization to afford 2.9 g (79%)of (t-Butoxycarbonyl-L-naphthylalanyl-L-alanine) amide. The BOC group was removed by treatment with 50% TFA in dichloromethane for 30 minutes. The solvent was removed on the rotary evaporator and the product was dissolved in toluene and the toluene removed on the rotary evaporator, this was repeated three times. The TFA salt (7.5 mmol) was taken up in dichloromethane, cooled to 0° C., and neutralized with triethylamine to yield (L-naphthylalanyl-L-alanine) amide. The free amine was coupled to N-(t-butoxycarbonyl)-L-leucine (1.8 g, 7.5 mmol) using the procedure in Example 3 to yield {N-(t-butoxycarbonyl)-L-leucylL-naphthylalanyl-L-alanine}amide. This tripeptide was deprotected with 50% TFA in dichloromethane and neutralized as previously described for afford 2.2 g of 2-Amino-4-methyl-pentanoic acid [1-(1-carbamoyl-ethylcarbamoyl)-2-naphthalen-2-yl-ethyl]-amide (88%).

2-Amino-4-methyl-pentanoic acid [1-(1-carbamoyl-ethylcarbamoyl)-2-naphthalen-2-yl-ethyl]-amide (100 mg, 0.25 mmol) was dissolved in DMP, triethylamine (30 mg, 0.3 mmol) and N-Benzyloxy-2-bromo-acetamide (75 mg, 0.3 mmol) was added and the mixture was stirred for 72 hours at 50° C. The reaction mixture was diluted with ethyl acetate and washed with saturated ammonium chloride (aq). The organic layers were dried over sodium sulfate and concentrated on the rotary evaporator. The crude residue was purified on silica gel (70% ethyl acetate in hexanes) to yield 90 mg of 2-[(Benzyloxycarbamoylmethyl)-amino]-4-methyl-pentanoic acid [1-(1-carbamoyl-ethylcarbamoyl)-2-naphthalen-2-yl-ethyl]-amide (65%).

The benzyl group was removed by palladium-catalyzed hydrogenolysis (10% Pd/C) in ethanol. The palladium was removed by filtration through a plug of celite. The ethanol was removed on the rotary evaporator on the title compound was re-crystallized from hot methanol to yield 55 mg of Compound 4 as a white solid (71%). Mp=205-212° C. (methanol) Ms: C₂₄H₃₃N₅O₅ calculated for (M+H⁺):472, found: 472.

### Example 19

### (tert-butoxy)-N-indan-2-ylcarboxamide

In an ice bath, indane-2-ylamine (1.94 mL, 15 mmol) and then added dioxane (20 mL), 1M NaOH (30 mmol), and tert-butyl (tert-butoxycarbonyloxy)formate (4.9 g, 22.5 mmol) respectively. After 1 hour, the pH was adjusted to 9, and stirred overnight. The product precipitated out and was collected by filtration using 1N HCl (20 mL), H₂O (20 mL), and Hexanes to the isolation of the title compound (3.34 g) in 95% yield.

¹H NMR (300 MHz, d₆-DMSO): δ 7.147 (5H, m), 4.180 (1H, m), 3.089 (2H, q), 2.75 (2H, q), 1.390 (9H, s).

### Example 20

### (tert-butoxy)-N-indan-2-yl-N-methylcarboxamide

Under dry conditions, the product from Example 19 (3.14 g, 13.4 mmol) was stirred in DMF (20mL). NaH (0.339 g, 10.1 mmol) was added to the solution and stirred for 30 min. Iodomethane (2 mL, 20.1 mmol) was added to the mixture and stirred for overnight. DMF was removed by rotavap and high vacuum. The residue was extracted with EtOAc and washed with NaCl (aq). EtOAc was dried over Na₂SO4, filtered, and concentrated to the isolation of the title product (1.2 g) in 36% yield. MS (M−57+H)⁺ 192.

### Example 21

### Compound 5: 2-(N-hydroxycarbamoylmethyl)(2R)—N—[(N-indan-2-yl-N-methylcarbamoyl)(4-phenylphenyl)methyl]-4-methylpentanamide

2-[(tert-butoxy)carbonylamino]-2-(4-phenylphenyl)acetic acid (9.8 g, 30 mmol) and phenylmethan-1-ol (4.67 mL, 45 mmol) in 100mL of methylene chloride was added NMM (6.58 mL, 60 mmol), then EDC (11.52 g, 60 mmol) and DMAP (732 mg, 6 mmol) at 0° C. The reaction mixture was stirred at room temperature for overnight. The methylene chloride was evaporated (rotavap) under vacuum. The crude residue went through acid base work up to yield 13.62 g (73%) of phenylmethyl 2-[(tert-butoxy)carbonylamino]-2-(4-phenylphenyl)acetate as a white solid. MS (M+H-Boc)⁺ 318.

The removal of t-Boc of phenylmethyl 2-[(tert-butoxy)carbonylamino]-2-(4-phenylphenyl)acetate (13.58 g, 32.5 mmol)and 4N HCl in Dioxane (60 mL) to yield 10.6 g (92%) of phenylmethyl 2-amino-2-(4-phenylphenyl)acetate hydrochloride as white solid. MS (M+H)⁺ 318.

Using the procedure of Example 3, phenylmethyl 2-amino-2-(4-phenylphenyl)acetate hydrochloride (9.4 g, 26.5 mmol), (2R)-2-(hydroperoxycarbonylmethyl)-4-methylpentanoic acid (5 g, 26.5 mmol), EDC (10.2 g, 53 mmol), HOBt (4 g, 26.5 mmol), NMM instead of DIEA (8.7 mL, 79.5 mmol) and dichloromethane (50 mL) to yield 11.4 g (88%) of methyl (3R)-5-methyl-3-(N-{[benzyloxycarbonyl](4-phenylphenyl)methyl}carbamoyl)hexanoate as a light yellow solid. MS (M+H)⁺ 488.

The removal of the benzyl group from the benzyl ester was done by using methyl (3R)-5-methyl-3-(N-{[benzyloxycarbonyl](4-phenylphenyl)methyl}carbamoyl)hexanoate (6 g, 12.3 mmol), 10% palladium on carbon (600 mg, 10% of ester), dry tetrahydrofuran (10 mL), methanol (150 mL) to yield 4.75 g (97%) of 2-{(2R)-2′-[(methoxycarbonyl)methyl]-4-methylpentanoylamino}-2-(4-phenylphenyl)acetic acid as a white solid. MS (M+H)⁺ 398.

Using the procedure of Example 3, Indan-2-ylmethylamine hydrochloride (694 mg, 3.77 mmol), 2-{(2R)-2-[(methoxycarbonyl)methyl]-4-methylpentanoylamino}-2-(4-phenylphenyl)acetic acid (1.5 g, 3.77 mmol), EDC (2.17 g, 11.31 mmol), HOBt (577 mg, 3.77 mmol), NMM instead of DIEA (1.65 mL, 15.08 mmol) and dichloromethane (50 mL) to yield 250 mg (13%) of methyl (3R)-3-{N—[(N-indan-2-yl-N-methylcarbamoyl)(4-phenylphenyl)methyl]carbamoyl}-5-methylhexanoate as a yellow liquid. MS (+H)⁺ 527.

Using the procedure of Example 2, KOH (1.6 g, 28.5 mmol) was dissolved in dry MeOH (8 mL). NH₂OH—HCl (1.25 g, 18 mmol) was dissolved in dry MeOH (12 mL) and cooled to 0° C. The KOH solution was poured into the NH₂OH—HCl solution and stirred for 1 hour. Methyl (3R)-3-{N—[(N-indan-2-yl-N-methylcarbamoyl)(4-phenylphenyl)methyl]carbamoyl}-5-methylhexanoates (246 mg, 0.46 mmol) was dissolved in dry MeOH (0.94 mL). The KOH/NH₂OH—HCl solution (3.74 mL) was filtered into the solution of Methyl (3R)-3-{N—[(N-indan-2-yl-N-methylcarbamoyl)(4-phenylphenyl)methyl]carbamoyl}-5-methylhexanoate and stirred for 1-2 hours at room temperature. Reaction completion was detected by LCMS. The reaction mixture was concentrated in the removal of MeOH. The residue was dissolved in H₂O (3mL) and acidified to pH=6 with 6N HCl, and neutralized with saturated NaHCO₃ (pH=9). The product precipitated and was collected by filtration. The crude product was purified by silica gel chromatography (water/methanol, 30:70) to the isolation of 2-(N-hydroxycarbamoylmethyl)(2R)—N—[(N-indan-2-yl-N-methylcarbamoyl)(4-phenylphenyl)methyl]-4-methylpentanamide (0.03 g) in 13% yield (diastereoisomeric mixture of 23M7 ratio).

R_{f}=0.65 (ethyl acetate/methanol, 4:1).

¹H NMR 1:1 Mixture (300 MHz, d₆-DMSO): δ 10.394 (1H, s), 8.714 (2H, m), 7.499 (9H, m), 7.157 (4H, m), 6.078 (0.5H, m), 5.85 (0.5H, m), 5.36 (0.5H, m), 4.965 (0.5H, m), 2.839 (8H, m) 2.148 (2H, m), 1.369 (2H, m), 0.835 (6H, m).

### Example 22

### [4-(3,4-dichlorophenyl)phenyl]methylamine

In a 10 mL glass tube were placed (4-bromophenyl)methylamine (0.186 g, 1.0 mmol), 3,4-dichlorophenylboronic acid (0.191 g, 1.0 mmol), bis(triphenylphosphine)palladium (11) chloride (0.035 g, 0.05 mmol), 1M Na₂CO₃ (2 mL), CH₃CN (2 mL) and a magnetic stir bar. The vessel was sealed with a septum and placed into the microwave cavity. Microwave irradiation was used, and the reaction mixture was keep at 150° C. for 250 seconds. After the mixture was allowed to cool to room temperature, the reaction vessel was opened and the contents were concentrated under vacuum. The crude solid residue washed by water, hexanes, and dried under vacuum to give 0.158 g (62%) of [4-(3,4-dichlorophenyl)phenyl]methylamine as yellow solid. MS (N+H)⁺ 252, 254.

### Example 23

### 2-[(ethoxycarbonyl)methyl]-4-methylpentanoic acid

To a solution of di-tert-butyl 2-(ethoxycabonylmethyl)malonate (15.10 g, 49.9 mmol) in 50 mL of dry DMF was added sodium hydride (1.998 g, 49.9 mmol) at room temperature under an nitrogen atmosphere. When evolution of hydrogen ceased, the 1-iodo-2-methylpropane (11.57 mL, 99.9 mmol) was added, and the mixture was stirred for overnight at 65° C. The DMF was evaporated under vacuum. The residue was diluted with water and extracted with ethyl acetate. The organic layer washed with brine, dried (Na₂SO₄), concentrated, and purified by flash chromatography (ethyl acetate/hexanes, 100% hexanes to 1:30) to give the light yellow oil of the tert-butyl ethyl 2-[(tert-butyl)oxycarbonyl]-2-(2-methylpropyl)butane-1,4-dioate (14.60 g) in 82% yield.

¹H NMR (300 MHz, CDCl₃): δ 4.12 (2H, q), 2.94 (2H, s), 1.90 (2H, d), 1.57-1.49 (1H, m), 1.45 (18H, s), 1.24 (3H, t), 0.89 (6H, d).

tert-Butyl ethyl 2-[(tert-butyl)oxycarbonyl]-2-(2-methylpropyl)butane-1,4-dioate (13.60 g, 37.9 mmol) in 50 mL of TFA was stirred at room temperature for 2 hours. After TFA was removed to leave 9.33 g of 2-[(ethoxycarbonyl)methyl]-2-(2-methylpropyl)propanedioic acid as dark yellow oil.

¹H NMR (300 MHz, CDCl₃): δ 9.28 (2H, brs), 4.15 (2H, q), 3.13 (2H, s), 1.90 (2H, d), 1.77-1.68 (1H, m), 1.25 (3H, t), 0.93 (6H, d).

The 2-[(ethoxycarbonyl)methyl]-2-(2-methylpropyl)propanedioic acid (9.33 g, 37.9 mmol) was heated in the kugelrohr oven at 150-155° C. for 20 minutes. When evolution of carbon dioxide ceased, the residue yellow oil was the 2-[(ethoxycarbonyl)methyl]-4-methylpentanoic acid (6.05 g, 79%).

¹H NMR (300 MHz, CDCl₃): δ 10.01 (1H, br s), 4.14 (2H, q), 2.95-2.90 (1H, m), 2.68 (1H, dd), 2.44 (1H, dd), 1.68-1.59 (2H, m), 1.34-1.27 (1H, m), 1.25 (3H, t), 1.00-0.90 (6H, m).

### Example 24

### Compound 6: N1-(3′,4′-Dichloro-biphenyl-4-ylmethyl)-N4-(hydroxyl)-2-isobutyl-succinamide

To the product from Example 22 (0.125 g, 0.49 mmoles) and the product from Example 23 (0.100 g, 0.49 mmoles) in 5mL of methylene chloride was added HOBt (0.067 g, 0.49 mmol), followed by NMM (0.11 mL, 0.99 mmol), then EDC (0.190 g, 0.49 mmol) at 0° C. The reaction mixture was stirred overnight at room temperature under nitrogen. The methylene chloride was evaporated (rotavap) under vacuum. The crude residue was purified by flash chromatography (ethyl acetate/hexanes, 100% hexanes to 1:4) to give 0.130 g (61%) of ethyl 3-(N {[4-(3,4-dichlorophenyl)phenyl]methyl}carbamoyl)-5-methylhexanoate as colorless oil. MS (M+H)⁺ 436, 438.

KOH (1.60 g, 28.5 mmol) was dissolved in dry MeOH (8 mL). NH₂OH—HCl (1.251 g, 18.0 mmol) was dissolved in dry MeOH (12 mL) and cooled to 0° C. The KOH solution was poured into the NH₂OH—HCl solution and stirred for 1 hour at 0° C. The ethyl 3-(N-{[4-(3,4-dichlorophenyl)phenyl]methyl}carbamoyl)-5-methylhexanoate (0.096 mg, 0.22 mmol) was dissolved in dry MeOH (0.44 mL). The KOH/NH₂OH—HCl solution (1.76 mL) was filtered into this solution and stirred for 1 h at room temperature. Reaction completion was detected by LC/MS. The reaction mixture was concentrated in the removal of MeOH. The residue was dissolved in H₂O (3mL) and acidified to pH=6 with 1N HCl, and neutralized with saturated NaHCO₃ (pH=9). The product precipitated and was collected by filtration. Purification of the product was done by recrystallization from 2-propanol, yielding the title product (0.08 g) in 86% yield. MS (M−H)-421, 423.

### Example 25

### 2-bromo-1-(2,3,4,5,6-pentafluorophenoxy)ethane

In a 10 mL glass tube were placed 2-(2,3,4,5,6-pentafluorophenoxy)ethan-1-ol (0.250 g, 1.1 mmol), NBS (0.215 g, 1.2 mmol), triphenylphosphine (0.316 g, 1.2 mmol), 4 mL of CH₃CN and a magnetic stir bar. The vessel was sealed with a septum and placed into the microwave cavity. Microwave irradiation was used, and the reaction mixture was keep at 125° C. for 700 seconds. After the mixture was allowed to cool to room temperature, the reaction vessel was opened and the contents were concentrated under vacuum. The crude residue was purified by flash chromatography (ethyl acetate/hexanes, 100% hexanes to 1:4) to give 0.330 g (99%) of 2-bromo-1-(2,3,4,5,6-pentafluorophenoxy)ethane as colorless oil.

¹H NMR (300 MHz, CDCl₃): δ 4.45 (2H, t), 3.62 (2H, t).

### Example 26

### Compound 7: 3-(1-Methylcarbamoyl-2-naphthalen-2-yl-ethylcarbamoyl)-5-pentafluorophenyloxy-pentanoic acid

2-[(ethoxycarbonyl)methyl]-4-(2,3,4,5,6-pentafluorophenoxy)butanoic acid: The title compound was prepared using the procedure for Example 23 from 2-bromo-1-(2,3,4,5,6-pentafluorophenoxy)ethane (2.00 g, 6.9 mmol), di-tert-butyl 2-(ethoxycabonylmethyl) malonate (2.08 g, 6.9 mmol) and sodium hydride (0.275 g, 6.9 mmol). Yield: 1.65 g (70%).

¹H NMR (300 MHz, CDCl₃): δ 4.26 (24, t), 4.16 (2H, q), 3.19 (1H, m), 2.80 (1H, dd), 2.63 (1H, dd), 2.22 (1H, m), 2.06 (1H, m), 1.26 (3H, t).

To a solution of 2-[(ethoxycarbonyl)methyl]-4-(2,3,4,5,6-pentafluorophenoxy)butanoic acid (0.132 g, 0.37 mmol) and (2S)-2-amino-N-methyl-3-(2-naphthyl)propanamide hydrochloride (0.098 g, 0.37 mmol) in 5 mL of methylene chloride was added HOBt (0.050 g, 0.37 mmol), followed by NMM (0.122 mL, 1.11 mmol), then EDC (0.142 g, 0.74 mmol) at 0° C. The reaction mixture was stirred at room temperature under nitrogen for overnight. The methylene chloride was evaporated (rotavap) under vacuum. The crude residue was purified by flash chromatography (ethyl acetate/hexanes, 100% hexanes to 2:1) to give 0.170 g (81%) of ethyl 3-{N-[(1S)-1-(N-methylcarbamoyl)-2-(2-naphthyl)ethyl]carbamoyl}-5-(2,3,4,5,6-pentafluorophenoxy)pentanoate as white solid. MS (M+H)⁺ 567.

To a solution of ethyl 3-{N-[(1S)-1-(N-methylcarbamoyl)-2-(2-naphthyl)ethyl]carbamoyl}-5-(2,3,4,5,6-pentafluorophenoxy)pentanoate (0.170 g, 0.30 mmol) in 10n2L of THF was added 20 mL of 0.25M LiOH solution (in MeOH/H₂O, 75/25). The mixture was stirred 2 hours at room temperature and concentrated in vacuo. The residue added water, then added 1N HCl aq. solution until pH<7. The solid was collected by filter and washed by water, then dried under vacuum give 0.160 g (99%) of 3-{N-[(1S)-1-(N-methylcarbamoyl)-2-(2-naphthyl)ethyl]carbamoyl}-5-(2,3,4,5,6-pentafluorophenoxy)pentanoic acid as white solid. mp. 68-69° C.; MS (M+H)⁺ 539; (M−H)⁻ 537.

### Example 27

### Compound 8: N4-Hydroxy-N1-(1-methylcarbamoyl-2-naphthalen-2-yl-ethyl)-2-(2-pentafluorophenyloxy-ethyl)-succinamide

To a solution of Compound 7 (0.122 g, 0.23 mmol, see Example 26) and 2H-3,4,5,6-tetrahydropyran-2-yloxyamine (0.027 g, 0.23 mmol) in 5mL of methylene chloride was added HOBt (0.031 g, 0.23 mmol), followed by NMM (0.0.05 mL, 0.45 nmol), then EDC (0.087 g, 0.45 mmol) at 0° C. The reaction mixture was stirred at room temperature under nitrogen for overnight. The methylene chloride was evaporated (rotavap) under vacuum. The residue was diluted with water and extracted with ethyl acetate. The ethyl acetate layer washed with 1N HCl (2×15 mL), saturated sodium bicarbonate (2×15 mL) and brine (15 mL), and dried over anhydrous sodium sulphate and rotovaped to give 0.130 g (89%) of N′-(2H-3,4,5,6-tetrahydropyran-2-yloxy)-N-[(1S)-1-(N-methylcarbamoyl)-2-(2-naphthyl)ethyl]-2-[2-(2,3,4,5,6-pentafluorophenoxy)ethyl]butane-1,4-diamide as white solid. MS (M−H)-636.

A mixture of N′-(2H-3,4,5,6-tetrahydropyran-2-yloxy)-N-[(1S)-1-(N-methylcarbamoyl)-2-(2-naphthyl)ethyl]-2-[2-(2,3,4,5,6-pentafluorophenoxy)ethyl]butane-1,4-diamide (0.130 g, 0.2 mmol) and PTSA (0.060 g) in dry methanol (35 mL) was stirred at room temperature for overnight. Then added 0.25 M LiOH solution until pH>7. The methanol was evaporated, and the residue was diluted with water and extracted with ethyl acetate. The organic layer washed with brine, dried (Na₂SO₄), and evaporated. The crude residue was purified by C-18 flash chromatography (H₂O/MeOH, 100% H₂O to 2:8) to give 0.076 g (69%) of 2-(N-hydroxycarbamoylmethyl)-N-[(1S)-1-(N-methylcarbamoyl)-2-(2-naphthyl)ethyl]-4-(2,3,4,5,6-pentafluorophenoxy)butanamide as white solid. MS (M+H)⁺ 554; (M−H)⁻ 552.

### Example 28

### Methyl (3R)-5-methyl-3-(N-{(9-methylcarbazol-3-yl)[N-benzylcarbamoyl]methyl}carbamoyl)hexanoate

In a 10 mL glass tube were placed (9-methylcarbazol-3-yl)formaldehyde (0.209 g, 1.0 mmol), 2M solution of ammonia in methanol (1 mL, 2.0 mmol), benzyl isocyanide (0.122 mL, 1 mmol), (2R)-2-[(methoxycarbonyl)methyl]-4-methylpentanoic acid (0.188 g, 1 mmol), CH₃OH (4 mL) and a magnetic stir bar. The vessel was sealed with a septum and placed into the microwave cavity. Microwave irradiation was used, and the reaction mixture was keep at 140° C. for 2400 seconds. After the mixture was allowed to cool to room temperature, the reaction vessel was opened and the contents were concentrated under vacuum. The crude residue was purified by flash chromatography (ethyl acetate/hexanes, 100% hexanes to 1:2) to give 0.126 g (25%) of methyl (3R)-5-methyl-3-(N-{(9-methylcarbazol-3-yl)[N-benzylcarbamoyl]methyl}carbamoyl)hexanoate. MS (M+H)⁺ 514.

### Example 29

### Compound 9: 2-(N-hydroxycarbamoylmethyl)(2R)-4-methyl-N-{(9-methylcarbazol-3-yl)[N-benzylcarbamoyl]methyl}pentanamide

KOH (1.60 g, 28.5 mmol) was dissolved in dry MeOH (8 mL). NH₂OH—HCl (1.251 g, 18.0 mmol) was dissolved in dry MeOH (12 mL) and cooled to 0° C. The KOH solution was poured into the NH₂OH—HCl solution and stirred for 1 hour at 0° C. The methyl (3R)-5-methyl-3-(N-{(9-methylcarbazol-3-yl)[N-benzylcarbamoyl]methyl}carbamoyl)hexanoate (0.126 mg, 0.24 mmol) was dissolved in dry MeOH (0.49 mL). The KOH/NH₂OH—HCl solution (1.96 mL) was filtered into this solution and stirred for 2 hours at room temperature. Reaction completion was detected by LC/MS. The reaction mixture was concentrated in the removal of MeOH. The residue was dissolved in H₂O (3 mL) and acidified to pH=6 with 1N HCl, and neutralized with saturated Na₂HCO₃ (pH=9). The product precipitated and was collected by filtration. The crude product was purified by C-18 flash chromatography (H₂O/MeOH, 100%, H₂O to 3:7) to give 0.100 g (81%) of 2-(N-hydroxycarbamoylmethyl)(2R)-4-methyl-N-{(9-methylcarbazol-3-yl)[N-benzylcarbamoyl]methyl}pentanamide. MS (M+H)⁺ 515; (M−H)-513.

### Example 30

### Compound 10: Trans-2-(N-{-1-[N-(-1-carbamoylpropyl)carbamoyl]-2-naphthylethyl}carbamoyl)cyclohexanecarboxylic acid

(2S)—N-((1S)-1-carbamoylpropyl)-2-[(tert-butoxy)carbonylamino]-3-naphthylpropan amide was prepared by treating (S)—N-Boc-1-naphthylalanine (630 mg, 2 mmol), and S-2-aminobutyramide (204 mg, 2 mmol) with EDC HCl (576 mg, 3 mmol), anhydrous 1-hydroxybenzotriazole (306 mg, 2 mmol) and Diisopropylethylamide (522 μL, 3 mmol) in DMF. The mixture was subjected to microwave heating in Personal Chemistry microwave synthesizer at 160° C. for 500 seconds. The solvent DMF was rotovaped under vacuum and the residue was taken in EtOAc and washed with 1N HCl (2×15 mL), saturated sodiumbicarbonate solution (2×15 mL) and brine (2×15 mL). The EtOAc solution was dried over anhydrous sodium sulphate and rotovaped. The residue on trituration with hexane gave a solid. Yield: 635 mg (79%). MS (M+H)⁺ 400; (M−45)⁻ 444.

(2S)—N-((1S)-1-carbamoylpropyl)-2-amino-3-naphthyl propanamide was prepared by treating (2S)—N-((1S)-1-carbamoylpropyl)-2-[(tert-butoxy)carbonylamino]-3-naphthylpropanamide (798 mg, 2 mmol) with 4N HCl in Dioxane (10 mL) and the mixture was stirred for 30 minutes. The Dioxane was rotovaped, the residue was triturated with ether and then suspended in EtOAc and washed with saturated sodium carbonate solution and then with brine. The EtOAc solution was dried over anhydrous sodium sulphate and rotovaped to get a solid. Yield: 300 mg (50%). This material was used without further purification.

(2S)—N-((1S)-1-carbamoylpropyl)-2-amino-3-naphthyl propanamide

A mixture of (2S)—N-((1S)-1-carbamoylpropyl)-2-amino-3-naphthyl propanamide (0.46 g) and trans-1,2-cyclohexanedicarboxylic anhydride (0.45 g) in dichloromethane were stirred overnight. The solvent was rotovaped, the residual solid washed with ethylacetate and filtered. Yield: 0.49 g (72%).

¹H NMR: (300 MHz, DMSO-d6): 11.97 δ (1H d); 8.80 δ (1H s); 8.16 δ (1H d); 7.92 δ (1H t); 7.63 δ (5H m) 7.48 δ (2H t); 7.34 δ (4H m); 7.00 δ (1H s); 4.20 δ (2H m); 3.30 δ (2H t); 2.74 δ (3H t); 2.16 δ (6H m); 156 δ (5H s); 0.90 δ (12H m).

A mixture of (2S)—N-((1S)-1-carbamoylpropyl)-2′-amino-3-naphthyl propanamide (0.46 g) and trans-1,2-cyclohexanedicarboxylic anhydride (0.45 g) in dichloromethane were stirred overnight. The solvent was rotovaped, the residual solid washed with ethylacetate and filtered. Yield: 0.49 g (72%).

¹H NMR: (300 MHz, DMSO-d6): 11.97 δ (1H d); 8.80 δ (1H s); 8.16 δ (1H d); 7.92 δ (1H t); 7.63 δ (5H m) 7.48 δ (2H t); 7.34 δ (4H m); 7.00 δ (1H s); 4.20 δ (2H m); 3.30 δ (2H t); 2.74 δ (3H t); 2.16 δ (6H m); 1.56 δ (5H s); 0.90 δ (12H m).

### Example 31

### Ethyl 3-[(hydrazinothioxomethyl)amino]benzoate

Hydrazine hydrate (0.51 mL, 10.4 mmol) was dissolved in 20 mL of ethanol. This solution was stirred at 0° C. and ethyl 3-isothiocyanatobenzoate (1.800 g, 8.7 mmol) was added dropwise. After complete addition ethyl 3-isothiocyanatobenzoate, the reaction mixture was warmed up to room temperature stirred for 2 hours. After being cooled to 0° C., the mixture was filtered and the solid washed by cold ethanol (10 mL). The solid was crystallized from ethanol to give 1.858 g (89%) of ethyl 3-[(hydrazinothioxomethyl)amino]benzoate as white solid.

¹H NMR (300 MHz, d₆-DMSO): δ 9.25 (1H, s), 8.29 (1H, s), 7.88 (1H, d), 7.68 (1H, d), 7.43 (1H, t), 4.96 (3H, br s), 4.31 (2H, q), 1.32 (3H, t).

### Example 32

### 3-[(5-{3-[3-(trifluoromethyl)phenoxy]phenyl}-1,3,4-thiadiazol-2-yl)amino]benzoate

A solution of {3-[3-(trifluoromethyl)phenoxy]phenyl}formaldehyde (0.556 g, 2.1 mmol) and ethyl 3-[(hydrazinothioxomethyl)amino]benzoate (0.500 g, 2.1 mmol) in dry ethanol (5 mL) under nitrogen refluxed 2 hours. After cooling to room temperature, the mixture was filtered and the solid washed by ethanol. The solid was suspension in dry ethanol (3.5 mL) and iron (III) chloride hexahydrate (1.690 g, 6.3 mmol) was added. The reaction mixture was refluxed for 4 hours, then cooling to room temperature. The solid was collected by filter and washed by ethanol, then crystallized from ethyl acetate/hexanes give 0.556 g (55%) of ethyl 3-[(5-{3-[3-(trifluoromethyl)phenoxy]phenyl}-1,3,4-thiadiazol-2-yl)amino]benzoate as yellow color solid. MS (M+H)⁺: 486; (M−H)⁻: 484.

### Example 33

### Ethyl 3-[aza(3-(2-phenylethyl)-5-{3-[3-(trifluoromethyl)phenoxy]phenyl}(1,3,4-thiadiazolin-2-ylidene))methyl]benzoate and ethyl 3-[(2-phenylethyl) (5-{3-[3-(trifluoromethyl)phenoxy]phenyl}(1,3,4-thiadiazol-2-t)amino]benzoate

To a solution of ethyl 3-[(5-{3-[3-(trifluoromethyl)phenoxy]phenyl}-1,3,4-thiadiazol-2-yl)amino]benzoate (0.300 g, 0.62 mmol) in 5mL of dry DMF was added potassium carbonate (0.171 g, 1.20 mmol) at room temperature under an nitrogen atmosphere. After 5 minute, (2-iodoethyl)benzene (0.27 mL, 1.86 mmol) was injected, and the solution was stirred at 50° C. for overnight. The DMF was evaporated (rotavap) under vacuum. The crude residue was purified by flash chromatography (ethyl acetate/hexanes, 100% hexanes to 1:2) to the isolation of the ethyl 3-[aza(3-(2-phenylethyl)-5-{3-[3-(trifluoromethyl)phenoxy]phenyl}(1,3,4-thiadiazolin-2-ylidene))methyl]benzoate (0.077 g) in 21% yield, R_{f}=0.52 (ethyl acetate/hexanes, 1:2); MS (M+H)⁺: 590 and the ethyl 3-[(2-phenylethyl)(5-{3-[3-(trifluoromethyl)phenoxy]phenyl}(1,3,4-thiadiazol-2-yl))amino]benzoate (0.037 g) in 10% yield, R_{f}=0.38 (ethyl acetate/hexanes, 1:2); MS (M+H)⁺: 590.

### Example 34

### Compound 11: 3-[aza(3-(2-phenylethyl)-5-{3-[3-(trifluoromethyl)phenoxy]phenyl}(1,3,4-thiadiazolin-2-ylidene))methyl]benzoic acid

To a solution of ethyl 3-[aza(3-(2-phenylethyl)-5-{3-[3-(trifluoromethyl)phenoxy]phenyl}(1,3,4-thiadiazolin-2-ylidene))methyl]benzoate (0.075 g, 0.13 mmol) in 4 mL of THF was added 10 mL of 0.25M LiOH solution (in MeOH/H₂O, 75/25). The mixture was stirred overnight at room temperature and concentrated in vacuo. The residue added water, then added 1N HCl aq. solution until pH<7. The solid was collected by filter and washed by water, then dried under vacuum give 0.068 g (95%) of 3-[aza(3-(2-phenylethyl)-5-{3-[3-(trifluoromethyl)phenoxy]phenyl}(1,3,4-thiadiazolin-2-ylidene))methyl]benzoic acid as white solid. mp. 58-59° C.; MS (M+H)⁺ 562; (M−H)-560.

### Example 35

### Compound 12: 3-[(2-phenylethyl)(5-{3-[3-(trifluoromethyl)phenoxy]phenyl}(1,3,4-thiadiazol-2-yl))amino]benzoic acid

To a solution of ethyl 3-[(2-phenylethyl)(5-{3-[3-(trifluoromethyl)phenoxy]phenyl}(1,3,4-thiadiazol-2-yl))amino]benzoate (0.035 g, 0.06 mmol) in 4 mL of THF was added 10 mL of 0.25M LiOH solution (in MeOH/H₂O, 75/25). The mixture was stirred overnight at room temperature and concentrated in vacuo. The residue added water, then added 1N HCl aq. solution until pH<7. The solid was collected by filter and washed by water, then dried under vacuum give 0.030 g (90%) of 3-[(2-phenylethyl)(5-{3-[3-(trifluoromethyl)phenoxy]phenyl}(1,3,4-thiadiazol-2-yl))amino]benzoic acid as light yellow solid. mp. 92-93° C.; MS (M+H)⁺ 562; (M−H)-560.

### Example 36

### Compound 13: 1-[aza(3-(2-phenylethyl)-5-{3-[3-(trifluoromethyl)phenoxy]phenyl}(1,3,4-thiadiazolin-2-ylidene))methyl]benzene-3-carbohydroxamic acid

To a solution of Compound 11 (0.054 g, 0.096 mmol, see Example 33) and 2H-3,4,5,6-tetrahydropyran-2-yloxyamine (0.012 g, 0.106 mmol) in 5 mL of methylene chloride was added HOBt (0.013 g, 0.096 mmol), followed by DMAP (0.023 g, 0.19 mmol), then EDC (0.037 g, 0.19 mmol) at 0° C. The reaction mixture was stirred at room temperature under nitrogen for overnight. The methylene chloride was evaporated (rotavap) under vacuum. The crude residue was purified by flash chromatography (ethyl acetate/hexanes, 100% hexanes to 1:2) to give 0.054 g (85%) of N-(2H-3,4,5,6-tetrahydropyran-2-yloxy){3-[aza(3-(2-phenylethyl)-5-{3-[3-(trifluoromethyl)phenoxy]phenyl}(1,3,4-thiadiazolin-2-ylidene))methyl]phenyl}carboxamide as colorless oil. MS (M+H)⁺ 661.

A mixture of N-(2H-3,4,5,6-tetrahydropyran-2-yloxy) {3-[aza(3-(2-phenylethyl)-5-{3-[3-(trifluoromethyl)phenoxy]phenyl}(1,3′,4-thiadiazolin-2-ylidene))methyl]phenyl}carboxamide (0.054 g, 0.08 mmol) and PTSA (0.030 g) in dry methanol (20 mL) was stirred at room temperature for overnight. Then added 0.25M LiOH solution until pH>7. The methanol was evaporated, and the residue was diluted with water and extracted with ethyl acetate. The organic layer washed with brine, dried (Na₂SO₄), and evaporated to give 0.042 g (90%) of 1-[aza(3-(2-phenylethyl)-5-{3-[3-(trifluoromethyl)phenoxy]phenyl}(1,3,4-thiadiazolin-2-ylidene))methyl]benzene-3-carbohydroxamic acid as yellow solid. MS (M+H)⁺ 577; (—H)-575.

### Example 37

### 1-{3-[(5-{3-[3-(trifluoromethyl)phenoxy]phenyl}-1,3,4-thiadiazol-2-yl)amino]phenyl}ethan-1-one

To a solution of {3-[3-(trifluoromethyl)phenoxy]phenyl}formaldehyde (8.29 mL, 40 mmol) in acetone (75 mL) at 0° C. was drop wise added Jones reagent (prepared from 5.34 g of CrO₃, 4.6 mL of conc. H₂SO₄ and 4.4 mL of H₂O) until the orange color persisted. The reaction mixture was slowly warmed up to room temperature and stirred for overnight. The isopropanol (0.5 mL) was added to the reaction mixture and stirred for 2 hours. The reaction mixture was passed through Celite and concentrated (rotavap) under vacuum. The crude residue was purified by flash chromatography (ethyl acetate/hexanes/acetic acid, 100% hexanes to 1:2:0.01) to isolate the 3-[3-(trifluoromethyl)phenoxy]benzoic acid (4.80 g) in 43% yield as white solid.

To a solution of 3-[3-(trifluoromethyl)phenoxy]benzoic acid (5.64 g, 20 mmol) and MeOH (0.81 mL, 20 mmol) in acetonitrile (50 mL) was drop wise added (trimethylsilyl)diazomethane (2M solution in hexane, 15.00 mL, 30 mmol) at room temperature. After stirring overnight, the acetic acid was added to the reaction mixture to quench the excess (trimethylsilyl)diazomethane. The reaction mixture was evaporated (rotavap) under vacuum. The crude residue was purified by flash chromatography (ethyl acetate/hexanes, 100% hexanes to 1:9) to give the methyl 3-[3-(trifluoromethyl)phenoxy]benzoate (5.40 g) in 92% yield as colorless oil.

¹H NMR: (300 MHz, d₁-CDCl₃) δ 7.85 (1H, d), 7.69 (1H, s), 7.49-7.43 (2H, m), 7.38 (1H, d), 7.30-7.23 (2H, m), 7.17 (1H, d), 3.91 (3H, s).

A mixture of methyl 3-[3-(trifluoromethyl)phenoxy]benzoate (1.00 g, 3.38 mmol) and hydrazine monohydrate (0.338 g; 6.78 mmol) was heated in ethanol (5mL) at reflux for overnight. The ethanol was evaporated (rotavap) under vacuum. The crude residue was washed with water and hexanes, then dried under vacuum to give 1-[3-(trifluoromethyl)phenoxy]benzene-3-carbohydrazide as white solid (0.950 g, 95%).

¹H NMR: (300 MHz, DMSO-d₆) δ 9.87 (1H, s), 7.68-7.60 (2H, m), 7.54-7.49 (3H, m), 7.36-7.31 (2H, m), 7.25 (1H, dd), 4.46 (2H, br s).

To a solution of 1-(3-Isothiocyanato-phenyl)-ethanone (0.180 g; 1 mmol) in toluene (10 mL) is added 1-[3-(trifluoromethyl)phenoxy]benzene-3-carbohydrazide (0.300 g; 1 mmol) under argon. The reaction mixture is heated at reflux for two hours. The mixture is filtered while the toluene still is warm. The solid is washed with hexanes and dried to yield N-({[(3-acetylphenyl)amino]thioxomethyl}amino) {3-[3-(trifluoromethyl)phenoxy]phenyl}carboxamide. The product is used for the next step without further purification.

To a slurry mixture of the above carbothioamide in toluene (5 mL) at 0° C. is dropped conc. H₂SO₄ (0.4 mL). The reaction mixture is stirred at room temperature for three hours. The ice-H₂O (50 mL) was added to the reaction mixture. The mixture is neutralized with NH₃H₂O until pH 8 and filtered. The solid product is recrystallized with MeOH/EA to yield 1-{3-[(5-{3-[3-(trifluoromethyl)phenoxy]phenyl}-1,3,4-thiadiazol-2-yl)amino]phenyl}ethan-1-one (0.158 g, 35%) as yellow solid. MP: 130-133° C.

¹H NMR (300 MHz, d₁-CDCl₃): δ 9.67 (1H, s), 8.09 (1H, s), 7.75-7.20 (10H, m), 7.10 (1H, d), 2.65 (3H, s).

### Example 38

### 1-{3-[(5-{3-[3-(trifluoromethyl)phenoxy]phenyl}-1,3,4-thiadiazol-2-yl)amino]phenyl}ethan-1-one and 1-{3-[aza(3-(2-phenylethyl)-5-{3-[3-(trifluoromethyl)phenoxy]phenyl}(1,3,4-thiadiazolin-2-ylidene))methyl]phenyl}ethan-1-one

To a solution of 1-{3-[(5-{3-[3-(trifluoromethyl)phenoxy]phenyl}-1,3,4-thiadiazol-2-yl)amino]phenyl}ethan-1-one (0.200 g, 0.44 mmol) in 4 mL of dry DMF was added potassium carbonate (0.121 g, 0.88 mmol) at room temperature under a nitrogen atmosphere. After 5 minute, (2-iodoethyl)benzene (0.19 mL, 1.30 mmol) was injected, and the solution was stirred at 50° C. for overnight. The DMF was evaporated (rotavap) under vacuum. The crude residue was purified by flash chromatography (ethyl acetate/hexanes, 100% hexanes to 1:2) to the isolation of the 1-{3-[aza(3-(2-phenylethyl)-5-{3-[3-(trifluoromethyl)phenoxy]phenyl}(1,3,4-thiadiazolin-2-ylidene))methyl]phenyl}ethan-1-one (0.066 g) in 27% yield, R_{f}=0.55 (ethyl acetate/hexanes, 1:2); MS M+H)⁺: 560 and the 1-{3-[(2-phenylethyl)(5-{3-[3-(trifluoromethyl)phenoxy]phenyl}(1,3,4-thiadiazol-2-yl))amino]phenyl}ethan-1-one (0.044 g) in 18% yield, R_{f}=0.35 (ethyl acetate/hexanes, 1:2); MS (M+H)⁺: 560.

### Example 39

### Compound 14: 2-((1E)-1-aza-2-{3-[(2-phenylethyl)(5-{3-[3-(trifluoromethyl)phenoxy]phenyl}(1,3,4-thiadiazol-2-yl))amino]phenyl}prop-1-phenyloxy)acetic acid

To a solution of 1-{3-[(5-{3-[3-(trifluoromethyl)phenoxy]phenyl}-1,3,4-thiadiazol-2-yl)amino]phenyl}ethan-1-one (0.200 g, 0.44 mmol) in 4 mL of dry DMF was added potassium carbonate (0.121 g, 0.88 mmol) at room temperature under a nitrogen atmosphere. After 5 minute, (2-iodoethyl)benzene (0.19 mL, 1.30 mmol) was injected, and the solution was stirred at 50° C. for overnight. The DMF was evaporated (rotavap) under vacuum. The crude residue was purified by flash chromatography (ethyl acetate/hexanes, 100% hexanes to 1:2) to the isolation of the 1-{3-[aza(3-(2-phenylethyl)-5-{3-[3-(trifluoromethyl)phenoxy]phenyl}(1,3,4-thiadiazolin-2-ylidene))methyl]phenyl}ethan-1-one (0.066 g) in 27% yield, R_{f}=0.55 (ethyl acetate/hexanes, 1:2); MS (M+H)⁺: 560 and the 1-{3-[(2-phenylethyl)(5-{3-[3-(trifluoromethyl)phenoxy]phenyl}(1,3,4-thiadiazol-2-yl))amino]phenyl}ethan-1-one (0.044 g) in 18% yield, R_{f}=0.35 (ethyl acetate/hexanes, 1:2); MS (M+H)⁺: 560.

### Example 40

### Compound 15: 2-((1E)-1-aza-2-{3-[aza(3-(2-phenylethyl)-5-{3-[3-(trifluoromethyl)phenoxy]phenyl}(1,3,4-thiadiazolin-2-ylidene))methyl]phenyl}prop-1-phenyloxy)acetic acid

To a solution of 1-{3-[aza(3-(2-phenylethyl)-5-{3-[3-(trifluoromethyl)phenoxy]phenyl}(1,3,4-thiadiazolin-2-ylidene))methyl]phenyl}ethan-1-one (0.066 g, 0.12 mmol) and carboxymethoxylamine hemihydrochloride (0.031 g, 0.14 mmol) in 5mL of ethanol was added triethylamine (0.016 mL, 0.14 mmol) at room temperature. The mixture was refluxed for overnight and concentrated in vacuo. The residue was diluted with water and extracted with ethyl acetate. The organic layer washed with brine, dried (Na₂SO₄), concentrated, and purified by flash chromatography (ethyl acetate/hexanes/acetic acid, 1:2:0 to 1:2:0.01) to the isolation of the 2-((1E)-1-aza-2-{3-[aza(3-(2-phenylethyl)-5-{3-[3-(trifluoromethyl)phenoxy]phenyl}(1,3,4-thiadiazolin-2-ylidene))methyl]phenyl}prop-1-phenyloxy)acetic acid (0.063 g) in 85% yield;

MS (M+H)⁺ 633; (M−H)⁻ 631.

The reaction described in above was repeated, but using 0.044 g (0.08 mmol) of 1-{3-[(2-phenylethyl)(5-{3-[3-(trifluoromethyl)phenoxy]phenyl}(1,3,4-thiadiazol-2-yl))amino]phenyl}ethan-1-one, 0.021 g (0.094 mmol) of carboxymethoxylamine hemihydrochloride, and 0.013mL (0.094 mmol) of triethylamine to yield 0.034 g (68%) of 2-((1E)-1-aza-2-{3-[(2-phenylethyl)(5-{3-[3-(trifluoromethyl)phenoxy]phenyl}(1,3,4-thiadiazol-2-yl))amino]phenyl}prop-1-phenyloxy)acetic acid; MS (M+H)⁺ 633.

### Example 41

### Methyl(2-morpholin-4-ylethyl)amine

A mixture of formic acid (67 mL) and Acetic anhydride (24 mL) was added drop wise to a solution 2-morpholin-4-ylethyl amine (6.5 g, 50 mmol) in formic acid (85% 60 mL). The mixture was stirred at room temperature for overnight and then at 50° C. for 2 hours. The solvents were rotovaped under reduced pressure and dried under high vacuum. This residue (N-(2-Morpholin-4-yl-ethyl)-formamide) contained 2 molecules of formic acid. This was used without purification in the next reaction. Yield-6.3 g (80%).

¹H NMR: (300 MHz, CDCL₃): 8.18 δ (1H s); 3.93 δ (4H m); 3.72 δ (2H q); 3.15 δ (6H m). MS, Observed (M+21W)⁺=160.

The N-(2-Morpholin-4-yl-ethyl)-formamide (6.25 g, 25 mmol) was added slowly over a period of 1 hour to a cold and dry suspension of Lithium aluminum hydride (2.85 g) in dry THF (100 mL). The mixture was stirred in cold for 2 hours and then overnight at room temperature. Small amount of water is added to decompose aluminum salt, the solid formed was filtered off, the solid washed with THF. The washings and filtrate were combined and the solvent removed in vacuo to yield the title compound.

Yield: 2.0 g (55%).

### Example 42

### Compound 16: N1-{Biphenyl-4-yl-[methyl-(2-morpholin-4-yl-ethyl)-carbamoyl]-methyl}-N4-hydroxy-2-isobutyl-succinamide

N1-{Biphenyl-4-yl-[methyl-(2-morpholin-4-yl-ethyl)-carbamoyl]-methyl}-N4-hydroxy-2-isobutyl-succinamide was prepared using the procedure as in Example 7 with 2-{(2R)-2-[(methoxycarbonyl)methyl]-4-methylpentanoylamino}-2-(4-phenylphenyl)acetic acid (1.985 g, 5 mmol), methyl(2-morpholin-4-ylethyl)amine (0.87 g, 6 mmol), EDC HCl (1.92 g, 10 mmol), anhydrous hdyroxbenzotriazole (0.68 g, 5 mmol), N-methylmorpholine (1.1 mL, 10 mmol), and methelenechloride (20 mL). After workup a methyl (3R)-5-methyl-3-(N-{[N-methyl-N-(2-morpholin-4-ylethyl)carbamoyl](4-phenylphenyl)methyl}carbamoyl)hexanoate was obtained as a white solid. Yield: 225 mg (10%). This intermediate (220 mg, 042 mmoles) was converted to the title compound using the procedure in Example 2. Yield: 33 mg (15%). MS: (M+H⁺): 525.

### Compound 42: 2-(N-hydroxycarbamoylmethyl)(2R)-4-methyl-N-({N-[(4-methylphenyl)methyl]carbamoyl(4-phenylphenyl)methyl)pentanamide

Methyl (3R)-5-methyl-3-[N-({N-[(4-methylphenyl)methyl]carbamoyl}(4-phenyl phenyl)methyl)carbamoyl]hexanoate was prepared from 2-{(2R)-2-[(methoxycarbonyl)methyl]-4-methylpentanoylamino}-2-(4-phenylphenyl)acetic acid (298 mg, 0.75 mmol), 4-methylbenzylamine (95 μL, 0.75 mmol), EDC HCl (288 mg, 1.5 mmol), HOBt (101 mg, 0.75 mmol), DIEA (261 μL, 1.5 mmol) and dichloromethane (10 mL) using the procedure from Example 3. Yield: 280 mg (56%).

2-(N-hydroxycarbamoylmethyl)(2R)-4-methyl-N-({N-[(4-methylphenyl)methyl]carbamoyl(4-phenylphenyl)methyl)pentanamide (47/53) was prepared from methyl (3R)-5-methyl-3-[N-({N-[(4-methylphenyl)methyl]carbamoyl}(4-phenyl phenyl)methyl)carbamoyl]hexanoate (250 mg, 0.5 mmol) using the procedure from Example 2. Yield: 220 mg (87%). MS (M+H⁺) 502.

### Example 43

### Compound 17: 2-(N-hydroxycarbamoylmethyl)(2R)-4-methyl-N-{(5-phenyl(2-thienyl))[N-benzylcarbamoyl]methyl}pentanamide

Prepared in a manner similar to that described in Example 28 using 0.188 g (1.0 mmol) of 5-phenylthiophene-2-carbaldehyde, 0.188 g (1 mmol) of (2R)-2-[(methoxycarbonyl)methyl]-4-methylpentanoic acid, 0.122 mL (1 mmol) of benzyl isocyanide and 1 mL (2 mmol) of 2M solution of ammonia in methanol to yield 0.088 g (18%) of methyl (3R)-5-methyl-3-(N-{(5-phenyl(2-thienyl))[N-benzylcarbamoyl]methyl}carbamoyl)hexanoate. MS (M+1)⁺ 493; (M+HCO₂⁻)⁻ 537.

Prepared in a manner similar to that described in Example 29 using 0.088 g (0.18 mmol) of methyl (3R)-5-methyl-3-(N-{(5-phenyl(2-thienyl))[N-benzylcarbamoyl]methyl}carbamoyl)hexanoate to yield 0.080 g (90%) of 2-(N-hydroxycarbamoylmethyl)(2R)—N-{(1R,S)(5-phenyl(2-thienyl))[N-benzylcarbamoyl]methyl}-4-methylpentanamide (1:1 mixture of diastereoisomers). MS (M+H)⁺ 494; (M−H)-492.

### Example 44

### Compound 18: 2-(N-hydroxycarbamoylmethyl)(2R)—N-{(9-ethylcarbazol-3-yl)[N-benzylcarbamoyl]methyl}-4-methylpentanamide

Prepared in a manner similar to that described in Example 28 using 0.223 g (1.0 mmol) of 9-ethylcarbazole-3-carbaldehyde, 0.188 g (1 mmol) of (2R)-2-[(methoxycarbonyl)methyl]-4-methylpentanoic acid, 0.122 mL (1 mmol) of benzyl isocyanide and 1 mL (2 mmol) of 2M solution of ammonia in methanol to yield 0.124 g (23%) of methyl (3R)-3-(N-{(9-ethylcarbazol-3-yl)[N-benzylcarbamoyl]methyl}carbamoyl)-5-methylhexanoate. MS (M+H)⁺ 528; (M+HCO₂⁻)⁻ 572.

Prepared in a manner similar to that described in Example 29 using 0.124 g (0.23 mmol) of methyl (3R)-3-(N-{(9-ethylcarbazol-3-yl)[N-benzylcarbamoyl]methyl}carbamoyl)-5-methylhexanoate to yield 0.088 g (71%) of 2-(N-hydroxycarbamoylmethyl)(2R)—N-{(1S,R)(9-ethylcarbazol-3-yl)[N-benzylcarbamoyl]methyl}-4-methylpentanamide (1:1 mixture of diastereoisomers). MS (M+H)⁺ 529; (M−H)-527.

### Example 45

### Compound 19: 2-(N-hydroxycarbamoylmethyl)(2R)—N-{(N-indan-2-ylcarbamoyl)[4-(3-methoxyphenyl)phenyl]methyl}-4-methylpentanamide

To solution of (2S)-2-[(tert-butoxy)carbonylamino]-2-(4-hydroxyphenyl)acetic acid (8.80 g, 32.9 mmol) in 40 mL of MeOH was added cesium carbonate (5.364 g, 16.5 mmol) at room temperature under an nitrogen atmosphere. When evolution of carbon dioxide ceased, the MeOH was evaporated under vacuum. The residue was dissolved in 40 mL of DMF and stirred with benzyl bromide (5.9 mL, 49.4 mmol) at room temperature for overnight. The NMF was evaporated under vacuum. The residue was diluted with water and extracted with ethyl acetate. The organic layer washed with brine, dried (Na₂SO₄), concentrated and purified by flash chromatography (ethyl acetate/hexanes, 100% hexanes to 1:2) to give the white solid of the phenylmethyl (2S)-2-[(tert-butoxy)carbonylamino]-2-(4-hydroxyphenyl)acetate (80.45 g) in 72% yield.

MS (M−H)-356.

To a mixture of phenylmethyl (2S)-2-[(tert-butoxy)carbonylamino]-2-(4-hydroxyphenyl)acetate (8.45 g, 23.6 mmol) in 22 mL of CH₂Cl₂ which contained pyridine (4.78 mL, 59.1 mmol) at −15° C. was added trifluoromethanesulfonic anhydride (4.77 mL, 28.4 mmol). The mixture was stirred for 5 mini the reaction quenched with water and the mixture washed with 0.5N NaOH (2×30 mL), 15% citric acid (2×30 mL) and brine. The organic layer was dried (Na₂SO₄), filtered, and concentrated to yield 10.60 g (92%) of phenylmethyl (2S)-2-[(tert-butoxy)carbonylamino]-2-{4-[(trifluoromethyl)sulfonyloxy]phenyl}acetate as light yellow solid.

¹H NMR (300 MHz, CDCl₃) δ 7.43 (2H, d), 7.32-7.16 (7H, m) 5.67 (1H, d), 5.40 (1H, d), 5.16 (2H, s), 1.43 (9H, s).

Tetrakis(triphenylphosphine)palladium(0) (0.071 g, 0.061 mmol) was added to a suspension of 3-methoxyphenylboronic acid (0.621 g, 4.1 mmol) and potassium carbonate (0.424 g, 3.1 mmol) in 12 mL of toluene. The reaction mixture was degassed and heated to 80° C. before adding the phenylmethyl (2S)-2-[(tert-butoxy)carbonylamino]-2-{4-[(trifluoromethyl)sulfonyloxy]phenyl}acetate (1.00 g, 2.0 mmol). The thick suspension was stirred at 80° C. for 2 hours and then filtered through Celite. The filtrate was concentrated and purified by flash chromatography (ethyl acetate/hexanes, 100% hexanes to 1:4) to give the white solid of the phenylmethyl (2S)-2-[(tert-butoxy)carbonylamino]-2-[4-(3-methoxyphenyl)phenyl]acetate (0.878 g) in 98% yield. MS (M−H)-446.

Prepared in a manner similar to that described in Example 4 using 0.878 g (1.96 mmol) of phenylmethyl (2S)-2-[(tert-butoxy)carbonylamino]-2-[4-(3-methoxyphenyl)phenyl]acetate, and 10 mL of 4M solution of HCl in 1,4-dioxane to yield 0.740 g (98%) of phenylmethyl (2S)-2-amino-2-[4-(3-methoxyphenyl)phenyl]acetate, hydrochloride. MS (M−Cl)⁺ 348.

Prepared in a manner similar to that described in Example 24 using 0.540 g (1.41 mmol) of phenylmethyl (2S)-2-amino-2-[4-(3-methoxyphenyl)phenyl]acetate, hydrochloride, 0.265 g (1.41 mmol) of (2R)-2-[(methoxycarbonyl)methyl]-4-methylpentanoic acid, 0.215 g (1.41 mmol) of HOBt, 0.539 g (2.81 mmol) of EDC, and 0.46 mL (4.22 mmol) of NMM to yield 0.692 g (95%) of methyl (3R)-3-(N-{(1S)[4-(3-methoxyphenyl)phenyl][benzyloxycarbonyl]methyl}carbamoyl)-5-methylhexanoate. MS (M+H)⁺ 518; (M+HCO₂)⁻ 562.

Prepared in a manner similar to that described in Example 21 using 0.692 g (1.34 mmol) of methyl (3R)-3-(N-{(1S)[4-(3-methoxyphenyl)phenyl][benzyloxycarbonyl]methyl}carbamoyl)-5-methylhexanoate, 0.151 g 10% palladium on carbon, and 30 mL of ethyl acetate to yield 0.567 g (99%) of 2-{(2R)-2-[(methoxycarbonyl)methyl]-4-methylpentanoylamino}(2S)-2-[4-(3-methoxyphenyl)phenyl]acetic acid.

MS (M+H)⁺ 428; (M−H)⁻ 426.

Prepared in a manner similar to that described in Example 24 using 0.467 g (1.09 mmol) of 2-{(2R)-2-[(methoxycarbonyl)methyl]-4-methylpentanoylamino}(2S)-2-[4-(3-methoxyphenyl)phenyl]acetic acid, 0.145 g (1.09 mmol) of indane-2-ylamine, 0.167 g (1.09 mmol) of HOBt, 0.419 g (2.18 mmol) of EDC, and 0.24mL (2.18 mmol) of NMM to yield 0.501 g (85%) of methyl (3R)-3-(N-{(N-indan-2-ylcarbamoyl)[4-(3-methoxyphenyl)phenyl]methyl}carbamoyl)-5-methylhexanoate. MS (+H)⁺ 543.

Prepared in a manner similar to that described in Example 29 using 0.501 g (0.92 mmol) of methyl (3R)-3-(N-{(N-indan-2-ylcarbamoyl)[4-(3-methoxyphenyl)phenyl]methyl}carbamoyl)-5-methylhexanoate to yield 0.414 g (82%) of 2-(N-hydroxycarbamoylmethyl) (2R)—N-{(1S,R)(N-indan-2-ylcarbamoyl)[4-(3-methoxyphenyl)phenyl]methyl}-4-methylpentanamide (1:1 mixture of diastereoisomers). MS (M+[)⁺ 544.

### Example 46

### Compound 20

### 2-(N-hydroxycarbamoylmethyl)(2R)—N-((1R){N-[2-(dimethylamino)ethyl]-N-benzylcarbamoyl}(4-phenylphenyl)methyl)-4-methylpentanamide

Following the procedure of Example 3, N′-benzyl-N,N-dimethylethylenediamine (673 mg, 3.77 mmol), 2-{(2R)-2-[(methoxycarbonyl)methyl]-4-methylpentanoylamino}-2-(4-phenylphenyl)acetic acid (1.5 g, 3.7 mmol), EDC (1.44 g, 7.54 mmol), HOBt (577 mg, 3.77 mmol), NMM instead of DIEA (0.828 mL, 7.54 mmol) and dichloromethane (50 mL) to yield 1.24 g (59%) of methyl (3R)-3-[N-({N-[2-(dimethylamino)ethyl]-N-benzylcarbamoyl}(4-phenylphenyl)methyl)carbamoyl]-5-methylhexanoate as a yellow liquid. MS (M+H)⁺ 558.

Using the procedure of Example 2, methyl (3R)-3-[N-({N-[2-(dimethylamino)ethyl]-N-benzylcarbamoyl}(4-phenylphenyl)methyl)carbamoyl]-5-methylhexanoate (257 mg, 0.46 n-mol). The crude product was purified by silica gel chromatography (water/methanol, 30:70) to the isolation of 2-(N-hydroxycarbamoylmethyl)(2R)—N-((1R) {N-[2-(dimethylamino)ethyl]-N-benzylcarbamoyl}(4-phenylphenyl)methyl)-4-methylpentanamide (45 mg) in 18% yield (less polar product), R_{f}=0.41 (methanol). MS (M+H)⁺ 544.

### Example 47

### Compound 21

### 2-(N-hydroxycarbamoylmethyl)(2R)—N-({N-[(2,6-dimethoxyphenyl)methyl]carbamoyl}(4-phenylphenyl)methyl)-4-methylpentanamide

Following the procedure of Example 3, 2,6-dimethoxybenzylamine (84 mg, 0.5 mmol), 2-{(2R)-2-[(methoxycarbonyl)methyl]-4-methylpentanoylamino}-2-(4-phenylphenyl)acetic acid (200 mg, 0.5 mmol), EDC (192 mg, 1 mmol), HOBt (77 mg, 0.5 mmol), NMM instead of DIEA (0.11 mL, 1 mmol) and dichloromethane (20 mL) to yield 257 mg (94%) of methyl (3R)-3-[N-({N-[(2,6-dimethoxyphenyl)methyl]carbamoyl}(4-phenylphenyl)methyl)carbamoyl]-5-methylhexanoate as an off white solid. MS (M+H)⁺ 547.

Using the procedure of Example 2, methyl (3R)-3-[N-({N-[(2,6-dimethoxyphenyl)methyl]carbamoyl}(4-phenylphenyl)methyl)carbamoyl]-5-methylhexanoate (240 mg, 0.44 mmol). The crude product was purified by heating in dichloromethane to the isolation of 2-(N-hydroxycarbamoylmethyl)(2R)—N-({N-[(2,6-dimethoxyphenyl)methyl]carbamoyl}(4-phenylphenyl)methyl)-4-methylpentanamide (180 mg) in 75% yield (diastereoisomeric mixture of 37/54.5 ratio), R_{f}=0.47 (ethyl acetate/methanol, 9:1). MS (M+H)⁺ 548.

### Example 48

### Compound 22

### 2-(N-hydroxycarbamoylmethyl)(2R)—N-[(1S)[N-methyl-N-(2-pyridylmethyl)carbamoyl](4-phenylphenyl)methyl]-4-methylpentanamide

Following the procedure of Example 3, methylpyridin-2-ylmethylamine dihydrochloride (49 mg, 0.25 mmol), 2-{(2R)-2-[(methoxycarbonyl)methyl]-4-methylpentanoylamino}-2-(4-phenylphenyl)acetic acid (100 mg 0.25 mmol), EDC (96 mg, 0.5 mmol), HOBt (38 mg, 025 mmol), NMM instead of DIEA (0.109 mL, 1 mmol) and dichloromethane (10 mL) to yield 100 mg (80%) of methyl (3R)-5-methyl-3-(N-{[N-methyl-N-(2-pyridylmethyl)carbamoyl](4-phenylphenyl)methyl}carbamoyl)hexanoate as a yellow liquid. MS (M+H)⁺ 502.

Using the procedure of Example 2, methyl (3R)-5-methyl-3-(N-{[N-methyl-N-(2-pyridylmethyl)carbamoyl](4-phenylphenyl)methyl}carbamoyl)hexanoate (99 mg, 0.197 mmol). The crude product was purified by silica gel chromatography (water/methanol, 40:60) to the isolation of 2-(N-hydroxycarbamoylmethyl)(2R)—N-{(1S)[N-methyl-N-(2-pyridylmethyl)carbamoyl](4-phenylphenyl)methyl}-4-methylpentanamide (10 mg) in 10% yield (diastereoisomeric mixture of 89/11 ratio), R_{f}=0.52 (ethyl acetate/methanol, 4:1). MS (M+H)⁺ 503.

### Example 49

### Compound 23

### 2-N-hydroxycarbamoylmethyl)(2R)—N-{(1R)[N-methyl-N-(2-pyridylmethyl)carbamoyl](4-phenylphenyl)methyl}-4-methylpentanamide

Using the procedure of Example 2, methyl (3R)-5-methyl-3-(N-{[N-methyl-N-(2-pyridylmethyl)carbamoyl](4-phenylphenyl)methyl}carbamoyl)hexanoate (99 mg, 0.197 mmol). The crude product was purified by silica gel chromatography (water/methanol, 30:70) to the isolation of 2-(N-hydroxycarbamoylmethyl)(2R)—N-{(1R)[N-methyl-N-(2-pyridylmethyl)carbamoyl](4-phenylphenyl)methyl}-4-methylpentanamide (15 mg) in 15% yield (less polar product), R_{f}=0.35 (ethyl acetate/methanol, 4:1). MS (M+H)⁺ 503.

### Example 50

### Compound 24: 2-(N-hydroxycarbamoylmethyl)(2R)-4-methyl-N-{[N-methyl-N-(2-(2-pyridyl)ethyl)carbamoyl](4-phenylphenyl)methylpentanamide

Following the procedure of Example 3,2-(2-methylaminoethyl)pyridine (34 mg, 0.25 mmol), 2-{(2R)-2-[(methoxycarbonyl)methyl]-4-methylpentanoylamino}-2-(4-phenylphenyl)acetic acid (100 mg, 0.25 mmol), EDC (96 mg, 0.5 mmol), HOBt (38 mg, 025 mmol), NMM instead of DIEA (0.055 mL, 0.5 mmol) and dichloromethane (10 mL) to yield 115 mg (89%) of methyl (3R)-5-methyl-3-(N-{[N-methyl-N-(2-(2-pyridyl)ethyl)carbamoyl](4-phenylphenyl)methyl}carbamoyl)hexanoate as a yellow liquid. MS (+H)⁺ 516.

Using the procedure of Example 2, methyl (3R)-5-methyl-3-(N-{[N-methyl-N-(2-(2-pyridyl)ethyl)carbamoyl](4-phenylphenyl)methyl}carbamoyl)hexanoate (113 mg, 0.22 mmol). The crude product was purified by silica gel chromatography (water/methanol, 30:70) to the isolation of 2-(N-hydroxycarbamoylmethyl)(2R)-4-methyl-N-{[N-methyl-N-(2-(2-pyridyl)ethyl)carbamoyl](4-phenylphenyl)methyl}pentanamide (13 mg) in 11% yield (diastereoisomeric mixture of 45/55 ratio), R_{f}=0.48 (ethyl acetate/methanol, 4:1). MS (M+H)⁺ 517.

### Example 51

### Compound 25: 2-[2-(N-hydroxycarbamoylmethyl)(2R)-4-methylpentanoylamino](2S)—N-indan-2-yl-N′-indan-2-ylpentane-1,5-diamide

Following the procedure of Example 3, boc-glu(obzl)-OH (1 g, 2.9 mmol), 2-aminoindan (395 mg, 2.9 mmol), EDC (1.11 g, 5.8 mmol), HOBt (444 mg, 2.9 mmol), NMM instead of DIEA (0.637 mL, 5.8 mmol) and dichloromethane (15 mL) to yield 900 mg (69%) of phenylmethyl (4S)-4-[(tert-butoxy)carbonylamino]-4-(N-indan-2-ylcarbamoyl)butanoate as a brown solid.

¹H NMR (300 MHz, d₆-DMSO): δ 8.121 (1H, d), 7.19 (9H, m), 6.86 (1H, d), 5.07 (2H, s), 4.44 (1H, q), 3.9 (1H, q), 3.16 (2H, q), 2.76 (2H, m), 2.35 (2H, t), 1.78 (2H, m), 1.35 (9H, s).

Using the procedure of Example 21, phenylmethyl (4S)-4-[(tert-butoxy)carbonylamino]-4-(N-indan-2-ylcarbamoyl)butanoate (890 mg, 1.96 mmol) 10% palladium on carbon (89 mg, 10% of ester), methanol (20 mL) to yield 700 mg (98%) of (4S)-4-[(tert-butoxy)carbonylamino]-4-(N-indan-2-ylcarbamoyl)butanoic acid as a white solid. (M−H)⁻ 361.

Following the procedure of Example 3, (4S)-4-[(tert-butoxy)carbonylamino]-4-(N-indan-2-ylcarbamoyl)butanoic acid (690 mg, 1.9 mmol), 2-aminoindan (252 mg, 1.9 mmol), EDC (730 mg, 3.8 mmol), HOBt (291 mg, 1.9 mmol), NMM instead of DIEA (0.417 mL, 3.8 mmol) and dichloromethane (15 mL) to yield 887 mg (98%) of (2S)-2-[(tert-butoxy)carbonylamino]-N-indan-2-yl-N′-indan-2-ylpentane-1,5-diamide as a off white solid.

¹H NMR (300 MHz, d₆-DMSO): δ 8.10 (2H, t), 7.15 (8H, m), 6.74 (2H, m), 3.84 (1H, m), 3.17 (3H, m), 2.74 (4H, m), 2.06 (2H, q), 1.71 (2H, m), 1.35 (9H, s).

Following the procedure of Example 4, (2S)-2-[(tert-butoxy)carbonylamino]-N-indan-2-yl-N′-indan-2-ylpentane-1,5-diamide (870 mg, 1.8 mmol) and 4N HCl/Dioxane (10mL) to yield 714 mg (96%) of (2S)-2-amino-N-indan-2-yl-N′-indan-2-ylpentane-1,5-diamide hydrochloride as a brown solid. (M+H-HCl)⁺ 378.

Following the procedure of Example 3, (2S)-2-amino-N-indan-2-yl-N′-indan-2-ylpentane-1,5-diamide hydrochloride (300 mg, 0.7 mmol), (2R)-2-[(methoxycarbonyl)methyl]-4-methylpentanoic acid (136 mg, 0.7 mmol), EDC (269 mg, 1.4 mmol), HOBt (107 mg, 0.7 mmol), NMM instead of DIEA (0.23mL, 2.1 mmol) and dichloromethane (10 mL) to yield 352 mg (92%) of methyl (3R)-3-{N-[(1S)-1,3-bis(N-indan-2-ylcarbamoyl)propyl]carbamoyl}-5-methylhexanoate as a off white solid. MS (M+H)⁺.

¹H NMR (300 MHz, d₆-DMSO): δ 8.04 (2H, d), 7.17 (8H, d), 4.43 (2H, m), 4.15 (1H, m), 3.49 (3H, m), 3.14 (4H, m), 2.71 (5H, m), 2.42 (2H, m), 2.061(2H, m), 1.76 (2H, m), 1.41 (2H, m), 1.1 (1H, m), 0.81 (6H, m)

Using the procedure of Example 2, methyl (3R)-3-{N-[(1S)-1,3-bis(N-indan-2-ylcarbamoyl)propyl]carbamoyl}-5-methylhexanoate (340 mg, 0.62 mmol) was converted to 2-[2-(N-hydroxycarbamoylmethyl)(2R)-4-methylpentanoylamino](2S)—N-indan-2-yl-N′-indan-2-ylpentane-1,5-diamide (320 mg) in 94% yield, R_{f}=0.63 (ethyl acetate/methanol, 4:1). MS (M−H)-547.

### Example 52

### Compound 26: 2-(N-hydroxycarbamoylmethyl)(2R)—N-{(1R)(4-phenylphenyl)[N-(2-pyridylmethyl)carbamoyl]methyl}-4-methylpentanamide

Following the procedure of Example 3,2-(aminomethyl)pyridine (54 mg, 0.5 mmol), 2-{(2R)-2-[(methoxycarbonyl)methyl]-4-methylpentanoylamino}-2-(4-phenylphenyl)acetic acid (200 mg, 0.5 mmol), EDC (192 mg, 1 mmol), HOBt (77 mg, 0.5 mmol), NMM instead of DIEA (0.11 mL, 1 mmol) and dichloromethane (10 mL) to yield 214 mg (88%) of methyl (3R)-5-methyl-3-(N-{(4-phenylphenyl)[N-(2-pyridylmethyl)carbamoyl]methyl}carbamoyl)hexanoate as a yellow solid. MS (+H)⁺ 488.

Using the procedure of Example 2, methyl (3R)-5-methyl-3-(N-{(4-phenylphenyl)[N-(2-pyridylmethyl)carbamoyl]methyl}carbamoyl)hexanoate (174 mg, 0.35 mmol). The crude product was purified by silica gel chromatography (water/methanol, 30:70) then purify by prep tlc to the isolation of 2-(N-hydroxycarbamoylmethyl)(2R)—N-{(1R)(4-phenylphenyl)[N-(2-pyridylmethyl)carbamoyl]methyl}-4-methylpentanamide (13 mg) in 8% yield (less polar product), R_{f}=0.58 (ethyl acetate/methanol, 4:1). MS (M−H)⁻ 487.

### Example 53

### Compound 27: 2-(N-hydroxycarbamoylmethyl)(2R)—N-{(1S)(4-phenylphenyl)[N-(2-pyridylmethyl)carbamoyl]methyl}-4-methylpentanamide

Using the procedure of Example 2, methyl (3R)-5-methyl-3-(N-{(4-phenylphenyl)[N-(2-pyridylmethyl)carbamoyl]methyl}carbamoyl)hexanoate (174 mg, 0.35 mmol). The crude product was purified by silica gel chromatography (water/methanol, 30:70) then purify by prep tlc to the isolation of 2-(N-hydroxycarbamoylmethyl)(2R)—N-{(1S)(4-phenylphenyl)[N-(2-pyridylmethyl)carbamoyl]methyl}-4-methylpentanamide (15 mg) in 9% yield (more polar product), R_{f}=0.5 (ethyl acetate/methanol, 4:1). MS (+H)⁺ 489.

### Example 54

### Compound 28: 2-(N-hydroxycarbamoylmethyl)(2R)—N-{(1R)[N-methyl-N-benzylcarbamoyl](4-phenylphenyl)methyl}-4-methylpentanamide

Following the procedure of Example 3, N-methylbenzylamine (61 mg, 0.5 mmol), 2-{(2R)-2-[(methoxycarbonyl)methyl]-4-methylpentanoylamino}-2-(4-phenylphenyl)acetic acid (200 mg, 0.5 mmol), EDC (192 mg, 1 mmol), HOBt (70 mg, 0.5 mmol), NMM instead of DIEA (0.184 mL, 1 mmol) and dichloromethane (5 mL) to give the product methyl (3R)-5-methyl-3-(N-{[benzyloxycarbonyl](4-phenylphenyl)methyl}carbamoyl)hexanoate as a yellow solid.

Using the procedure of Example 2, methyl (3R)-5-methyl-3-(N-{[benzyloxycarbonyl](4-phenylphenyl)methyl}carbamoyl)hexanoate (200 mg, 0.4 mmol). The crude product was purified by silica gel chromatography (ethyl acetate/methanol, 9:1) to the isolation of 2-(N-hydroxycarbamoylmethyl)(2R)—N-{(1R)[N-methyl-N-benzylcarbamoyl](4-phenylphenyl)methyl}-4-methylpentanamide (5 mg) in 2% yield (less polar product), R_{f}=0.55 (ethyl acetate/methanol, 9:1). MS (M+H)⁺ 502.

### Example 55

### Compound 29: 2-(N-hydroxycarbamoylmethyl)(2R)—N-{(1S)[N-methyl-N-benzylcarbamoyl](4-phenylphenyl)methyl}-4-methylpentanamide

Using the procedure of Example 2, methyl (3R)-5-methyl-3-(-{[benzyloxycarbonyl](4-phenylphenyl)methyl}carbamoyl)hexanoate (200 mg, 0.4 mmol). The crude product was purified by silica gel chromatography (ethyl acetate) to the isolation of 2-(N-hydroxycarbamoylmethyl)(2R)—N-{(1S)[N-methyl-N-benzylcarbamoyl](4-phenylphenyl)methyl}-4-methylpentanamide (14 mg) in 4% yield (more polar product), R_{f}=0.74 (ethyl acetate/methanol, 9:1). MS (M+H)⁺ 502.

### Example 56

### Compound 30: 4-({2-[2-(N-hydroxycarbamoylmethyl)(2R)-4-methylpentanoylamino]-2-(4-phenylphenyl)acetylamino}methyl)benzoic acid

Using the procedure of Example 2, 4-[(2-{(2R)-2-[(methoxycarbonyl)methyl]-4-methylpentanoylamino}-2-(4-phenylphenyl)acetylamino)methyl]benzoic acid (155 mg, 0.29 mmol). The crude product was purified by silica gel chromatography (methanol/water, 70:30) to the isolation of 4-({2-[2-(N-hydroxycarbamoylmethyl)(2R)-4-methylpentanoylamino]-2-(4-phenylphenyl)acetylamino}methyl)benzoic acid (48 mg) in 48% yield, R_{f}=0.55 (ethyl acetate/methanol, 4:1). MS (M+H)⁺ 531.9.

### Example 57

### Compound 31: 3-(N-hydroxycarbamoyl)(2R)-2-methyl-N-{[N-benzylcarbamoyl](4-phenylphenyl)methyl}propanamide

Using the procedure of Example 28, (R)-(+)-2-methylsuccinic acid 4-methyl ester (112 mg, 0.77 mmol), ammonia (0.77 mL, 1.54 mmol), 4-biphenylcarboxaldehyde (140 mg, 0.77 mmol), and benzyl isocyanide (90 mg, 0.77 mmol). The crude residue was purified by flash chromatography (dichloromethane/methanol, 100:2) to yield 263 mg (77%) of methyl (3R)-3-(N-{[N-benzylcarbamoyl](4-phenylphenyl)methyl}carbamoyl)butanoate as a yellow solid. MS (M+H)⁺ 445.

Using the procedure of Example 2, methyl (3R)-3-(N-{[N-benzylcarbamoyl](4-phenylphenyl)methyl}carbamoyl)butanoate (250 mg, 0.56 mmol). The crude product was purified by silica gel chromatography (methanol/water, 70:30) to the isolation of 3-(N-hydroxycarbamoyl)(2R)-2-methyl-N-{[N-benzylcarbamoyl](4-phenylphenyl)methyl}propanamide (16 mg) in 6% yield, R_{f}=0.35 (ethyl acetate/methanol, 9:1).

MS (M+H)⁺ 446.

### Example 58

### Compound 32

### 2-(N-hydroxycarbamoylmethyl)(2R)—N-(1R,S)(1R,S) {N-[(3-methoxyphenyl)methyl]carbamoyl}(4-phenylphenyl)methyl)-4-methylpentanamide

Methyl (3R)-3-[N-((1R,S) {N-[(3-methoxyphenyl)methyl]carbamoyl}(4-phenylphenyl)methyl)carbamoyl]-5-methylhexanoate was prepared following the procedure from Example 3 using 2-[(2R)-2-[(methoxycarbonyl)methyl]-4-methylpentanoylamino}-2-(4-phenylphenyl)acetic acid (see Example 21) (200 mg, 0.5 mmol), 3-methoxybenzylamine (70 mg, 0.5 mmol), EDC HCl (192 mg, 1 mmol), HOBt (68 mg, 0.5 mmol), DIEA (184 μl, 1 mmol) in Dichloromethane (5 mL). Yield: 210 mg (81%). MS: (M+H⁺) 517.

2-(N-hydroxycarbamoylmethyl)(2R)—N-((1R,S) {N-[(3-methoxyphenyl)methyl]carbamoyl}(4-phenylphenyl)methyl)-4-methylpentanamide (43/57) was prepared from Methyl(3R)-3-[N-({N-[(3-methoxyphenyl)methyl]carbamoyl}(4-phenylphenyl)methyl)carbamoyl]-5-methylhexanoate (206 mg, 0.4 mmol) using the procedure from Example 2. A mixture of two diastereoisomers was obtained. Yield: 170 mg (82%).

MS: (M+H) 518.

### Example 59

### Compound 33: 2-N-hydroxycarbamoylmethyl)(2R)—N-((1R,S){N-[(4-{[(tert-butoxy)carbonylamino]methyl}phenyl)methyl]carbamoyl}(4-phenylphenyl)methyl)-4-methylpentanamide

Methyl (3R)-3-[N-((1R,S) {N-[(4-{[(tert-butoxy)carbonylamino]methyl}phenyl)methyl]carbamoyl}(4-phenylphenyl)methyl)carbamoyl]-5-methylhexanoate was prepared using the procedure in Example 3 with 2-{(2R)-2-[(methoxycarbonyl)methyl]-4-methyl pentanoylamino}-2-(4-phenylphenyl)acetic acid (200 mg, 0.5 mmol), 1-(N-boc-aminomethyl)-4-(aminomethyl)benzene (120 mg, 0.5 mmol), EDC HCl (192 mg, 1 mmol), HOBt (68 mg, 0.5 mmol), DIEA (174 μL, 1 mmol) and dichloromethane (5 mL). Yield: 260 mg (85%). MS: (M+H) 616.

2-(N-hydroxycarbamoylmethyl)(2R)—N-((1R,S) {N-[(4-{[(tert-butoxy)carbonylamino]methyl}phenyl)methyl]carbamoyl}(4-phenylphenyl)methyl)-4-methylpentanamide (57/43) was prepared from methyl(3R)-3-[N-((1R,S) {N-[(4-{[(tert-butoxy)carbonyl amino]methyl}phenyl)methyl]carbamoyl}(4-phenylphenyl)methyl)carbamoyl]-5-methyl hexanoate (246 mg, 0.4 mmol) using the procedure from Example 2.

Yield: 150 mg (60%). MS: (M+H) 617.

### Example 60

### Compound 34: 2-(N-hydroxycarbamoylmethyl)(2R)—N-{[N-((11S)-1-phenylethyl)carbamoyl](3-phenylphenyl)methyl}-4-methylpentanamide

Using the procedure in Example 28, (2R)-2-[(methoxycarbonyl)methyl]-4-methylpentanoic acid (145 mg, 0.77 mmol), ammonia (0.77 mL, 1.54 mmol), 3-phenylbenzaldehyde (140 mg, 0.77 mmol), and (S)-(−)-alpha-methylbenzyl isocyanide (140 mg, 0.77 mmol). The crude residue was purified by flash chromatography (dichloromethane/methanol, 100:1.5) to yield 194 mg (50%) of methyl (3R)-3-(N-{[N-((1S)-1-phenylethyl)carbamoyl](3-phenylphenyl)methyl}carbamoyl)-5-methylhexanoate as a yellow solid. MS (M+H)⁺ 501.

Using the procedure in Example 2, methyl (3R)-3-(N-{[N-((1S)-1-phenylethyl) carbamoyl](3-phenylphenyl)methyl}carbamoyl]-5-methylhexanoate (188 mg, 0.376 mmol). The crude product was purified by hot isopropanol to the isolation of 2-(N-hydroxycarbamoylmethyl)(2R)—N-{[N-((1S)-1-phenylethyl)carbamoyl](3-phenylphenyl)methyl}-4-methylpentanamide (43 mg) in 23% yield, R_{f}=0.69 (ethyl acetate/methanol, 9:1). MS (M+H)⁺ 502.

### Example 61

### Compound 35: 2-(N-hydroxycarbamoylmethyl)(2R)—N-({N-[(3-methylphenyl)methyl]carbamoyl}(4-phenylphenyl)methyl)hexanamide

Following the procedure of Example 3, 3-methylbenzylamine (242 mg, 2 mmol), N-Boc-amino-biphenyl acetic acid (654 mg, 2 mmol), EDC (768 mg, 4 mmol), HOBt (306 mg, 2 mmol), NMM instead of DIEA (0.439 mL, 4 mmol) and dichloromethane (15 mL) to yield 569 mg (66%) of 2-[(tert-butoxy)carbonylamino]-N-[(3-methylphenyl)methyl]-2-(4-phenylphenyl)acetamide as a yellow solid.

Following the procedure of Example 4, 2-[(tert-butoxy)carbonylamino]-N-[(3-methylphenyl)methyl]-2-(4-phenylphenyl)acetamide (556 mg, 1.3 mmol) to yield 418 mg (88%) of 2-amino-N-[(3-methylphenyl)methyl]-2-(4-phenylphenyl)acetamide hydrochloride as a yellow solid.

Following the procedure of Example 3, 2-amino-N-[(3-methylphenyl)methyl]-2-(4-phenylphenyl)acetamide hydrochloride (400 mg, 1.1 mmol), (2R)-2-[(ethoxycarbonyl)methyl]hexanoic acid (221 mg, 1.09 mmol), EDC (419 mg, 2.1 mmol), HOBt (167 mg, 1.09 mmol), NMM instead of DIEA (0.359 mL, 3.2 mmol) and dichloromethane (10mL) to yield 510 mg (91%) of ethyl (3R)-3-[N-({N-[(3-methylphenyl)methyl]carbamoyl}(4-phenylphenyl)methyl)carbamoyl]heptanoate as an yellow solid.

Using the procedure of Example 2, ethyl (3R)-3-[N-({N-[(3-methylphenyl)methyl]carbamoyl}(4-phenylphenyl)methyl)carbamoyl]heptanoate (250 mg, 0.48 mmol). The crude product was recrystallized in isopropanol and dichloromethane to the isolation of 2-(N-hydroxycarbamoylmethyl)(2R)—N-({N-[(3-methylphenyl)methyl]carbamoyl}(4-phenylphenyl)methyl)hexanamide (147 mg) in 61% yield.

R_{f}=0.53 (ethyl acetate/methanol, 9:1). MS (M+H)⁺ 502.

### Example 62

### Compound 36: 2-(N-hydroxycarbamoylmethyl)(2R)—N-{fluoren-2-yl[N-benzylcarbamoyl]methyl}-4-methylpentanamide

Prepared in a manner similar to that described in Example 1 using 0.291 g (1.5 mmol) of fluorene-2-carbaldehyde, 0.282 g (1.5 mmol) of (2R)-2-[(methoxycarbonyl)methyl]-4-methylpentanoic acid, 0.182 mL (1.5 mmol) of benzyl isocyanide and 1.5 mL (3.0 mmol) of 2M solution of ammonia in methanol to yield 0.396 g (53%) of methyl (3R)-3-(N-{fluoren-2-yl[N-benzylcarbamoyl]methyl}carbamoyl)-5-methylhexanoate.

MS (M+H)⁺ 499; (+HCO₂⁻)⁻ 543.

Prepared in a manner similar to that described in Example 29 using 0.078 g (0.16 mmol) of methyl (3R)-3-(N-{fluoren-2-yl[N-benzylcarbamoyl]methyl}carbamoyl)-5-methylhexanoate to yield 0.035 g (44%) of 2-(N-hydroxycarbamoylmethyl)(2R)—N-{(1S,R)fluoren-2-yl[N-benzylcarbamoyl]methyl}-4-methylpentanamide (3:2 mixture of diastereoisomers). MS (M+1)⁺ 500; (M−H)⁻ 498.

### Example 63

### Compound 37: 2-(N-hydroxycarbamoylmethyl)(2R)-4-methyl-N-methyl-N-{[N-benzylcarbamoyl](4-phenylphenyl)methyl}pentanamide

Prepared in a manner similar to that described in Example 1 using 0.273 g (1.5 mmol) of 4-phenylbenzaldehyde, 0.282 g (1.5 mmol) of (2R)-2-[(methoxycarbonyl)methyl]-4-methylpentanoic acid, 0.182 mL (1.5 mmol) of benzyl isocyanide and 1.5 mL (3.0 mmol) of 2M solution of methylamine in methanol to yield 0.565 g (75%) of methyl (3R)-5-methyl-3-(N-methyl-N-{[N-benzylcarbamoyl](4-phenylphenyl)methyl}carbamoyl)hexanoate. MS (M−H)-499.

Prepared in a manner similar to that described in Example 29 using 0.164 g (0.33 mmol) of methyl (3R)-5-methyl-3-(N-methyl-N-{[N-benzylcarbamoyl](4-phenylphenyl)methyl}carbamoyl)hexanoate to yield 0.121 g (73%) of 2-(N-hydroxycarbamoylmethyl)(2R)—N-{(1S,R)[N-benzylcarbamoyl](4-phenylphenyl)methyl}-4-methyl-N-methylpentanamide (1:3 mixture of diastereoisomers). MS (M−H)⁻ 500.

### Example 64

### Compound 38

### 2-(N-hydroxycarbamoylmethyl)(2R)—N-((1R,S){N-[(3,4-dimethylphenyl)methyl]carbamoyl}(4-phenylphenyl)methyl)-4-methylpentanamide

Methyl (3R)-3-[N-({N-[(3,4-dimethylphenyl)methyl]carbamoyl}(4-phenylphenyl)methyl)carbamoyl]-5-methylhexanoate was prepared from 2-{(2R)-2-[(methoxy carbonyl)methyl]-4-methyl pentanoylamino}-2-(4-phenylphenyl)acetic acid (397 mg, 1 mmol), 2,3-dimethylbenzylamine (135 mg, 1 mmol), EDC HCl (384 mg, 2 mmol), HOBt (135 mg, 1 mmol), DIEA (384 μL, 2 mmol), and dichloromethane (5mL) using the procedure of Example 3. Yield: 290 mg (57%). MS: (M+H⁺) 515.

2-(N-hydroxycarbamoylmethyl)(2R)—N-((1R,S){N-[(3,4-dimethylphenyl)methyl]carbamoyl}(4-phenylphenyl)methyl)-4-methylpentanamide (32/68) was prepared from methyl (3R)-3-[N-({N-[(3,4-dimethylphenyl)methyl]carbamoyl}(4-phenylphenyl)methyl)carbamoyl]-5-methyl hexanoate (257 mg, 0.5 mmol) using the procedure from Example 2. Yield: 150 mg (58%). MS: (M+H⁺) 516.

### Example 65

### Compound 39: 2-N-hydroxycarbamoylmethyl)(2R)—N-[(1R,S)(N-indan-2-ylcarbamoyl)(4-phenylphenyl)methyl]hexanamide

Methyl (2R)-2-({N-[(2,4-dimethoxyphenyl)methyl]-N-[(4-methoxyphenyl)methoxy]carbamoylmethyl)hexanoate was prepared using the procedures from Example 3 using (r)-2-butylsuccinic acid-1-methyl ester (5.0 g, 26.5 mmol), [(2,4-dimethoxy phenyl)methyl][(4-methoxyphenyl)methoxy]amine (9.09 g, 30 mmol), EDC HCl (10.18 g, 53 mmol), HOBt (3.58 g, 26.5 mmol), DIEA (9.22 mL, 53 mmol), and dichloromethane (100 mL). Yield: 11.0 g (88%). MS: (M+H) 474.

(2R)-2-({N-[(2,4-dimethoxyphenyl)methyl]-N-[(4-methoxyphenyl)methoxy]carbamoyl}methyl)hexanoic acid, sodium salt was prepared by from methyl (2R)-2-({N-[(2,4-dimethoxyphenyl)methyl]-N-[(4-methoxyphenyl)methoxy]carbamoylmethyl) hexanoate (10.4 g, 22 mmol) using the procedure from Example, 13. Yield: 8.0 g (75%). MS: (M+H⁺-Na) 460.

2-[(tert-butoxy)carbonylamino]-N-indan-2-yl-2-(4-phenylphenyl)acetamide was prepared by heating the mixture of N-boc-amino-biphenyl acetic acid (654 mg, 2.0 mmol), 2-aminoindan (258 μL, 2 mmol) EDC HCl (768 mg, 4 mmol), HOBt (270 mg, 2 mmol), DIEA (696 μL, 4 mmol), dimethylformamide (5mL). The mixture was heated to 160° C. for 600 seconds using microwaves. The DMF was rotovaped, the residue was taken in EtOAc and washed with 1N HCl (2×15 mL), saturated sodium carbonate solution (2×15 mL), finally by brine (2×15mL). The EtOAc solution was dried over anhydrous sodium sulphate and rotovaped. The residue on triturating with hexanes gave a solid. Yield: 0.79 g (88%). MS: (M+HCO₂⁻)-487.

2-amino-N-indan-2-yl-2-(4-phenylphenyl)acetamide, hydrochloride was prepared from 2-[(tert-butoxy)carbonylamino]-N-indan-2-yl-2-(4-phenylphenyl)acetamide (665 mg, 1.5 mmol) using 4N HCl/dioxane (10mL), using the procedure from Example 4. Yield: 550 mg (97%).

(2R)—N′-[((1R,S)2,4-dimethoxyphenyl)methyl]-2-butyl-N—[(N-indan-2-ylcarbamoyl)(4-phenyl phenyl)methyl]-N′-[(4-methoxyphenyl)methoxy]butane-1,4-diamide was prepared from 2R)-2-({N-[(2,4-dimethoxyphenyl)methyl]-N-[(4-methoxyphenyl)methoxy]carbamoyl}methyl)hexanoic acid (481 mg, 1 mmol) 2-amino-N-indan-2-yl-2-(4-phenyl phenyl)acetamide, hydrochloride (379 mg, 1 mmol), EDC HCl (384 mg, 2 mmol), HOBt (135 mg, 1 mmol), DIEA (384 μL, 2 mmol) and dichloromethane (5 mL). Yield 710 mg (91%).

2-(N-hydroxycarbamoylmethyl)(2R)—N-[(1R,S)(N-indan-2-ylcarbamoyl)(4-phenyl phenyl)methyl]hexanamide (59/41) was prepared from (2R)—N′-[((1R,S)2,4-dimethoxyphenyl)methyl]-2-butyl-N—[(N-indan-2-ylcarbamoyl)(4-phenyl phenyl)methyl]-N′-[(4-methoxy phenyl)methoxy]butane-1,4-diamide (220 mg, 0.28 mmol) using the procedure from Example 2. Yield: 100 mg (45%). MS (M+H⁺) 514.

### Example 66

### Compound 40: 2-(N-hydroxycarbamoylmethyl)(2R)-4-methyl-N-[(4-phenylphenyl)methyl]pentanamide

Prepared in a manner similar to that described in Example 24 using 1.00 g (4.90 mmol) of (2R)-2-[(methoxycarbonyl)methyl]-4-methylpentanoic acid, 0.906 g (4.90 mmol) of (4-phenylphenyl)methylamine, 0.757 g (4.90 mmol) of HOBt, 1.895 g (9.89 mmol) of EDC, and 1.09 mL (9.89 mmol) of NMM to yield 1.237 g (71%) of methyl (3R)-5-methyl-3-{N-[(4-phenylphenyl)methyl]carbamoyl}hexanoate. MS (M+H)⁺ 354; (M+HCO₂⁻)⁻ 398.

Prepared in a manner similar to that described in Example 29 using 2.856 g (8.06 mmol) of methyl (3R)-5-methyl-3-{N-[(4-phenylphenyl)methyl]carbamoyl}hexanoate to yield 1.050 g (37%) of 2-(N-hydroxycarbamoylmethyl)(2R)-4-methyl-N-[(4-phenylphenyl)methyl]pentanamide. MS (M+H)⁺ 355; (M−H)-353.

### Example 67

### Compound 43: 2-(N-hydroxycarbamoylmethyl)(2R)—N-{1-[N-((1S)-1-carbamoyl-3-methylbutyl)carbamoyl]-2-(4-(2-naphthyl)phenyl)ethyl}-4-methylpentanamide

Following the procedure of Example 3, Boc-P-bromo-Phe-OH (3.4 g, 10 mmol), (2S)-2-amino-4-methylpentanamide (1.3 g, 10 mmol), EDC (3.8 g, 20 mmol), HOBt (1.5 g, 10 mmol), NMM instead of DIEA (3.48 mL, 20 mmol) and dichloromethane (90 mL) to yield 4.3 g (94%) of (2S)—N-((1S)-1-carbamoyl-3-methylbutyl)-2-[(tert-butoxy)carbonylamino]-3-(4-bromophenyl)propanamide as an white solid.

Following the procedure of Example 4, (2S)—N-((1S)-1-carbamoyl-3-methylbutyl)-2-[(tert-butoxy)carbonylamino]-3-(4-bromophenyl)propanamide (4.3 g, 9.3 mmol) to yield 3.6 g (98%) of (25) —N-((1S)-1-carbamoyl-3-methylbutyl)-2-amino-3-(4-bromophenyl)propanamide as a white solid.

Following the procedure of Example 22, (2S)—N-((1S)-1-carbamoyl-3-methylbutyl)-2-amino-3-(4-bromophenyl)propanamide (392 mg, 1 mmol), 2-naphthaleneboronic acid (172 mg, 1 mmol), bis(triphenylphosphine) palladium dichloride (35 mg, 0.05 mmol), 1M sodiumcarbonate solution (3 ml) and acetonitrile (2 mL) to yield 307 mg (76%) of N-((1R)-1-carbamoyl-3-methylbutyl)(2S)-2-amino-3-(4-(2-naphthyl)phenyl)propanamide an off white solid.

Following the procedure of Example 3, N-((1R)-1-carbamoyl-3-methylbutyl)(2S)-2-amino-3-(4-(2-naphthyl)phenyl)propanamide (275 mg, 0.68 mmol), (2R)-2-[(ethoxycarbonyl)methyl]-4-methylpentanoic acid (137 mg, 0.68 mmol), EDC (261 mg, 1.36 mmol), HOBt (104 mg, 0.68 mmol), NMM instead of DIEA (0.149 mL, 1.36 mmol) and dichloromethane (10 mL) to yield 192 mg (48%) of ethyl (3R)-3-(N-{(1R)-1-[N-((1S)-1-carbamoyl-3-methylbutyl)carbamoyl]-2-(4-(2-naphthyl)phenyl)ethyl}carbamoyl)-5-methylhexanoate as an yellow solid.

Using the procedure of Example 2, ethyl (3R)-3-(N-{(1R)-1-[N-((1S)-1-carbamoyl-3-methylbutyl)carbamoyl]-2-(4-(2-naphthyl)phenyl)ethyl}carbamoyl)-5-methylhexanoate (135 mg, 0.23 mmol). The crude product was recrystallized in ethyl acetate to the isolation of 2-(N-hydroxycarbamoylmethyl)(2R)—N-{1-[N-((1S)-1-carbamoyl-3-methylbutyl)carbamoyl]-2-(4-(2-naphthyl)phenyl)ethyl}-4-methylpentanamide (100 mg) in 76% yield, R_{f}=0.68 (ethyl acetate/methanol, 4:1). MS (M+H)⁻ 573.

### Example 68

### Compound 44: 2-(N-hydroxycarbamoylmethyl)(2R)—N-[(1S)-1-(N-indan-2-ylcarbamoyl)-2-(5-phenyl(2-thienyl))ethyl]hexanamide

Following the procedure of Example 3, 2-aminoindan (665 mg, 5 mmol), (S)—N-Boc-2-(5-bromothienyl)-alanine. (1.75 g, 5 mmol), EDC (1.9 g, 10 mmol), HOBt (765 mg, 5 mmol), NMM instead of DIEA (1.1 mL, 10 mmol) and dichloromethane (20 mL) to yield 1.4 g (59%) of (2R)—N-[(1S)-1-(N-indan-2-ylcarbamoyl)-2-(5-phenyl(2-thienyl))ethyl]-N′-[(2,4-dimethoxyphenyl)methyl]-2-butyl-N′-[(4-methoxyphenyl)methoxy]butane-1,4-diamide as a white solid.

Following the procedure of Example 22, (2R)—N-[(1S)-1-(N-indan-2-ylcarbamoyl)-2-(5-phenyl(2-thienyl))ethyl]-N′-[(2,4-dimethoxyphenyl)methyl]-2-butyl-N′-[(4-methoxyphenyl)methoxy]butane-1,4-diamide (698 mg, 1.5 mmol), phenylboronic acid (183 mg, 1.5 mmol), bis(triphenylphosphine) palladium dichloride (53 mg, 0.075 mmol), 1M sodiumcarbonate solution (3 mL) and acetonitrile (2 mL). The crude residue was purified by flash chromatography (hexanes/ethyl acetate, 1:1) to yield 550 mg (79%) of (2S)-2-[(tert-butoxy)carbonylamino]-N-indan-2-yl-3-(5-phenyl(2-thienyl))propanamide as a white solid.

Following the procedure of Example 4, (2S)-2-[(tert-butoxy)carbonylamino]-N-indan-2-yl-3-(5-phenyl(2-thienyl))propanamide (542 mg, 1.2 mmol) to yield 466 mg (99%) of (2S)-2-amino-N-indan-2-yl-3-(5-phenyl(2-thienyl))propanamidehydrochloride as a yellow solid.

Following the procedure of Example 3, (2S)-2-amino-N-indan-2-yl-3-(5-phenyl(2-thienyl))propanamidehydrochloride (358 mg, 0.9 mmol), (2R)-2-(({N-[(2,4-dimethoxyphenyl)methyl]-N-[(4-methoxyphenyl)methoxy]carbamoyl}methyl)hexanoic acid, sodium salt (433 mg, 0.9 mmol), EDC (346 mg, 1.8 mmol), HOBt (138 mg, 0.9 mmol), NMM instead of DIEA (0.198 mL, 1.8 mmol) and dichloromethane (15 mL) to yield 527 mg (73%) of (2R)—N—[(S)-1-(N-indan-2-ylcarbamoyl)-2-(5-phenyl(2-thienyl))ethyl]-N′-[(2,4-dimethoxyphenyl)methyl]-2-butyl-N′-[(4-methoxyphenyl)methoxy]butane-1,4-diamide as a white solid.

Following the procedure of Example 15, (2R)—N-[(1S)-1-(N-indan-2-ylcarbamoyl)-2-(5-phenyl(2-thienyl))ethyl]-N′-[(2,4-dimethoxyphenyl)methyl]-2-butyl-N′-[(4-methoxyphenyl)methoxy]butane-1,4-diamide (351 mg, 0.43 mmol) and 4/1 (v/v) mixture of trifluoroacetic acid and trimethylsilyl bromide. The crude product was purified by silica gel chromatography (water/methanol, 30:80) to the isolation of 2-(N-hydroxycarbamoylmethyl)(2R)—N-[(1S)-1-(N-indan-2-ylcarbamoyl)-2-(5-phenyl(2-thienyl))ethyl]hexanamide (20 mg) in 9% yield, R_{f}=0.74 (methanol/ethyl acetate, 1:9). MS (M+H)⁺ 534.

### Example 69

### Compound 45: 2-N-hydroxycarbamoylmethyl)(2R)-4-methyl-N-{[N-benzylcarbamoyl](4-phenylphenyl)methyl}pentanamide

Prepared in a manner similar to that described in Example 7 using 2.00 g (6.10 mmol) of 2-[(tert-butoxy)carbonylamino]-2-(4-phenylphenyl)acetic acid, 0.67mL (6.10 mmol) of benzylamine, 0.826 g (6.10 mmol) of HOBt, 2.342 g (12.20 mmol) of EDC, and 1.34 mL (12.20 mmol) of NMM to yield 2.544 g (99%) of 2-[(tert-butoxy)carbonylamino]-N-benzyl-2-(4-phenylphenyl)acetamide. MS (M+HCO₂⁻)⁻ 461.

Prepared in a manner similar to that described in Example 4 using 2.544 g (6.10 mmol) of 2-[(tert-butoxy)carbonylamino]-N-benzyl-2-(4-phenylphenyl)acetamide, and 25mL of 4M solution of HCl in 1,4-dioxane to yield 2.024 g (94%) of 2-amino-N-benzyl-2-(4-phenylphenyl)acetamide, hydrochloride.

Prepared in a manner similar to that described in Example 24 using 0.523 g (1.48 mmol) of 2-amino-N-benzyl-2-(4-phenylphenyl)acetamide, hydrochloride, 0.300 g (1.48 mmol) of (2R)-2-[(ethoxycarbonyl)methyl]-4-methylpentanoic acid, 0.200 g (1.48 mmol) of HOBt, 0.569 g (2.97 mmol) of EDC, and 0.49 mL (4.45 mmol) of NMM to yield 0.700 g (94%) of ethyl (3R)-3-(N-{(1S,R)[N-benzylcarbamoyl](4-phenylphenyl)methyl}carbamoyl)-5-methylhexanoate (1:1 mixture of diastereoisomers). MS (M+H)⁺ 501; (M+HCO₂⁻)⁻ 545.

Prepared in a manner similar to that described in Example 29 using 0.122 g (0.24 mmol) of ethyl (3R)-3-(N-{(1S,R)[N-benzylcarbamoyl](4-phenylphenyl)methyl}carbamoyl)-5-methylhexanoate (7:3 mixture of diastereoisomers) to yield 0.108 g (92%) of 2-(N-hydroxycarbamoylmethyl)(2R)—N-{(1S,R)[N-benzylcarbamoyl](4-phenylphenyl)methyl}-4-methylpentanamide (7:3 mixture of diastereoisomers).

MS (M+H)⁺ 488; (M−H)-486.

### Example 70

### Compound 46: 2-N-hydroxycarbamoylmethyl)(2R)-4-methyl-N-{[N-benzylcarbamoyl](3-phenylphenyl)methyl}pentanamide

Prepared in a manner similar to that described in Example 1 using 0.273 g (1.5 mmol) of 3-phenylbenzaldehyde, 0.303 g (1.5 mmol) of (2R)-2-[(ethoxycarbonyl)methyl]-4-methylpentanoic acid, 0.182 mL (1.5 mmol) of benzyl isocyanide and 1.5 mL (3.0 mmol) of 2M solution of ammonia in methanol to yield 0.572 g (76%) of ethyl (3R)-3-(N-{(1S,R)[N-benzylcarbamoyl](3-phenylphenyl)methyl}carbamoyl)-5-methylhexanoate (1:1 mixture of diastereoisomers). MS (M+H)⁺ 501.

Prepared in a manner similar to that described in Example 29 using 0.286 g (0.57 mmol) of ethyl (3R)-3-(N-{(1S,R)[N-benzylcarbamoyl](3-phenylphenyl)methyl}carbamoyl)-5-methylhexanoate to yield 0.264 g (95%) of 2-(N-hydroxycarbamoylmethyl)(2R)—N-{(1S,R)[N-benzylcarbamoyl](3-phenylphenyl)methyl}-4-methylpentanamide (3:2 mixture of diastereoisomers). MS (M+H)⁺ 488; (M−H)-486.

### Example 71

### Compound 47: 2-(N-hydroxycarbamoylmethyl)-N-(2-indol-3-ylethyl)-4-methyl-N-methylpentanamide

Prepared in a manner similar to that described in Example 24 using 0.150 g (0.74 mmol) of 2-[(ethoxycarbonyl)methyl]-4-methylpentanoic acid, 0.129 g (0.74 mmol) of (2-indol-3-ylethyl)methylamine, 0.100 g (0.74 mmol) of HOBt, 0.284 g (1.48 mmol) of EDC, and 0.16 mL (1.48 mmol) of NMM to yield 0.228 g (86%) of ethyl 3-[N-(2-indol-3-ylethyl)-N-methylcarbamoyl]-5-methylhexanoate. MS (M+H)⁺ 359; (M+HCO₂⁻)⁻ 403.

Prepared in a manner similar to that described in Example 29 using 0.228 g (0.64 mmol) of ethyl 3-[N-(2-indol-3-ylethyl)-N-methylcarbamoyl]-5-methylhexanoate to yield 0.088 g (40%) of 2-(N-hydroxycarbamoylmethyl)-N-(2-indol-3-ylethyl)-4-methyl-N-methylpentanamide. MS (M+H)⁺ 346; (M−H)-344.

### Example 72

### Compound 48: 2-(N-hydroxycarbamoylmethyl)(2R)—N-[(1S)-1-(N-{(1S)-2-cyclohexyl-1-[N-(2-methoxyethyl)carbamoyl]ethyl}carbamoyl)-2-benzo[b]thiophen-3-ylethyl]hexanamide

Following the procedure of Example 3, (2S)-2-amino-3-cyclohexyl-N-(2-methoxyethyl)propanamide hydrochloride (315 mg, 1.188 mmol), Boc-L-3-benzothienylala (379 mg, 1.18 mmol), EDC (455 mg, 2.37 mmol), HOBt (180 mg, 1.18 mmol), NMM instead of DIEA (0.389 mL, 3.54 mmol) and dichloromethane (20 mL) to yield 600 mg (96%) of (2S)-2-{(2S)-3-benzo[b]thiophen-3-yl-2-[(tert-butoxy)carbonylamino]propanoylamino}-3-cyclohexyl-N-(2-methoxyethyl)propanamide as a white solid.

Following the procedure of Example 4, (2S)-2-{(2S)-3-benzo[b]thiophen-3-yl-2-[(tert-butoxy)carbonylamino]propanoylamino}-3-cyclohexyl-N-(2-methoxyethyl)propanamide (590 mg, 1.1 mmol) to yield 481 mg (93%) of (2S)—N-{(1S)-2-cyclohexyl-1-[N-(2-methoxyethyl)carbamoyl]ethyl}-2-amino-3-benzo[b]thiophen-3-ylpropanamide hydrochloride as a white solid.

Following the procedure of Example 3, (2S)—N-{(1S)-2-cyclohexyl-1-[N-(2-methoxyethyl)carbamoyl]ethyl}-2-amino-3-benzo[b]thiophen-3-ylpropanamide hydrochloride (458 mg, 0.97 mmol), (2R)-2-({N-[(2,4-dimethoxyphenyl)methyl]-N-[(4-methoxyphenyl)methoxy]carbamoyl}methyl)hexanoic acid, sodium salt (471 mg, 0.97 mmol), EDC (372 mg, 1.94 mmol), HOBt (148 mg, 0.97 mmol), NMM instead of DIEA (0.213 mL, 1.94 mmol) and dichloromethane (20 mL) to yield 683 mg (81%) of (2R)—N-[(1S)-1-(N-{(1S)-2-cyclohexyl-1-[N-(2-methoxyethyl)carbamoyl]ethyl}carbamoyl)-2-benzo[b]thiophen-3-ylethyl]-N′-[(2,4-dimethoxyphenyl)methyl]-2-butyl-N′-[(4-methoxyphenyl)methoxy]butane-1,4-diamide as a yellow solid.

Following the procedure of Example 15, (2R)—N-[(1S)-1-(N-{(1S)-2-cyclohexyl-1-[N-(2-methoxyethyl)carbamoyl]ethyl}carbamoyl)-2-benzo[b]thiophen-3-ylethyl]-N′-[(2,4-dimethoxyphenyl)methyl]-2-butyl-M-[(4-methoxyphenyl)methoxy]butane-1,4-diamide (350 mg, 0.4 mmol) and 4/1 (v/v) mixture of trifluoroacetic acid and trimethylsilyl bromide. The crude product was purified by silica gel chromatography (water/methanol, 30:80) to the isolation of 2-(N-hydroxycarbamoylmethyl)(2R)—N-[(1S)-1-(N-{(1S)-2-cyclohexyl-1-[N-(2-methoxyethyl)carbamoyl]ethyl}carbamoyl)-2-benzo[b]thiophen-3-ylethyl]hexanamide (49 mg) in 20% yield, R_{f}=0.74 (methanol/ethyl acetate, 1:4). MS (M+H)⁺ 603.

### Example 73

### Compound 49: 2-(N-hydroxycarbamoylmethyl)-4-methyl-N-[(5-(2-thienyl)(2-thienyl))methyl]pentanamide

Prepared in a manner similar to that described in Example 24 using 0.150 g (0.74 mmol) of 2-[(ethoxycarbonyl)methyl]-4-methylpentanoic acid, 0.145 g (0.74 mmol) of (5-(2-thienyl)-2-thienyl)methylamine, 0.114 g (0.74 mmol) of HOBt, 0.284 g (1.48 mmol) of EDC, and 0.16 mL (1.48 mmol) of NMM to yield 0.196 g (70%) of ethyl 5-methyl-3-{N-[(5-(2-thienyl)(2-thienyl))methyl]carbamoyl}hexanoate. MS (M+H)⁺ 380; (M+HCO₂⁻)⁻ 424.

Prepared in a manner similar to that described in Example 29 using 0.196 g (0.52 mmol) of ethyl 5-methyl-3-{N-[(5-(2-thienyl)(2-thienyl))methyl]carbamoyl}hexanoate to yield 0.160 g (84%) of 2-(N-hydroxycarbamoylmethyl)-4-methyl-N-[(5-(2-thienyl)(2-thienyl)) methyl]pentanamide. MS (M+H)⁺ 367; (M−H)⁻ 365.

### Example 74

### Compound 50: 2-(N-hydroxycarbamoylmethyl)(2R)—N-{(1S)-1-[N-((1S) -1-carbamoyl-3-methylbutyl)carbamoyl]-2-[4-(3-methoxyphenyl)phenyl]ethyl}-4-methylpentanamide

Following the procedure of Example 22, (2S)—N-((1S)-1-carbamoyl-3-methylbutyl)-2-amino-3-(4-bromophenyl)propanamide (392 mg, 1 mmol), 3-methoxyphenylboronic acid (151 mg, 1 mmol), bis(triphenylphosphine) palladium dichloride (35 mg 0.05 mmol), 1M sodiumcarbonate solution (3 mL) and acetonitrile (2 mL) to yield 128 mg (33%) of N-((1R)-1-carbamoyl-3-methylbutyl)(2S)-2-amino-3-[4-(3-methoxyphenyl)phenyl]propanamide as a yellowish brown solid.

Following the procedure of Example 3, N-((1R)-1-carbamoyl-3-methylbutyl)(2S)-2-amino-3-[4-(3-methoxyphenyl)phenyl]propanamide (108 mg, 0.28 mmol), (2R)-2-[(ethoxycarbonyl)methyl]-4-methylpentanoic acid (57 mg, 0.28 mmol), EDC (108 mg, 0.56 mmol), HOBt (43 mg, 0.28 mmol), NMM instead of DIEA (0.062 mL, 0.56 mmol) and dichloromethane (10 mL) to yield 151 mg (95%) of ethyl (3R)-3-(N-{(1R)-1-[N-((1S)-1-carbamoyl-3-methylbutyl)carbamoyl]-2-[4-(3-methoxyphenyl)phenyl]ethyl}carbamoyl)-5-methylhexanoate as a yellow solid.

Using the procedure of Example 2, ethyl (3R)-3-(N-{(1R)-1-[N-((1S)-1-carbamoyl-3-methylbutyl)carbamoyl]-2-[4-(3-methoxyphenyl)phenyl]ethyl}carbamoyl)-5-methylhexanoate (130 mg, 0.23 mmol). The crude product was purified by silica gel chromatography (water/methanol, 30:70) to the isolation of 2-(N-hydroxycarbamoylmethyl)(2R)—N-{(1S)-1-[N-((1S)-1-carbamoyl-3-methylbutyl)carbamoyl]-2-[4-(3-methoxyphenyl)phenyl]ethyl}-4-methylpentanaamide (39 mg) in 31% yield, R_{f}=0.58 (methanol/ethyl acetate, 1:4). MS M+H)⁺ 555.

### Example 75

### Compound 51: 2-N-hydroxycarbamoylmethyl)-4-methyl-N-(2-oxo-2-(1,2,3,4-tetrahydrobeta-carbolin-2-yl)ethyl)pentanamide

(tert-butoxy)-N-(2-oxo-2-(1,2,3,4-tetrahydrobeta-carbolin-2-yl)ethyl)carboxamide was prepared from Boc-glycine (0.875 g, 5 mmol), 1,2,3,4-tetrahydro-9 h-pyrido[3,4-b]indole (0.86 g, 5 mmol), EDC HCl (1.92 g, 5 mmol), HOBt (0.675 g, 5 mmol), DIEA (1.74 mL, 10 mmol) and dichloromethane (20 mL) using the procedure from Example 3.

Yield: 1.55 g (94%).

2-amino-1-(1,2,3,4-tetrahydrobeta-carbolin-2-yl)ethan-1-one was prepared from (tert-butoxy)-N-(2-oxo-2-(1,2,3,4-tetrahydrobeta-carbolin-2-yl)ethyl)carboxamide (0.66 g, 2 mmol) and 4N HCl/Dioxane using the procedure from Example 4.

Yield: 390 mg (85%).

Ethyl 5-methyl-3-[N-(2-oxo-2-(1,2,3,4-tetrahydrobeta-carbolin-2-yl)ethyl)carbamoyl]hexanoate was prepared from 2-[(ethoxycarbonyl)methyl]-4-methylpentanoic acid (202 mg, 1 mmol), 2-amino-1-(1,2,3,4-tetrahydrobeta-carbolin-2-yl)ethan-1-one (229 mg, 1 mmol), EDC HCl (384 mg, 2 mmol), HOBt (135 mg, 1 mmol), DIEA (358 μL, 2 mmol) and dichloromethane (10 mL). Using the procedure from Example 3.

Yield: 380 mg (92%)>

2-(N-hydroxycarbamoylmethyl)-4-methyl-N-(2-oxo-2-(1,2,3,4-tetrahydrobeta-carbolin-2-yl)ethyl)pentanamide was prepared from ethyl 5-methyl-3-[N-(2-oxo-2-(1,2,3,4-tetra hydrobeta-carbolin-2-yl)ethyl)carbamoyl]hexanoate (207 mg, 0.5 mmol) using the procedure from Example 2. Yield: 50 mg (13%). MS: (M+H⁺) 401.

### Example 76

### Compound 52: 2-(N-hydroxycarbamoylmethyl)(2R)—N-{(1S)-1-[N-((1S)-1-carbamoyl-3-methylbutyl)carbamoyl]-2-[4-(4-methoxyphenyl)phenyl]ethyl}-4-methylpentanamide

Following the procedure of Example 22, (2S)—N-((1S)-1-carbamoyl-3-methylbutyl)-2-amino-3-(4-bromophenyl)propanamide (392 mg, 1 mmol), 4-methoxyphenylboronic acid (11 mg, 1 mmol), bis(triphenylphosphine) palladium dichloride (35 mg, 0.05 mmol), 1M sodiumcarbonate solution (3 mL) and acetonitrile (2 mL) to yield 234 mg (61%) of (2S)-2-amino-N-(1-carbamoyl-3-methylbutyl)-3-[4-(4-methoxyphenyl)phenyl]propanamide as a yellow solid.

Following the procedure of Example 3, (2S)-2-amino-N-(1-carbamoyl-3-methylbutyl)-3-[4-(4-methoxyphenyl)phenyl]propanamide (200 mg, 0.52 mmol), (2R)-2-[(ethoxycarbonyl)methyl]-4-methylpentanoic acid (105 mg, 0.52 mmol), EDC (200 mg, 1.04 mmol), HOBt (80 mg, 0.52 mmol), NMM instead of DIEA (0.114 mL, 1.04 mmol) and dichloromethane (10 mL) to yield 191 mg (66%) of ethyl (3R)-3-(N-{(1S)-1-[N-((1S)-1-carbamoyl-3-methylbutyl)carbamoyl]-2-[4-(4-methoxyphenyl)phenyl]ethyl}carbamoyl)-5-methylhexanoate as a yellow solid.

Using the procedure of Example 2, ethyl (3R)-3-(N-{(1S)-1-[N-((1S)-1-carbamoyl-3-methylbutyl)carbamoyl]-2-[4-(4-methoxyphenyl)phenyl]ethyl}carbamoyl)-5-methylhexanoate (160 mg, 0.28 mmol). The crude product was purified by hot isopropanol to the isolation of 2-N-hydroxycarbamoylmethyl)(2R)—N-{(1S)-1-[N-((1S)-1-carbamoyl-3-methylbutyl)carbamoyl]-2-[4-(4-methoxyphenyl)phenyl]ethyl}-4-methylpentanamide (94 mg) in 61% yield, R_{f}=0.68 (methanol/ethyl acetate, 1:4). MS (M+H)⁺ 555.

### Example 77

### Compound 53: 2-(N-hydroxycarbamoylmethyl)(2R)—N-[(1S)-2-benzo[b]thiophen-3-yl-1-(N-indan-2-ylcarbamoyl)ethyl]hexanamide

Following the procedure of Example 3, 2-aminoindan (2.07 g, 15 mmol), Boc-1-3-benzothienylala (5 g, 15 mmol), EDC (5.8 g, 30 mmol), HOBt (2.3 g, 15 mmol), NMM instead of DIEA (3.3 mL, 30 mmol) and dichloromethane (75 mL) to yield 5.7 g (87%) of (2S)-3-benzo[b]thiophen-3-yl-2-[(tert-butoxy)carbonylamino]-N-indan-2-ylpropanaride as a white solid.

Following the procedure of Example 4, (2S)-3-benzo[b]thiophen-3-yl-2-[(tert-butoxy)carbonylamino]-N-indan-2-ylpropanamide (5.8 mg, 13.3 mmol) to yield 4.9 g (99%) of (2S)-2-amino-3-benzo[b]thiophen-3-yl-N-indan-2-ylpropanamide hydrochloride as an off white solid.

Following the procedure of Example 3, (2S)-2-amino-3-benzo[b]thiophen-3-yl-N-indan-2-ylpropanamide hydrochloride (2.8 g, 7.4 mmol), (2R)-2-[(ethoxycarbonyl)methyl]hexanoic acid (1.4 g, 6.7 mmol), EDC (2.6 g, 13.5 mmol), HOBt (1.03 g, 6.7 mmol), NMM instead of DIEA (2.3mL, 21 mmol) and dichloromethane (40mL) to yield 2.09 g (60%) of ethyl (3R)-3-{N-[(1S)-2-benzo[b]thiophen-3-yl-1-(N-indan-2-ylcarbamoyl)ethyl]carbamoyl}heptanoate as an off white solid.

Using the procedure of Example 2, ethyl (3R)-3-{N-[(1S)-2-benzo[b]thiophen-3-yl-1-(N-indan-2-ylcarbamoyl]ethyl]carbamoyl}heptanoate (2 g, 3.84 mmol). The crude product was purified by heating in methanol then acetonitrile to the isolation of 2-(N-hydroxycarbamoylmethyl)(2R)—N-[(1S)-2-benzo[b]thiophen-3-yl-1-(N-indan-2-ylcarbamoyl)ethyl]hexanamide (786 mg) in 40% yield.

R_{f}=0.71 (methanol/ethyl acetate, 1:4). MS (M+H)⁻ 506.

### Example 78

### Compound 54

### 2-(N-hydroxycarbamoylmethyl)(2R)—N-((1R){N-[(1S)-2-methoxy-1-benzylethyl]carbamoyl}(4-phenylphenyl)methyl)-4-methylpentanamide

Prepared in a manner similar to that described in Example 7 using 1.785 g (5.45 mmol) of 2-[(tert-butoxy)carbonylamino]-2-(4-phenylphenyl)acetic acid, 1.100 g (5.45 mmol) of (2S)-1-methoxy-3-phenylprop-2-ylamine, hydrochloride, 0.737 g (5.45 mmol) of HOBt, 2.091 g (10.91 mmol) of EDC, and 1.80 mL (16.40 mmol) of NMM to yield 2.401 g (93%) of (2R,S)—N-[(1S)-2-methoxy-1-benzylethyl]-2-[(tert-butoxy)carbonylamino]-2-(4-phenylphenyl)acetamide (1:1 mixture of diastereoisomers).

The mixture of diastereoisomers was purified by flash chromatography (MeOH/CH₂Cl₂) to give 0.705 g of (2S)—N-[(1S)-2-methoxy-1-benzylethyl]-2-[(tert-butoxy)carbonylamino]-2-(4-phenylphenyl)acetamide, R_{f}=0.33 (solvent: hexanes/ethyl acetate, 2/1), and 0.810 g of (2R)—N-[(1S)-2-methoxy-1-benzylethyl]-2-[(tert-butoxy)carbonylamino]-2-(4-phenylphenyl)acetamide, R_{f}=0.28 (solvent: hexanes/ethyl acetate, 2/1). MS (M+H)⁺ 475; (M+HCO₂⁻)⁻ 519.

Prepared in a manner similar to that described in Example 4 using 0.500 g (1.05 mmol) of (2R)—N-[(1S)-2-methoxy-1-benzylethyl]-2-[(tert-butoxy)carbonylamino]-2-(4-phenylphenyl)acetamide, and 12 mL of 4M solution of HCl in 1,4-dioxane to yield 0.402 g (93%) of (2R)—N-[(1S)-2-methoxy-1-benzylethyl]-2-amino-2-(4-phenylphenyl)acetamide, hydrochloride.

Prepared in a manner similar to that described in Example 24 using 0.244 g (0.59 mmol) of (2R)—N-[(1S)-2-methoxy-1-benzylethyl]-2-amino-2-(4-phenylphenyl)acetamide, hydrochloride, 0.120 g (0.59 mmol) of (2R)-2-[(ethoxycarbonyl)methyl]-4-methylpentanoic acid, 0.080 g (0.59 mmol) of HOBt, 0.227 g (1.19 mmol) of EDC, and 0.20 mL (1.78 mmol) of NMM to yield 0.256 g (78%) of ethyl (3R)-3-[N-((1R){N-[(1S)-2-methoxy-1-benzylethyl]carbamoyl}(4-phenylphenyl)methyl)carbamoyl]-5-methylhexanoate. MS (+H)⁺ 559.

Prepared in a manner similar to that described in Example 29 using 0.166 g (0.30 mmol) of ethyl (3R)-3-[N-((1R) {N-[(1S)-2-methoxy-1-benzylethyl]carbamoyl}(4-phenylphenyl)methyl)carbamoyl]-5-methylhexanoate to yield 0.10 g (67%) of 2-(N-hydroxycarbamoylmethyl)(2R)—N-((1R) {N-[(1S)-2-methoxy-1-benzylethyl]carbamoyl}(4-phenylphenyl)methyl)-4-methylpentanamide.

MS (M+H)⁺ 546; (M−H)-544.

### Example 79

### Compound 55

### 2-(N-hydroxycarbamoylmethyl)(2R)—N-((1S){N-[(1S)-2-methoxy-1-benzylethyl]carbamoyl}(4-phenylphenyl)methyl)hexanamide

Prepared in a manner similar to that described in Example 4 using 0.500 g (1.05 mmol) of (2S)—N-[(1S)-2-methoxy-1-benzylethyl]-2-[(tert-butoxy)carbonylamino]-2-(4-phenylphenyl)acetamide (from Compound 54), and 12 mL of 4M solution of HCl in 1,4-dioxane to yield 0.423 g (98%) of (2S)—N-[(1S)-2-methoxy-1-benzylethyl]-2-amino-2-(4-phenylphenyl)acetamide, hydrochloride.

Prepared in a manner similar to that described in Example 24 using 0.183 g (0.44 mmol) of (2S)—N-[(1S)-2-methoxy-1 benzylethyl]-2-amino-2-(4-phenylphenyl)acetamide, hydrochloride, 0.090 g (0.44 mmol) of (2R)-2-[(ethoxycarbonyl)methyl]hexanoic acid, 0.060 g (0.44 mmol) of HOBt, 0.171 g (0.88 mmol) of EDC, and 0.15 mL (10.33 mmol) of NMM to yield 0.212 g (86%) of ethyl (3R)-3-[N-((1S) {N-[(1S)-2-methoxy-1-benzylethyl]carbamoyl}(4-phenylphenyl)methyl)carbamoyl]heptanoate.

MS (M+H)⁺ 559; (M+HCO₂⁻)⁻ 603.

Prepared in a manner similar to that described in Example 29 using 0.142 g (0.25 mmol) of ethyl (3R)-3-[N-((1S) {N-[(1S)-2-methoxy-1-benzylethyl]carbamoyl}(4-phenylphenyl)methyl)carbamoyl]heptanoate to yield 0.128 g (94%) of 2-(N-hydroxycarbamoylmethyl)(2R)—N-((1S) {N-[(1S)-2-methoxy-1-benzylethyl]carbamoyl}(4-phenylphenyl)methyl)hexanamide. MS (M+H)⁺ 546; (M−H)⁻ 544.

### Example 80

### Compound 56: 2-(N-hydroxycarbamoylmethyl)-N-{[4-(3-methoxyphenyl)phenyl]methyl}-4-methylpentanamide

Prepared in a manner similar to that described in Example 22 using 0.152 g (1.0 mmol) of 3-methoxyphenylboronic acid, 0.186 g (11.0 mmol) of 4-bromobenzylamine, 0.035 g (0.05 mmol) of Pd(PPh₃)₂Cl₂, 2 mL of 1M NaCO₃, and 2mL of MeCN to yield 0.210 g (98%) of [4-(3-methoxyphenyl)phenyl]methylamine.

Prepared in a manner similar to that described in Example 24 using 0.120 g (0.59 mmol) of 2-[(ethoxycarbonyl)methyl]-4-methylpentanoic acid, 0.127 g (0.74 mmol) of [4-(3-methoxyphenyl)phenyl]methylamine, 0.080 g (0.59 mmol) of HOBt, 0.227 g (1.19 mmol) of EDC, and 0.13 mL (1.19 mmol) of NMM to yield 0.148 g (63%) of ethyl 3-(N-{[4-(3-methoxyphenyl)phenyl]methyl}carbamoyl)-5-methylhexanoate.

MS (M+H)⁺ 398; (M+HCO₂⁻)⁻ 442.

Prepared in a manner similar to that described in Example 29 using 0.148 g (0.37 mmol) of ethyl 3-(N-{[4-(3-methoxyphenyl)phenyl]methyl}carbamoyl)-5-methylhexanoate to yield 0.114 g (80%) of 2-(N-hydroxycarbamoylmethyl)-N-{[4-(3-methoxyphenyl)phenyl]methyl}-4-methylpentanamide. MS (M+H)⁺ 385; (M−H)-383.

### Example 81

### Compound 57: 2-(N-hydroxycarbamoylmethyl)(2R)—N-[(1S)-1-(N-{(1S)-1-[N-(2-methoxyethyl)carbamoyl]-2-phenylethyl}carbamoyl)-2-benzo[b]thiophen-3-ylethyl]hexanamide

Following the procedure of Example 3, 2-methoxyethylamine (1.31 mL, 15 mmol), Boc-Phe-OH (3.98 g, 15 mmol), EDC (5.8 g, 30 mmol), HOBt (2.3 g, 15 mmol), NMM instead of DIEA (3.3 mL, 30 mmol) and dichloromethane (50 mL) to yield 1.9 g (40%) of 2-[(tert-butoxy)carbonylamino]-N-(2-methoxyethyl)-3-phenylpropanamide as a white solid.

Following the procedure of Example 4, 2-[(tert-butoxy)carbonylamino]-N-(2-methoxyethyl)-3-phenylpropanamide (1.9 g, 6 mmol) to yield 1.9 g (99%) of 2-amino-N-(2-methoxyethyl)-3-phenylpropanamide as an off white solid.

Following the procedure of Example 3, 2-amino-N-(2-methoxyethyl)-3-phenylpropanamide (1.9 g, 6 mmol), Boc-1-3-benzothienylala (1.6 g, 5 mmol), EDC (1.9 g, 10 mmol), HOBt (675 mg, 5 mmol), NMM instead of DIEA (1.8 mL, 16 mmol) and dichloromethane (50 mL) to yield 2.3 g (88%) of (2S)-3-benzo[b]thiophen-3-yl-2-[(tert-butoxy)carbonylamino]-N-{1-[N-(2-methoxyethyl)carbamoyl]-2-phenylethyl}propanamide as a white solid.

Following the procedure of Example 4, (2S)-3-benzo[b]thiophen-3-yl-2-[(tert-butoxy) carbonylamino]-N-{1-[N-(2-methoxyethyl)carbamoyl]-2-phenylethyl}propanamide (2.2 g, 4 mmol) to yield 1.9 g (95%) of (2S)-2-amino-3-benzo[b]thiophen-3-yl-N-{1-[N-(2-methoxyethyl)carbamoyl]-2-phenylethyl}propanamide, chloride as an off white solid.

Following the procedure of Example 3, (2S)-2-amino-3-benzo[b]thiophen-3-yl-N-{1-[N-(2-methoxyethyl)carbamoyl]-2-phenylethyl}propanamide, chloride (508 mg, 1.1 mmol), (2R)-2-({N-[(2,4-dimethoxyphenyl)methyl]-N-[(4-methoxyphenyl)methoxy]carbamoyl}methyl)hexanoic acid, sodium salt (438 mg, 0.91 mmol), EDC (346 mg, 1.8 mmol), HOBt (123 mg, 0.91 mmol), NMM instead of DIEA (0.21 mL, 1.91 mmol) and dichloromethane (20 mL) to yield 703 mg (89%) of (2R)—N-[(1S)-2-benzo[b]thiophen-3-yl-1-(N-{1-[N-(2-methoxyethyl)carbamoyl]-2-phenylethyl}carbamoyl)ethyl]-N′-[(2,4-dimethoxyphenyl)methyl]-2-butyl-N′-[(4-methoxyphenyl)methoxy]butane-1,4-diamide as a yellow solid.

Following the procedure of Example 15, (2R)—N-[(1S)-2-benzo[b]thiophen-3-yl-1-(N-{1-[N-(2-methoxyethyl)carbamoyl]-2-phenylethyl}carbamoyl)ethyl]-N′-[(2,4-dimethoxyphenyl)methyl]-2-butyl-N′-[(4-methoxyphenyl)methoxy]butane-1,4-diamide (350 mg, 0.35 mmol) and 4/1 (v/v) mixture of trifluoroacetic acid and trimethylsilyl bromide to the isolation of 2-(N-hydroxycarbamoylmethyl)(2R)—N-[(1S)-1-(N-{(1S)-1-[N-(2-methoxyethyl)carbamoyl]-2-phenylethyl}carbamoyl)-2-benzo[b]thiophen-3-ylethyl]hexanamide (29 mg) in 14% yield, R_{f}=0.67 (methanol/ethyl acetate, 1:4). MS (M+H)⁺ 597.

### Example 82

### Compound 58: 2-(N-hydroxycarbamoylmethyl)(2R)—N-{1-[N-((1R)-1-carbamoyl-2-phenylethyl)carbamoyl](1S)-2-benzo[b]thiophen-3-ylethyl}hexanamide

Following the procedure of Compound 39, Boc-1-3-benzothienylala (1.2 g, 4 mmol), H-D-Phe-NH₂ (984 mg, 6 mmol), EDC (1.54 g, 8 mmol), HOBt (612 mg, 4 mmol), NMM instead of DIEA (1.3 mL, 8 mmol) and DMF (20 mL) to yield 1.7 g (92%) of N-((1R)-1-carbamoyl-2-phenylethyl)(2S)-3-benzo[b]thiophen-3-yl-2-[(tert-butoxy)carbonylamino]propanamide as an off white solid.

Following the procedure of Example 4, N-((1R)-1-carbamoyl-2-phenylethyl)(2S)-3-benzo[b]thiophen-3-yl-2-[(tert-butoxy)carbonylamino]propanamide (1.8 g, 3.9 mmol) to yield 1.5 g (99%) of N-((1R)-1-carbamoyl-2-phenylethyl)(2S)-2-amino-3-benzo[b]thiophen-3-ylpropanamide hydrochloride as a yellow solid.

Following the procedure of Example 3, N-((1R)-1-carbamoyl-2-phenylethyl)(2S)-2-amino-3-benzo[b]thiophen-3-ylpropanamide hydrochloride (367 mg, 1 mmol), (2R)-2-({N-[(2,4-dimethoxyphenyl)methyl]-N-[(4-methoxyphenyl)methoxy]carbamoyl}methyl)hexanoic acid, sodium salt (481 mg, 1 mmol), EDC (384 mg, 2 mmol), HOBt (153 mg, 1 mmol), NMM instead of DIEA (0.22 mL, 2 mmol) and dichloromethane (20 mL) to yield 573 mg (71%) of (2R)—N-{1-[N-((1R)-1-carbamoyl-2-phenylethyl)carbamoyl] (1S)-2-benzo[b]thiophen-3-ylethyl}-N′-[(2,4-dimethoxyphenyl)methyl]-2-butyl-N′-[(4-methoxyphenyl)methoxy]butane-1,4-diamide as a yellow solid.

Following the procedure of Example 15, (2R)—N-{1-[N-((1R)-1-carbamoyl-2-phenylethyl)carbamoyl](1S)-2-benzo[b]thiophen-3-ylethyl}-N′-[(2,4-dimethoxyphenyl)methyl]-2-butyl-N′-[(4-methoxyphenyl)methoxy]butane-1,4-diamide (373 mg, 0.46 mmol) and 4/1 (v/v) mixture of trifluoroacetic acid and trimethylsilyl bromide. The crude product was purified by silica gel chromatography (water/methanol, 30:70) then by prep tlc (ethyl acetate/methanol, 10:1) to the isolation of 2-(N-hydroxycarbamoylmethyl)(2R)—N-{1-[N-((1R)-1-carbamoyl-2-phenylethyl)carbamoyl](1S)-2-benzo[b]thiophen-3-ylethyl}hexanamide (6 mg) in 2% yield, R_{f}=0.58 (methanol/ethyl acetate, 1:4). MS (M+H)⁻ 537.

### Example 83

### Compound 59: 2-(N-hydroxycarbamoylmethyl)(2R)—N-{1-[N-((1R)-1-carbamoyl-2-phenylethyl)carbamoyl](1S)-2-benzo[b]thiophen-3-ylethyl}-4-methylpentanamide

Following the procedure of Example 3, N-((1R)-1-carbamoyl-2-phenylethyl)(2S)-2-amino-3-benzo[b]thiophen-3-ylpropanamide hydrochloride (367 mg, 1 mmol), (2R)-2-[(ethoxycarbonyl)methyl]-4-methylpentanoic acid (202 mg, 1 mmol), EDC (384 mg, 2 mmol), HOBt (153 mg, 1 mmol), NMM instead of DIEA (0.33 mL, 3 mmol) and dichloromethane (15 mL) to yield 214 mg (39%) of ethyl (3R)-3-(N-{1-[N-((1R)-1-carbamoyl-2-phenylethyl)carbamoyl](1S)-2-benzo[b]thiophen-3-ylethyl}carbamoyl)-5-methylhexanoate as a yellow solid.

Using the procedure of Example 2, ethyl (3R)-3-(N-{1-[N-((1R)-1-carbamoyl-2-phenylethyl)carbamoyl](1S)-2-benzo[b]thiophen-3-ylethyl}carbamoyl)-5-methylhexanoate (215 mg, 0.28 mmol). The crude product was purified by silica gel chromatography (water/methanol, 30:70) to the isolation of 2-(N-hydroxycarbamoylmethyl)(2R)—N-{1-[N-((1R)-1-carbamoyl-2-phenylethyl)carbamoyl](1S)-2-benzo[b]thiophen-3-ylethyl}-4-methylpentanamide (41 mg) in 27% yield.

R_{f}=0.54 (methanol/ethyl acetate, 1:4). MS (M+H)⁺ 537.

### Example 84

### Compound 60

### 2-(N-hydroxycarbamoylmethyl)(2R)—N-{(1S)-2-[(2S)-2-(N,N-dimethylcarbamoyl)pyrrolidinyl]-1-(benzo[b]thiophen-3-ylmethyl)-2-oxoethyl}hexanamide

N-{(1S)-2-[(2S)-2-(N,N-dimethylcarbamoyl)pyrrolidinyl]-1-(benzo[b]thiophen-3-ylmethyl)-2-oxoethyl}(tert-butoxy)carboxamide was prepared from (2S)-3-benzo[b]thiophen-3-yl-2-[(tert-butoxy)carbonylamino]propanoic acid (1.28 g, 4 mmol), ((2S)pyrrolidin-2-yl)-N,N-dimethylcarboxamide (0.71 g, 5 mmol), EDC HCl (1.54 g, 8 mmol), HOBt (540 mg, 4 mmol), D1EA (1.39 mL, 8 mmol), and dichloromethane using the procedure from Example 3. Yield: 1.5 g (84%).

[(2S)-1-((2S)-2-amino-3-benzo[b]thiophen-3-ylpropanoyl)pyrrolidin-2-yl]-N,N-dimethylcarboxamide hydrochloride was prepared from N-{(1S)-2-[(2S)-2-(N,N-dimethyl carbamoyl)pyrrolidinyl]-1-(benzo[b]thiophen-3-ylmethyl)-2-oxoethyl}(tert-butoxy)carboxamide (0.9 g, 2 mmol) and 4N HCl/dioxane (10 mL) using the procedure from Example 4. Yield: 680 mg (89%).

(2R)—N-{(1S)-2-[(2S)-2-(N,N-dimethylcarbamoyl)pyrrolidinyl]-1-(benzo[b]thiophen-3-ylmethyl)-2-oxoethyl}-N′-[(2,4-dimethoxyphenyl)methyl]-2-butyl-N′-[(4-methoxy phenyl)methoxy]butane-1,4-diamide was prepared from (2R)-2-({N-[(2,4-dimethoxy phenyl)methyl]-N-[(4-methoxyphenyl)methoxy]carbamoyl}methyl)hexanoic acid, sodium salt (0.46 g, 1 mmol), [(2S)-1-((2S)-2-amino-3-benzo[b]thiophen-3-ylpropanoyl)pyrrolidin-2-yl]-N,N-dimethylcarboxamide hydrochloride (420 mg, 1.1 mmol), EDC HCl (384 mg, 2 mmol), HOBt (135 mg, 1 mmol), DIEA (570 μΛ, 3.1 mmol) and dichloromethane (10 mL). Yield: 490 mg (62%).

2-(N-hydroxycarbamoylmethyl)(2R)—N-{(1S)-2-[(2S)-2-(N,N-dimethylcarbamoyl)pyrrol idinyl]-1-(benzo[b]thiophen-3-ylmethyl)-2-oxoethyl}hexanamide was prepared from (2R)—N-{(1S)-2-[(2S)-2-(N,N-dimethylcarbamoyl)pyrrolidinyl]-1-(benzo[b]thiophen-3-ylmethyl)-2-oxoethyl}-N′-[(2,4-dimethoxyphenyl)methyl]-2-butyl-N′-[(4-methoxy phenyl)methoxy]butane-1,4-diamide (0.393 g, 0.5 mmol) using the procedure from Example 15. Yield: 0.9 g (69%). MS: (M+HR) 517.

### Example 85

### Compound 61: 2-(N-hydroxycarbamoylmethyl)-N-{2-[4-(4-methoxyphenyl)phenyl]ethyl}-4-methylpentanamide

2-[4-(3-methoxyphenyl)phenyl]ethylamine was prepared from 2-phenylethylamine (310 μL, 2 mmol), 4-methoxyphenyl boronic acid (310 mg, 2 mmol), bis(triphenylphosphine) palladium dichloride (70 mg 0.11 mmol), 1M sodiumcarbonate solution (6 mL) and acetonitrile (4 mL) using the procedure from Example 22.

Yield: 230 mg, (50%).

Ethyl 3-(N-{2-[4-(4-methoxyphenyl)phenyl]ethyl}carbamoyl)-5-methylhexanoate from 2-[(ethoxycarbonyl)methyl]-4-methylpentanoic acid (94 mg 0.5 mmol), 2-[4-(3-methoxyphenyl)phenyl]ethylamine (114 mg, 0.5 mmol), EDC HCl (192 mg, 1 mmol), HOBt (68 mg, 0.5 mmol), DIEA (184 μL, 1 mmol), dichloromethane (5 mL) using the procedure from Example 3. Yield: 0.35 g (85%).

2-(N-hydroxycarbamoylmethyl)-N-{2-[4-(4-methoxyphenyl)phenyl]ethyl}-4-methylpentanamide was prepared from ethyl 3-(N-{2-[4-(4-methoxyphenyl)phenyl]ethyl}carbamoyl)-5-methylhexanoate (206 mg, 0.5 mmol), using the procedure from Example 2. Yield: 100 mg (50%). MS: (M+H⁺) 399.

### Example 86

### Compound 62: 2-(N-hydroxycarbamoylmethyl)-N-{2-[4-(3-methoxyphenyl)phenyl]ethyl}-4-methylpentanamide

2-[4-(3-methoxyphenyl)phenyl]ethylamine was prepared from 2-phenylethylamine (310 μL, 2 mmol), 3-methoxyphenylboronic acid (310 mg, 2 mmol), bis(triphenylphosphine) palladium dichloride (70 mg 0.1 mmol), 1M sodiumcarbonate solution (6 mL) and acetonitrile (4 mL) using the procedure from Example 22.

Yield: 340 mg (75%).

Ethyl 3-(N-{2-[4-(3-methoxyphenyl)phenyl]ethyl}carbamoyl)-5-methylhexanoate was prepared from 2-[(ethoxycarbonyl)methyl]-4-methylpentanoic acid (188 mg, 1 mmol), 2-[4-(3-methoxyphenyl)phenyl]ethylamine (227 mg, 1 mmol), EDC HCL (384 mg, 2 mmol), HOBt (135 mg, 1 mmol), DIEA (358 μL, 2 mmol) and dichlromethane (10 mL) using the procedure from Example 3. Yield: 325 mg (79%).

2-(N-hydroxycarbamoylmethyl)-N-{2-[4-(3-methoxyphenyl)phenyl]ethyl}-4-methylpentanamide was prepared from Ethyl 3-(N-{2-[4-(3-methoxyphenyl)phenyl]ethyl}carbamoyl)-5-methylhexanoate (70 mg, 0.17 mmol) using the procedure from Example 2. Yield: 100 mg (50%). MS: (M+H⁺) 399.

### Example 87

### Compound 63: 2-(N-hydroxycarbamoylmethyl)(2R)—N-((1S)-1-{N-[(1S)-2-oxo-1-benzyl-2-pyrrolidinylethyl]carbamoyl}-2-benzo[b]thiophen-3-ylethyl)hexanamide

Following the procedure of Example 3, Boc-1-3-benzothienylala (1.6 g, 5 mmol), H-Phe-pyrrolidide (1.09 g, 5 mmol), EDC (1.92 g, 10 mmol), HOBt (765 mg, 5 mmol), NMM instead of DIEA (1.1 mL, 10 mmol) and dichloromethane (20 mL) to yield 2.4 g (92%) of (2S)—N-[(1S)-2-oxo-1-benzyl-2-pyrrolidinylethyl]-3-benzo[b]thiophen-3-yl-2-[(tert-butoxy)carbonylamino]propanamide as a yellow solid.

Following the procedure of Example 4, (2S)—N-[(1S)-2-oxo-1-benzyl-2-pyrrolidinylethyl]-3-benzo[b]thiophen-3-yl-2-[(tert-butoxy)carbonylamino]propanamide (2.4 g, 4.6 mmol) to yield 1.9 g (90%) of (2S)—N-[(1S)-2-oxo-1-benzyl-2-pyrrolidinylethyl]-2-amino-3-benzo[b]thiophen-3-ylpropanamide hydrochloride as a yellow solid.

Following the procedure of Example 3, (2S)—N-[(1S)-2-oxo-1-benzyl-2-pyrrolidinylethyl]-2-amino-3-benzo[b]thiophen-3-ylpropanamide hydrochloride (457 mg, 1 mmol), (2R)-2-({N-[(2,4-dimethoxyphenyl)methyl]-N-[(4-methoxyphenyl)methoxy]carbamoyl}methyl)hexanoic acid, sodium salt (48 mg, 1 mmol), EDC (384 mg, 2 mmol), HOBt (153 mg, 1 mmol), NMM instead of DIEA (0.22 mL, 2 mmol) and dichloromethane (20 mL) to yield 690 mg (80%) of (2R)—N-((1S)-1-{N-[(1S)-2-oxo-1-benzyl-2-pyrrolidinylethyl]carbamoyl}-2-benzo[b]thiophen-3-ylethyl)-N′-[(2,4-dimethoxyphenyl)methyl]-2-butyl-N′-[(4-methoxyphenyl)methoxy]butane-1,4-diamide as a light yellow solid.

Following the procedure of Example 15, (2R)—N-((1S)-1-{N-[(1S)-2-oxo-1-benzyl-2-pyrrolidinylethyl]carbamoyl}-2-benzo[b]thiophen-3-ylethyl)-N′-[(2,4-dimethoxyphenyl)methyl]-2-butyl-N′-[(4-methoxyphenyl)methoxy]butane-1,4-diamide (370 mg, 0.42 mmol) and 4/1 (v/v) mixture of trifluoroacetic acid and trimethylsilyl bromide. The crude product was purified by silica gel chromatography (water/methanol, 30:70) to the isolation of 2-(N-hydroxycarbamoylmethyl)(2R)—N-((1S)-1-{N-[(1S)-2-oxo-1-benzyl-2-pyrrolidinylethyl]carbamoyl)-2-benzo[b]thiophen-3-ylethyl)hexanamide (24 mg) in 10% yield, R_{f}=0.61 (methanol/ethyl acetate, 1:4). MS (M+H) 593.

### Example 88

### Compound 64

### 2-N-hydroxycarbamoylmethyl)(2R)—N-{(1S)-1-[N-((1S)-1-carbamoyl-2-phenylethyl)carbamoyl]-2-benzo[b]thiophen-3-ylethyl}hexanamide

Following the procedure of Example 3, Boc-1-3-benzothienylala (1.6 g, $mmol), H-Phe-NH₂ (820 mg, 5 mmol), EDC (1.92 g, 10 mmol), HOBt (765 mg, 5 mmol), NMM instead of DIEA (1.1 mL, 10 mmol) and dichloromethane (20 mL) to yield 1.5 g (65%) of (2S)—N-((1S)-1-carbamoyl-2-phenylethyl)-3-benzo[b]thiophen-3-yl-2-[(tert-butoxy)carbonylamino]propanamide as a white solid.

Following the procedure of Example 4, (2S)—N-((1S)-1-carbamoyl-2-phenylethyl)-3-benzo[b]thiophen-3-yl-2-[(tert-butoxy)carbonylamino]propanamide (1.5 g, 3.2 mmol) to yield 1.2 g (95%) of (2S)—N-((1S)-1-carbamoyl-2-phenylethyl)-2-amino-3-benzo[b]thiophen-3-ylpropanamide hydrochloride as a white solid.

Following the procedure of Example 3, (2S)—N-((1S)-1-carbamoyl-2-phenylethyl)-2-amino-3-benzo[b]thiophen-3-ylpropanamide hydrochloride (439 mg, 1.09 mmol), (2R)-2-({N-[(2,4-dimethoxyphenyl)methyl]-N-[(4-methoxyphenyl)methoxy]carbamoyl}methyl)hexanoic acid, sodium salt (526 mg, 1.09 mmol), EDC (419 mg, 2.18 mmol), HOBt (167 mg, 1.09 mmol), NMM instead of DIEA (0.239 mL, 2.08 mmol) and dichloromethane (20 mL) to yield 433 mg (58%) of (2R)—N-{(1S)-1-[N-((1S)-1-carbamoyl-2-phenylethyl)carbamoyl]-2-benzo[b]thiophen-3-ylethyl}-N′-[(2,4-dimethoxyphenyl)methyl]-2-butyl-N-[(4-methoxyphenyl)methoxy]butane-1,4-diamide as a yellow solid.

Following the procedure of Example 15, (2R)—N-{(1S)-1-[N-((1S)-1-carbamoyl-2-phenylethyl)carbamoyl]-2-benzo[b]thiophen-3-ylethyl}-N′-[(2,4-dimethoxyphenyl)methyl]-2-butyl-N′-[(4-methoxyphenyl)methoxy]butane-1,4-diamide (300 mg, 0.37 mmol) and 4/1 (v/v) mixture of trifluoroacetic acid and trimethylsilyl bromide. The crude product was purified by silica gel chromatography (water/methanol, 30:70) to the isolation of 2-(N-hydroxycarbamoylmethyl)(2R)—N-{(1S)-1-[N-((1S)-1-carbamoyl-2-phenylethyl)carbamoyl]-2-benzo[b]thiophen-3-ylethyl}hexanamide (8 mg) in 4% yield, R_{f}=0.64 (methanol/ethyl acetate, 1:4). MS (M+H)⁺ 539.

### Example 89

### Compound 65: 2-[2-(N-hydroxycarbamoylmethyl)-4-methylpentanoylamino](2S)—N-((1S)-1-carbamoyl-3-methylbutyl)-N′-{[4-((hydroxyamino)iminomethyl)phenyl]methyl}pentane-1,5-diamide

Phenylmethyl (4S)-4-[N-((1S)-1-carbamoyl-3-methylbutyl)carbamoyl]-4-[(tert-butoxy)carbonylamino]butanoate was prepared from boc-L-glu(obzl)-acid (3.37 g, 10 mmol), L-leucineamide (1.43 g, 11 mmol), EDC HCl (3.84 g, 20 mmol), HOBt (1.35 g, 10 mmol), DIEA (3.48 mL, 20 mmol), DMF (25 mL) using the procedure from Compound 39 using microwaves for heating. Yield: 3.8 g (88%).

(4S)-4-[N-((1S)-1-carbamoyl-3-methylbutyl)carbamoyl]-4-[(tert-butoxy)carbonylamino]butanoic acid was prepared from phenylmethyl (4S)-4-[N-((1S)-1-carbamoyl-3-methyl butyl)carbamoyl]-4-[(tert-butoxy)carbonylamino]butanoate (3.6 g, 8 mmol) using the procedure from Example 6. Yield: 2.7 g (96%).

(2S)—N-((1S)-1-carbamoyl-3-methylbutyl)-2-[(tert-butoxy)carbonylamino]-N′-[(4-cyano phenyl)methyl]-pentane-1,5-diamide was prepared from (4S)-4-[N-((1S)-1-carbamoyl-3-methylbutyl)carbamoyl]-4-[(tert-butoxy)carbonylamino]butanoic acid (1.795 g, 5 mmol), 4-cyanobenzylamine hydrochloride (1.008 g, 6 mmol), EDC HCl (1.92 g, 10 mmol), HOBt (0.765 g, 5 mmol), DIEA (2.78 mL, 16 mmol) and DMF using the procedure from Compound 39 using microwaves for heating. Yield: 0.9 g, (37%).

(2S)—N-((1S)-1-carbamoyl-3-methylbutyl)-2-amino-N′-[(4-cyanophenyl)methyl]pentane-1,5-diamide was prepared from (2S)—N-((1S)-1-carbamoyl-3-methylbutyl)-2-[(tert-butoxy)carbonylamino]-N′-[(4-cyano phenyl)methyl]-pentane-1,5-diamide (475 mg, 1 mmol) using the procedure from Example 4. Yield: 200 mg, (54%).

Ethyl 3-[N-((1S)-1-[N-((1S)-1-carbamoyl-3-methylbutyl)carbamoyl]-3-{N-[(4-cyano phenyl)methyl]-carbamoyl}propyl)carbamoyl]-5-methylhexanoate was prepared from 2-[(ethoxycarbonyl)methyl]-4-methylpentanoic acid (202 mg, 1 mmol), (2S)—N-((1S)-1-carbamoyl-3-methylbutyl)-2-amino-N-[(4-cyanophenyl)methyl]pentane-1,5-diamide (187 mg, 0.5 mmol), EDC HCl (384 mg, 2.0 mmol), HOBt (135 mg, 1 mmol), DIEA 348 μL, 2 mmol), and dichloromethane (100 mL) using the procedure from Example 3. Yield: 250 mg (89%).

2-[2-(N-hydroxycarbamoylmethyl)-4-methylpentanoylamino](2S)—N-((1S)-1-carbamoyl-3-methylbutyl)-N′-{[4-((hydroxyamino)iminomethyl)phenyl]methyl}pentane-1,5-diamide was prepared from ethyl 3-[N-((1S)-1-[N-((1S)-1-carbamoyl-3-methylbutyl)carbamoyl]-3-{N-[(4-cyano phenyl)methyl]-carbamoyl}propyl)carbamoyl]-5-methylhexanoate using the procedure from Example 2.

Yield: 12 mg (7%). MS: (M+H⁺) 578.

2-[4-(3-methylphenyl)phenyl]ethylamine was prepared from 4-bromophenethylamine (400 mg, 2 mmol), 3-tolylboronic acid (270 mg, 2 mmol), bis(triphenylphosphine) palladium dichloride (70 mg, O. 1 mmol), 1M sodiumcarbonate solution (6 mL) and acetonitrile (4 mL) using the procedure from Example 22. Yield: 0.2 g (47%).

Ethyl 5-methyl-3-(N-{2-[4-(3-methylphenyl)phenyl]ethyl}carbamoyl)hexanoate was prepared from 2-[(ethoxycarbonyl)methyl]-4-methylpentanoic acid (101 mg, 0.5 mmol), 2-[4-(3-methylphenyl)phenyl]ethylamine (105 mg, 0.5 mmol), EDC HCl (192 mg, 1 mmol), HOBt (67 mg, 0.5 mmol), DIEA (174 μL, 1 mmol), and dichloromethane (5mL) using the procedure in Example 3. Yield: 158 mg (40%).

### Example 90

### Compound 66: 2-(N-hydroxycarbamoylmethyl)-4-methyl-N-{2-[4-(3-methylphenyl)phenyl]ethyl}pentanamide

2-(N-hydroxycarbamoylmethyl)-4-methyl-N-{2-[4-(3-methylphenyl)phenyl]ethyl}pentanamide was prepared from Ethyl 5-methyl-3-(N-{2-[4-(3-methylphenyl)phenyl]ethyl}carbamoyl)hexanoate (115 mg, 0.29 mmol) using the procedure from Example 2. Yield: 20 mg, (18%). MS: (M+X) 383.

### Example 91

### Compound 67: 2-(N-hydroxycarbamoylmethyl)-N-{(1S)-2-benzo[b]thiophen-3-yl-1-[N-(2-(2H-3,4,5,6-tetrahydropyran-4-yl)ethyl)carbamoyl]ethyl}-4-methylpentanamide

Following the procedure of Example 3, Boc-1-3-benzothienylala (321 mg, 1 mmol), 4-(2-aminoethyl)tetrahydropyran hydrochloride (165 mg, 1 mmol), EDC (384 mg, 2 mmol), HOBt (153 mg, 1 mmol), NMM instead of DIEA (0.329 mL, 3 mmol) and dichloromethane (10 mL) to yield 369 mg (65%) of N-(2-(2H-3,4,5,6-tetrahydropyran-4-yl)ethyl)(2S)-3-benzo[b]thiophen-3-yl-2-[(tert-butoxy)carbonylamino]propanamide as a yellow solid.

Following the procedure of Example 4, N-(2-(2H-3,4,5,6-tetrahydropyran-4-yl)ethyl) (2S)-3-benzo[b]thiophen-3-yl-2-[(tert-butoxy)carbonylamino]propanamide (305 mg, 0.7 mmol) to yield 227 mg (88%) of N-(2-(2H-3,4,5,6-tetrahydropyran-4-yl)ethyl)(2S)-2-amino-3-benzo[b]thiophen-3-ylpropanamide hydrochloride as a yellow solid.

Following the procedure of Example 3, 2-[(ethoxycarbonyl)methyl]-4-methylpentanoic acid (110 mg, 0.54 mmol), N-(2-(2H-3,4,5,6-tetrahydropyran-4-yl)ethyl)(2S)-2-amino-3-benzo[b]thiophen-3-ylpropanamide hydrochloride (200 mg, 0.54 mmol), EDC (207 mg, 1.08 mmol), HOBt (83 mg, 0.54 mmol), NMM instead of DIEA (0.177 mL, 1.62 mmol) and dichloromethane (10 mL) to yield 109 mg (38%) of methyl 3-(N-{(1S)-2-benzo[b]thiophen-3-yl-1-[N-(2-(2H-3,4,5,6-tetrahydropyran-4-yl)ethyl)carbamoyl]ethyl}carbamoyl)-5-methylhexanoate as a yellow solid.

Using the procedure of Example 2, methyl 3-(N-{(1S)-2-benzo[b]thiophen-3-yl-1-[N-(2-(2H-3,4,5,6-tetrahydropyran-4-yl)ethyl)carbamoyl]ethyl}carbamoyl)-5-methylhexanoate (108 mg, 0.21 mmol). The crude product was purified by silica gel chromatography (water/methanol, 30:70) to the isolation of 2-(N-hydroxycarbamoylmethyl)-N-{(1S)-2-benzo[b]thiophen-3-yl-1-[N-(2-(2H-3,4,5,6-tetrahydropyran-4-yl)ethyl)carbamoyl]ethyl}-4-methylpentanamide (8 mg) in 8% yield (more polar product), R_{f}=0.53 (methanol/ethyl acetate, 1:4). MS (M+H)⁺ 504.

### Example 92

### Compound 68: 2-N-hydroxycarbamoylmethyl)-N-[(2,3-dimethylindol-5-yl)methyl]-4-methylpentanamide

Prepared in a manner similar to that described in Example 24 using 0.126 g (0.62 mmol) of 2-[(ethoxycarbonyl)methyl]-4-methylpentanoic acid, 0.109 g (0.62 mmol) of (2,3-dimethylindol-5-yl)methylamine, 0.084 g (0.62 mmol) of HOBt, 0.239 g (1.25 mmol) of EDC, and 0.14 mL (1.25 mmol) of NMM to yield 0.074 g (33%) of ethyl 3-{N-[(2,3-dimethylindol-5-yl)methyl]carbamoyl}-5-methylhexanoate.

MS (M+H)⁺ 359; (M−H)-357.

Prepared in a manner similar to that described in Example 29 using 0.074 g (0.21 mmol) of ethyl 3-{N-[(2,3-dimethylindol-5-yl)methyl]carbamoyl}-5-methylhexanoate to yield 0.036 g (50%) of 2-(N-hydroxycarbamoylmethyl)-N-[(2,3-dimethylindol-5-yl)methyl]-4-methylpentanamide. MS (M+H)⁺ 346; (M−H)-344.

### Example 93

### Compound 69: 2-(N-hydroxycarbamoylmethyl)(2R)—N-{(1S)-1-[N ((1S)-1-carbamoyl-3-methylbutyl)carbamoyl]-2-benzo[b]thiophen-3-ylethyl}-4-methylpentanamide

Following the procedure of Compound 39, Boc-1-3-benzothienylala (1.6 g, 5 mmol), (2S)-2-amino-4-methylpentanamide (975 mg, 7.5 mmol), EDC (1.9 g, 10 mmol), HOBt (765 mg, 5 mmol), DIEA (1.7 mL, 10 mmol) and DMF (20 mL) to yield 1.8 g (83%) (2S)—N-((1S)-1-carbamoyl-3-methylbutyl)-3-benzo[b]thiophen-3-yl-2-[(tert-butoxy)carbonylamino]propanamide as a white solid.

Following the procedure of Example 4, (2S)—N-((1S)-1-carbamoyl-3-methylbutyl)-3-benzo[b]thiophen-3-yl-2-[(tert-butoxy)carbonylamino]propanamide (1.7 g, 3.9 mmol) to yield 1.4 g of (2S)—N-((1S)-1-carbamoyl-3-methylbutyl)-2-amino-3-benzo[b]thiophen-3-ylpropanamide as a yellow solid.

Following the procedure of Example 3, (2S)—N-((1S)-1-carbamoyl-3-methylbutyl)-2-amino-3-benzo[b]thiophen-3-ylpropanamide (163 mg, 0.49 mmol), 2-[(ethoxycarbonyl)methyl]-4-methylpentanoic acid (100 mg, 0.49 mmol), EDC (188 mg, 0.98 mmol), HOBt (75 mg, 0.49 mmol), DIEA (0.17 mL, 0.98 mmol) and dichloromethane (15 mL) to yield 207 mg (82%) of ethyl 3-(N-{(1S)-1-[N-((1S)-1-carbamoyl-3-methylbutyl)carbamoyl]-2-benzo[b.]thiophen-3-ylethyl}carbamoyl)-5-methylhexanoate as a white solid.

Using the procedure of Example 2, ethyl 3-(N-{(1S)-1-[N-((1S)-1-carbamoyl-3-methylbutyl)carbamoyl]-2-benzo[b]thiophen-3-ylethyl}carbamoyl)-5-methylhexanoate (134 mg, 0.21 mmol). The crude product was purified by silica gel chromatography (water/methanol, 30:70) to the isolation of 2-(N-hydroxycarbamoylmethyl)(2R)—N-{(1S)-1-[N-((1S)-1-carbamoyl-3-methylbutyl)carbamoyl]-2-benzo[b]thiophen-3-ylethyl}-4-methylpentanamide (2.8 mg) in 2% yield.

R_{f}=0.48 (methanol/ethyl acetate, 1:4). MS (M+H)⁺ 505;

### Example 94

### Compound 70: 2-(N-hydroxycarbamoylmethyl)-4-methyl-N-[(5-(2-pyridyl)(2-thienyl))methyl]pentanamide

Prepared in a manner similar to that described in Example 24 using 0.100 g (0.49 mmol) of 2-[(ethoxycarbonyl)methyl]-4-methylpentanoic acid, 0.130 g (0.49 mmol) of [5-(2-pyridinyl)-2-thienyl]methylamine dihydrochloride, 0.067 g (0.49 mmol) of HOBt, 0.190 g (0.99 mmol) of EDC, and 0.22 mL (1.98 mmol) of NMM to yield 0.158 g (86%) of ethyl 5-methyl-3-{N-[(5-(2-pyridyl)(2-thienyl))methyl]carbamoyl}hexanoate. MS (M+H)⁺ 375; (M+HCO₂⁻)⁻ 419.

Prepared in a manner similar to that described in Example 29 using 0.144 g-(0.38 mmol) of ethyl 5-methyl-3-{N-[(5-(2-pyridyl)(2-thienyl))methyl]carbamoyl}hexanoate to yield 0.118 g (86%) of 2-(N-hydroxycarbamoylmethyl)-4-methyl-N-[(5-(2-pyridyl)(2-thienyl))methyl]pentanamide. MS (M+H)⁺ 362; (M−H)-360.

### Example 95

### Compound 71: 2-(N-hydroxycarbamoylmethyl)-4-methyl-N-[(4-(2-thienyl)phenyl)methyl]pentanamide

Prepared in a manner similar to that described in Example, 24 using 0.100 g (0.49 mmol) of 2-[(ethoxycarbonyl)methyl]-4-methylpentanoic acid, 0.094 g (0.49 mmol) of [5-(2-pyridinyl)-2-thienyl]methylamine dihydrochloride, 0.067 g (0.49 mmol) of HOBt, 0.190 g (0.99 mmol) of EDC, and 0.11 mL (0.99 mmol) of NMM to yield 0.152 g (83%) of ethyl 5-methyl-3-{N-[(4-(2-thienyl)phenyl)methyl]carbamoyl}hexanoate.

MS (M+H)⁺ 374; (+HCO₂⁻)⁻ 418.

Prepared in a manner similar to that described in Example 29 using 0.116 g (0.31 mmol) of ethyl 5-methyl-3-{N-[(4-(2-thienyl)phenyl)methyl]carbamoyl}hexanoate to yield 0.086 g (77%) of 2-(N-hydroxycarbamoylmethyl)-4-methyl-N-[(4-(2-thienyl)phenyl)methyl]pentanamide. MS (M+H)⁺ 361; (M−H)-359.

### Example 96

### Compound 72: 2-(N-hydroxycarbamoylmethyl)-4-methyl-N-[(4-(1,2,3-thiadiazol-4-yl)phenyl)methyl]pentanamide

Prepared in a manner similar to that described in Example 24 using 0.100 g (0.49 mmol) of 2-[(ethoxycarbonyl)methyl]-4-methylpentanoic acid, 0.113 g (0.49 mmol) of 4-(1,2,3-thiadiazol-4-yl)benzylamine hydrochloride, 0.067 g (0.49 mmol) of HOBt, 0.190 g (0.99 mmol) of EDC, and 0.16 mL (1.48 mmol) of NMM to yield 0.178 g (97%) of ethyl 5-methyl-3-{N-[(4-(1,2,3-thiadiazol-4-yl)phenyl)methyl]carbamoyl}hexanoate. MS (M+H)⁺ 376; (M+HCO₂⁻)⁻ 420.

Prepared in a manner similar to that described in Example 29 using 0.148 g (0.39 mmol) of ethyl 5-methyl-3-{N-[(4-(1,2,3-thiadiazol-4-yl)phenyl)methyl]carbamoyl}hexanoate to yield 0.121 g (86%) of 2-(N-hydroxycarbamoylmethyl)-4-methyl-N-[(4-(1,2,3-thiadiazol-4-yl)phenyl)methyl]pentanamide. MS (M+H)⁺ 363; (M−H)-361.

### Example 97

### Compound 73: 2-(N-hydroxycarbamoylmethyl)(2R)—N-{(1S)-1-[N-((1S)-1-carbamoyl-3-methylbutyl)carbamoyl]-2-benzo[b]thiophen-3-ylethyl}hexanamide

Following the procedure of Example 3, 2-[(tert-butoxy)carbonylaminooxy]acetic acid (333 mg, 1 mmol), (2R)-2-({N-[(2,4-dimethoxyphenyl)methyl]-N-[(4-methoxyphenyl)methoxy]carbamoyl}methyl)hexanoic acid, sodium salt (481 mg, 1 mmol), EDC (384 mg, 2 mmol), HOBt (153 mg, 1 mmol), DIEA (0.174 mL, 1 mmol) and dichloromethane (10mL) to yield 595 mg (77%) of (2R)—N-{(1S)-1-[N-((1S)-1-carbamoyl-3-methylbutyl)carbamoyl]-2-benzo[b]thiophen-3-ylethyl})N-[(2,4-dimethoxyphenyl)methyl]-2-butyl-N′-[(4-methoxyphenyl)methoxy]butane-1,4-diamide as a white solid.

Following the procedure of Example 15, (2R)—N-{(1S)-1-[N-((1S)-1-carbamoyl-3-methylbutyl)carbamoyl]-2-benzo[b]thiophen-3-ylethyl}-N′-[(2,4-dimethoxyphenyl)methyl]-2-butyl-N′-[(4-methoxyphenyl)methoxy]butane-1,4-diamide (500 mg, 0.65 mmol) and 4/1 (v/v) mixture of trifluoroacetic acid and trimethylsilyl bromide. The crude product was purified hot methanol to the isolation of 2-(N-hydroxycarbamoylmethyl)(2R)—N-{(1S)-1-[N-((1S)-1-carbamoyl-3-methylbutyl)carbamoyl]-2-benzo[b]thiophen-3-ylethyl}hexanamide (26 mg) in 8% yield, R_{f}=0.48 (methanol/ethyl acetate, 1:4).

MS (M+H)⁺ 0.505

### Compound 74: 2-(N-hydroxycarbamoylmethyl)(2R)—N-{(1S)-1-[N-((1S)-1-carbamoyl-3-methylbutyl)carbamoyl]-2-(3-phenylphenyl)ethyl}hexanamide

(2S)—N-((1S)-1-carbamoyl-3-methylbutyl)-2-[(tert-butoxy)carbonylamino]-3-(3-bromo phenyl)propanamide was prepared from (2S)-2-[(tert-butoxy)carbonylamino]-3-(3-bromophenyl)propanoic acid (1.03 g, 3 mmol), (2S)-2-amino-4-methylpentanamide (0.455 g, 3.5 mmol), EDC HCl (1.152 g, 6 mmol), HOBt (0.405 g, 3 mmol), DIEA (1.04 mL, 6 mmol) and DMF (10 mL) using the procedure from Compound 39.

Yield: 1.15 g, (83%).

(2S)—N-((1S)-1-carbamoyl-3-methylbutyl)-2-amino-3-(3-bromophenyl)propanamide was prepared from 2S)—N-((1S)-1-carbamoyl-3-methylbutyl)-2-[(tert-butoxy)carbonylamino]-3-(3-bromo phenyl)propanamide (1.03 g, 2.25 mmol) and 4N HCl/dioxane (10 mL) using the procedure from Example 4. Yield: 750 mg (73%).

(2R)—N-{(1S)-1-[N-((1S)-1-carbamoyl-3-methylbutyl)carbamoyl]-2-(3-bromophenyl)ethyl}-N′-[(2,4-dimethoxyphenyl)methyl]-2-butyl-N′-[(4-methoxyphenyl)methoxy]butane-1,4-diamide was prepared from (2R)-2-({N-[(2,4-dimethoxyphenyl)methyl]-N-[(4-methoxyphenyl)methoxy]carbamoyl}methyl)hexanoic acid, sodium salt (962 mg, 2 mmol), (2S)—N-((1S)-1-carbamoyl-3-methylbutyl)-2-amino-3-(3-bromophenyl)propan-amide (712 mg, 2 moles), EDC HCl (768 mg, 4 mmol), HOBt (270 mg, 2 mmol), DIEA (284 μL, 2 mmol) and DMF (10mL) using the procedure in Compound 39.

Yield: 1.15 g (72%).

(2R)—N-{(1S)-1-[N-((1S)-1-carbamoyl-3-methylbutyl)carbamoyl]-2-(3-phenylphenyl)ethyl}-N′-[(2,4-dimethoxyphenyl)-methyl]-2-butyl-N′-[(4-methoxyphenyl)methoxy]butane-1,4-diamide was prepared from (2R)—N-{(1S)-1-[N-((1S)-1-carbamoyl-3-methylbutyl)carbamoyl]-2-(3-bromophenyl)ethyl}-N′-[(2,4-dimethoxyphenyl)methyl]-2-butyl-N′-[(4-methoxyphenyl)methoxy]butane-1,4-diamide (0.8 g, 1 mmol), and phenylboronic acid (214 mg, 1 mmol), bis(triphenylphosphine) palladium dichloride (35 mg, 0.05 mmol), 1M sodiumcarbonate solution (3 mL) and acetonitrile (2 mL) using the procedure from Example 22. Yield: 0.4 g, (50%).

2-(N-hydroxycarbamoylmethyl)(2R)—N-{(1S)-1-[N-((1S)-1-carbamoyl-3-methylbutyl)carbamoyl]-2-(3-phenylphenyl)ethyl}hexanamide was prepared from (2R)—N-{(1S)-1-[N-((1S)-1-carbamoyl-3-methylbutyl)carbamoyl]-2-(3-phenylphenyl)ethyl}-N′-[(2,4-di-methoxyphenyl)-methyl]-2-butyl-N′-[(4-methoxyphenyl)methoxy]butane-1,4-diamide (250 mg, 0.3 mmol) using the procedure from Example 15.

Yield: 30 mg (19%). MS: (M−H) 523.

### Example 98

### Compound 75

### 3-(N-hydroxycarbamoyl)(2R)—N-{(1S)-1-[N-((1S)-1-carbamoyl-2-methylbutyl)carbamoyl]-2-(4-phenylphenyl)ethyl}-2-methylpropanamide

Prepared in a manner similar to that described in Example 24 using 0.075 g (0.51 mmol) of (R)-(+)-2-methylsuccinic acid 4-methyl ester, 0.200 g (0.51 mmol) of (2S)—N-((1S,2S)-1-carbamoyl-2-methylbutyl)-2-amino-3-(4-phenylphenyl)propanamide hydrochloride (See Compound 86), 0.069 g (0.51 mmol) of HOBt, 0.197 g (1.02 mmol) of EDC, and 0.17 mL (1.54 mmol) of NMM to yield 0.222 g (90%) of methyl (3R)-3-(N-{(1S)-1-[N-((1S)-1-carbamoyl-2-methylbutyl)carbamoyl]-2-(4-phenylphenyl)ethyl}carbamoyl)butanoate. MS (M+H)⁺ 482; (+HCO₂⁻)⁻ 526.

Prepared in a manner similar to that described in Example 29 using 0.222 g (0.46 mmol) of methyl (3R)-3-(N-{(1S)-1-[N-((1S)-1-carbamoyl-2-methylbutyl)carbamoyl]-2-(4-phenylphenyl)ethyl}carbamoyl)butanoate to yield 0.218 g (98%) of 3-(N-hydroxycarbamoyl)(2R)—N-{(1S)-1-[N-((1S)-1-carbamoyl-2-methylbutyl)carbamoyl]-2-(4-phenylphenyl)ethyl}-2-methylpropanamide. MS (M+H)⁺ 483; (M−H)⁻ 481.

### Example 99

### Compound 76: 2-N-hydroxycarbamoylmethyl)-4-methyl-N-[(3-phenylphenyl)methyl]pentanamide

Prepared in a manner similar to that described in Example 24 using 0.120 g (0.59 mmol) of 2-[(ethoxycarbonyl)methyl]-4-methylpentanoic acid, 0.109 g (0.59 mmol) of 3-phenylbenzylamine, 0.080 g (0.59 mmol) of HOBt, 0.227 g (1.19 mmol) of EDC, and 0.13 mL (1.19 mmol) of NMM to yield 0.192 g (89%) of ethyl 5-methyl-3-{N-[(3-phenylphenyl)methyl]carbamoyl}hexanoate. MS (M+H)⁺ 368; (M+HCO₂⁻)⁻ 412.

Prepared in a manner similar to that described in Example 29 using 0.192 g (0.52 mmol) of ethyl 5-methyl-3-{N-[(3-phenylphenyl)methyl]carbamoyl}hexanoate to yield 0.144 g (78%) of 2-(N-hydroxycarbamoylmethyl)-4-methyl-N-[(3-phenylphenyl)methyl]pentanamide. MS (M+H)⁺ 355; (M−H)⁻ 353.

### Example 100

### Compound 77: 2-(N-hydroxycarbamoylmethyl)-4-methyl-N-[(4-phenylphenyl)methyl]pentanamide

Prepared in a manner similar to that described in Example 24 using 0.120 g (0.59 mmol) of 2-[(ethoxycarbonyl)methyl]-4-methylpentanoic acid, 0.109 g (0.59 mmol) of 4-phenylbenzylamine, 0.080 g (0.59 mmol) of HOBt, 0.227 g (1.19 mmol) of EDC, and 0.13 mL (1.19 mmol) of NMM to yield 0.198 g (91%) of ethyl 5-methyl-3-{N-[(4-phenylphenyl)methyl]carbamoyl}hexanoate. MS (M+H)⁺ 368; (M+HCO₂⁻)⁻ 412.

Prepared in a manner similar to that described in Example 29 using 0.132 g (0.36 mmol) of ethyl 5-methyl-3-{N-[(4-phenylphenyl)methyl]carbamoyl}hexanoate to yield 0.092 g (72%) of 2-(N-hydroxycarbamoylmethyl)-4-methyl-N-[(4-phenylphenyl)methyl]pentanamide. MS (M+H)⁺ 355; (M−H)⁻ 353.

### Example 101

### Compound 78: 2-(N-hydroxycarbamoylmethyl)(2S)—N-{(1S)-1-[N-methyl-N-benzylcarbamoyl]-2-(2-naphthyl)ethyl}-4-methylpentanamide

Prepared in a manner similar to that described in Example 24 using 1.00 g (3.17 mmol) of (S)-(−)-2-(tert-butoxycarbonylamino)-3-(2-naphthyl)propanoic acid, 0.384 g (3.17 mmol) of N-methylbenzylamine, 0.428 g (3.17 mmol) of HOBt, 1.216 g (6.34 mmol) of EDC, and 0.697 mL (6.34 mmol) of NMM to yield 1.220 g (92%) of [1-(Benzyl-methyl-carbamoyl)-2-naphthalen-2-yl-ethyl]-carbamic acid tert-butyl ester. MS (M+H)⁺ 419.

Prepared in a manner similar to that described in Example 4 using 1.200 g (2.87 mmol) of [1-(Benzyl-methyl-carbamoyl)-2-naphthalen-2-yl-ethyl]-carbamic acid tert-butyl ester, and 20 mL of 4M solution of HCl in 1,4-dioxane to yield 1.005 g (99%) of 1-(Benzyl-methyl-carbamoyl)-2-naphthalen-2-yl-ethyl-ammonium.

Prepared in a manner similar to that described in Example 24 using 0.386 g (1.09 mmol) of 1-(Benzyl-methyl-carbamoyl)-2-naphthalen-2-yl-ethyl-ammonium; chloride, 0.220 g (1.09 mmol) of 2-[(ethoxycarbonyl)methyl]-4-methylpentanoic acid, 0.147 g (1.09 mmol) of HOBt, 0.417 g (2.18 mmol) of EDC, and 0.3 mL (3.26 mmol) of NMM to yield 0.454 g (83%) of ethyl (3R,S)-3-(N-{(1S)-1-[N-methyl-N-benzylcarbamoyl]-2-(2-naphthyl)ethyl}carbamoyl)-5-methylhexanoate (1:1 mixture of diastereoisomers).

The mixture of diastereoisomers was purified by flash chromatography (EtOAc/hexanes) to give 0.200 g of ethyl (3R)-3-(N-{(1S)-1-[N-methyl-N-benzylcarbamoyl]-2-(2-naphthyl)ethyl}carbamoyl)-5-methylhexanoate, R_{f}=0.23 (solvent: hexanes/ethyl acetate, 2/1), MS (M+H)⁺ 503; (M+HCO₂⁻)-547; and 0.206 g of ethyl (3S)-3-(N-{(1S)-1-[N-methyl-N-benzylcarbamoyl]-2-(2-naphthyl)ethyl}carbamoyl)-5-methylhexanoate, R_{f}=0.18 (solvent: hexanes/ethyl acetate, 2/1). MS (M+H)⁺ 503.

Prepared in a manner similar to that described in Example 29 using 0.168 g (0.33 mmol) of ethyl (3S)-3-(N-{(S)-1-[N-methyl-N-benzylcarbamoyl]-2-(2-naphthyl)ethyl}carbamoyl)-5-methylhexanoate to yield 0.149 g (92%) of 2-(N-hydroxycarbamoylmethyl)(2S)—N-{(1S)-1-[N-methyl-N-benzylcarbamoyl]-2-(2-naphthyl)ethyl}-4-methylpentanamide. MS (+11)⁺ 490; (M−H)⁻ 488.

### Example 102

### Compound 79: 2-(N-hydroxycarbamoylmethyl)-N-[(1S)-2-isoindolin-2-yl-1-(2-naphthylmethyl)-2-oxoethyl]-4-methylpentanamide

Prepared in a manner similar to that described in Example 24 using 1.00 g (3.17 mmol) of (S)-(−)-2-(tert-butoxycarbonylamino)-3-(2-naphthyl)propanoic acid, 0.360 g (3.17 mmol) of isoindoline, 0.428 g (3.17 mmol) of HOBt, 1.216 g (6.34 mmol) of EDC, and 0.697 mL (6.34 mmol) of NMM to yield 1.210 g (91%) of [2-(1,3-Dihydro-isoindol-2-yl)-1-naphthalen-2-ylmethyl-2-oxo-ethyl]-carbamic acid tert-butyl ester. MS (M+H)⁺ 417.

Prepared in a manner similar to that described in Example 4 using 1.200 g (2.88 mmol) of [2-(1,3-Dihydro-isoindol-2-yl)-1-naphthalen-2-ylmethyl-2-oxo-ethyl]-carbamic acid tert-butyl ester, and 20 mL of 4M solution of HCl in 1,4-dioxane to yield 0.98 g (96%) of 2-(1,3-Dihydro-isoindol-2-yl)-1-naphthalen-2-ylmethyl-2-oxo-ethyl-ammonium; chloride.

Prepared in a manner similar to that described in Example 24 using 0.272 g (0.77 mmol) of 2-(1,3-Dihydro-isoindol-2-yl)-1-naphthalen-2-ylmethyl-2-oxo-ethyl-ammonium; chloride, 0.156 g (0.77 mmol) of 2-[(ethoxycarbonyl)methyl]-4-methylpentanoic acid, 0.104 g (0.77 mmol) of HOBt, 0.296 g (1.54 mmol) of EDC, and 0.25 mL (2.3 mmol) of NMM to yield 0.348 g (960%) of ethyl (3R,S)-3-{N-[(1S)-2-isoindolin-2-yl-1-(2-naphthylmethyl)-2-oxoethyl]carbamoyl}-5-methylhexanoate (1:1 mixture of diastereoisomers). MS (M+H)⁺ 501; (M+HCO₂⁻)⁻ 545.

Prepared in a manner similar to that described in Example 29 using 0.348 g (0.70 mmol) of ethyl (3R,S)-3-(N-[(1S)-2-isoindolin-2-yl-1-(2-naphthylmethyl)-2-oxoethyl]carbamoyl}-5-methylhexanoate (1:1 mixture of diastereoisomers) to yield 0.278 g (82%) of 2-(N-hydroxycarbamoylmethyl)(2R,S)—N-[(1S)-2-isoindolin-2-yl-1-(2-naphthylmethyl)-2-oxoethyl]-4-methylpentanamide (1:1 mixture of diastereoisomers).

MS (M+H)⁺ 488; (M−H)-486.

### Example 103

### Compound 80: 2-N-hydroxycarbamoylmethyl)(2R)—N-{(1S)-1-[N-methyl-N-benzylcarbamoyl]-2-(2-naphthyl)ethyl}-4-methylpentanamide

Prepared in a manner similar to that described in Example 29 using 0.128 g (0.25 mmol) of ethyl (3R)-3-(N-{(1S)-1-[N-methyl-N-benzylcarbamoyl]-2-(2-naphthyl)ethyl}carbamoyl)-5-methylhexanoate (from Compound 78) to yield 0.109 g (89%) of 2-(N-hydroxycarbamoylmethyl)(2R)—N-{(1S)-1-[N-methyl-N-benzylcarbamoyl]-2-(2-naphthyl)ethyl}-4-methylpentanamide. MS (M+H)⁺ 490; (M−H)-488.

### Example 104

### Compound 81: 3-(N-hydroxycarbamoyl)(2S)—N-{(1S)-1-[N-((1S)-1-carbamoyl-3-methylbutyl)carbamoyl]-2-naphthylethyl}-2-(cyclobutylmethyl)propanamide and Compound 82: 3-(N-hydroxycarbamoyl)(2R)—N-{(1S)-1-[N-((1S)-1-carbamoyl-3-methylbutyl)carbamoyl]-2-naphthylethyl}-2-(cyclobutylmethyl)propanamide

Prepared in a manner similar to that described in Example 23 using 3.00 g (9.90 mmol) of tert-butyl ethyl 2-[(tert-butyl)oxycarbonyl]butane-1,4-dioate, 2.23 mL (19.80 mmol) of cyclobutylmethyl bromide, 0.397 g (9.90 mmol) of NaH to yield 3.252 g (88%) of 2-tert-Butoxycarbonyl-2-cyclobutylmethyl-succinic acid 1-tert-butyl ester 4-ethyl ester.

¹H NMR (300 MHz, CDCl₃) δ 4.11 (2H, q), 2.85 (2H, s), 2.33-2.21 (1H, m), 2.05 (2H, d), 2.03-1.99 (2H, m), 1.74-1.59 (4H, m), 1.45 (18H, s), 1.24 (3H, t).

Prepared in a manner similar to that described in Example 23 using 1.50 g (4.05 mmol) of 2-tert-Butoxycarbonyl-2-cyclobutylmethyl-succinic acid 1-tert-butyl ester 4-ethyl ester, 10mL of TFA to yield 1.00 g (96%) of 2-Carboxy-2-cyclobutylmethyl-succinic acid 4-ethyl ester.

¹H NMR (300 MHz, CDCl₃) δ 8.20 (2H, br s), 4.15 (2H, q), 3.09 (2H, s), 2.40-2.35 (1H, m), 2.11-2.03 (2H, m), 2.05 (2H, d), 1.90-1.61 (4H, m), 1.25 (3H, t).

Prepared in a manner similar to that described in Example 23 using 1.00 g (3.87 mmol) of 2-Carboxy-2-cyclobutylmethyl-succinic acid 4-ethyl ester to yield 0.501 g (60%) of 2-Cyclobutylmethyl-succinic acid 4-ethyl ester.

¹H NMR (300 MHz, CDCl₃) δ 10.02 (1H, br s), 4.14 (2H, q), 2.82-2.75 (1H, m), 2.67 (1H, dd), 2.45-2.31 (2H, m), 2.10-2.02 (2H, m), 1.88-1.60 (6H, m), 1.25 (3H, t).

Prepared in a manner similar to that described in Example 24 using 0.425 g (1.20 mmol) of (2S)—N-((1S)-1-carbamoyl-3-methylbutyl)-2-amino-3-naphthylpropanamide, chloride (from Compound 96), 0.250 g (1.20 mmol) of 2-Cyclobutylmethyl-succinic acid 4-ethyl ester, 0.158 g (1.20 mmol) of HOBt, 0.447 g (2.30 mmol) of EDC, and 0.42 mL (3.5 mmol) of NMM to yield 0.534 g (85%) of ethyl (3R,S)-3-(N-{(1S)-1-[N-((1S)-1-carbamoyl-3-methylbutyl)carbamoyl]-2-naphthylethyl}carbamoyl)-4-cyclobutylbutanoate (1:1 mixture of diastereoisomers). MS (M+H)⁺ 524; (M+HCO₂⁻)⁻ 568.

Prepared in a manner similar to that described in Example 29 using 0.284 g (0.54 mmol) of ethyl (3R,S)-3-(N-{(1S)-1-[N-((1S)-1-carbamoyl-3-methylbutyl)carbamoyl]-2-naphthylethyl}carbamoyl)-4-cyclobutylbutanoate (1:1 mixture of diastereoisomers) to yield 0.254 g (92%) of 3-(N-hydroxycarbamoyl)(2R,S)—N-{(1S)-1-[N-((1S)-1-carbamoyl-3-methylbutyl)carbamoyl]-2-naphthylethyl}-2-(cyclobutylmethyl)propanamide (1:1 mixture of diastereoisomers). The mixture of diastereoisomers was purified by C-18 flash chromatography (MeOH/H₂O) to give 0.015 g of 3-(N-hydroxycarbamoyl)(2R)—N-{(1S)-1-[N-((1S)-1-carbamoyl-3-methylbutyl)carbamoyl]-2-naphthylethyl}-2-(cyclobutylmethyl)propanamide, R_{f}=0.45 (solvent: CHCl₃/MeOH/NH₄OH, 90/10/1), MS (M+H)⁺ 511; (—H)-509; and 0.018 g of 3-(N-hydroxycarbamoyl)(2S)—N-{(1S)-1-[N-((1S)-1-carbamoyl-3-methylbutyl)carbamoyl]-2-naphthylethyl}-2-(cyclobutylmethyl)propanamide, R_{f}=0.42 (solvent: CHCl₃/MeOH/NH₄OH, 90/10/1).

MS (M+H)⁺ 511; (M−H)⁻ 509.

### Example 105

### Compound 83

### 2-(N-hydroxycarbamoylmethyl)-N-(2-indol-3-ylethyl)-4-methylpentanamide

Prepared in a manner similar to that described in Example 24 using 0.228 g (1.10 mmol) of 2-[(ethoxycarbonyl)methyl]-4-methylpentanoic acid, 0.181 g (1.10 mmol) of tryptamime, 0.152 g (1.10 mmol) of HOBt, 0.432 g (2.30 mmol) of EDC, and 0.25 mL (2.30 mmol) of NMM to yield 0.380 g (99%) of ethyl 3-[N-(2-indol-3-ylethyl)carbamoyl]-5-methylhexanoate. MS (M+H)⁺ 345; (M+HCO₂⁻)⁻ 389.

Prepared in a manner similar to that described in Example 29 using 0.380 g (1.10 mmol) of ethyl 3-[N-(2-indol-3-ylethyl)carbamoyl]-5-methylhexanoate to yield 0.259 g (71%) of 2-(N-hydroxycarbamoylmethyl)-N-(2-indol-3-ylethyl)-4-methylpentanamide.

MS (+H)⁺ 332; (M−H)-330.

### Example 106

### Compound 84

### 2-(N-hydroxycarbamoylmethyl)-4-Methyl-N-[2-(4-phenylphenyl)ethyl]pentanamide

Prepared in a manner similar to that described in Example 24 using 0.228 g (1.10 mmol) of 2-[(ethoxycarbonyl)methyl]-4-methylpentanoic acid, 0.222 g (1.10 mmol) of 2-(4-biphenyl)ethylamine, 0.152 g (1.10 mmol) of HOBt, 0.432 g (2.30 mmol) of EDC, and 0.25 mL (2.30 mmol) of NMM to yield 0.388 g (92%) of ethyl 5-methyl-3-(N-[2-(4-phenylphenyl)ethyl]carbamoyl}hexanoate. MS (M+H)⁺ 382; (M+HCO₂)⁻ 426.

Prepared in a manner similar to that described in Example 29 using 0.188 g (0.49 mmol) of ethyl 5-methyl-3-{N-[2-(4-phenylphenyl)ethyl]carbamoyl}hexanoate to yield 0.123 g (68%) of 2-(N-hydroxycarbamoylmethyl)-4-methyl-N-[2-(4-phenylphenyl)ethyl]pentanamide. MS (M+H)⁺ 369; (N—H)⁻ 367.

### Example 107

### Compound 85

### 2-(N-hydroxycarbamoylmethyl)-N-{(1S)-1-[N-((1S)-1-carbamoyl-2-indol-3-ylethyl)carbamoyl]-2-indol-3-ylethyl}-4-methylpentanamide

(2S)—N-((1S)-1-carbamoyl-2-indol-3-ylethyl)-2-amino-3-indol-3-ylpropanamide was prepared by stirring (2S)—N-((1S)-1-carbamoyl-2-indol-3-ylethyl)-3-indol-3-yl-2-[(phenyl methoxy)carbonylamino]propanamide (0.84 g, 1.6 mmol) in Methanol (30 mL) along with 10% palladium on carbon (200 mg) in hydrogen atmosphere for overnight. The Palladium/carbon was filtered off, the filtrate was rotovaped and dried in vacuum to get the product. Yield: 0.58 g (94%).

Ethyl 3-(N-{(1S)-1-[N-((1S)-1-carbamoyl-2-indol-3-ylethyl)carbamoyl]-2-indol-3-ylethyl}carbamoyl)-5-methylhexanoate was prepared from 2-[(ethoxycarbonyl)methyl]-4-methylpentanoic acid (0.28 g, 1.4 mmol), (2S)—N-((1S)-1-carbamoyl-2-indol-3-ylethyl)-2-amino-3-indol-3-ylpropanamide (0.58 g, 1.5 mmol), EDC HCl (0.54 g, 2.8 mmol), HOBt (0.19 g, 1.4 mmol), DIEA (487 μl, 2.8 mmol) and DMF using the procedure from Compound 39. Yield: 0.75 g (91%).

2-(N-hydroxycarbamoylmethyl)-N-{(1S)-1-[N-((1S)-1-carbamoyl-2-indol-3-ylethyl)carbamoyl]-2-indol-3-ylethyl}-4-methylpentanamide was prepared from Ethyl 3-(N-{(1S)-1-[N-((1S)-1-carbamoyl-2-indol-3-ylethyl)carbamoyl]-2-indol-3-ylethyl}carbamoyl)-5-methylhexanoate (0.29 g, 0.5 mmol) using the procedure from Compound 88. Yield: 20 mg (7%). MS: (M−H⁺) 559.

### Example 108

### Compound 86: 2-(N-hydroxycarbamoylmethyl)-N-{(1S)-1-[N-((1S)-1-carbamoyl-2-methylbutyl)carbamoyl]-2-(4-phenylphenyl)ethyl}-4-methylpentanamide

Following the procedure of Example 3,3-(4-biphenylyl)-n-(tert-butoxycarbonyl)-1-alanine (1 g, 2.9 mmol), H-IIE-NH₂HCl (722 mg, 1.5 mmol), EDC (1.11 g, 2 mmol), HOBt (444 mg, 1 mmol), DIEA (1.73mL, 3.5 mmol) and dichloromethane (10 mL) to yield 1.05 g (80%) of (2S)—N-((1S,2S)-1-carbamoyl-2-methylbutyl)-2-[(tert-butoxy)carbonylamino]-3-(4-phenylphenyl)propanamide as a white solid.

Following the procedure of Example 4, (2S)—N-((1S,2S)-1-carbamoyl-2-methylbutyl)-2-[(tert-butoxy)carbonylamino]-3-(4-phenylphenyl)propanamide (1 g, 2.2 mmol) to yield 355 mg of (2S)—N-((1S,2S)-1-carbamoyl-2-methylbutyl)-2-amino-3-(4-phenylphenyl)propanamide as a white solid.

Following the procedure of Example 3, (2S)—N-((1S,2S)-1-carbamoyl-2-methylbutyl)-2-amino-3-(4-phenylphenyl)propanamide (307 mg, 0.87 mmol), 2-[(ethoxycarbonyl)methyl]-4-methylpentanoic acid (176 mg, 0.87 mmol), EDC (334 mg, 1.74 mmol), HOBt (133 mg, 0.87 mmol), DIEA (0.374 mL, 1.74 mmol) and dichloromethane (10 mL) to yield 315 mg (67%) of ethyl 3-(N-{(1S)-1-[N-((1S,2S)-1-carbamoyl-2-methylbutyl)carbamoyl]-2-(4-phenylphenyl)ethyl}carbamoyl)-5-methylhexanoate as a yellow solid.

Using the procedure of Example 2, ethyl 3-(N-{(1S)-1-[N-((1S,2S)-1-carbamoyl-2-methylbutyl)carbamoyl]-2-(4-phenylphenyl)ethyl}carbamoyl)-5-methylhexanoate (153 mg, 0.28 mmol). The crude product was purified by silica gel chromatography (water/methanol, 30:70) to the isolation of 2-(N-hydroxycarbamoylmethyl)-N-{(1S)-1-[N-((1S)-1-carbamoyl-2-methylbutyl)carbamoyl]-2-(4-phenylphenyl)ethyl}-4-methylpentanamide (5 mg) in 3% yield, R_{f}=0.53 (methanol/ethyl acetate, 1:4). MS (M+H)⁺ 525.

### Example 109

### Compound 88

### 2-(N-hydroxycarbamoylmethyl)-N-{(1S)[N-((1S)-1-carbamoyl-3-methylbutyl)carbamoyl](4-phenylphenyl)methyl}-4-methylpentanamide

Prepared in a manner similar to that described in Example 7 using 1.100 g (2.40 mmol) of 2-[(fluoren-9-ylmethoxy)carbonylamino]-2-(4-phenylphenyl)acetic acid, 0.319 g (2.40 mmol) of (2S)-2-amino-4-methylpentanamide, 0.331 g (2.40 mmol) of HOBt, 0.938 g (40.90 mmol) of EDC, and 0.54 mL (4.90 mmol) of NMM to yield 1.300 g (96%) of (2S,R)—N-((1S)-1-carbamoyl-3-methylbutyl)-2-[(fluoren-9-ylmethoxy)carbonylamino]-2-(4-phenylphenyl)acetamide (11 mixture of diastereoisomers).

To a solution of (2S,R)—N-((1S)-1-carbamoyl-3-methylbutyl)-2-[(fluoren-9-ylmethoxy)carbonylamino]-2-(4-phenylphenyl)acetamide (1:1 mixture of diastereoisomers) (1.30 g, 2.30 mmol) in 15 mL of DMF was added 1-octanethiol (0.48 mL, 2.78 mmol), follow by 1M solution of tetrabutylammonium fluoride in TEF (3.50 mL, 3.47 mmol) at room temperature. The reaction mixture stirred at room temperature for 1 hour. The reaction mixture was concentrated under vacuum. The residue was purified by flash chromatography (ethyl acetate/hexanes/methanol, 2/1/0 to 10/0/1) to give 0.250 g (32%) of (2S)—N-((1S)-1-carbamoyl-3-methylbutyl)-2-amino-2-(4-phenylphenyl)acetamide, R_{f}=0.63 (solvent: MeOH/ethyl acetate, 2/3), 0.204 g (26%) of (2R)—N-((1S)-1-carbamoyl-3-methylbutyl)-2-amino-2-(4-phenylphenyl)acetamide, R_{f}=0.38 (solvent: MeOH/ethyl acetate, 2/3), and 0.253 g (32%) of mixture of two diastereoisomers.

Prepared in a manner similar to that described in Example 7 using 0.130 g (0.38 mmol) of (2S)—N-((1S)-1-carbamoyl-3-methylbutyl)-2-amino-2-(4-phenylphenyl)acetamide, 0.077 g (0.38 mmol) of 2-[(ethoxycarbonyl)methyl]-4-methylpentanoic acid, 0.052 g (0.38 mmol) of HOBt, 0.147 g (0.77 mmol) of EDC, and 0.084 mL (0.77 mmol) of NMM to yield 0.196 g (99%) of ethyl (3R,S)-3-(N-{(1S)[N-((1S)-1-carbamoyl-3-methylbutyl)carbamoyl](4-phenylphenyl)methyl}carbamoyl)-5-methylhexanoate (1:1 mixture of diastereoisomers). MS (M+H)⁺ 524.

To a solution of NH₂OH—HCl (0.094 g, 1.30 mmol) in 0.17 mL of H₂O was added 5.33M solution of KOH in H₂O (0.51 mL, 2.70 mmol) and stirred for 10 min. at 0° C. This solution was added to a stirred solution, cooled to 0-5° C., of ethyl (3R,S)-3-(N-{(1S)[N-((1S)-1-carbamoyl-3-methylbutyl)carbamoyl](4-phenylphenyl)methyl}carbamoyl)-5-methylhexanoate (1:1 mixture of diastereoisomers) (0.196 g, 0.37 mmol) in 3 mL of THF and the reaction emulsion was stirred at 0° C. for overnight. After acidification of the reaction solution at 0° C. to pH=5 with 1N HCl, the reaction mixture was concentrated under vacuum. The residue was purified by C-18 flash chromatography (H₂O/methanol) to give 0.705 g (37%) of 2-(N-hydroxycarbamoylmethyl)(2R,S)—N-{(1S)[N-((1S)-1-carbamoyl-3-methylbutyl)carbamoyl](4-phenylphenyl)methyl}-4-methylpentanamide (2:3 mixture of diastereoisomers). MS (M+H)⁺ 511; M−H)-509.

### Example 110

### Compound 89: 2-(N-hydroxycarbamoylmethyl)-N-[2-benzo[b]thiophen-3-yl-1-(N-methylcarbamoyl)ethyl]-4-methylpentanamide

Prepared in a manner similar to that described in Example 24 using 0.663 g (2.07 mmol) of (2R)-3-benzo[b]thiophen-3-yl-2-[(tert-butoxy)carbonylamino]propanoic acid, 0.280 g (4.1 mmol) of methylamine hydrochloride, 0.280 g (2.07 mmol) of HOBt, 0.780 g (4.1 mmol) of EDC, and 0.90 mL (8.2 mmol) of NMM to yield 0.618 g (90%) of (2S)-3-benzo[b]thiophen-3-yl-2-[(tert-butoxy)carbonylamino]-N-methylpropanamide.

Prepared in a manner similar to that described in Example 4 using 1.200 g (3.59 mmol) of (2S)-3-benzo[b]thiophen-3-yl-2-[(tert-butoxy)carbonylamino]-N-methylpropanamide, and 15 mL of 4M solution of HCl in 1,4-dioxane to yield 0.716 g (85%) of (2S)-2-amino-3-benzo[b]thiophen-3-yl-N-methylpropanamide, hydrochloride.

Prepared in a manner similar to that described in Example 24 using 0.250 g (1.07 mmol) of (2S)-2-amino-3-benzo[b]thiophen-3-yl-N-methylpropanamide, hydrochloride, 0.215 g (1.07 mmol) of 2-[(ethoxycarbonyl)methyl]-4-methylpentanoic acid, 0.150 g (1.07 mmol) of HOBt, 0.405 g (2.14 mmol) of EDC, and 0.50 mL (4.5 mmol) of NMM to yield 0.356 g (80%) of ethyl (3R,S)-3-{N-[(1R)-2-benzo[b]thiophen-3-yl-1-(N-methylcarbamoyl)ethyl]carbamoyl}-5-methylhexanoate (1:1 mixture of diastereoisomers).

Prepared in a manner similar to that described in Compound 88 using 0.356 g (0.85 mmol) of ethyl (3R,S)-3-{N-[(1R)-2-benzo[b]thiophen-3-yl-1-(N-methylcarbamoyl)ethyl]carbamoyl}-5-methylhexanoate (1:1 mixture of diastereoisomers) to yield 0.030 g (9%) of 2-(N-hydroxycarbamoylmethyl)(2R,S)—N-[(1R)-2-benzo[b]thiophen-3-yl-1-(N-methylcarbamoyl)ethyl]-4-methylpentanamide (1:1 mixture of diastereoisomers). MS (M+H)⁺ 406; (M−H)⁻ 404.

### Example 111

### Compound 90: 2-(N-hydroxycarbamoylmethyl)(2R)—N-{(1S)-1-[N-((1S,2S)-1-carbamoyl-2-methylbutyl)carbamoyl]-2-naphthylethyl}hexanamide

Following the procedure of Example 3, (2S)—N-((1S)-1-carbamoyl-2-methylbutyl)-2-amino-3-naphthylpropanamide (209 mg, 0.64 mmol), (2R)-2-({N-[(2,4-dimethoxyphenyl)methyl]-N-[(4-methoxyphenyl)methoxy]carbamoyl}methyl)hexanoic acid (355 mg, 0.77 mmol), EDC (246 mg, 1.28 mmol), HOBt (98 mg, 0.64 mmol), DIEA (0.223 mL, 1.28 mmol) and dichloromethane (10 mL) to yield 338 mg (69%) of (2R)—N-{(1S)-1-[N-((1S)-1-carbamoyl-2-methylbutyl)carbamoyl]-2-naphthylethyl}-N′-[(2,4-dimethoxyphenyl)methyl]-2-butyl-N′-[(4-methoxyphenyl)methoxy]butane-1,4-diamide as an off white solid.

Following the procedure of Example 15, (2R)—N-{(1S)-1-[N-((1S)-1-carbamoyl-2-methylbutyl)carbamoyl]-2-naphthylethyl}-N′-[(2,4-dimethoxyphenyl)methyl]-2-butyl-N′-[(4-methoxyphenyl)methoxy]butane-1,4-diamide (100 mg, 0.13 mmol) and 4/1 (v/v) mixture of trifluoroacetic acid and trimethylsilyl bromide. The crude product was washed with methanol to the isolation of 2-(N-hydroxycarbamoylmethyl)(2R)—N-{(1S)-1-[N-((1S,2S)-1-carbamoyl-2-methylbutyl)carbamoyl]-2-naphthylethyl}hexanamide (4 mg) in 6% yield, R_{f}=0.31 (methanol/ethyl acetate, 1:4). MS (M+H)⁺ 499.

### Example 112

### Compound 91

### N-{2-(N-hydroxycarbamoyl)(1R)-1-[(2,3,4,5,6-pentafluorophenyl)methyl]ethyl}(2R)-2-[(tert-butoxy)carbonylamino]-3-(2-naphthyl)propanamide

(3R)-3-[(tert-butoxy)carbonylamino]-4-(2,3,4,5,6-pentafluorophenyl)-N-(phenyl methoxy)butanamide was prepared from boc-(r)-3-amino-4-pentafluorophenyl butanoic acid (1.01 g, 2.75 mmol), O-benzylhydroxylamine (0.68 g, 5.5 mmol), EDC HCl (1.06 g, 5.5 mmol), HOBt (0.42 g, 5.5 mmol), DIEA (0.96 mL, 5.5 mmol) and DMF (15 mL) using the procedure of Compound 39. Yield: 1.1 g (84%).

(3R)-3-amino-4-(2,3,4,5,6-pentafluorophenyl)-N-(phenylmethoxy)butanamide was prepared from (3R)-3-[(tert-butoxy)carbonylamino]-4-(2,3,4,5,6-pentafluorophenyl)-N-(phenylmethoxy)butanamide (0.71 g, 1.5 mmol) and 4N HCl/dioxane (10 mL) using the procedure from Example 4. Yield: 0.25 g (45%).

(3R)-3-{(2R)-2-[(tert-butoxy)carbonylamino]-3-(2-naphthyl)propanoylamino}-4-(2,3,4,5,6-pentafluorophenyl)-N-(phenylmethoxy)butanamide was prepared from boc-D-2-naphthylalanine (315 mg, 1 mmol), (3R)-3-amino-4-(2,3,4,5,6-pentafluorophenyl)-N-(phenylmethoxy)butanamide (225 mg, 0.6 mmol), EDC HCl (384 mg, 2 mmol), HOBt (135 mg, 1 mmol), DIEA (348 mg, 2 mmol), dichloromethane (10 mL) using the procedure from Example 3. Yield: 360 mg (54%).

N-{2-(N-hydroxycarbamoyl)(1R)-1-[(2,3,4,5,6-pentafluorophenyl)methyl]ethyl}(2R)-2-[(tert-butoxy)carbonylamino]-3-(2-naphthyl)propanamide was prepared by stirring the solution of (3R)-3-{(2R)-2-[(tert-butoxy)carbonylamino]-3-(2-naphthyl)propanoyl amino}-4-(2,3,4,5,6-pentafluorophenyl)-N-(phenylmethoxy)butanamide (200 mg, 0.3 mmol), in methanol, in presence of 10% palladium/carbon in hydrogen atmosphere overnight. The palladium/carbon was filtered off. The filtrate on evaporation gave a solid. Yield: 85 mg (64%). MS: (M+H⁺-boc group) 482.

### Example 113

### Compound 92: 2-(N-hydroxycarbamoylmethyl)-N-{(1S)-2-(2-naphthyl)-1-[N-benzylcarbamoyl]ethyl}octanamide

Prepared in a manner similar to that described in Example 23 using 1.00 g (3.31 mmol) of tert-butyl ethyl 2-[(tert-butyl)oxycarbonyl]butane-1,4-dioate, 1.40 g (6.60 mmol) of iodohexane, 0.132 g (3.31 mmol) of NaH to yield 1.22 g (95%) of tert-butyl ethyl 2-[(tert-butyl)oxycarbonyl]-2-hexylbutane-1,4-dioate.

Prepared in a manner similar to that described in Example 23 using 1.22 g (3.160 mmol) of tert-butyl ethyl 2-[(tert-butyl)oxycarbonyl]-2-hexylbutane-1,4-dioate, and 10 mL of TFA to yield 0.823 g (95%) of 2-[(ethoxycarbonyl)methyl]-2-hexylpropanedioic acid.

Prepared in a manner similar to that described in Example 23 using 0.823 g (3.00 mmol) of 2-[(ethoxycarbonyl)methyl]-2-hexylpropanedioic acid to yield 0.590 g (86%) of 2-[(ethoxycarbonyl)methyl]octanoic acid.

Prepared in a manner similar to that described in Example 24 using 0.250 g (0.74 mmol) of (2S)-2-amino-3-(2-naphthyl)-N-benzylpropanamide, hydrochloride (from Compound 100), 0.170 g (0.74 mmol) of 2-[(ethoxycarbonyl)methyl]octanoic acid, 0.100 g (0.74 mmol) of HOBt, 0.280 g (1.47 mmol) of EDC, and 0.24 mL (2.19 mmol) of NMM to yield 0.225 g (59%) of ethyl (3R,S)-3-(N-{(1S)-2-(2-naphthyl)-1-[N-benzylcarbamoyl]ethyl}carbamoyl)nonanoate (1:1 mixture of diastereoisomers).

Prepared in a manner similar to that described in Compound 88 using 0.225 g (0.43 mmol) of ethyl (3R,S)-3-(N-{(1S)-2-(2-naphthyl)-1-[N-benzylcarbamoyl]ethyl}carbamoyl)nonanoate (1:1 mixture of diastereoisomers) to yield 0.030 g (14%) of 2-(N-hydroxycarbamoylmethyl)(2R,S)—N-{(1S)-2-(2-naphthyl)-1-[N-benzylcarbamoyl]ethyl}octanamide (1:1 mixture of diastereoisomers). MS (M+H)⁺ 504; (—I)-502.

### Example 114

### Compound 93: 2-(N-hydroxycarbamoylmethyl)-N-{(1S)-2-(2-naphthyl)-1-[N-benzylcarbamoyl]ethyl}heptanamide

Prepared in a manner similar to that described in Example 23 using 2.00 g (6.62 mmol) of tert-butyl ethyl 2-[(tert-butyl)oxycarbonyl]butane-1,4-dioate, 2.62 g (13.23 mmol) of iodopentane, 0.265 g (6.62 mmol) of NaH to yield 2.02 g (82%) of tert-butyl ethyl 2-[(tert-butyl)oxycarbonyl]-2-pentylbutane-1,4-dioate.

Prepared in a manner similar to that described in Example 23 using 2.02 g (5.43 mmol) of tert-butyl ethyl 2-[(tert-butyl)oxycarbonyl]-2-pentylbutane-1,4-dioate, and 15 mL of TFA to yield 1.383 g (98%) of 2-[(ethoxycarbonyl)methyl]-2-pentylpropanedioic acid.

Prepared in a manner similar to that described in Example 23 using 1.383 g (5.32 mmol) of 2-[(ethoxycarbonyl)methyl]-2-pentylpropanedioic acid to yield 1.11 g (97%) of 2-[(ethoxycarbonyl)methyl]heptanoic acid.

Prepared in a manner similar to that described in Example 24 using 0.250 g (0.74 mmol) of (2S)-2-amino-3-(2-naphthyl)-N-benzylpropanamide, hydrochloride (from Compound 100), 0.160 g (0.74 mmol) of 2-[(ethoxycarbonyl)methyl]heptanoic acid, 0.100 g (0.74 mmol) of HOBt, 0.280 g (1.47 mmol) of EDC, and 0.24 mL (2.19 mmol) of NMM to yield 0.230 g (62%) of ethyl (3R,S)-3-(N-{(1S)-2-(2-naphthyl)-1-[N-benzylcarbamoyl]ethyl}carbamoyl)octanoate (1:1 mixture of diastereoisomers).

Prepared in a manner similar to that described in Compound 88 using 0.230 g (0.46 mmol) of ethyl (3R,S)-3-(N-{(1S)-2-(2-naphthyl)-1-[N-benzylcarbamoyl]ethyl}carbamoyl)octanoate (1:1 mixture of diastereoisomers) to yield 0.032 g (14%) of 2-(N-hydroxycarbamoylmethyl)(2R,S)—N-{(1S)-2-(2-naphthyl)-1-[N-benzylcarbamoyl]ethyl}heptanamide (1:1 mixture of diastereoisomers). MS (M+H)⁺ 490; (M−H)-488.

### Example 115

### Compound 94: 2-(N-hydroxycarbamoylmethyl)(2R)—N-{(1S)-1-[N-((1S)-1-carbamoylethyl)carbamoyl]-2-naphthylethyl}-4-methylpentanamide

Following the procedure of Example 3, (2S)-2-amino-N-(carbamoylethyl)-3-naphthylpropanamide (120 mg, 0.42 mmol), (2R)-2-({N-[(2,4-dimethoxyphenyl)methyl]-N-(phenylmethoxy)carbamoyl}methyl)-4-methylpentanoic acid, sodium salt (243 mg, 0.5 mmol), EDC (192 mg, 1 mmol), HOBt (77 mg, 0.5 mmol), DIEA (0.087 mL, 0.5 mmol) and dichloromethane (15 mL) to yield 331 mg (95%) of (2R)—N-{(1S)-1-[N-(carbamoylethyl)carbamoyl]-2-naphthylethyl}-N′-[(2,4-dimethoxyphenyl)methyl]-2-(2-methylpropyl)-N′-(phenylmethoxy)butane-1,4-diamide as a white solid.

(2R)—N-{(1S)-1-[N-(carbamoylethyl)carbamoyl]-2-naphthylethyl}-N′-[(2,4-dimethoxyphenyl)methyl]-2-(2-methylpropyl)-N′-(phenylmethoxy)butane-1,4-diamide (141 mg, 0.2 mmol) and 4/1 (v/v) mixture of trifluoroacetic acid and trimethylsilyl bromide were stirred under nitrogen for five minutes. The reaction was complete by LC after 3 hours. Added methanol and concentrated. The crude residue was treated with ether to give a precipitate to yield 90 mg (82%) of (2R)—N-{(1S)-1-[N-(carbamoylethyl)carbamoyl]-2-naphthylethyl}-2-(2-methylpropyl)-N′-(phenyethoxy)butane-1,4-diamide as a white solid.

Following the procedure of Example 91, (2R)—N-{(1S)-1-[N-(carbamoylethyl)carbamoyl]-2-naphthylethyl}-2-(2-methylpropyl)-N′-(phenylmethoxy)butane-1,4-diamide (80 mg, 0.15 mmol). The crude product was purified by silica gel chromatography (water/methanol, 40:60) to the isolation of 2-(N-hydroxycarbamoylmethyl)(2R)—N-{(1S)-1-[N-((1S)-1-carbamoylethyl)carbamoyl]-2-naphthylethyl}-4-methylpentanamide (9 mg) in 13% yield, R_{f}=0.4 (methanol/ethyl acetate, 1:4). MS (M+H)⁻ 455.

### Example 116

### Compound 95: 2-(N-hydroxycarbamoylmethyl)-N-{(1S)-2-(2-naphthyl)-1-[N-(2-phenylethyl)carbamoyl]ethyl}-4-methylpentanamide

Prepared in a manner similar to that described in Example 24 using 1.00 g (3.177 mmol) of (S)-(−)-2-(tert-butoxycarbonylamino)-3-(2-naphthyl)propanoic acid, 0.40 mL (3.177 mmol) of phenethylamine, 0.428 g (3.17 mmol) of HOBt, 1.216 g (6.34 mmol) of EDC, and 0.697 mL (6.34 mmol) of NMM to yield 1.290 g (96%) of (S)-(2-Naphthalen-2-yl-1-phenethylcarbamoyl-ethyl)-carbamic acid tert-butyl ester.

MS (M+H)⁺ 419; (M+HCO₂⁻)⁻ 463.

Prepared in a manner similar to that described in Example 4 using 1.200 g (2.87 mmol) of (S)-(2-Naphthalen-2-yl-1-phenethylcarbamoyl-ethyl)-carbamic acid tert-butyl ester, and 15mL of 4M solution of HCl in 1,4-dioxane to yield 0.966 g (95%) of (2S)-2-amino-3-(2-naphthyl)-N-(2-phenylethyl)propanamide, hydrochloride.

Prepared in a manner similar to that described in Example 24 using 0.246 g (0.69 mmol) of (2S)-2-amino-3-(2-naphthyl)-N-(2-phenylethyl)propanamide, hydrochloride, 0.140 g (0.69 mmol) of 2-[(ethoxycarbonyl)methyl]-4-methylpentanoic acid, 0.094 g (0.69 mmol) of HOBt, 0.266 g (1.39 mmol) of EDC, and 0.23 mL (2.09 mmol) of NMM to yield 0.242 g (70%) of ethyl (3R,S)-3-(N-{(1S)-2-(2-naphthyl)-1-[N-(2-phenylethyl)carbamoyl]ethyl}carbamoyl)-5-methylhexanoate (1:1 mixture of diastereoisomers).

MS (M+H)⁺ 503; (+HCO₂⁻)⁻ 547.

Prepared in a manner similar to that described in Compound 88 using 0.242 g (0.48 mmol) of ethyl (3R,S)-3-(N-{(1S)-2-(2-naphthyl)-1-[N-(2-phenylethyl)carbamoyl]ethyl}carbamoyl)-5-methylhexanoate (1:1 mixture of diastereoisomers) to yield 0.110 g (47%) of 2-(N-hydroxycarbamoylmethyl)(2R,S)—N-{(1S)-2-(2-naphthyl)-1-[N-(2-phenylethyl)carbamoyl]ethyl}-4-methylpentanamide (1:1 mixture of diastereoisomers).

MS (M+H)⁺ 490; (M−H)⁻ 488.

### Example 117

### Compounds 96 and 138: 2-(N-hydroxycarbamoylmethyl)(2R)—N-{(1S)-1-[N-((1S)-1-carbamoyl-3-methylbutyl)carbamoyl]-2-naphthylethyl}-4-methylpentanamide and 2-(N-hydroxycarbamoylmethyl)(2S)—N-{(1S)-1-[N-((1S)-1-carbamoyl-3-methylbutyl)carbamoyl]-2-naphthylethyl}-4-methylpentanamide

(2S)—N-((1S)-1-carbamoyl-3-methylbutyl)-2-[(tert-butoxy)carbonylamino]-3-naphthyl propanamide was prepared from (2S)-2-[(tert-butoxy)carbonylamino]-3-naphthyl propanoic acid (0.63 g, 2 mmol), (2S)-2-amino-4-methylpentanamide (0.26 g, 2 mmol), EDC HCl (0.77 g, 4 mmol), HOBt (0.27 g, 2 mmol), DIEA (0.35 mL, 2 mmol) and DMF (16 mL) using the procedure in Compound 39. Yield: 0.75 g (88%).

(2S)—N-((1S)-1-carbamoyl-3-methylbutyl)-2-amino-3-naphthylpropanamide, was prepared from (2S)—N-((1S)-1-carbamoyl-3-methylbutyl)-2-[(tert-butoxy)carbonyl amino]-3-naphthyl propanamide (0.43 g, 1 mmol) and 4N HCl/dioxane (10 mL) using the procedure from Example 4. Yield: 0.34 g (92%).

ethyl 3-(N-{(1S)-1-[N-((1S)-1-carbamoyl-3-methylbutyl)carbamoyl]-2-naphthylethyl}carbamoyl)-5-methylhexanoate was prepared from 2-[(ethoxycarbonyl)methyl]-4-methyl pentanoic acid (0.20 g, 1 mmol), (2S)—N-((1S)-1-carbamoyl-3-methylbutyl)-2-amino-3-naphthylpropanamide, (0.22 g, 0.6 mmol), EDC HCl (0.38 g, 2 mmol), HOBt (0.135 g, 1 mmol), DIEA (0.35 mL, 2 mmol) and dichloromethane (10mL) using the procedure from Example 3. Yield: 028 g (91%).

2-(N-hydroxycarbamoylmethyl)(2R)—N-{(1S)-1-[N-((1S)-1-carbamoyl-3-methylbutyl)carbamoyl]-2-naphthylethyl}-4-methylpentanamide (Compound 96) and 2-(N-hydroxycarbamoylmethyl)(2S)—N-{(1S)-1-[N-((1S)-1-carbamoyl-3-methylbutyl)carbamoyl]-2-naphthylethyl}-4-methylpentanamide (Compound 138).

The title compounds were prepared from ethyl 3-(N-{(1S)-1-[N-((1S)-1-carbamoyl-3-methylbutyl)carbamoyl]-2-naphthylethyl}carbamoyl)-5-methylhexanoate (0.16 g, 0.3 mmol) using the procedure from Compound 88. The two isomers were separated using C-18 reverse phase silica gel using the mixtures of methanol and water as eluents.

2-(N-hydroxycarbamoylmethyl)(2R)—N-{(1S)-1-[N-((1S)-1-carbamoyl-3-methylbutyl)carbamoyl]-2-naphthylethyl}-4-methylpentanamide (Compound 96) has R_{f}=1.875° in HPLC column. MS: (M+H⁺) 499.

¹H NMR: (300 MHz, DMSO-d₆): 10.41 δ (1H bs); 8.71 δ (1H bs); 8.33 δ (1H d); 8.26 δ (1H d); 8.20 δ (2H dm); 7.93 δ (1H m) 7.78 δ (2H m); 7.38 δ (2H m); 7.10 δ (1H m); 7.00 δ (1H m) 4.61 δ (1H m) 4.26 δ (1H m); 3.63 δ (1H m); 3.40 δ (1H m); 3.24 δ (2H m) 2.64 δ (1H m); 1.90 δ (1H m); 1.43 δ (4H m); 0.94 δ (6H m). 0.75 δ (3H dm); 0.73 δ (3H m).

2-(N-hydroxycarbamoylmethyl)(2S)—N-{(1S)-1-[N-((1S)-1-carbamoyl-3-methylbutyl)carbamoyl]-2-naphthylethyl}-4-methylpentanamide (Compound 138) has Rf=2.158′ in HPLC column. MS: (M+H⁺) 499.

¹H NMR: (300 MHz, DMSO-d₆): 10.56 δ (1H bs); 8.79 δ (1H bs); 8.67 δ (1H d); 8.21 δ (1H d); 8.13 δ (1H dm); 7.93 δ (1H d) 7.78 δ (1H m); 7.54 δ (3H m); 7.38 δ (1H m); 7.09 δ (1H m) 7.01 δ (1H d); 4.46 (11H m) 4.41 δ (1H m); 3.85 δ (1H m); 3.30 δ (1H m); 3.04 δ (1H m) 2.59 δ (1H m); 2.15 δ (1H m); 1.99 δ (1H m); 1.86 δ (1H m); 1.68 δ (1H m); 1.56 δ (1H m); 1.06 δ (1H m); 0.92 δ (7H m); 0.75 δ (6H, m).

### Example 118

### Compound 98: 2-N-hydroxycarbamoylmethyl)(2R)—N-{(1R)-1-[N-((1S) -1-carbamoylethyl)carbamoyl]-2-(2-naphthyl)ethyl}-4-methylpentanamide

Following the procedure of Example 4, (2R)—N-((1S)-1-carbamoylethyl)-2-[(tert-butoxy)carbonylamino]-3-(2-naphthyl)propanamide (1.4 g, 3.6 mmol) to yield 576 mg (56%) of (2R)—N-((1S)-1-carbamoylethyl)-2-amino-3-(2-naphthyl)propanamide as a white solid.

Following the procedure of Example 3, (2R)—N-((1S)-1-carbamoylethyl)-2-amino-3-(2-naphthyl)propanamide (143 mg, 0.5 mmol), (2R)-2-({N-[(2,4-dimethoxyphenyl)methyl]-N-(phenylmethoxy)carbamoyl}methyl)-4-methylpentanoic acid, sodium salt (243 mg, 0.5 mmol), EDC (192 mg, 1 mmol), HOBt (77 mg, 0.5 mmol), DIEA (0.087 mL, 0.5 mmol) and dichloromethane (15 mL) to yield 220 mg (63%) of (2R)—N-{(1R)-1-[N-((1S)-1-carbamoylethyl)carbamoyl]-2-(2-naphthyl)ethyl}-N′-[(2,4-dimethoxyphenyl)methyl]-2-(2-methylpropyl)-N′-(phenylmethoxy)butane-1,4-diamide as a white solid.

(2R)—N-{(1R)-1-[N-((1S)-1-carbamoylethyl)carbamoyl]-2-(2-naphthyl)ethyl}-N′-[(2,4-dimethoxyphenyl)methyl]-2-(2-methylpropyl)-N′-(phenylmethoxy)butane-1,4-diamide (100 mg, 0.14 mmol) and 4/1 (v/v) mixture of trifluoroacetic acid and trimethylsilyl bromide were stirred under nitrogen for five minutes. The reaction was complete by LC after 3 hours. Added methanol and concentrated. The crude residue was treated with ether to give a precipitate to yield 99 mg of (2R)—N-{(1R)-1-[N-((1S)-1-carbamoylethyl)carbamoyl]-2-(2-naphthyl)ethyl}-2-(2-methylpropyl)-N′-(phenylmethoxy)butane-1,4-diamide as a pink solid.

Following the procedure of Example 91, (2R)—N-{(1R)-1-[N-((1S)-1-carbamoylethyl)carbamoyl]-2-(2-naphthyl)ethyl}-2-(2-methylpropyl)-N′-(phenylmethoxy)butane-1,4-diamide (86 mg, 0.15 mmol). The crude product was purified by silica gel chromatography (water/methanol, 30:70) to the isolation of 2-(N-hydroxycarbamoylmethyl)(2R)—N-{((R)-1-[N-((1S)-1-carbamoylethyl)carbamoyl]-2-(2-naphthyl)ethyl}-4-methylpentanamide (12 mg) in 13% yield, R_{f}=0.16 (methanol/chloroform, 5:95). MS (M+H)⁺ 457.

### Example 119

### Compound 99: 2-(N-hydroxycarbamoylmethyl)-N-[(1S)-1-(N-methylcarbamoyl)-2-(2-naphthyl)ethyl]-4-methylpentanamide

Prepared in a manner similar to that described in Example 24 using 3.00 g (9.52 mmol) of (S)-(−)-2-(tert-butoxycarbonylamino)-3-(2-naphthyl)propanoic acid, 1.28 g (19.10 mmol) of methylamine hydrochloride, 1.29 g (9.52 mmol) of HOBt, 3.61 g (19.00 mmol) of EDC, and 4.2 mL (38.26 mmol) of NMM to yield 2.81 g (90%) of (2S)-2-[(tert-butoxy)carbonylamino]-N-methyl-3-(2-naphthyl)propanamide.

Prepared in a manner similar to that described in Example 4 using 2.81 g (8.57 mmol) of (2S)-2-[(tert-butoxy)carbonylamino]-N-methyl-3-(2-naphthyl)propanamide, and 15mL of 4M solution of HCl in 1,4-dioxane to yield 1.50 g (66%) of (2S)-2-amino-N-methyl-3-(2-naphthyl)propanamide, hydrochloride.

Prepared in a manner similar to that described in Example 24 using 0.200 g (0.75 mmol) of (2S)-2-amino-N-methyl-3-(2-naphthyl)propanamide, hydrochloride, 0.152 g (0.75 mmol) of 2-[(ethoxycarbonyl)methyl]-4-methylpentanoic acid, 0.103 g (0.75 mmol) of HOBt, 0.300 g (1.50 mmol) of EDC, and 0.32 mL (2.80 mmol) of NMM to yield 0.250 g (81%) of ethyl (3R,S)-3-{N-[(1S)-1-(N-methylcarbamoyl)-2-(2-naphthyl)ethyl]carbamoyl}-5-methylhexanoate (1:1 mixture of diastereoisomers).

Prepared in a manner similar to that described in Compound 88 using 0.250 g (0.61 mmol) of ethyl (3R,S)-3-{N-[(1R)-1-(N-methylcarbamoyl)-2-(2-naphthyl)ethyl]carbamoyl}-5-methylhexanoate (1:1 mixture of diastereoisomers) to yield 0.029 g (12%) of 2-(N-hydroxycarbamoylmethyl)(2R,S)—N-[(1S)-1-(N-methylcarbamoyl)-2-(2-naphthyl)ethyl]-4-methylpentanamide (1:1 mixture of diastereoisomers). MS (M+H)⁺ 400; (M−H)-398.

### Example 120

### Compound 100: 2-(N-hydroxycarbamoylmethyl)-N-{(1S)-2-(2-naphthyl)-1-[N-benzylcarbamoyl]ethyl}-4-methylpentanamide

Prepared in a manner similar to that described in Example 24 using 1.00 g (3.17 mmol) of (S)-(−)-2-(tert-butoxycarbonylamino)-3-(2-naphthyl)propanoic acid, 0.355 mL (3.17 mmol) of benzylamine, 0.428 g (3.17 mmol) of HOBt, 1.216 g (6.34 mmol) of EDC, and 0.697 mL (6.34 mmol) of NMM to yield 1.192 g (92%) of (2S)-2-[(tert-butoxy)carbonylamino]-3-(2-naphthyl)-N-benzylpropanamide. MS (M+H)⁺ 405; (M+HCO₂⁻)⁻ 449.

Prepared in a manner similar to that described in Example 4 using 1.100 g (2.72 mmol) of (2S)-2-[(tert-butoxy)carbonylamino]-3-(2-naphthyl)-N-benzylpropanamide, and 15 mL of 4M solution of HCl in 1,4-dioxane to yield 0.920 g (99%) of (2S)-2-amino-3-(2-naphthyl)-N-benzylpropanamide, hydrochloride.

Prepared in a manner similar to that described in Example 24 using 0.219 g (0.64 mmol) of (2S)-2-amino-3-(2-naphthyl)-N-benzylpropanamide, hydrochloride, 0.130 g (0.64 mmol) of 2-[(ethoxycarbonyl)methyl]-4-methylpentanoic acid, 0.087 g (0.64 mmol) of HOBt, 0.247 g (1.30 mmol) of EDC, and 0.21 mL (1.90 mmol) of NMM to yield 0.246 g (79%) of ethyl (3R,S)-3-(N-{(1S)-2-(2-naphthyl)-1-[N-benzylcarbamoyl]ethyl}carbamoyl)-5-methylhexanoate (1:1 mixture of diastereoisomers). MS (M+H)⁺ 489; (M+HCO₂⁻)⁻ 533.

Prepared in a manner similar to that described in Compound 88 using 0.246 g (0.50 mmol) of ethyl (3R)-3-N-{(1S)-2-(2-naphthyl)-1-[N-benzylcarbamoyl]ethyl}carbamoyl)-5-methylhexanoate (1:1 mixture of diastereoisomers) to yield 0.021 g (9%) of 2-(N-hydroxycarbamoylmethyl)(2R,S)—N-{(1S)-2-(2-naphthyl)-1-[N-benzylcarbamoyl]ethyl}-4-methylpentanamide (1:1 mixture of diastereoisomers). MS (+H)⁺ 476; (M−H)-474.

### Example 121

### Compound 101: 2-(N-hydroxycarbamoylmethyl)-N-[(1S,2R)-1-(N-methylcarbamoyl)-2-(phenylmethoxy)propyl]-4-methylpentanamide

Prepared in a manner similar to that described in Example 24 using 2 g (6.5 mmol) of Boc-1-thr(bzl)-OH, 0.87 g (13 mmol) of methylamine hydrochloride, 0.873 g (6.5 mmol) of HOBt, 2.46 g (13 mmol) of EDC, and 2.8 mL (26 mmol) of NMM to yield (2S,3R)-2-[(tert-butoxy)carbonylamino]-N-methyl-3-(phenylmethoxy)butanamide. Without father purification the product was used in the next reaction

Prepared in a manner similar to that described in Example 4 using of (2S,3R)-2-[(tert-butoxy)carbonylamino]-N-methyl-3-(phenylmethoxy)butanamide, and 15mL of 4M solution of HCl in 1,4-dioxane to yield 1.5 g (88%) of (2S,3R)-2-amino-N-methyl-3-(phenylmethoxy)butanamide, chloride.

Prepared in a manner similar to that described in Example 24 using 0.100 g (0.38 mmol) of 2S,3R)-2-amino-N-methyl-3-(phenylmethoxy)butanamide, chloride, 0.15 g (0.74 mmol) of 2-[(ethoxycarbonyl)methyl]-4-methylpentanoic acid, 0.050 g (0.37 mmol) of HOBt, 0.150 g (0.79 mmol) of EDC, and 0.2 mL (1.5 mmol) of NMM to yield 0.125 g (81%) of ethyl 3-{N-[(1S,2R)-1-(N-methylcarbamoyl)-2-(phenylmethoxy)propyl]carbamoyl}-5-methylhexanoate.

Prepared in a manner similar to that described in Example 34 using 0.100 g (0.25 mmol) ethyl 3-{N-[(1S,2R)-1-(N-methylcarbamoyl)-2-(phenylmethoxy)propyl]carbamoyl}-5-methylhexanoate to yield 0.035 g (37%) of 3-{N-[(1S,2R)-1-(N-methylcarbamoyl)-2-(phenylmethoxy)propyl]carbamoyl}-5-methylhexanoic acid.

Prepared in a manner similar to that described in Example 24 using 0.035 g (0.09 mmol) of, 3-{N-[(1S,2R)-1-(N-methylcarbamoyl)-2-(phenylmethoxy)propyl]carbamoyl}-5-methylhexanoic acid, 0.015 g (0.09 mmol) of o-benzylhydroxylamine hydrochloride, 0.012 g (0.08 mmol) of HOBt, 0.035 g (0.18 mmol) of EDC, and 0.02 mL (0.18 mmol) of NMM to yield 0.040 g (92%) of N-[(1S,2R)-1-(N-methylcarbamoyl)-2-(phenylmethoxy)propyl]-2-(2-methylpropyl)-N′-(phenylmethoxy)butane-1,4-diamide.

Prepared in a manner similar to that described in Example 21 using 0.040 g (0.082 mmol) of N-[(1S,2R)-1-(N-methylcarbamoyl)-2-(phenylmethoxy)propyl]-2-(2-methylpropyl)-N′-(phenylmethoxy)butane-1,4-diamide and palladium on charcoal (0.01 g) to give 0.03 g (93%) of 2-(N-hydroxycarbamoylmethyl)-N-[(11S,2R)-1-(N-methylcarbamoyl)-2-(phenylmethoxy)propyl]-4-methylpentanamide (1:1 mixture of diastereoisomers). MS (M+H)⁺ 394; (M−H)-392.

### Example 122

### Compound 102

### 2-(N-hydroxycarbamoylmethyl)-N-(2-indol-3-ylethyl)-4-phenylbutanamide

Prepared in a manner similar to that described in Example 23 using 4.00 g (13.20 mmol) of tert-butyl ethyl 2-[(tert-butyl)oxycarbonyl]butane-1,4-dioate, 1.92 mL (13.20 mmol) of (2-iodoethyl)benzene, 0.592 g (13.20 mmol) of NaH to yield 4.564 g (85%) of tert-butyl ethyl 2-[(tert-butyl)oxycarbonyl]-2-(2-phenylethyl)butane-1,4-dioate.

¹HNMR (300 MHz, CDCl₃) δ 7.30-7.15 (5×, m), 4.13 (2H, q), 2.96 (2H, s), 2.59-2.53 (2H, m), 2.23-2.17 (2H, m), 1.48 (18H, s), 1.25 (3H, t).

Prepared in a manner similar to that described in Example 23 using 2.00 g (4.90 mmol) of tert-butyl ethyl 2-[(tert-butyl)oxycarbonyl]-2-(2-phenylethyl)butane-1,4-dioate, 0.5 mL of Et₃SiH, and 12 mL of TFA to yield 1.419 g (98%) of 2-[(ethoxycarbonyl)methyl]-2-(2-phenylethyl)propanedioic acid.

¹H NMR (300 MHz, CDCl₃) δ 8.30 (2H, br s), 7.31-7.13 (55H, m), 4.16 (2H, q), 3.18 (2H, s), 2.68-2.62 (2H, m), 2.27-2.20 (2H, m), 1.26 (3H, t).

Prepared in a manner similar to that described in Example 23 using 1.419 g (4.82 mmol) of 2-[(ethoxycarbonyl)methyl]-2-(2-phenylethyl)propanedioic acid to yield 1.151 g (95%) of 2-[(ethoxycarbonyl)methyl]-4-phenylbutanoic acid.

¹H NMR (300 MHz, CDCl₃) δ 10.50 (1H, br s), 7.29-7.17 (5H, m), 4.14 (2H, q), 2.94-2.88 (1H, m), 2.80-2.66 (3H, m), 2.51 (1H, dd), 2.08-2.01 (1H, m), 1.92-1.82 (1H, m), 1.25 (3H, t).

Prepared in a manner similar to that described in Example 24 using 0.104 g (0.42 mmol) of 2-[(ethoxycarbonyl)methyl]-4-phenylbutanoic acid, 0.067 g (0.42 mmol) of 2-indol-3-ylethylamine, 0.056 g (0.42 mmol) of HOBt, 0.159 g (0.83 mmol) of EDC, and 0.09 mL (0.83 mmol) of NMM to yield 0.158 g (97%) of ethyl 3-[N-(2-indol-3-ylethyl)carbamoyl]-5-phenylpentanoate.

Prepared in a manner similar to that described in Example 26 using 0.158 g (0.40 mmol) of ethyl 3-[N-(2-indol-3-ylethyl)carbamoyl]-5-phenylpentanoate to yield 0.094 g (63%) of 3-[2-(1H-Indol-3-yl)-ethylcarbamoyl]-5-phenyl-pentanoic acid, lithium salt.

Prepared in a manner similar to that described in Example 24 using 0.074 g (0.20 mmol) of 3-[2-(1H-Indol-3-yl)-ethylcarbamoyl]-5-phenyl-pentanoic acid, lithium salt, 0.032 g (0.20 mmol) of O-benzylhydroxylamine hydrochloride, 0.027 g (0.20 mmol) of HOBt, 0.077 g (0.4 mmol) of EDC, and 0.044 mL (0.4 mmol) of NMM to yield 0.078 g (83%) of N-(2-indol-3-ylethyl)-2-(2-phenylethyl)-N′-(phenylmethoxy)butane-1,4-diamide. MS (M+H)⁺ 470; (M−H)⁻ 468.

Prepared in a manner similar to that described in Example 21 using 0.077 g (0.16 mmol) of N-(2-indol-3-ylethyl)-2-(2-phenylethyl)-N′-(phenylmethoxy)butane-1,4-diamide and 0.023 g of 10% of Pd/C to yield 0.053 g (88%) of 2-(N-hydroxycarbamoylmethyl)-N-(2-indol-3-ylethyl)-4-phenylbutanamide. MS (M+H)⁺ 380; (M−H)⁻ 378.

### Example 123

### Compound 103: 2-(N-hydroxycarbamoylmethyl)-N-{(1S)-1-[N-((1S)-1-carbamoyl-2-methylbutyl)carbamoyl]-2-naphthylethyl}-4-methylpentanamide

(2S)—N-((1S)-1′-carbamoyl-3-methylbutyl)-2-[(tert-butoxy)carbonylamino]-3-naphthylpropanamide was prepared from (2S)-2-[(tert-butoxy)carbonylamino]-3-naphthylpropanoic acid (0.63 g, 2 mmol), (2S,3S)-2-amino-3-methylpentanamide, hydrochloride (0.50 g, 3 mmol), EDC HCl (0.77 g, 4 mmol), HOBt (0.23 g, 2 mmol), DIEA (1.22 mL, 7 mmol) and dichloromethane (20 mL) using the procedure from Example 3.

Yield: 0.69 g (80%).

(2S)—N-((1S)-1-carbamoyl-2-methylbutyl)-2-amino-3-naphthylpropanamide was prepared from (2S)—N-((1S)-1-carbamoyl-3-methylbutyl)-2-[(tert-butoxy)carbonylamino]-3′-naphthylpropanamide (0.60 g, 1.4 mmol), and 4N HCl/dioxane (10 mL) using the procedure as in Example 4. Yield: 0.25 g (54%).

N-{(1S)-1-[N-((1S)-1-carbamoyl-2-methylbutyl)carbamoyl]-2-naphthylethyl}-N′-[(2,4-dimethoxyphenyl)methyl]-2-(2-methylpropyl)-N′-(phenylmethoxy)butane-1,4-diamide was prepared from sodium salt 2-({N-[(2,4-dimethoxyphenyl)methyl]-N-(phenyl methoxy)carbamoyl}methyl)-4-methylpentanoic acid (0.23 g, 0.5 mmol), (2S)—N-((1S)-1-carbamoyl-2-methylbutyl)-2-amino-3-naphthylpropanamide (0.17 g, 0.5 mmol), EDC HCl (192 mg, 11.0 mmol), HOBt (135 mg, 11.0 mmol), DIEA (87 μL, 0.5 mmol), dichloromethane (5 mL) using the procedure from Example 3. Yield: 0.30 g (81%).

N-{(1S)-1-[N-((1S)-1-carbamoyl-2-methylbutyl)carbamoyl]-2-naphthylethyl}-2-(2-methylpropyl)-N′-(phenylmethoxy)butane-1,4-diamide was prepared by stirring N-{(1S)-1-[N-((1S)-1-carbamoyl-2-methylbutyl)carbamoyl]-2-naphthylethyl}-N′-[(2,4-dimethoxy phenyl)methyl]-2-(2-methylpropyl)-N-(phenylmethoxy)butane-1,4-diamide (0.10 g, 0.135 mmol) with trifluoro acetic acid/trimethylsilylbromide 4/1 (1.0 mL) at room temperature under nitrogen for 3 hours. The solvent was rotovaped, the residue was triturated with ether to obtain a solid. Yield: 75 mg (94%).

2-(N-hydroxycarbamoylmethyl)-N-{(1S)-1-[N-((1S)-1-carbamoyl-2-methylbutyl)carbamoyl]-2-naphthylethyl}-4-methylpentanamide was prepared by stirring a methanol (10 mL) solution of N-{(1S)-1-[N-((1S)-1-carbamoyl-2-methylbutyl)carbamoyl]-2-naphthylethyl}-2-(2-methylpropyl)-N′-(phenylmethoxy)butane-1,4-diamide (75 mg, 0.13 mmol) with 10% palladium/carbon (25 mg) in presence of hydrogen. The product was purified by RP C-18 column using methanol and water mixtures as eluents. The product contained mostly the single isomer of (91/9) 2-(N-hydroxycarbamoylmethyl)(2R)—N-{(1S)-1-[N-((1S)-1-carbamoyl-2-methylbutyl)carbamoyl]-2-naphthylethyl}-4-methylpentanamide. MS: (+H) 499.

### Example 124

### Compound 104: 7-aza-6-oxo-7-(3-(2-phenylethyl)-5-{3-[3-(trifluoromethyl)phenoxy]phenyl(1,3,4-thiadiazolin-2-ylidene))heptanoic acid

3-[3-(trifluoromethyl)phenoxy]benzaldehyde (2.5 g, 9.4 mmol) and thiosemicarbazide (0.85 g, 9.4 mmol) in 25 mL ethanol was placed in microwave at 160° C. for 5 mins to give 2.8 g (88%) of [((1E)-1-aza-2-{3-[3-(trifluoromethyl)phenoxy]phenyl}vinyl)amino]aminomethane-1-thione

Prepared in a manner similar to that described in Example 32 using 2.8 g (8.25 mmol) of [((1E)-1-aza-2-{3-[3-(trifluoromethyl)phenoxy]phenyl}vinyl)amino]aminomethane 1-thione to yield 2.7 g (99%) of 5-{3-[3-(trifluoromethyl)phenoxy]phenyl}-1,3,4-thiadiazole-2-ylamine.

5-{3-[3-(trifluoromethyl)phenoxy]phenyl}-1,3,4-thiadiazole-2-ylamine (2.7 g, 8 mmol) and trifluoroacetic anhydride (1.8 mL, 8 mmol) were stirred in 20 mL dichloromethane at room temperature for overnight to yield 3.3 g (95%) of 2,2,2-trifluoro-N-(5-{3-[3-(trifluoromethyl)phenoxy]phenyl}(1,3,4-thiadiazol-2-yl))acetamide.

Prepared in a manner similar to that described in Example 33 using 2.79 g (6.44 mmol) of 2,2,2-trifluoro-N-(5-{3-[3-(trifluoromethyl)phenoxy]phenyl}(1,3,4-thiadiazol-2-yl))acetamide to yield 1.25 g (36%) of 1-aza-3,3,3-trifluoro-1-(3-(2-phenylethyl)-5-{3-[3-(trifluoromethyl)phenoxy]phenyl}(1,3,4-thiadiazolin-2-ylidene))acetone.

1-aza-3,3,3-trifluoro-1-(3-(2-phenylethyl)-5-{3-[3-(trifluoromethyl)phenoxy]phenyl}(1,3,4-thiadiazolin-2-ylidene))acetone (1.25 g 2.32 mmol) and potassium carbonate (0.4 g, 2.32 mmol) were stirred in methanol (20 mL) for overnight to yield 0.85 g (83%) of 3-(2-phenylethyl)-5-{3-[3-(trifluoromethyl)phenoxy]phenyl}-1,3,4-thiadiazolin-2-imine.

3-(2-phenylethyl)-5-{3-[3-(trifluoromethyl)phenoxy]phenyl}-1,3,4-thiadiazolin-2-imine (0.2 g, 0.45 mmol), 5-(chloroformyl)valeric acid methyl ester (0.16 g, 0.89 mmol), DMAP (0.11 g, 0.9 mmol) in 20 mL dichloromethane were stirred at room temperature for 2 hours. The crude residue washed with brine and extracted with dichloromethane. Dichloromethane was dried over sodium sulfate, filtered, and concentrated to yield 0.2 g (76%) of methyl 7-aza-6-oxo-7-(3-(2-phenylethyl)-5-{3-[3-(trifluoromethyl)phenoxy]phenyl}(1,3,4-thiadiazolin-2-ylidene))heptanoate.

Prepared in a manner similar to that described in Example 34 using 0.200 g (0.34 mmol) methyl 7-aza-6-oxo-7-(3-(2-phenylethyl)-5-{3-[3-(trifluoromethyl)phenoxy]phenyl}(1,3,4-thiadiazolin-2-ylidene))heptanoate to yield 0.18 g (90%) of 5-{3-Phenethyl-5-[3-(3-trifluoromethyl-phenoxy)-phenyl]-3H-[1,3,4]thiadiazol-2-ylidenecarbamoyl}-pentanoic acid as a yellow solid. MS (M+H)⁺ 570; (M−H)-568:

### Example 125

### Compound 105: Mixture of 2-[(2-(N-hydroxycarbamoyl)(1S,2S)cyclohexyl)carbonylamino](2S)—N-((1S)-1-carbamoylpropyl)-3-naphthylpropanamide and 2-[(2-(N-hydroxycarbamoyl)(1R,2R)cyclohexyl)carbonylamino](2S)—N-((1S)-1-carbamoylpropyl)-3-naphthyl propanamide

(2S)—N-((1S)-1-carbamoylpropyl)-2-[(tert-butoxy)carbonylamino]-3-naphthylpropan amide was prepared from (2S)-2-[(tert-butoxy)carbonylamino]-3-naphthylpropanoic acid (0.63 g, 2 mmol), (2S)-2-aminobutanamide (0.20 g, 2 mmol), EDC HCl (0.58 g, 3 mmol), HOBt (0.31 g′, 2 mmol), DEA (0.52 mL, 3 mmol), and 20 mL using the procedure from Compound 39. Yield: 0.64 g (79%).

(2S)—N-((1S)-1-carbamoylpropyl)-2-amino-3-naphthylpropanamide was prepared by stirring (2S)—N-((1S)-1-carbamoylpropyl)-2-[(tert-butoxy)carbonylamino]-3-naphthylpropan amide (0.80 g, 2 mmol) in 4N HCl/dioxane (10 mL) using the procedure from Example 4. Yield: 0.30 g (50%).

Mixture of (1R,2R)-2-(N-{(1S)-1-[N-((1S)-1-carbamoylpropyl)carbamoyl]-2-naphthylethyl}carbamoyl)cyclohexanecarboxylic acid and 2-(N-{(1S)-1-[N-((1S)-1-carbamoylpropyl)carbamoyl]-2-naphthylethyl}carbamoyl)(1S,2S)cyclohexanecarboxylic acid were prepared by stirring a mixture of trans-1,2-cyclohexanedicarboxylic anhydride (0.46 g, 3 mmol) and (2S)—N-((1S)-1-carbamoylpropyl)-2-amino-3-naphthylpropan amide (0.45 g, 2 mmol) in dichloromethane (10 mL) for overnight. The dichloromethane was rotovaoped, the residue was taken in EtOAc and filtered. The solid was the 1/1 mixture of two diasteroismers. Yield: 0.50 g (72%).

Mixture of Diastereo isomers (2S)—N-((1S)-1-carbamoylpropyl)-2-((1S,2S)-2-[N-(phenylmethoxy)carbamoyl]cyclohexyl}-carbonylamino)-3-naphthylpropanamide was prepared from mixture of (1R,2R)-2-(N-{(1S)-1-[N-((1S)-1-carbamoylpropyl)carbamoyl]-2-naphthylethyl}carbamoyl)cyclohexanecarboxylic acid and 2-(N-{(1S)-1-[N-((1S)-1-carbamoylpropyl)carbamoyl]-2-naphthylethyl}carbamoyl)(1S,2S) cyclo hexanecarboxylic acid (0.45 g, 1 mmol), O-benzylhydroxylamine (0.25 g, 2 mmol), EDC HCl (0.39 g, 2 mmol), HOBt (0.153 g, 1 mmol), DIEA (0.35 mL, 2 mmol), DMF (5 mL) using the procedure from Example 3. Yield: 175 mg (31%).

Mixture of 2-[(2-(N-hydroxycarbamoyl)(1S,2S)cyclohexyl)carbonylamino](2S)—N-((1S)-1-carbamoylpropyl)-3-naphthylpropanamide and 2-[(2-(N-hydroxycarbamoyl)(1R, 2R)cyclohexyl)carbonylamino](2S)—N-((1S)-1-carbamoylpropyl)-3-naphthyl propanamide was prepared by stirring the mixture of Diastereo isomers (2S)—N-((1S)-1-carbamoylpropyl)-2-({(1S,2S)-2-[N-(phenylmethoxy)carbamoyl]cyclohexyl}-carbonylamino)-3-naphthylpropanamide (0.17 g, 0.3 mmol) in acetic acid (10 mL) and 10% palladium/carbon (50 mg) for overnight in presence of hydrogen. The next day, the palladium/carbon was filtered off and acetic was removed under vacuum to get a solid with faint orange color. Yield: 0.10 g (71%). MS: (M+H⁺) 469.

### Example 126

### Compound 128: 2-{(1E)-1-aza-2-[3-({5-[3-(3-methoxyphenoxy)phenyl](1,3,4-thiadiazol-2-yl)}amino)phenyl]prop-1-enyloxy}acetic acid

A mixture of 3-bromoanisole (10.00 g, 53.5 mmol), methyl 3-hydroxybenzoate (8.14 g, 53.5 mmol) and potassium carbonate (14.78 g, 106.9 mmol) in dry pyridine (75 mL) were stirred under argon at room temperature. Copper (II) oxide (8.51 g, 106.9 mmol) was added and the reaction mixture refluxed for 65 hours. After cooling to room temperature the mixture was added CH₂Cl₂ (50 mL) and filtered through celite. The filter cake was washed with fresh CH₂Cl₂ (50 mL). The combined organics were concentrated in vacuo. The residue was purified by flash chromatography (ethyl acetate/hexanes, 1:10 to 1:4) to yield 8.77 g (64%) of methyl 3-(3-methoxyphenoxy)benzoate as colorless oil.

Prepared in a manner similar to that described in Example 37 using 8.77 g (34 mmol) of methyl 3-(3-methoxyphenoxy)benzoate and 6.59 mL (136 mmol) of hydrazine hydrate to yield 8.25 g (94%) of 1-(3-methoxyphenoxy)benzene-3-carbohydrazide.

Prepared in a manner similar to that described in Example 37 using 2.00 g (7.70 mmol) of 1-(3-methoxyphenoxy)benzene-3-carbohydrazide and 1.374 g (7.70 mmol) of 3-acetylphenyl isothiocyanate to yield 3.20 g (95%) of N-({[(3-acetylphenyl)amino]thioxomethyl}amino)[3-(3-methoxyphenoxy)phenyl]carboxamide.

Prepared in a manner similar to that described in Example 37 using 3.20 g (7.35 mmol) of N-({[(3-acetylphenyl)amino]thioxomethyl}amino)[3-(3-methoxyphenoxy)phenyl]carboxamide and 2.95 g (15.50 mmol) of p-toluenesulfonic acid monohydrate (replace the conc. H₂SO₄) to yield 1.882 g (61%) of 1-[3-({5-[3-(3-methoxyphenoxy)phenyl]-1,3,4-thiadiazol-2-yl}amino)phenyl]ethan-1-one. MS (M+H)⁺ 418; (M−H)⁻ 416.

Prepared in a manner similar to that described in Example 40 using 0.050 g (0.12 mmol) of 1-[3-({5-[3-(3-methoxyphenoxy)phenyl]-1,3,4-thiadiazol-2-yl}amino)phenyl]ethan-1-one, 0.018 g (0.14 mmol) of carboxymethoxylamine hemihydrochloride and 0.02 mL (0.14 mmol) of triethylamine to yield 0.011 g (19%) of 2-{(1E)-1-aza-2-[3-({5-[3-(3-methoxyphenoxy)phenyl](1,3,4-thiadiazol-2-yl)}amino)phenyl]prop-1-phenyloxy}acetic acid. MS (+H)⁺ 491; (M−H)⁻ 489.

### Example 127

### Compound 129: 4-{[3-({5-[5-methoxy-3-(phenylmethoxy)phenyl]-1,3,4-thiadiazol-2-yl}amino)phenylthio]methyl}benzoic acid

3-aminothiophenol (1.1 mL, 20 mmol), methyl 4-(bromomethyl)benzoate (5.04 g, 22 mmol), 1M NaOH (25 mL) in methanol (50 mL) were stirred for 1 hour. Concentrated reaction mixture. The crude residue was purified by flash chromatography (hexanes/ethyl acetate, 4:1) to yield 1.9 g (32%) of methyl 4-[(3-aminophenylthio)methyl]benzoate as a white solid.

### Methyl 4-[(3-aminophenylthio)methyl]benzoate (1.8 g, 6.6 mmol), dichloromethane (68mL), water (90 mL), and thiophosgene (1.03 mL, 13.4 mmol) were stirred for 24 hours. Removed dichloromethane. Washed with water and extracted with dichloromethane.

Dried dichloromethane over sodium sulfate, filtered, and concentrated to yield 2 g (96%) of methyl 4-[(3-isothiocyanatophenylthio)methyl]benzoate as a brown liquid. Following the procedure of Example 31, methyl 4-[(3-isothiocyanatophenylthio) methyl]benzoate (1.9 g, 6 mmol), (0.58 mL, 12 mmol), and toluene instead of ethanol to yield 790 mg (38%) of methyl 4-({3-[(hydrazinothioxomethyl)amino]phenylthio}methyl)benzoate as a white solid.

Methyl 4-(f{3-[(hydrazinothioxomethyl)amino]phenylthio}methyl)benzoate (190 mg, 0.54 mmol) and 3-methoxy-5-(phenylmethoxy)benzaldehyde (190 mg, 0.54 mmol) in ethanol were reluxed for 4 hours to yield 199 mg (65%) of methyl 4-[(3-{[({(1E)-1-aza-2-[5-methoxy-3-(phenylmethoxy)phenyl]vinyl}amino)thioxomethyl]amino}phenylthio) methyl]benzoate as a white solid.

Following the procedure of Example 32, methyl 4-[(3-f{[({(1E)-1-aza-2-[5-methoxy-3-(phenylmethoxy)phenyl]vinyl}amino)thioxomethyl]amino}phenylthio)methyl]benzoate (176 mg, 0.3 mmol) to yield 103 mg (61%) of methyl 4-{[3-({5-[5-methoxy-3-(phenylmethoxy)phenyl]-1,3,4-thiadiazol-2-yl}amino)phenylthio]methyl}benzoate as a light brown solid.

Following the procedure of Example 34, methyl 4-{[3-({5-[5-methoxy-3-(phenylmethoxy)phenyl]-1,3,4-thiadiazol-2-yl}amino)phenylthio]methyl}benzoate (30 mg, 0.05 mmol) to the isolation of 4-{[3-({5-[5-methoxy-3-(phenylmethoxy)phenyl]-1,3,4-thiadiazol-2-yl}amino)phenylthio]methyl}benzoic acid (25 mg) in 93% yield, R_{f}=0.44 (chloroform/methanol/acetic acid, 85:10:5). MS (M+H)⁺ 556.

### Example 128

### Compound 130: 2-((1E)-1-aza-2-{3-[aza(3-methyl-5-{3-[3-(trifluoromethyl)phenoxy]phenyl}(1,3,4-thiadiazolin-2-ylidene))methyl]phenyl}prop-1-phenyloxy)acetic acid

1-{3-[aza(3-methyl-5-{3-[3-(trifluoromethyl)phenoxy]phenyl}(1,3,4-thiadiazolin-2-ylidene))methyl]phenyl}ethan-1-one was prepared from 1-{3-[(5-{3-[3-(trifluoromethyl)phenoxy]phenyl}-1,3,4-thiadiazol-2-yl)amino]phenyl}ethan-1-one (0.20 g, 0.45 mmol), 1M solution of potassium tert-butoxide (0.45 mL, 0.45 mmol), methyliodide (0.28 mL, 4.5 mmol) using the procedure from Example 38. Yield: 15 mg (7%).

2-((1E)-1-aza-2-{3-[aza(3-methyl-5-{3-[3-(trifluoromethyl)phenoxy]phenyl}(1,3,4-thiadiazolin-2-ylidene))methyl]phenyl}prop-1-phenyloxy)acetic acid was prepared from 1-{3-[aza(3-methyl-5-{3-[3-(trifluoromethyl)phenoxy]phenyl}(1,3,4-thiadiazolin-2-ylidene)) methyl]phenyl}ethan-1-one (20 mg, 0.043 mmol), carboxymethoxylamine hemihydrochloride (11 mg, 0.09 mmol), triethylamine (7 μL, 0.05 mmol) and ethanol (2 mL) using the procedure from Example 41. Yield: 16 mg (70%).

MS: (M+X) 543 and (M−H)-541.

### Example 129

### Compound 131: 3-({5-[5-({3-[(4-carboxyphenyl)methylthio]phenyl}amino)(1,3,4-thiadiazol-2-yl)]-3-(phenylmethoxy)phenoxy}methyl)benzoic acid

Methyl 4-({3-[(hydrazinothioxomethyl)amino]phenylthio}methyl)benzoate (223 mg, 0.75 mmol) and methyl 3-{[5-carbonyl-3-(phenylmethoxy)phenoxy]methyl}benzoate (282 mg, 0.75 mmol) in ethanol were reluxed for overnight to yield 529 mg (99%) of methyl 3-({3-[(1E)-2-aza-2-({[(3-{[4-(methoxycarbonyl)phenyl]methylthio}phenyl)amino]thioxomethyl}amino)vinyl]-5-(phenylmethoxy)phenoxy}methyl)benzoate as a brown liquid.

Following the procedure of Example 32, methyl 3-({3-[(1E)-2-aza-2-({[(3-{[4-(methoxycarbonyl)phenyl]methylthio}phenyl)amino]thioxomethyl}amino)vinyl]-5-(phenylmethoxy)phenoxy}methyl)benzoate (529 mg, 0.75 mmol) to yield 112 mg (21%) of methyl 3-[(5 {5-[(3-{[4-(methoxycarbonyl)phenyl]methylthio}phenyl)amino](1,3,4-thiadiazol-2-yl)}-3-(phenylmethoxy)phenoxy)methyl]benzoate as an off white solid.

Following the procedure of Example 34, methyl 3-[(5-{5-[(3-{[4-(methoxycarbonyl)phenyl]methylthio}phenyl)amino](1,3,4-thiadiazol-2-yl)}-3-(phenylmethoxy)phenoxy)methyl]benzoate (100 mg, 0.14 mmol). The crude residue was recrystallized in ethanol to the isolation of 3-({5-[5-({3-[(4-carboxyphenyl)methylthio]phenyl}amino)(1,3,4-thiadiazol-2-yl)]-3-(phenylmethoxy)phenoxy}methyl)benzoic acid (34 mg) in 36% yield. R_{f}=0.69 (chloroform/methanol/acetic acid, 85:10:5). MS (M+H)⁻ 674.

### Example 130

### Compound 132: 4-({5-[3,5-bis(phenylmethoxy)phenyl]-1,3,4-thiadiazol-2-yl}amino)benzoic acid

Following the procedure of Example 31, 4-methoxycarbonylphenyl isothiocyanate (193 mg, 1 mmol), hydrazine hydrate (0.097 mL, 2 mmol), and using toluene instead of ethanol to yield 192 mg (85%) of methyl 4-[(hydrazinothioxomethyl)amino]benzoate as a white solid.

Methyl 4-[(hydrazinothioxomethyl)amino]benzoate (180 mg, 0.8 mmol) and 3,5-dibenzyloxybenzaldehyde (254 mg, 0.8 mmol) in ethanol were reluxed for 2 hours. As the reaction was cooled, precipitate formed to yield 302 mg (72%) of methyl 4-{[({(1E)-1-aza-2-[3,5-bis(phenylmethoxy)phenyl]vinyl}amino)thioxomethyl]amino}benzoate as a white solid.

Following the procedure of Example 32, methyl 4-{[({(1E)-1-aza-2-[3,5-bis(phenylmethoxy)phenyl]vinyl}amino)thioxomethyl]amino}benzoate (289 mg, 0.55 mmol) to yield 225 mg (78%) of methyl 4-({5-[3,5-bis(phenylmethoxy)phenyl]-1,3,4-thiadiazol-2-yl}amino)benzoate as a light brown solid.

Following the procedure of Example 34, methyl 4-({5-[3,5-bis(phenylmethoxy)phenyl]-1,3,4-thiadiazol-2-yl}amino)benzoate (209 mg, 0.4 mmol) to the isolation of 4-({5-[3,5-bis(phenylmethoxy)phenyl]-1,3,4-thiadiazol-2-yl}amino)benzoic acid (93 mg) in 97% yield, R_{f}=0.22 (ethyl acetate/hexanes, 1:1). MS (M+H)⁻ 509.

### Example 131

### Compound 133: 1-({4-[(5-{3-[3-(trifluoromethyl)phenoxy]phenyl}-1,3,4-thiadiazol-2-yl)amino]phenyl}carbonyl)piperidine-4-carboxylic acid

Ethyl 1-({3-[(5-{3-[3-(trifluoromethyl)phenoxy]phenyl}-1,3,4-thiadiazol-2-yl)amino]phenyl}carbonyl)piperidine-4-carboxylate was prepared from 3-[(5-{3-[3-(trifluoromethyl)phenoxy]phenyl}-1,3,4-thiadiazol-2-yl)amino]benzoic acid (70 mg, 0.15 mmol), ethyl isonipecotate (24 mg, 0.155 mmol), EDC HCl (35 mg, 0.18 mmol), HOBt (21 mg, 0.15 mmol), DIEA (26 μl 0.15 mmol) dichloromethane (2.0 mL) using the procedure from Example 3. Yield: 73 mg (80%).

1-({4-[(5-{3-[3-(trifluoromethyl)phenoxy]phenyl}-1,3,4-thiadiazol-2-yl)amino]phenyl}carbonyl)piperidine-4-carboxylic acid was prepared by saponification of ethyl 1-({3-[(5-{3-[3-(trifluoromethyl)phenoxy]phenyl}-1,3,4-thiadiazol-2-yl)amino]phenyl}carbonyl)piperidine-4-carboxylate (76 mg, 0.13 mmol) and lithiumhydroxide (3 mg, 0.13 mmol) using the procedure from Example 34. Yield: 24 mg (33%).

MS: (M+H) 591 and (M-CF₃CO₂)⁻ 681.

### Example 132

### Compound 135: 3-{[3-(5-{[3-(5-carboxypentylthio)phenyl]amino}(1,3,4-thiadiazol-2-yl))-5-(phenylmethoxy)phenoxy]methyl}benzoic acid

3-aminothiophenol (1.7 mL, 16 mmol), ethyl 6-bromohexanoate (3.6 g, 16 mmol), 1M NaOH (16 mL) in ethanol (30 mL) were stirred for thirty minutes. Concentrated reaction mixture. The crude residue washed with 0.1N NaOH and extracted with ethyl acetate. Ethyl acetate layer washed with water, brine, dried over sodium sulfate, filtered, and concentrated. The crude residue was purified by flash chromatography (hexanes/ethyl acetate, 9:1) to yield 2 g (48%) of ethyl 6-(3-aminophenylthio)hexanoate as a yellow solid.

Ethyl 6-(3-aminophenylthio)hexanoate (1.8 g, 6.7 mmol), dichloromethane (68 mL), water (90 mL), and thiophosgene (1.03 mL, 13.4 mmol) were stirred for 24 hours. Removed dichloromethane. Washed with water and extracted with dichloromethane. Dried dichloromethane over sodium sulfate, filtered, and concentrated to yield 2 g (97%) of ethyl 6-(3-isothiocyanatophenylthio)hexanoate as a brown liquid.

Following the procedure of Example 31, ethyl 6-(3-isothiocyanatophenylthio)hexanoate (1.5 g, 4.8 mmol), hydrazine hydrate (0.47 mL, 9.7 mmol), and toluene instead of ethanol to yield 1.65 g (99%) of ethyl 6-{3-[(hydrazinothioxomethyl)amino]phenylthio}hexanoate as a yellow solid.

Ethyl 6-{3-[(hydrazinothioxomethyl)amino]phenylthio}hexanoate (225 mg, 0.75 mmol) and methyl 3-{[5-carbonyl-3-(phenylmethoxy)phenoxy]methyl}benzoate (282 mg, 0.75 mmol) in ethanol were reluxed for 3 hours to yield 500 mg (95%) of ethyl 6-{3-[({[(1E)-1-aza-2-(5-{[3-(methoxycarbonyl)phenyl]methoxy}-3-(phenylmethoxy)phenyl)vinyl]amino}thioxomethyl)amino]phenylthio}hexanoate as a yellowish brown solid.

Following the procedure of Example 32, ethyl 6-{3-[({[(1E)-1-aza-2-(5-{[3-(methoxycarbonyl)phenyl]methoxy}-3-(phenylmethoxy)phenyl)vinyl]amino}thioxomethyl)amino]phenylthio}hexanoate (490 mg, 0.7 mmol). The crude residue was purified by flash chromatography (ethyl acetate/hexanes, 1:1) to yield 71 mg (14%) of ethyl 6-(3-{[5-(3-{[3-(methoxycarbonyl)phenyl]methoxy}-5-(phenylmethoxy)phenyl)-1,3,4-thiadiazol-2-yl]amino}phenylthio)hexanoate as an off white solid.

Following the procedure of Example 34, ethyl 6-(3-{[5-(3-{[3-(methoxycarbonyl)phenyl]methoxy}-5-(phenylmethoxy)phenyl)-1,3,4-thiadiazol-2-yl]amino}phenylthio)hexanoate (100 mg, 0.14 mmol) to the isolation of 3-{[3-(5-{[3-(5-carboxypentylthio)phenyl]amino}(1,3,4-thiadiazol-2-yl))-5-(phenylmethoxy)phenoxy]methyl}benzoic acid (38 mg) in 41% yield, R_{f}=0.53 (chloroform/methanol/acetic acid, 85:10:5).

MS (M+H)⁻ 653.

### Example 133

### Compound 137: 3-[(5-{3,5-bis[3-(trifluoromethyl)phenoxy]phenyl}-1,3,4-thiadiazol-2-yl)amino]butanoic acid

Prepared in a manner similar to that described in Compound 128 using 10.00 g (59.4 mmol) of methyl 3,5-dihydroxybenzoate, 8.4 mL (59.4 mmol) of 3-bromobenzotrifluoride, 16.44 g (118.8 mmol) of potassium carbonate and 9.46 g (118.8 mmol) of Copper (I) oxide to yield 4.77 g (18%) of methyl 3,5-bis[3-(trifluoromethyl)phenoxy]benzoate and 3.00 g (16%) of methyl 5-oxy-3-[3-(trifluoromethyl)phenoxy]benzoate.

Prepared in a manner similar to that described in Example 37 using 4.77 g (10.5 mmol) of methyl 3,5-bis[3-(trifluoromethyl)phenoxy]benzoate and 0.76 mL (15.7 mmol) of hydrazine hydrate to yield 4.35 g (91%) of 3,5-bis[3-(trifluoromethyl)phenoxy]benzenecarbohydrazide.

Prepared in a manner similar to that described in Example 37 using 0.300 g (0.66 mmol) of 3,5-bis[3-(trifluoromethyl)phenoxy]benzenecarbohydrazide and 0.114 g (0.66 mmol) of ethyl 3-isothiocyanatobutyrate to yield 0.138 g (34%) of ethyl 3-[(5-{3,5-bis[3-(trifluoromethyl)phenoxy]phenyl}-1,3,4-thiadiazol-2-yl)amino]butanoate.

Prepared in a manner similar to that described in Example 35 using 0.040 g (0.065 mmol) of ethyl 3-[(5-{3,5-bis[3-(trifluoromethyl)phenoxy]phenyl}-1,3,4-thiadiazol-2-yl)amino]butanoate to yield 0.032 g (84%) of 3-[(5-{3,5-bis[3-(trifluoromethyl)phenoxy]phenyl}-1,3,4-thiadiazol-2-yl)amino]butanoic acid. MS (M+Na)⁺ 606; (M−H)⁻ 582.

1. A method for preventing or treating anthrax infections by inhibiting Anthrax Lethal Factor activity comprising
administering a compound of the formula: 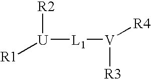
wherein U and V are, independently, C, N, or C(CH₃), L1 is a linker and R1, R2, R3 and
R4 are each independently selected substituent groups, as follows:
R1 is Z(CHR5)ₙY where n is 0 to 4,
Z is a bond, S, CO, O, —SO, SO₂, NH, NR11, SO₂NR11, NR11SO₂, 1,2-phenylene, 1,3-phenylene, 1,4-phenylene, 1,2-cyclohexylidene;
Y is a group known to bind to zinc, including CONR11OH, COOH, SH, ArSH, NHCOCH₂SH, 2-hydroxybenzoate (linked at the 3,4,5, or 6-position), 2-hydroxypyridinecarboxylate (linked at the 3,4,5, or 6-position, with the ring nitrogen at any unsubstituted position), CF₂P═O(OH)₂, C(CH₃)═NOCH₂COOH, C(CH₂OH)═NOCH₂COOH, NHCO(CHR11)ₘSH (where m=1 to 4), PO(OH)₂, PO(R11)OH, SO₂NR11OH, or NH(OH)COR11, or is derivatized to form a prodrug that is capable of undergoing conversion to a zinc-binding moiety,
R5 and R11 are, independently, H, CH₃, amino, hydroxy, alkoxy, alkylthio, alkyl (C2-C10), branched alkyl (C3-C10), alkylthio (C1-C7), alkylthioalkyl (C2-C8), arylthio, alkylamino(C1-C7), amino, arylamino, aryl, heteroaryl, arylalkyl, heterarylalkyl, arylalkenyl, heterarylalkenyl, arylalkynyl, or heterarylalkynyl, and where R1 can be further substituted with one or more of the following: NH₂, OH, halogen, alkyl, CONH₂, CONHOH, C(NH)NH₂, C(NH)NHOH, NHC(NH)NH₂, CN, NO₂, NR6R7 where R6 and R7 are H or alkyl and optionally form a ring, or R5 can form a ring with R2 or with R11;
R2 is H, isobutyl, n-butyl, pentyl, methyl, alkyl (C1-C10), branched alkyl (C3-C10), cycloalkyl, cycloalkylmethyl (C3-C9 cycle), Ar(CH₂)ₙ (where n is 0 to 4, Ar is phenyl, aryl, heteroaryl), phenethyl, arylalkenyl, heterarylalkenyl, arylalkynyl, heterarylalkynyl, alkenyl (C2-C8), alkynyl (C2-C8), pentafluorophenoxyethyl, pentafluorophenylmethyl, cycloalkenyl (C4-C10), alkylthio, arylthio, alkylamino, arylamino, aryl, dichlorophenyl, or R2 can form a ring with R5, R11, L1, or R3, and R2, R5 and R11 can be substituted with one or more of the following: NH₂, OH, halogen, alkyl, CF₃, CF₃O, CF₃S, alkoxy, alkylthio, SO₂alkyl (C1-C4), CONH₂, CONHOH, CH)NH₂, CN, NO₂, C(NH)NHOH, NHC(NH)NH₂, or NR6R7 where R6 and R7 are H or alkyl and can form a ring;
R3 is H, phenethyl, alkyl (C1-C10), branched alkyl (C1-C10), aryl, phenyl substituted with aryl or heteroaryl at the 2-, 3-, or 4-positions, benzyloxy, pyrrolyl substituted with 1-2 aryl groups, 2-aryl-1,3,4 thiadiazolyl, heteroaryl (including thiophenyl), -L2Ar where Ar includes 1-naphthyl, 2-naphthyl, 4-phenylphenyl, 5-(2-thienyl)-2-thienyl, 4-(3′-methoxyphenyl)phenyl, 4-(4′-methoxyphenyl)phenyl, 3-indolyl, phenyl, t-butyl, indolyl 3-phenylphenyl, indolyl, 2,3-dimethyl-5-indolyl, benzothiophenyl, 4-(1,2,3-thiadiazol-4-yl)phenyl, 4-(2-thienyl)phenyl, 5-(2-pyridyl)-2-thienyl, 1-(2-napthyl)vinylaminoalkyl, N-hydroxybenzamidin-4-yl, 2-methylcarbazol-3-yl, 2-ethylcarbazol-3-yl, aryl or heteroaryl and L2 is a linker chosen from the following, in both orientations: bond, CH₂, (CH₂)₂, CH₂NHCH₂, CH₂CH₂CONHCH₂, CH₂CH₂CONHCH₂CH₂, 1,1 vinylidene, 1,2-vinylidene, CO, CH₂CH₂NHCH₂, CH₂CH₂CH₂NHCH₂, CH₂NHCH₂CH₂, (CH₂)q where q=3 to 7, (CHR9)r where r=1 to 7 and R9 is independently H, alkyl (C1-C10), branched alkyl (C3-C10), cycloalkyl (C3-C10), cycloalkylalkyl (C4-C14), alkyl thio, amino, alkyl amino, dialkylamino, (CHR9)sX(CHR9)t where s+t=0 to 8, X is O, S, CO, SO, SO₂, NH, CONH, NHCO, SO₂NH, NHSO₂ or NR9 and R9 is independently H, alkyl (C1-C10), branched alkyl (C3-C10), cycloalkyl (C3-C10), cycloalkylalkyl (C4-C14), acyl, alkyl thio, amino, alkyl amino, or dialkylamino, and R9 also includes N-linked heterocycles such as piperidine, pyrroline, (1,2,3,4-)tetahydrobetacarbolin-2-yl, R15 is H, alkyl (C1-C4), branched alkyl (C3-C5), or cycloalkyl(C3-C5), carbon-carbon single bonds in R8 can optionally be substituted with double or triple bonds, and where R3 can form a ring with R2, L1, or R4, or R3, R9 and R15 are further substituted with one or more of the following NH₂, OH, halogen, N(CH₃)₂, alkyl, CF₃, CF₃O, CF₃S, alkoxy, alkylthio, CONH₂, CONHOH, C(NH)NH₂, CN, NO₂, C(NH)NHOH, NHC(NH)NH₂, aryloxy, trifluoromethylphenyloxy, carboxyalkyl (C2-C8), (Carboxyphenyl)methylthio, carboxyalkylthio (C2-C8), carboxyphenyl, NR6R7 where R6 and R7 are H or alkyl or can form a ring;
R4 is H, alkyl (C1-C10), branched alkyl (C1-C10), arylalkyl, heteroarylalkyl, CONR10R16 where R10 is H, methyl, alkyl (C2-C10), branched alkyl (C3-C10), benzyl, phenethyl, arylalkyl, heteroarylalkyl, alkanoyl (C2-C8), branched alkanoyl, aroyl (C6-C12), heteroaroyl (C2-C10), isopropyl, CONR16R12; and where R12 and R16 are, independently, H, methyl, alkyl, benzyl, 2-phenylethyl, 2-indanyl, 2-morpholinylethyl, (2,6)-dimethoxylbenzyl, dimethylaminoethyl, (2-pyridyl)methyl, 2-(2-pyridyl)ethyl, 4-carboxybenzyl, 1-phenylethyl, CH(CONH₂)CH₂C6H₅, CH(CONH₂)CH₂CH(CH₃)₂, CH(CONH₂)CH(CH₃)CH₂CH₃, CH(CONH₂)CHCH₃ CH(CH₂OCH₃)CH₂C6H₅, CH(CONHCH₂CH₂OCH₃)CH₂cyclohexyl, aryl, arylalkyl, heteroaryl, heteroarylalkyl, aminoalkyl, hydroxyalkyl, (trifluoromethylphenoxy)phenyl. NR16R12 can optionally form an N-linked monocyclic or bicyclic heterocyclic ring, including but not limited to 1,2-dihydroisoindole, octahydroisoindole, morpholine, piperidine, piperazine, N-alkyl piperazine (C1-C4), homopiperazine, 3-pyrroline, pyrrolidine, tetrahydroisoquinoline, octahydropyrrolo[3,4-C]pyrrole, L-proline, L-proline dimethylamide, D-proline, D-proline dimethylamide, and thiazolidine, or
R4 can form a ring with L1 or R3, and R4, R6, R7, R10, R11, R12 and R16 can be further substituted, independently, with 1 to 3 of the following substitutents: NH₂, OH, F, Cl, Br, methyl, alkyl, aryl, cycloalkyl (C3-C6), heterocycloalkyl, heteroaryl, CF₃, CF₃O, CF₃S, CF3, aryloxy, trifluoromethylphenoxy, alkoxy, alkylthio, CONH₂, CN, NO₂, CONHOH, C(NH)NH₂, C(NH)NHOH, NHC(NH)NH₂, NR6R7 where R6 and R7 are H or alkyl, or can form a ring; and
L1 is a linker including the following, in either orientation: single bond, double bond, CONH, NHCO, N(CH₃)CO, CON(CH₃), CH₂NH, NHCH₂, CH═CH, C(NH₂)═N, N═C(NH₂), arylene (linked 1,2-; 1,3-; or 1,4), heteroarylene (linked 1,2-; 1,3-; or 1,4), ethynyl, CH═CF, CF═CH, CF═CF, CH₂CH₂, C(CH₃)═CH, CH═C(CH₃), SO₂NH, SO₂, COCH₂, CH₂CO, CNOHCH₂, CH₂CNOH, C(CF₃)═CH, CH═C(CF₃), SO₂CH₂, CH₂SO₂, SOCH₂, CH₂SO, CH₂CHOH, CHOHCH₂, lower cycloalkyl (C3-C6), or CHOHCHOH, or where L1 can be substituted with one or more of the following: NH₂, OH, halogen, alkyl, CF₃, CF₃O, CF₃S, alkoxy, alkylthio, CONH₂, CONHOH, C(NH)NH₂, C(NH)NHOH, NHC(NH)NH₂, NR6R7 where R6 and R7 are H or alkyl and optionally form a ring.

2. A pharmaceutical composition useful for preventing or treating anthrax infections by inhibiting Anthrax Lethal Factor activity comprising
a compound of the formula: 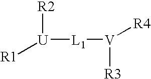
wherein U and V are, independently, C, N, or C(CH₃), L1 is a linker and R1, R2, R3 and R4 are each independently selected substituent groups, as follows:
R1 is Z(CHR5)ₙY where n is 0 to 4,
Z is a bond, S, CO, O, SO, SO₂, NH, NR11, SO₂NR11, NR11SO₂, 1,2-phenylene, 1,3-phenylene, 1,4-phenylene, 1,2-cyclohexylidene;
Y is a group known to bind to zinc, including CONR110H, COOH, SH, ArSH, NHCOCH₂SH, 2-hydroxybenzoate (linked at the 3,4,5, or 6-position), 2-hydroxypyridinecarboxylate (linked at the 3,4,5, or 6-position, with the ring nitrogen at any unsubstituted position), CF₂P═O(OH)₂, C(CH₃)═NOCH₂COOH, C(CH₂OH)═NOCH₂COOH, NHCO(CHR11)ₘSH (where m=1 to 4), PO(OH)₂, PO(R11)OH, SO₂NR11OH, or NH(OH)COR11, or is derivatized to form a prodrug that is capable of undergoing conversion to a zinc-binding moiety,
R5 and R11 are, independently, H, CH₃, amino, hydroxy, alkoxy, alkylthio, alkyl (C2-C10), branched alkyl (C3-C10), alkylthio (C1-C7), alkylthioalkyl (C2-C8), arylthio, alkylamino(C1-C7), amino, arylamino, aryl, heteroaryl, arylalkyl, heterarylalkyl, arylalkenyl, heterarylalkenyl, arylalkynyl, or heterarylalkynyl,
and where R1 can be further substituted with one or more of the following: NH₂, OH, halogen, alkyl, CONH₂, CONHOH, C(NH)NH₂, C(NH)NHOH, NHC(NH)NH₂, CN, NO₂, NR6R7 where R6 and R7 are H or alkyl and optionally form a ring, or R5 can form a ring with R2 or with R11;
R2 is H, isobutyl, n-butyl, pentyl, methyl, alkyl (C1-C10), branched alkyl (C3-C10), cycloalkyl, cycloalkylmethyl (C3-C9 cycle), Ar(CH₂)ₙ (where n is 0 to 4, Ar is phenyl, aryl, heteroaryl), phenethyl, arylalkenyl, heterarylalkenyl, arylalkynyl, heterarylalkynyl, alkenyl (C2-C8), alkynyl (C2-C8), pentafluorophenoxyethyl, pentafluorophenylmethyl, cycloalkenyl (C4-C10), alkylthio, arylthio, alkylamino, arylamino, aryl, dichlorophenyl, or R2 can form a ring with R5, R11, L1, or R3, and R2, R5 and R11 can be substituted with one or more of the following: NH₂, OH, halogen, alkyl, CF₃, CF₃O, CF₃S, alkoxy, alkylthio, SO₂alkyl (C1-C4), CONH₂, CONHOH, C(NH)NH₂, CN, NO₂, C(NH)NHOH, NHC(NH)NH₂, or NR6R7 where R6 and R7 are H or alkyl and can form a ring;
R3 is H, phenethyl, alkyl (C1-C10), branched alkyl (C1-C10), aryl, phenyl substituted with aryl or heteroaryl at the 2-, 3-, or 4-positions, benzyloxy, pyrrolyl substituted with 1-2 aryl groups, 2-aryl-1,3,4 thiadiazolyl, heteroaryl (including thiophenyl), -L2Ar where Ar includes 1-naphthyl, 2-naphthyl, 4-phenylphenyl, 5-(2-thienyl)-2-thienyl, 4-(3′-methoxyphenyl)phenyl, 4-(4′-methoxyphenyl)phenyl, 3-indolyl, phenyl, t-butyl, indolyl 3-phenylphenyl, indolyl, 2,3-dimethyl-5-indolyl, benzothiophenyl, 4-(1,2,3-thiadiazol-4-yl)phenyl, 4-(2-thienyl)phenyl, 5-(2-pyridyl)-2-thienyl, 1-(2-napthyl)vinylaminoalkyl, N-hydroxybenzamidin-4-yl, 2-methylcarbazol-3-yl, 2-ethylcarbazol-3-yl, aryl or heteroaryl and L2 is a linker chosen from the following, in both orientations: bond, CH₂, (CH₂)₂, CH₂NHCH₂, CH₂CH₂CONHCH₂, CH₂CH₂CONHCH₂CH₂, 1,1 vinylidene, 1,2-vinylidene, CO, CH₂CH₂NHCH₂, CH₂CH₂CH₂NHCH₂, CH₂NHCH₂CH₂, (CH₂)_{q} where q=3 to 7, (CHR9)ᵣ where r=1 to 7 and R9 is independently H, alkyl (C1-C10), branched alkyl (C3-C10), cycloalkyl (C3-C10), cycloalkylalkyl (C4-C14), alkyl thio, amino, alkyl amino, dialkylamino, (CHR9)sX(CHR9)t where s+t=0 to 8, X is O, S, CO, SO, SO₂, NH, CONH, NHCO, SO₂NH, NHSO₂ or NR9 and R9 is independently H, alkyl (C1-C10), branched alkyl (C3-C10), cycloalkyl (C3-C10), cycloalkylalkyl (C4-C14), acyl, alkyl thio, amino, alkyl amino, or dialkylamino, and R9 also includes N-linked heterocycles such as piperidine, pyrroline, (1,2,3,4-)tetahydrobetacarbolin-2-yl, R15 is H, alkyl (C1-C4), branched alkyl (C3-C5), or cycloalkyl(C3-C5), carbon-carbon single bonds in R8 can optionally be substituted with double or triple bonds, and where R3 can form a ring with R2, L1, or R4, or R3, R9 and R15 are further substituted with one or more of the following NH₂, OH, halogen, N(CH₃)₂, alkyl, CF₃, CF₃O, CF₃S, alkoxy, alkylthio, CONH₂, CONHOH, C(H)NH₂, CN, NO₂, C(NH)NHOH, NHC(NH)NH₂, aryloxy, trifluoromethylphenyloxy, carboxyalkyl (C2-C8), (Carboxyphenyl)methylthio, carboxyalkylthio (C2-C8), carboxyphenyl, NR6R7 where R6 and R7 are H or alkyl or can form a ring;
R4 is H, alkyl (C1-C10), branched alkyl (C1-C10), arylalkyl, heteroarylalkyl, CONR10R16 where R10 is H, methyl, alkyl (C2-C10), branched alkyl (C3-C10), benzyl, phenethyl, arylalkyl, heteroarylalkyl, alkanoyl (C2-C8), branched alkanoyl, aroyl (C6-C12), heteroaroyl (C2-C10), isopropyl, CONR16R12; and where R12 and R16 are, independently, H, methyl, alkyl, benzyl, 2-phenylethyl, 2-indanyl, 2-morpholinylethyl, (2,6)-dimethoxylbenzyl, dimethylaminoethyl, (2-pyridyl)methyl, 2-(2-pyridyl)ethyl, 4-carboxybenzyl, 1-phenylethyl, CH(CONH₂)CH₂C6H₅, CH(CONH₂)CH₂CH(CH₃)₂, CH(CONH₂)CH(CH₃)CH₂CH₃, CH(CONH₂)CHCH₃ CH(CH₂OCH₃)CH₂C6H₅, CH(CONHCH₂CH₂OCH₃)CH₂cyclohexyl, aryl, arylalkyl, heteroaryl, heteroarylalkyl, aminoalkyl, hydroxyalkyl, (trifluoromethylphenoxy)phenyl. NR16R12 can optionally form an N-linked monocyclic or bicyclic heterocyclic ring, including but not limited to 1,2-dihydroisoindole, octahydroisoindole, morpholine, piperidine, piperazine, N-alkyl piperazine (C1-C4), homopiperazine, 3-pyrroline, pyrrolidine, tetrahydroisoquinoline, octahydropyrrolo[3,4-C]pyrrole, L-proline, L-proline dimethylamide, D-proline, D-proline dimethylamide, and thiazolidine, or
R4 can form a ring with L1 or R3, and R4, R6, R7, R10, R11, R12 and R16 can be further substituted, independently, with 1 to 3 of the following substitutents: NH₂, OH, F, Cl, Br, methyl, alkyl, aryl, cycloalkyl (C3-C6), heterocycloalkyl, heteroaryl, CF₃, CF₃O, CF₃S, CF₃, aryloxy, trifluoromethylphenoxy, alkoxy, alkylthio, CONH₂, CN, NO₂, CONHOH, C(NH)NH₂, C(NH)NHOH, NHC(NH)NH₂, NR6R7 where R6 and R7 are H or alkyl, or can form a ring; and
L1 is a linker including the following, in either orientation: single bond, double bond, CONH, NHCO, N(CH₃)CO, CON(CH₃), CH₂NH, NHCH₂, CH═CH, C(NH₁₂)═N, N═C(NH₂), arylene (linked 1,2-; 1,3-; or 1,4), heteroarylene (linked 1,2-; 1,3-; or 1,4), ethynyl, CH═CF, CF═CH, CF═CF, CH₂CH₂, C(CH₃)═CH, CH═C(CH₂), SO₂NH, SO₂, COCH₂, CH₂CO, CNOHCH₂, CH₂CNOH, C(CF₃)═CH, CH═C(CF₃), SO₂CH₂, CH₂SO₂, SOCH₂, CH₂SO, CH₂CHOH, CHOHCH₂, lower cycloalkyl (C3-C6), or CHOHCHOH, or where L1 can be substituted with one or more of the following: NH₂, OH, halogen, alkyl, CF₃, CF₃O, CF₃S, alkoxy, alkylthio, CONH₂, CONHOH, C(NH)NH₂, C(NH)NHOH, NHC(NH)NH₂, NR6R7 where R6 and R7 are H or alkyl and optionally form a ring, together with a pharmaceutically acceptable carrier.
